(19) United States
(12) Patent Application Publication
(10) Pub. No.: US 2005/0181009 A1
(43) Pub. Date: Aug. 18, 2005

(54) Implantable sensors and implantable pumps and anti-scarring agents

(75) Inventors: William L. Hunter, Vancouver (CA); David M. Gravett, Vancouver (CA); Philip M. Toleikis, Vancouver (CA); Arpita Maiti, Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 11/001,787

(22) Filed: Dec. 1, 2004

Related U.S. Application Data
(63) Continuation of application No. 10/996,352, filed on Nov. 22, 2004.
Application No. 10/996,352 is a continuation-in-part of application No. 10/986,231, filed on Nov. 10, 2004.
Application No. 10/996,352 is a continuation-in-part of application No. 10/986,230, filed on Nov. 10, 2004.
(60) Provisional application No. 60/586,861, filed on Jul. 9, 2004.
(60) Provisional application No. 60/578,471, filed on Jun. 9, 2004.
(60) Provisional application No. 60/526,541, filed on Dec. 3, 2003.
(60) Provisional application No. 60/525,226, filed on Nov. 24, 2003.
(60) Provisional application No. 60/523,908, filed on Nov. 20, 2003.
(60) Provisional application No. 60/524,023, filed on Nov. 20, 2003.

(51) Int. Cl. A61F 2/00

(52) U.S. Cl. 424/423

(57) ABSTRACT

Pumps and sensors for contact with tissue are used in combination with an anti-scarring agent (e.g., a cell cycle inhibitor) in order to inhibit scarring that may otherwise occur when the pumps and sensors are implanted within an animal.

### CROSS-REFERENCE TO RELATED APPLICATIONS

This application is a Continuation-in-Part of U.S. application Ser. No. 10/986,231, filed Nov. 10, 2004; and Ser. No. 10/986,230, filed Nov. 10, 2004. This application also claims the benefit under 35 U.S.C. 119(e) of U.S. Provisional Application Ser. Nos. 60/586,861, filed Jul. 9, 2004; 60/578,471, filed Jun. 9, 2004; 60/526,541, filed Dec. 3, 2003; 60/525,226, filed Nov. 24, 2003; 60/523,908, filed Nov. 20, 2003; and 60/524,023, filed Nov. 20, 2003, which applications are incorporated herein by reference in their entireties.

### BACKGROUND OF THE INVENTION

1. Field of the Invention

The present invention relates generally to implantable sensors, drug-delivery devices and drug-delivery pump, and more specifically, to compositions and methods for preparing and using such devices to make them resistant to overgrowth by inflammatory and fibrous scar tissue.

2. Description of the Related Art

Implantable drug delivery devices and pumps are a means to provide prolonged, site-specific release of a therapeutic agent for the management of a variety of medical conditions. Drug delivery implants and pumps are generally utilized when a localized pharmaceutical impact is desired (i.e., the condition affects only a specific region) or when systemic delivery of the agent is inefficient or ineffective and leads toxicity, severe side effects, inactivation of the drug prior to reaching the target tissue, poor symptom/disease control, and/or addiction to the medication. Implantable pumps can also deliver systemic drug levels in a constant, regulated manner for extended periods and help patients avoid the “peaks and valleys” of blood-level drug concentrations associated with intermittent systemic dosing. For many patients this can lead to better symptom control (the dosage can often be titrated to the severity of the symptoms), superior disease management (particularly for insulin delivery in diabetics), and lower drug requirements (particularly for pain medications). Innumerable drug delivery devices, implants and pumps have been developed for an array of specific medical conditions and the particular construction and delivery mechanism of the device depends on the particular treatment. For example, drug delivery implants and pumps have been used in a variety of clinical applications, including programmable insulin pumps for the treatment of diabetes, intrathecal (in the spine) pumps to administer narcotics (e.g., morphine, fentanyl) for the relief of pain (e.g., cancer, back problems, HIV, post-surgery), local and systemic delivery of chemotherapy for the treatment of cancer (e.g., hepatic artery 5-FU infusion for liver tumors), medications for the treatment of cardiac conditions (e.g., anti-arrhythmic drugs for cardiac rhythm abnormalities), intrathecal delivery of anti-spasmotic drugs (e.g., baclofen) for spasticity in neurological disorders (e.g., Multiple Sclerosis, spinal cord injuries, brain injury, cerebral palsy), or local/regional antibiotics for infection management (e.g., osteomyelitis, septic arthritis).

Typically, most drug delivery pumps are implanted subcutaneously (under the skin in an easy to access, but discrete location) and consist of a pump unit with a drug reservoir and a flexible catheter through which the drug is delivered to the target tissue. The pump stores and releases prescribed amounts of medication via the catheter to achieve therapeutic drug levels either locally or systemically (depending upon the application). The center of the pump has a self-sealing access port covered by a septum such that a needle can be inserted percutaneously (through both the skin and the septum) to refill the pump with medication as required. There are generally two types of implantable drug delivery pumps. Constant-rate pumps are usually powered by gas and are designed to dispense drugs under pressure as a continual dosage at a preprogrammed, constant rate. The amount and rate of drug flow are regulated by the length of the catheter used, temperature and altitude, and they are best when unchanging, long-term drug delivery is required. Although limited, these pumps have the advantage of being simple, having few moving parts, not requiring battery power and possessing a longer lifespan. Programmable-rate pumps utilize a battery-powered pump and a constant pressure reservoir to deliver drugs on a periodic basis in a manner that can be programmed by the physician or the patient. For the programmable infusion device, the drug may be delivered in small, discrete doses based on a programmed regimen which can be altered according to an individual's clinical response. Programmable drug delivery pumps may be in communication with an external transmitter which programs the prescribed dosing regimen, including the rate, time and amount of each dose, via low-frequency waves that are transmitted through the skin. Programmable-rate pumps are more widely used and provide superior dosimetry, but because of their complexity, they require more maintenance and have a shorter lifespan.

The clinical function of an implantable drug delivery device or pump depends upon the device, particularly the catheter, being able to effectively maintain intimate anatomical contact with the target tissue (e.g., the sudural space in the spinal cord, the arterial lumen, the peritoneum) and not becoming encapsulated or obstructed by scar tissue. Unfortunately, in many instances when these devices are implanted in the body, they are subject to a “foreign body” response from the surrounding host tissues. The body recognizes the implanted device as foreign, which triggers an inflammatory response followed by encapsulation of the implant with fibrous connective tissue. Scarring (i:e., fibrosis) can also result from trauma to the anatomical structures and tissue surrounding the implant during implantation of the device. Lastly, fibrous encapsulation of the device can occur even after a successful implantation if the device is manipulated (some patients continuously “fiddle” with a subcutaneous implant) or irritated by the daily activities of the patient. For drug delivery pumps, the catheter tip or lumen may become obstructed by scar tissue which may cause the flow of drug to slowdown or cease completely. Alternatively, the catheter can become encapsulated by scar (i.e., the body “walls off” the device with fibrous tissue) so that the drug is incompletely delivered to the target tissue (i.e., the scar prevents proper drug movement from the catheter to the tissues on the other side of the capsule). Either of these developments may lead to inefficient or incomplete drug flow to the desired target tissues or organs (and loss of clinical benefit), while the second can also lead to local drug accumulation (in the capsule) and additional clinical complications (e.g., local drug toxicity; drug sequestration followed by sudden “dumping” of large amounts of drug into the surrounding tissues). Additionally, the tissue surrounding the implantable pump or catheter can be inadvertently damaged from the inflammatory foreign body response leading to loss of function and/or tissue damage (e.g., scar tissue in the spinal canal causing pain or obstructing the flow of cerebrospinal fluid).

A device that is frequently (but not always) used in association with a drug delivery pump is an implantable sensor device. An implantable sensor is a device used to detect changes in body function and/or levels of key physiological metabolites, chemistry, hormones or biological factors. Implantable sensors may be used to sense a variety of physical and/or physiological properties, including, but not limited to, optical, mechanical, chemical, electrochemical, temperature, strain, pressure, magnetism, acceleration, ionizing radiation, acoustic wave or chemical changes. Often sensor technology is combined with implantable drug delivery pumps such that the sensor receives a signal and then, in turn, uses this information to modulate the release kinetics of a drug. The most widely pursued application of this technology is the production of a closed-loop “artificial pancreas” which can continuously detect blood glucose levels (through an implanted sensor) and provide feedback to an implantable pump to modulate the administration of insulin to a diabetic patient. Other representative examples of implantable sensors include, blood/tissue glucose monitors, electrolyte sensors, blood constituent sensors, temperature sensors, pH sensors, optical sensors, amperometric sensors, pressure sensors, biosensors, sensing transponders, strain sensors, activity sensors and magnetoresistive sensors. Much like the problem facing drug delivery pumps described above, proper clinical functioning of an implanted sensor is dependent upon intimate anatomical contact with the target tissues and/or body fluids. Scarring around the implanted device may degrade the electrical components and characteristics of the device-tissue interface, and the device may fail to function properly. For example, when a “foreign body” response occurs and the implanted sensor becomes encapsulated by scar (i.e., the body “walls off” the sensor with fibrous tissue), the sensor receives inaccurate biological information. If the sensor is detecting conditions inside the capsule, and these conditions are not consistent with those outside the capsule (which is frequently the case), it will produce inaccurate readings. Similarly if the scar tissue alters the flow of physical or chemical information to the detection mechanism of the sensor, the information it processes will not be reflective of those present in the target tissue.

### BRIEF SUMMARY OF THE INVENTION

Briefly stated, the present invention discloses pharmaceutical agents which inhibit one or more aspects of the production of excessive fibrous (scar) tissue. In one aspect, the present invention provides compositions for delivery of selected therapeutic agents via medical devices or implants containing sensors or drug delivery pumps, as well as methods for making and using these implants and devices. Compositions and methods are described for coating sensors or pumps with drug-delivery compositions such that the pharmaceutical agent is delivered in therapeutic levels over a period sufficient to prevent the drug delivery catheter and/or the implanted sensor from being encapsulated in fibrous tissue to improve and/or prolong device function. Alternatively, locally administered compositions (e.g., topicals, injectables, liquids, gels, sprays, microspheres, pastes, wafers) containing an inhibitor of fibrosis are described that can be applied to the tissue adjacent to the implanted pump (particularly the delivery catheter) and/or the implanted sensor, such that the fibrosis-inhibitor is delivered in therapeutic levels over a period sufficient to prevent the delivery catheter or sensor from being occluded or encapsulated by fibrous tissue. And finally, numerous specific implantable pumps, sensors and combined devices are described that produce superior clinical results as a result of being coated with agents that reduce excessive scarring and fibrous tissue accumulation as well as other related advantages.

Within one aspect of the invention, drug-coated or drug-impregnated implants and medical devices are provided which reduce fibrosis in the tissue surrounding the implanted drug delivery pump or sensor, or inhibit scar development on the device/implant surface (particularly the drug delivery catheter lumen and the sensor surface), thus enhancing the efficacy of the procedure. For example, fibrous tissue can reduce or obstruct the flow of therapeutic agents from the catheter to the target tissue, or prevent the implanted sensor from detecting accurate readings. Within various embodiments, fibrosis is inhibited by local or systemic release of specific pharmacological agents that become localized to the tissue adjacent to the implanted device.

The repair of tissues following a mechanical or surgical intervention, such as the implantation of a pump or sensor, involves two distinct processes: (1) regeneration (the replacement of injured cells by cells of the same type and (2) fibrosis (the replacement of injured cells by connective tissue). There are several general components to the process of fibrosis (or scarring) including: infiltration of inflammatory cells and the inflammatory response, migration and proliferation of connective tissue cells (such as fibroblasts or smooth muscle cells), deposition of extracellular matrix (ECM), formation of new blood vessels (angiogenesis), and remodeling (maturation and organization of the fibrous tissue). As utilized herein, “inhibits (reduces) fibrosis” may be understood to refer to agents or compositions which decrease or limit the formation of fibrous tissue (i.e., by reducing or inhibiting one or more of the processes of inflammation, connective tissue cell migration or proliferation, ECM production, angiogenesis, and/or remodeling). In addition, numerous therapeutic agents described in this invention will have the additional benefit of also reducing tissue regeneration where appropriate.

Within certain embodiments of the invention, an implant or device (e.g., a sensor or pump) is adapted to release an agent that inhibits fibrosis through one or more of the mechanisms cited herein. Within certain other embodiments of the invention, an implant or device contains an agent that while remaining associated with the implant or device, inhibits fibrosis between the implant or device and the tissue where the implant or device is placed by direct contact between the agent and the tissue surrounding the implant or device.

Within related aspects of the present invention, implanted pumps and sensors are provided comprising an implant or device, wherein the implant or device releases an agent which inhibits fibrosis in vivo. “Release of an agent” refers to any statistically significant presence of the agent, or a subcomponent thereof, which has disassociated from the implant/device and/or remains active on the surface of (or within) the device/implant. Within yet other aspects of the present invention, methods are provided for manufacturing a medical device or implant, comprising the step of coating (e.g., spraying, dipping, wrapping, or administering drug through) a medical device or implant. Additionally, the implant or medical device can be constructed so that the device itself is comprised of materials which inhibit fibrosis in or around the implant. A wide variety of implantable pumps and sensors may be utilized within the context of the present invention, depending on the site and nature of treatment desired.

Within various embodiments of the invention, the implanted pump or sensor is further coated with a composition or compound, which delays the onset of activity of the fibrosis-inhibiting agent for a period of time after implantation. Representative examples of such agents include heparin, PLGA/MePEG, PLA, and polyethylene glycol. Within further embodiments, the fibrosis-inhibiting implant or device is activated before, during, or after deployment (e.g., an inactive agent on the device is first activated to one that reduces or inhibits an in vivo fibrotic reaction).

Within various embodiments of the invention, the tissue surrounding the implanted pump (particularly the drug delivery catheter) and/or sensor is treated with a composition or compound that contains an inhibitor of fibrosis. Locally administered compositions (e.g., topicals, injectables, liquids, gels, sprays, microspheres, pastes, wafers) or compounds containing an inhibitor of fibrosis are described that can be applied to the surface of, or infiltrated into, the tissue adjacent to the pump or sensor, such that the pharmaceutical agent is delivered in therapeutic levels over a period sufficient to prevent the drug delivery catheter and/or sensor from being obstructed or encapsulated by fibrous tissue. This can be done in lieu of coating the device or implant with a fibrosis-inhibitor, or done in addition to coating the device or implant with a fibrosis-inhibitor. The local administration of the fibrosis-inhibiting agent can occur prior to, during, or after implantation of the pump or sensor itself.

Within various embodiments of the invention, an implanted pump or sensor is coated on one aspect, portion or surface with a composition which inhibits fibrosis, as well as being coated with a composition or compound which promotes scarring on another aspect, portion or surface of the device (i.e., to affix the body of the device into a particular anatomical space). Representative examples of agents that promote fibrosis and scarring include silk, silica, crystalline silicates, bleomycin, quartz dust, neomycin, talc, metallic beryllium and oxides thereof, retinoic acid compounds, copper, leptin, growth factors, a component of extracellular matrix; fibronectin, collagen, fibrin, or fibrinogen, polylysine, poly(ethylene-co-vinylacetate), chitosan, N-carboxybutylchitosan, and RGD proteins; vinyl chloride or a polymer of vinyl chloride; an adhesive selected from the group consisting of cyanoacrylates and crosslinked poly(ethylene glycol)—methylated collagen; an inflammatory cytokine (e.g., TGFβ, PDGF, VEGF, bFGF, TNFα, NGF, GM-CSF, IGF-1, IL-1, IL-1-β, IL-8, IL-6, and growth hormone); connective tissue growth factor (CTGF) as well as analogues and derivatives thereof.

Also provided by the present invention are methods for treating patients undergoing surgical, endoscopic or minimally invasive therapies where an implanted pump or sensor is placed as part of the procedure. As utilized herein, it may be understood that “inhibits fibrosis” refers to a statistically significant decrease in the amount of scar tissue in or around the device or an improvement in the interface between the implant (catheter and/or sensor) and the tissue, which may or may not lead to a permanent prohibition of any complications or failures of the device/implant.

The pharmaceutical agents and compositions are utilized to create novel drug-coated implants and medical devices that reduce the foreign body response to implantation and limit the growth of reactive tissue on the surface of, into, or around the device, such that performance is enhanced. Implantable pumps and sensors coated with selected pharmaceutical agents designed to prevent scar tissue overgrowth and improve electrical conduction can offer significant clinical advantages over uncoated devices.

For example, in one aspect the present invention is directed to implantable pumps and sensors that comprise a medical implant and at least one of (i) an anti-scarring agent and (ii) a composition that comprises an anti-scarring agent. The agent is present so as to inhibit scarring that may otherwise occur when the implant is placed within an animal. In another aspect the present invention is directed to methods wherein both an implant and at least one of (i) an anti-scarring agent and (ii) a composition that comprises an anti-scarring agent, are placed into an animal, and the agent inhibits scarring that may otherwise occur. These and other aspects of the invention are summarized below.

Thus, in various independent aspects, the present invention provides a device, comprising an implantable pump and/or sensor and an anti-scarring agent or a composition comprising an anti-scarring agent, wherein the agent inhibits scarring. These and other devices are described in more detail herein. In each of the aforementioned devices, in separate aspects, the present invention provides that: the agent is a cell cycle inhibitor; the agent is an anthracycline; the agent is a taxane; the agent is a podophyllotoxin; the agent is an immunomodulator; the agent is a heat shock protein 90 antagonist; the agent is a HMGCoA reductase inhibitor; the agent is an inosine monophosphate dehydrogenase inhibitor; the agent is an NF kappa B inhibitor; the agent is a P38 MAP kinase inhibitor. These and other agents are described in more detail herein.

In additional aspects, for each of the aforementioned devices combined with each of the aforementioned agents, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

In addition to devices, the present invention also provides methods. For example, in additional aspects of the present invention, for each of the aforementioned devices, and for each of the aforementioned combinations of the devices with the anti-scarring agents, the present invention provides methods whereby a specified device is implanted into an animal, and a specified agent associated with the device inhibits scarring that may otherwise occur. Each of the devices identified herein may be a “specified device”, and each of the anti-scarring agents identified herein may be an “anti-scarring agent”, where the present invention provides, in independent embodiments, for each possible combination of the device and the agent.

The agent may be associated with the device prior to the device being placed within the animal. For example, the agent (or composition comprising the agent) may be coated onto an implant, and the resulting device then placed within the animal. In addition, or alternatively, the agent may be independently placed within the animal in the vicinity of where the device is to be, or is being, placed within the animal. For example, the agent may be sprayed or otherwise placed onto, adjacent to, and/or within the tissue that will be contacting the medical implant or may otherwise undergo scarring. To this end, the present invention provides placing an implantable pump and/or sensor and an anti-scarring agent or a composition comprising an anti-scarring agent into an animal host, wherein the agent inhibits scarring.

In each of the aforementioned methods, in separate aspects, the present invention provides that: the agent is a cell cycle inhibitor; the agent is an anthracycline; the agent is a taxane; the agent is a podophyllotoxin; the agent is an immunomodulator; the agent is a heat shock protein 90 antagonist; the agent is a HMGCoA reductase inhibitor; the agent is an inosine monophosphate dehydrogenase inhibitor; the agent is an NF kappa B inhibitor; the agent is a P38 MAP kinase inhibitor. These and other agents which can inhibit fibrosis are described in more detail herein.

In additional aspects, for each of the aforementioned methods used in combination with each of the aforementioned agents, it is, for each combination, independently disclosed that the agent may be present in a composition along with a polymer. In one embodiment of this aspect, the polymer is biodegradable. In another embodiment of this aspect, the polymer is non-biodegradable. Other features and characteristics of the polymer, which may serve to describe the present invention for every combination of device and agent described above, are set forth in greater detail herein.

These and other aspects of the present invention will become evident upon reference to the following detailed description and attached drawings. In addition, various references are set forth herein which describe in more detail certain procedures and/or compositions (e.g., polymers), and are therefore incorporated by reference in their entirety.

### BRIEF DESCRIPTION OF THE DRAWINGS

FIG. 1 is a diagram showing how a cell cycle inhibitor acts at one or more of the steps in the biological pathway.

FIG. 2 is a graph showing the results for the screening assay for assessing the effect of mitoxantrone on nitric oxide production by THP-1 macrophages.

FIG. 3 is a graph showing the results for the screening assay for assessing the effect of Bay 11-7082 on TNF-alpha production by THP-1 macrophages.

FIG. 4 is a graph showing the results for the screening assay for assessing the effect of rapamycin concentration for TNFα production by THP-1 macrophages.

FIG. 5 is graph showing the results of a screening assay for assessing the effect of mitoxantrone on proliferation of human fibroblasts.

FIG. 6 is graph showing the results of a screening assay for assessing the effect of rapamycin on proliferation of human fibroblasts.

FIG. 7 is graph showing the results of a screening assay for assessing the effect of paclitaxel on proliferation of human fibroblasts.

FIG. 8 is a picture that shows an uninjured carotid artery from a rat balloon injury model.

FIG. 9 is a picture that shows an injured carotid artery from a rat balloon injury model.

FIG. 10 is a picture that shows a paclitaxel/mesh treated carotid artery in a rat balloon injury model.

FIG. 11A schematically depicts the transcriptional regulation of matrix metalloproteinases.

FIG. 11B is a blot which demonstrates that IL-1 stimulates AP-1 transcriptional activity.

FIG. 11C is a graph which shows that IL-1 induced binding activity decreased in lysates from chondrocytes which were pretreated with paclitaxel.

FIG. 11D is a blot which shows that IL-1 induction increases collagenase and stromelysin in RNA levels in chondrocytes, and that this induction can be inhibited by pretreatment with paclitaxel.

FIGS. 12A-H are blots that show the effect of various anti-microtubule agents in inhibiting collagenase expression.

FIG. 13 is a graph showing the results of a screening assay for assessing the effect of paclitaxel on smooth muscle cell migration.

FIG. 14 is a graph showing the results of a screening assay for assessing the effect of geldanamycin on IL-1β production by THP-1 macrophages.

FIG. 15 is a graph showing the results of a screening assay for assessing the effect of geldanamycin on IL-8 production by THP-1 macrophages.

FIG. 16 is a graph showing the results of a screening assay for assessing the effect of geldanamycin on MCP-1 production by THP-1 macrophages.

FIG. 17 is graph showing the results of a screening assay for assessing the effect of paclitaxel on proliferation of smooth muscle cells.

FIG. 18 is graph showing the results of a screening assay for assessing the effect of paclitaxel for proliferation of the murine RAW 264.7 macrophage cell line.

FIG. 19 is a bar graph showing the area of granulation tissue in carotid arteries exposed to silk coated perivascular polyurethane (PU) films relative to arteries exposed to uncoated PU films.

FIG. 20 is a bar graph showing the area of granulation tissue in carotid arteries exposed to silk suture coated perivascular PU films relative to arteries exposed to uncoated PU films.

FIG. 21 is a bar graph showing the area of granulation tissue in carotid arteries exposed to natural and purified silk powder and wrapped with perivascular PU film relative to a control group in which arteries are wrapped with perivascular PU film only.

FIG. 22 is a bar graph showing the area of granulation tissue (at 1 month and 3 months) in carotid arteries sprinkled with talcum powder and wrapped with perivascular PU film relative to a control group in which arteries are wrapped with perivascular PU film only.

### DETAILED DESCRIPTION OF THE INVENTION

Definitions

Prior to setting forth the invention, it may be helpful to an understanding thereof to first set forth definitions of certain terms that are used hereinafter.

“Medical device”, “implant”, “device”, “medical device,” “medical implant”, “implant/device”, and the like are used synonymously to refer to any object that is designed to be placed partially or wholly within a patient's body for one or more therapeutic or prophylactic purposes such as for restoring physiological function, alleviating symptoms associated with disease, delivering therapeutic agents, detecting changes (or levels) in the internal environment, and/or repairing or replacing or augmenting etc. damaged or diseased organs and tissues. While medical devices are normally composed of biologically compatible synthetic materials (e.g., medical-grade stainless steel, titanium and other metals; exogenous polymers, such as polyurethane, silicon, PLA, PLGA), other materials may also be used in the construction of the medical device or implant. Specific medical devices and implants that are particularly useful for the practice of this invention include devices and implants designed to deliver therapeutic levels of a drug to a target tissue (drug delivery pumps) and/or sensors designed to detect changes in body function and/or levels of key physiological metabolites, chemistry, hormones or biological factors.

“Implantable sensor” refers to a medical device that is implanted in the body to detect blood or tissue levels of a particular chemical (e.g., glucose, electrolytes, drugs, hormones) and/or changes in body chemistry, metabolites, function, pressure, flow, physical structure, electrical activity or other variable parameter. Implantable sensors may have one or more electrodes that extend into the external environment to sense a variety of physical and/or physiological properties, including, but not limited to, optical, mechanical, baro, chemical and electrochemical properties. Sensors may be used to detect information, for example, about temperature, strain, pressure, magnetic, acceleration, ionizing radiation, acoustic wave or chemical changes (e.g., blood constituents, such as glucose). For example for the detection of glucose levels, the sensor may utilize an enzyme-based electrochemical sensor, a glucose-responsive hydrogel combined with a pressure sensor, microwires with electrodes, radiofrequency microelectronics and a glucose affinity polymer combined with physical and biochemical sensor technology, and near or mid infrared light emission combined with optical spectroscopy detectors to name a few. Representative examples of implantable sensors include, blood/tissue glucose monitors, electrolyte sensors, blood constituent sensors, temperature sensors, pH sensors, optical sensors, amperometric sensors, pressure sensors, biosensors, sensing transponders, strain sensors, activity sensors and magnetoresistive sensors.

“Drug-delivery pump” refers to a medical device that includes a pump which is configured to deliver a biologically active agent (e.g., a drug) at a regulated dose. These devices are implanted within the body and may include an external transmitter for programming the controlled release of drug, or alternatively, may include an implantable sensor that provides the trigger for the drug delivery pump to release drug as physiologically required. Drug-delivery pumps may be used to deliver virtually any agent, but specific examples include insulin for the treatment of diabetes, medication for the relief of pain, chemotherapy for the treatment of cancer, anti-spastic agents for the treatment of movement and muscular disorders, or antibiotics for the treatment of infections. Representative examples of drug delivery pumps for use in the practice of the invention include, without limitation, constant flow drug delivery pumps, programmable drug delivery pumps, intrathecal pumps, implantable insulin delivery pumps, implantable osmotic pumps, ocular drug delivery pumps and implants, metering systems, peristaltic (roller) pumps, electronically driven pumps, elastomeric pumps, spring-contraction pumps, gas-driven pumps (e.g., induced by electrolytic cell or chemical reaction), hydraulic pumps, piston-dependent pumps and non-piston-dependent pumps, dispensing chambers, infusion pumps, passive pumps, infusate pumps and osmotically-driven fluid dispensers.

“Fibrosis,” “scarring,” or “fibrotic response” refers to the formation of fibrous (scar) tissue in response to injury or medical intervention. Therapeutic agents which inhibit fibrosis or scarring can do so through one or more mechanisms including: inhibiting the inflammatory response, inhibiting migration or proliferation of connective tissue cells (such as fibroblasts, smooth muscle cells, and vascular smooth muscle cells), inhibiting angiogenesis, reducing ECM production (or promoting ECM breakdown), and/or inhibiting tissue remodeling. In addition, numerous therapeutic agents described in this invention will have the additional benefit of also reducing tissue regeneration (the replacement of injured cells by cells of the same type) when appropriate.

“Inhibit fibrosis”, “reduce fibrosis”, “fibrosis-inhibitor”, “inhibits scar”, “reduces scar”, “anti-fibrosis”, “anti-scarring” and the like are used synonymously to refer to the action of agents or compositions which result in a statistically significant decrease in the formation of fibrous tissue that may be expected to occur in the absence of the agent or composition.

“Inhibitor” refers to an agent which prevents a biological process from occurring or slows the rate or degree of occurrence of a biological process. The process may be a general one such as scarring or refer to a specific biological action such as, for example, a molecular process resulting in release of a cytokine.

“Antagonist” refers to an agent which prevents a biological process from occurring or slows the rate or degree of occurrence of a biological process. While the process may be a general one, typically this refers to a drug mechanism where the drug competes with a molecule for an active molecular site or prevents a molecule from interacting with the molecular site. In these situations, the effect is that the molecular process is inhibited.

“Agonist” refers to an agent which stimulates a biological process or rate or degree of occurrence of a biological process. The process may be a general one such as scarring or refer to a specific biological action such as, for example, a molecular process resulting in release of a cytokine.

“Anti-microtubule agents” may be understood to include any protein, peptide, chemical, or other molecule which impairs the function of microtubules, for example, through the prevention or stabilization of polymerization. Compounds that stabilize polymerization of microtubules are referred to herein as “microtubule stabilizing agents.” A wide variety of methods may be utilized to determine the anti-microtubule activity of a particular compound, including for example, assays described by Smith et al. (Cancer Lett 79 (2): 213-219, 1994) and Mooberry et al., (Cancer Lett. 96 (2): 261-266, 1995).

“Host”, “person”, “subject”, “patient” and the like are used synonymously to refer to the living being (human or animal) into which a device of the present invention is implanted.

“Implanted” refers to having completely or partially placed a device within a host. A device is partially implanted when some of the device reaches, or extends to the outside of, a host.

“Release of an agent” refers to a statistically significant presence of the agent, or a subcomponent thereof, which has disassociated from the implant/device and/or remains active on the surface of (or within) the device/implant.

“Biodegradable” refers to materials for which the degradation process is at least partially mediated by, and/or performed in, a biological system. “Degradation” refers to a chain scission process by which a polymer chain is cleaved into oligomers and monomers. Chain scission may occur through various mechanisms, including, for example, by chemical reaction (e.g., hydrolysis) or by a thermal or photolytic process. Polymer degradation may be characterized, for example, using gel permeation chromatography (GPC), which monitors the polymer molecular mass changes during erosion and drug release. Biodegradable also refers to materials may be degraded by an erosion process mediated by, and/or performed in, a biological system. “Erosion” refers to a process in which material is lost from the bulk. In the case of a polymeric system, the material may be a monomer, an oligomer, a part of a polymer backbone, or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1 (1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage; (2) polymers that are initially water insoluble are solubilized by hydrolysis, ionization, or pronation of a pendant group; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance spectroscopy (EPR), NMR imaging, and recording mass loss during an erosion experiment. For microspheres, photon correlation spectroscopy (PCS) and other particles size measurement techniques may be applied to monitor the size evolution of erodible devices versus time.

As used herein, “analogue” refers to a chemical compound that is structurally similar to a parent compound, but differs slightly in composition (e.g., one atom or functional group is different, added, or removed). The analogue may or may not have different chemical or physical properties than the original compound and may or may not have improved biological and/or chemical activity. For example, the analogue may be more hydrophilic or it may have altered reactivity as compared to the parent compound. The analogue may mimic the chemical and/or biologically activity of the parent compound (i.e., it may have similar or identical activity), or, in some cases, may have increased or decreased activity. The analogue may be a naturally or non-naturally occurring (e.g., recombinant) variant of the original compound. An example of an analogue is a mutein (i.e., a protein analogue in which at least one amino acid is deleted, added, or substituted with another amino acid). Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound that is structurally similar to a parent compound and (actually or theoretically) derivable from that parent compound. A “derivative” differs from an “analogue” in that a parent compound may be the starting material to generate a “derivative,” whereas the parent compound may not necessarily be used as the starting material to generate an “analogue.” A derivative may or may not have different chemical or physical properties of the parent compound. For example, the derivative may be more hydrophilic or it may have altered reactivity as compared to the parent compound. Derivatization (i.e., modification) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups, for example carboxylic acid groups, can form, for example, alkali metal salts or alkaline earth metal salts (e.g., sodium salts, potassium salts, magnesium salts and calcium salts, and also salts with physiologically tolerable quaternary ammonium ions and acid addition salts with ammonia and physiologically tolerable organic amines such as, for example, triethylamine, ethanolamine or tris-(2-hydroxyethyl)amine). Basic groups can form acid addition salts, for example with inorganic acids such as hydrochloric acid, sulfuric acid or phosphoric acid, or with organic carboxylic acids and sulfonic acids such as acetic acid, citric acid, benzoic acid, maleic acid, fumaric acid, tartaric acid, methanesulfonic acid or p-toluenesulfonic acid. Compounds which simultaneously contain a basic group and an acidic group, for example a carboxyl group in addition to basic nitrogen atoms, can be present as zwitterions. Salts can be obtained by customary methods known to those skilled in the art, for example by combining a compound with an inorganic or organic acid or base in a solvent or diluent, or from other salts by cation exchange or anion exchange.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. For example, “a” polymer refers to one polymer or a mixture comprising two or more polymers. As used herein, the term “about” means±15%.

As discussed above, the present invention provides compositions, methods and devices relating to medical devices and implants (specifically implantable pumps and sensors), which greatly increase their ability to inhibit the formation of reactive scar tissue on, or around, the surface of the device or implant. Described in more detail below are methods for constructing medical devices or implants, compositions and methods for generating medical devices and implants which inhibit fibrosis, and methods for utilizing such medical devices and implants.

A. Clinical Applications of Implantable Sensor and Pump Devices Which Include and Release a Fibrosis-inhibiting Agent

1. Implantable Sensors

In one aspect, implantable sensors that include an anti-scarring agent are provided that can be used to detect physiological levels or changes in the body. There are numerous sensor devices where the occurrence of a fibrotic reaction will adversely affect the functioning of the device or the biological problem for which the device was implanted or used. Proper clinical functioning of an implanted sensor is dependent upon intimate anatomical contact with the target tissues and/or body fluids. Scarring around the implanted device may degrade the electrical components and characteristics of the device-tissue interface, and the device may fail to function properly. The formation of scar tissue between the sensing device and the adjacent (target) tissue can prevent the flow of physical, chemical and/or biological information (e.g., fluid levels, drug levels, metabolite levels, glucose levels, pressure etc.) from reaching the detection mechanism of the sensor. Similarly if a “foreign body” response occurs and causes the implanted sensor to become encapsulated by scar (i.e., the body “walls off” the sensor with fibrous tissue), the sensor will receive biological information that is not reflective of the organism as a whole. If the sensor is detecting conditions inside the capsule (i.e., levels detected in a microenvironment), and these conditions are not consistent with those outside the capsule (i.e., within the body as a whole—the microenvironment), it will record information that is not representative of systemic levels.

Sensors or transducers may be located deep within the body for monitoring a variety of physiological properties, such as temperature, pressure, strain, fluid flow, metabolite levels (e.g., electrolytes, glucose), drug levels, chemical properties, electrical properties, magnetic properties, and the like. Representative examples of implantable sensors for use in the practice of the invention include, blood and tissue glucose monitors, electrolyte sensors, blood constituent sensors, temperature sensors, pH sensors, optical sensors, amperometric sensors, pressure sensors, biosensors, sensing transponders, strain sensors, activity sensors and magnetoresistive sensors.

Numerous types of implantable sensors and transducers have been described. For example, the implantable sensor may be a micro-electronic device that is implanted around the large bowels to control bowel function by detecting rectal contents and stimulating peristaltic contractions to empty the bowels when it is convenient. See, e.g., U.S. Pat. No. 6,658,297. The implantable sensor may be used to measure pH in the GI tract. A representative example of such a pH sensing device is the BRAVO pH Monitoring System from Medtronic, Inc. (Minneapolis, Minn.). The implantable sensor may be part of a GI catheter or probe that includes a sensor portion connected to an electrical or optical measurement device and a sensitive polymeric material that undergoes an irreversible change when exposed to cumulative action of an external medium. See, e.g., U.S. Pat. No. 6,006,121. The implantable sensor may be a component of a central venous catheter (CVC) (e.g., a jugular vein catheter) system. For example, the device may be composed of a catheter body having at least one oxygen sensor and a distal heat exchange region in which the catheter body is formed with coolant supply and return lumens to provide heat exchange within a body to prevent overheating due to severe brain trauma or ischemia due to stroke. See, e.g., U.S. Pat. No. 6,652,565. A CVC may include a thermal mass and a temperature sensor to measure blood temperature. See, e.g., U.S. Pat. No. 6,383,144.

Several specific implantable sensor devices and treatments will be described in greater detail including:

a. Blood and Glucose Monitors

Glucose monitors are used to detect changes in blood glucose, specifically for the management and treatment of patients with diabetes mellitus. Diabetes is a metabolic disorder of glucose metabolism that afflicts tens of millions of people in the developed countries of the world. This disease is characterized by the inability of the body to properly utilize and metabolize carbohydrates, particularly glucose. Normally, the finely-tuned balance between glucose in the blood and glucose in the bodily tissue cells is maintained by insulin, a hormone produced by the pancreas. If the pancreas becomes defective and insulin is produced in inadequate amounts to reduce blood glucose levels (Type I diabetes), or if the body becomes insensitive to the glucose-lowering effects of insulin despite adequate pancreatic insulin production (Type II diabetes), the result is diabetes. Accurate detection of blood glucose levels is essential to the management of diabetic patients because the dosage and timing of administration of insulin and/or other hypoglycemic agents are titrated depending upon changes in glucose levels in response to the medication. If the dosage is too high, blood glucose levels drop too low, resulting in confusion and potentially even loss of consciousness. If the dosage is too low, blood glucose levels rise too high, leading to excessive thirst, urination, and changes in metabolism known as ketoacidosis. If the timing of medication administration is incorrect, blood glucose levels can fluctuate wildly between the two extremes—a situation that is thought to contribute to some of the long-term complications of diabetes such as heart disease, kidney failure and blindness. Since in the extreme, all these conditions can be life threatening, careful and continuous monitoring of glucose levels is a critical aspect of diabetes management. One way to detect changes in glucose levels and to continuously sense when levels of glucose become too high or too low in diabetes patients is to implant a glucose sensor. As the glucose sensor detects changes in the blood glucose levels, insulin can be administered by external injection or via an implantable insulin pump to maintain blood glucose levels within an acceptable physiologic range.

Numerous types of blood and tissue glucose monitors are suitable for use in the practice of the invention. For example, the glucose monitor may be delivered to the vascular system transluminally using a catheter on a stent platform. See, e.g., U.S. Pat. No. 6,442,413. The glucose monitor may be composed of glucose sensitive living cells that monitor blood glucose levels and produce a detectable electrical or optical signal in response to changes in glucose concentrations. See, e.g., U.S. Pat. Nos. 5,101,814 and 5,190,041. The glucose monitor may be a small diameter flexible electrode implanted subcutaneously which may be composed of an analyte-responsive enzyme designed to be an electrochemical glucose sensor. See, e.g., U.S. Pat. Nos. 6,121,009 and 6,514,718. The implantable sensor may be a closed loop insulin delivery system whereby there is a sensing means that detects the patient's blood glucose level based on electrical signals and then stimulates either an insulin pump or the pancreas to supply insulin. See, e.g., U.S. Pat. Nos. 6,558,345 and 6,093,167. Other glucose monitors are described in, for e.g., U.S. Pat. Nos. 6,579,498; 6,565,509 and 5,165,407. Minimally invasive glucose monitors include the GLUCOWATCH G2 BIOGRAPHER from Cygnus Inc. (see cygn.com); see, e.g., U.S. Pat. Nos. 6,546,269; 6,687,522; 6,595,919 and U.S. Patent Application Nos. 20040062759A1; 20030195403A1; and 20020091312A1.

Numerous commercially available blood and tissue glucose sensor devices are suitable for the practice of this invention. Although virtually any implantable glucose sensor may be utilized, several specific commercial and development stage examples are described below for greater clarity.

The CONTINUOUS GLUCOSE MONITORING SYSTEM (CGMS) from Medtronic MiniMed, Inc. (Northridge, Calif.; see minimed.com); see, e.g., U.S. Pat. Nos. 6,520,326; 6,424,847; 6,360,888; 5,605,152; 6,804,544; and U.S. Patent Application No. 20040167464A1. The CGMS system is surgically implanted in the subcutaneous tissue of the abdomen and stores tissue glucose readings every 5 minutes. Coating the sensor with a fibrosis-inhibiting agent may prolong the activity of this device because it often must be removed after several days (approximately 3), in part because it loses its sensitivity as a result of the local tissue reaction to the device.

The CONTINUOUS GLUCOSE MONITORING DEVICE from TheraSense (Alameda, Calif., see therasense.com) which utilizes a disposable, miniaturized electrochemical sensor that is inserted under the patient's skin using a spring-loaded insertion device. The sensor measures glucose levels in the interstitial fluid every five minutes, with the ability to store results for future analysis. See, e.g., U.S. 20040186365A1; U.S. 20040106858A1 and U.S. 20030176183A1. Even though the device can store up to a month of data and has alarms for high and low glucose levels, it must be replaced every few days because it loses its accuracy as a result of the foreign body reaction to the implant. Utilizing this sensor in combination with a fibrosis-inhibiting agent may prolong its activity, enhance its performance and reduce the frequency of replacement. Another electrochemical sensor that may benefit from the present invention is the multilayered implantable electrochemical sensor from Isense (Portland, Oreg.). This system consists of a semipermeable membrane, a catalytic membrane which generates an electrical current in the presence of glucose, and a specificity membrane to reduce interference from other substances.

The SMSI glucose sensor (Sensors for Medicine and Sciences, Inc., Montgomery County, Md.; see s4ms.com) is designed to be implanted under the skin in a short outpatient procedure. The sensor is designed to automatically measure interstitial glucose every few minutes, without any user intervention. The sensor implant communicates wirelessly with a small external reader, allowing the user to monitor glucose levels continuously or on demand. The reader is designed to be able to track the rate of change of glucose levels and warn the user of impending hypo- or hyperglycemia. The operational life of the sensor implant is about 6-12 months, after which it may be replaced.

Animas Corporation (West Chester, Pa.; animascorp.com) is developing an implantable glucose sensor that measures the near-infrared absorption of blood based on spectroscopy or optical sensing placed around a vein. The Animas glucose monitor may be tied to an insulin infusion pump to provide a closed-loop control of blood glucose levels. Scar tissue over the sensor distorts the ability of the device to correctly gather optical information and may thus benefit from use in combination with a fibrosis inhibiting agent.

DexCom, Inc. (San Diego, Calif.; see dexcom.com) is developing their Continuous Glucose Monitoring System which is an implantable sensor that wirelessly transmits continuous blood glucose readings to an external receiver. The receiver displays the current glucose value every 30 seconds, as well as one-hour, three-hour and nine-hours trended values, and sounds an alert when a high or low glucose excursion is detected. This device features an implantable sensor that is placed in the subcutaneous tissue and continuously monitors tissue (interstitial fluid) glucose levels for both type 1 and type 2 diabetics. This device may also include a unique microarchitectural arrangement in the sensor region that allows accurate data to be obtained over long periods of time. Glucose monitoring devices and associated systems that are developed by DexCom, Inc. are described in, for example, U.S. Pat. Nos. 6,741,877; 6,702,857 and 6,558,321. Unfortunately, even though the battery and circuitry of monitoring devices allows long-term functioning, a foreign body response and/or encapsulation of the implant affect the ability of the device to detect glucose levels accurately for prolonged periods in a percentage of implants. Combining this device with an inhibitor of fibrosis (e.g., by coating the implant and/or sensor with the agent, incorporating the agent into the polymers that make up the implant, and/or infiltrating it into the tissue surrounding the implant) may allow it to accurately detect glucose levels for longer periods of time after implantation, reduce the number of devices that fail and decrease the incidence of replacement.

Also of particular interest in the practice of this invention is glucose monitoring systems that utilize a glucose-responsive polymer as part of their detection mechanism. M-Biotech (Salt Lake City, Utah) is developing a continuous monitoring system that consists of subcutaneous implantation of a glucose-responsive hydrogel combined with a pressure transducer. See, e.g., U.S. Pat. Nos.; and The hydrogel responds to changes in glucose concentration by either shrinking or swelling and the expansion or contraction is detected by the pressure transducer. The transducer converts the information into an electrical signal and sends a wireless signal to a display device. Cybersensors (Berkshire, UK) produces a capsule-like sensor implanted under the skin and an external receiver/transmitter that captures the data and powers the capsule via RF signals (see, e.g., GB 2335496 and U.S. Pat. No. 6,579,498) Issued by the UK Patent and Trademark Office). The sensor capsule is composed of a glucose affinity polymer and contains a physical sensor and an RF microchip; the entire capsule is further enclosed in a semipermeable membrane. The glucose affinity polymer exhibits rheological changes when exposed to glucose (in the range of 3-15 nM) by becoming thinner and less viscous as glucose concentrations increase. This reversible reaction can be detected by the physical sensor and converted into a signal. These aforementioned systems offer an excellent opportunity for combining the implanted sensor with fibrosis-inhibiting agents and compositions. Not only can the agent be coated onto the surface of the sensor or infiltrated into the tissue surrounding the sensor, but it can also be incorporated into the glucose-responsive hydrogels and polymers that make up the implant.

Another glucose sensing device is under development by Advanced Biosensors (Mentor, Ohio) that consists of small (150 μm wide by 2 mm long), biocompatible, silicon-based needles that are implanted under the skin. The device senses glucose levels in the dermis and transmits data wirelessly. Unfortunately, a foreign body response and/or encapsulation of the implant affect the ability of the device to detect glucose levels accurately for longer than 7 days. Combining this device with an inhibitor of fibrosis may allow it to accurately detect glucose levels for longer periods of time and extend the effective lifespan of the device.

Regardless of the specific design features of implantable blood, tissue, or interstitial fluid glucose sensor devices, for accurate detection of physical, chemical and/or physiological properties, the device must be accurately positioned adjacent to the tissue. In particular, the detector of the sensing mechanism must be exposed to glucose levels that are identical to (or representative of) those found in the bloodstream. If excessive scar tissue growth or extracellular matrix deposition occurs around the device, this can impair the movement of glucose from the tissue to the detector and render it ineffective. Similarly if a “foreign body” response occurs and causes the implanted glucose sensor to become encapsulated by fibrous tissue, the sensor will be detecting glucose levels in the capsule. If glucose levels inside the capsule are not consistent with those outside the capsule (i.e., within the body as a whole), it will record information that is not representative of systemic levels. This can cause the physician or the patient to administer the wrong dosage of hypoglycemic drugs (such as insulin) with potentially serious consequences. Blood, tissue or interstitial fluid glucose sensor devices that release a therapeutic agent able to reduce scarring and/or encapsulation of the implant can increase the efficiency and accuracy of glucose detection, minimize insulin dosing errors, assist in the maintenance of correct blood glucose levels, increase the duration that these devices function clinically, and/or reduce the frequency of implant replacement. In one aspect, the device includes blood, tissue and interstitial fluid glucose monitoring devices that are coated with an anti-scarring agent or a composition that includes an anti-scarring agent. The fibrosis-inhibiting agent can also be incorporated into, and released from, the components of the implanted sensor. This embodiment is particularly useful for implants employing glucose-responsive polymers and hydrogels (that can be drug-loaded with an active agent) as well as those utilizing a semi-permeable membrane around the sensor (which can also be loaded with a fibrosis-inhibiting agent). As an alternative to this, or in addition to this, a composition that includes an anti-scarring agent can be infiltrated into the tissue surrounding where the glucose sensor is, or will be, implanted.

b. Pressure and Stress Sensors

In another aspect, the implantable sensor may be a pressure monitor. Pressure monitors may be used to detect increasing pressure or stress within the body. Implantable pressure transducers and sensors are used for temporary or chronic use in a body organ, tissue or vessel for recording absolute pressure. Many different designs and operating systems have been proposed and placed into temporary or chronic use for patients with a variety of medical conditions. Indwelling pressure sensors for temporary use of a few days or weeks are available, however, chronically or permanently implantable pressure sensors have also been used. Pressure sensors may detect many types of bodily pressures, such as, but not limited to blood pressure and fluid flow, pressure within aneurysm sacs, intracranial pressure, and mechanical pressure associated with bone fractures.

Numerous types of pressure monitors are suitable for use in the practice of the invention. For example, the implantable sensor may detect body fluid absolute pressure at a selected site and ambient operating temperature by using a lead, sensor module, sensor circuit (including electrical conductors) and means for providing voltage. See, e.g., U.S. Pat. No. 5,535,752. The implantable sensor may be an intracranial pressure monitor that provides an analogue data signal which is converted electronically to a digital pulse. See, e.g., U.S. Pat. No. 6,533,733. The implantable sensor may be a barometric pressure sensor enclosed in an air chamber which is used for deriving reference pressure data for use in combination with an implantable medical device, such as a pacemaker. See, e.g., U.S. Pat. No. 6,152,885. The implantable sensor may be adapted to be inserted into a body passageway to monitor a parameter related to fluid flow through an endoluminal implant (e.g., stent). See, e.g., U.S. Pat. No. 5,967,986. The implantable sensor may be a passive sensor with an inductor-capacitor circuit having a resonant frequency which is adapted for the skull of a patient to sense intracranial pressure. See, e.g., U.S. Pat. No. 6,113,553. The implantable sensor may be a self-powered strain sensing system that generates a strain signal in response to stresses that may be produced at a bone fixation device. See, e.g., U.S. Pat. No. 6,034,296. The implantable sensor may be a component of a perfusion catheter. The catheter may include a wire electrode and a lumen for perfusing saline around the wire, which is designed for measuring a potential difference across the GI wall and for simultaneous measurement of pressure. See, e.g., U.S. Pat. No. 5,551,425. The implantable sensor may be part of a CNS device; for example, an intracranial pressure sensor which is mounted within the skull of a body at the situs where the pressure is to be monitored and a means of transmitting the pressure externally from the skull. See, e.g., U.S. Pat. No. 4,003,141. The implantable sensor may be a component of a left ventricular assist device. For example, the VAD may be a blood pump adapted to be joined in flow communication between the left ventricle and the aorta using an inlet flow pressure sensor and a controller that may adjust speed of pump based on sensor feedback. See, e.g., U.S. Pat. No. 6,623,420. Numerous commercially available and experimental pressure and stress sensor devices are suitable for the practice of the invention. By way of illustration, a selection of these devices and implants are described in the following paragraphs.

A device from CardioMEMS (Atlanta, Ga.; @cardiomems.com, a partnership between the Georgia Institute of Technology and the Cleveland Clinic) which can be inserted into an aneurysm sac to monitor pressure within the sac and thereby alert a medical specialist to the filing of the sac with fluid, possibly to rupture-provoking levels. Endovascular aneurysm repair (EVAR) is often performed using a stent graft which isolates the aneurysm from the circulation. However, persistent leakage of blood into the aneurysm sac results in ongoing pressure build-up in the sac and a resultant risk of rupture. The CardioMEMS device is implanted into the aneurysm sac after EVAR to monitor pressure in the isolated sac in order to detect which patients are at increasing risk of rupture. The pressure sensor features an inductive-capacitive resonant circuit with a variable capacitor. Since capacitance varies with the pressure in the environment in which the capacitor is placed, it can detect changes in local pressure. Data is generated by using external excitation systems that induce an oscillating current in the sensor and detecting the frequency of oscillation (which is then used to calculate pressure). Unfortunately, even though the circuitry allows long-term functioning, a foreign body response and/or encapsulation of the implant affect the ability of the device to detect accurate pressure levels in the aneurysm (i.e., the device detects the pressure in the microenvironment of the capsule, not of the aneurysm sac as a whole). Combining this device with an inhibitor of fibrosis (e.g., by coating the implant and/or sensor with the agent, incorporating the agent into the polymers that make up the implant, and/or infiltrating it into the sac surrounding the implant) may allow it to accurately detect pressure levels for longer periods of time after implantation and reduce the number of devices that fail.

MicroStrain Inc. (Williston, Vt., @microstrain.com) has developed a family of wireless implantable sensors for measuring strain, position and motion within the body. These sensors can measure, for example, eye tremor, depth of corneal implant, orientation sensor for improved tooth crown prep, mayer ligament strains, spinal ligament strains, vertebral bone strains, elbow ligament strains, emg and ekg data, 3DM-G for measurement of orientation and motion, wrist ligament strains, hip replacement sensors for measuring micromotion, implant subsidence, knee ligament strain, ankle ligament strain, Achilles tendon strain, foot arch support strains, force within foot insoles. The company provides a knee prosthesis that can measure in vivo compressive forces and transmit the data in real time. Patents describing this technology, and components used in the manufacture of devices for this technology include U.S. Pat. Nos. 6,714,763; 6,625,517; 6,622,567; 6,588,282; 6,529,127; 6,499,368; 6,433,629; 5,887,351; 5,777,467; 5,497,147; and 4,993,428. U.S. Patent Applications describing this technology, and components used in the manufacture of devices for this technology include 20040113790; 20040078662; 20030204361; 20030158699; 20030047002; 20020190785; 20020170193; 20020088110; 20020085174; 20010054317; and 20010033187.

Mesotec (Hannover, Germany; @mesotec.com), in collaboration with several German institutes (e.g., Fraunhofer Institute of Microelectronic Circuits and Systems), has developed an implantable intraocular pressure sensor system, called the MESOGRAPH, which can continuously monitor intraocular pressure. This is desirable, e.g., in order to identify the onset of glaucoma. The CMOS-based sensor can be implanted during standard surgical procedures and is inductively linked to an external unit integrated into a spectacle frame. The glasses are in turn linked via a cable to a portable data logger. Data is relayed upstream to the glasses using a modulated RF carrier operating at 13.56 MHz and a switchable load, while power comes downstream to the sensor. By varying the diameter of the polysilicon diaphragms in the on-chip micromechanical vacuum gap capacitors, the pressure range to which the sensor responds can be adapted between 50 kNm-2 and 3.5 MNm-2. The device consists of a fine, foldable coil for telemetric coupling and a very small miniaturized pressure sensor. The sensor is manufactured on a micro-technological basis and serves for continuous, long-term reading and monitoring of intraocular pressure. Chip and coil are integrated in modified soft intraocular lenses, which can be implanted in the patient's eye during today's common surgical procedures. Unfortunately, the device often fails after initially successful implantation because a foreign body response and/or encapsulation of the implant affect the ability of it to detect accurate pressure levels in the eye (i.e., the device detects the pressure in the microenvironment of the capsule surrounding the implant, not intraocular pressure as a whole). Combining this device with an inhibitor of fibrosis (e.g., by coating the implant and/or sensor with the agent, incorporating the agent into the polymers that make up the implant, and/or infiltrating it into the eye tissue surrounding the implant) may allow it to accurately detect pressure levels for longer periods of time after implantation and reduce the number of devices that fail.

Regardless of the specific design features of the pressure or stress sensor, for accurate detection of physical and/or physiological properties (such as pressure), the device must be accurately positioned within the tissue and receive information that is representative of conditions as a whole. If excessive scar tissue growth or extracellular matrix deposition occurs around the device, the sensor may receive erroneous information that compromises its efficacy or the scar tissue may block the flow of biological information to the sensor. For example, many devices fail after initially successful implantation because encapsulation of the implant causes it to detect nonrelevant pressure levels (i.e., the device detects the pressure in the microenvironment of the capsule surrounding the implant, not the pressure of the larger environment). Pressure and stress sensing devices that release a therapeutic agent able to reduce scarring can increase the efficiency of detection and increase the duration that these devices function clinically. In one aspect, the device includes implantable sensor devices that are coated with an anti-scarring agent or a composition that includes an anti-scarring agent. The fibrosis-inhibiting agent can also be incorporated into, and released from, the components (such as polymers) that are part of the structure of the implanted sensor. As an alternative to this, or in addition to this, a composition that includes an anti-scarring agent can be infiltrated into the tissue surrounding where the device is, or will be, implanted.

c. Cardiac Sensors

In another aspect, the implantable sensor may be a device configured to detect properties in the heart or in cardiac muscle tissue. Cardiac sensors are used to detect parameters associated with the performance of the heart as monitored at any given time point along a prolonged time period. Typically, monitoring of the heart is often conducted to detect changes associated with heart disease, such as chronic heart failure (CHF). By monitoring patterns associated with heart function, deterioration based on hemodynamic changes can be detected (parameters such as cardiac output, ejection fraction, pressure, ventricular wall motion, etc.). This constant direct monitoring is central to disease management in patients that present with CHF. By monitoring hemodynamic measures directly using implantable sensors, a hemodynamic crisis can be detected and the appropriate medications and interventions selected.

Numerous types of cardiac sensors are suitable for use in the practice of the invention. For example, the implantable sensor may be an activity sensor incorporating a magnet and a magnetoresistive sensor that provides a variable activity signal as part of a cardiac device. See, e.g., U.S. Pat. Nos. 6,430,440 and 6,411,849. The implantable sensor may monitor blood pressure in a heart chamber by emitting wireless communication to a remote device. See, e.g., U.S. Pat. No. 6,409,674. The implantable sensor may be an accelerometer-based cardiac wall motion sensor which transduces accelerations of cardiac tissue to a cardiac stimulation device by using electrical signals. See, e.g., U.S. Pat. No. 5,628,777. The implantable sensor may be implanted in the heart's cavity with an additional sensor implanted in a blood vessel to detect pressure and flow within heart's cavity. See, e.g., U.S. Pat. No. 6,277,078.

Commercially available cardiac sensor devices suitable for the practice of the invention include Biotronik's (Biotronik GmbH & Co., Berlin, Germany, see biotronik.com) CARDIAC AIRBAG ICD SYSTEM is a rhythm monitoring device that offers rescue shock capability delivering 30 Joule shock therapies for up to 3 episodes of ventricular fibrillation. In addition to the rescue shock capability the system can also provide bradycardia pacing and VT monitoring. The PROTOS family of pacemakers from Biotronik (see biotronikusa.com) also incorporates pacing sensor capability called Closed Loop Simulation.

Blood flow and tissue perfusion monitors can be used to monitor noncardiac tissue as well. Researchers at Oak Ridge National Laboratory have developed a wireless sensor that monitors blood flow to a transplanted organ for the early detection of transplant rejection.

Medtronic (Minneapolis, Minn.; see medtronic.com) is developing their CHRONICLE implantable product, which is designed to continuously monitor a patient's intracardiac pressures, heart rate and physical activity using a sensor placed directly in the heart's chamber. The patient periodically downloads this information to a home-based device that transmits this physiologic data securely over the Internet to a physician.

Regardless of the specific design features of the cardiac sensor, for accurate detection of physical and/or physiological properties (such as pressure, flow rates, etc.), the device must be accurately positioned within the heart muscle, chambers or great vessels and receive information that is representative of conditions as a whole. If excessive scar tissue growth or extracellular matrix deposition occurs around the sensing device, the sensor may receive erroneous information that compromises its efficacy, or the scar tissue may block the flow of biological information to the detector mechanism of the sensor. For example, many cardiac monitoring devices fail after initially successful implantation because encapsulation of the implant causes it to detect nonrelevant levels (i.e., the device detects conditions in the microenvironment of the capsule surrounding the implant, not the pressure of the larger environment). Cardiac sensing devices that release a therapeutic agent able to reduce scarring can increase the efficiency of detection and increase the duration that these devices function clinically. In one aspect, the device includes implantable sensor devices that are coated with an anti-scarring agent or a composition that includes an anti-scarring agent. The fibrosis-inhibiting agent can also be incorporated into, and released from, the components (such as polymers) that are part of the structure of the implanted cardiac sensor. As an alternative to this, or in addition to this, a composition that includes an anti-scarring agent can be infiltrated into the tissue surrounding where the device is, or will be, implanted.

d. Respiratory Sensors

In another aspect, the implantable sensor may be a device configured to detect properties in the respiratory system. Respiratory sensors may be used to detect changes in breathing patterns. For example, a respiratory sensor may be used to detect sleep apnea, which is an airway disorder. There are two kinds of sleep apnea. In one condition, the body fails to automatically generate the neuromuscular stimulation necessary to initiate and control a respiratory cycle at the proper time. In the other condition, the muscles of the upper airway contract during the time of inspiration and thus the airway becomes obstructed. The cardiovascular consequences of apnea include disorders of cardiac rhythm (bradycardia, auriculoventricular block, ventricular extrasystoles) and hemodynamic disorders (pulmonary and systemic hypertension). This results in a stimulatory metabolic and mechanical effect on the autonomic nervous system and the potential to ultimately lead to increased morbidity. To treat this condition, implantable sensors may be used to monitor respiratory functioning to detect an apnea episode so the appropriate response (e.g., electrical stimulation to the nerves of the upper airway muscles) or other treatment can be provided.

Numerous types of respiratory sensors are suitable for use in the practice of the invention. For example, the implantable sensor may be a respiration element implanted in the thoracic cavity which is capable of generating a respiration signal as part of a ventilation system for providing gas to a host. See, e.g., U.S. Pat. No. 6,357,438. The implantable sensor may be composed of a sensing element connected to a lead body which is inserted into bone (e.g., manubrium) that communicates with the intrathoracic cavity to detect respiratory changes. See, e.g., U.S. Pat. No. 6,572,543.

Regardless of the specific design features of the respiratory sensor, for accurate detection of physical and/or physiological properties, the device must be accurately positioned adjacent to the tissue. If excessive scar tissue growth or extracellular matrix deposition occurs around the pulmonary function or airway sensing device, the sensor may receive erroneous information that compromises its efficacy, or the scar tissue may block the flow of biological information to the detector mechanism of the sensor. For example, many pulmonary function sensing devices fail after initially successful implantation because encapsulation of the implant causes it to detect nonrelevant levels (i.e., the device detects conditions in the microenvironment of the capsule surrounding the implant, not the functioning of the respiratory system as whole). Respiratory sensing devices that release a therapeutic agent able to reduce scarring can increase the efficiency of detection and increase the duration that these devices function clinically. In one aspect, the device includes implantable sensor devices that are coated with an anti-scarring agent or a composition that includes an anti-scarring agent. The fibrosis-inhibiting agent can also be incorporated into, and released from, the components (such as polymers) that are part of the structure of the implanted respiratory sensor. As an alternative to this, or in addition to this, a composition that includes an anti-scarring agent can be infiltrated into the tissue surrounding where the device is, or will be, implanted.

e. Auditory Sensors

In another aspect, the implantable sensor may be a device configured to detect properties in the auditory system. Auditory sensors are used as part of implantable hearing systems for rehabilitation of pure sensorineural hearing losses, or combined conduction and inner ear hearing impairments. Hearing systems may include an implantable sensor which delivers an electrical signal which is processed by an implanted processor and delivered to an implantable electromechanical transducer which acts on the middle or inner ear. The auditory sensor acts as the microphone of the hearing system and acts to convert the incident airborne sound into an electrical signal.

Numerous types of auditory sensors as part of a hearing system are suitable for use in the practice of the invention. For example, the implantable sensor may generate an electrical audio signal as part of a hearing system for rehabilitation of hearing loss. See, e.g., U.S. Pat. No. 6,334,072. The implantable sensor may be a capacitive sensor which is mechanically or magnetically coupled to a vibrating auditory element, such as the malleus, which detects the time-varying capacitance values resulting from the vibrations. See, e.g., U.S. Pat. No. 6,190,306. The implantable sensor may be an electromagnetic sensor having a permanent magnet and a coil and a time-varying magnetic flux linkage based on the vibrations which are provided to an output stimulator for mechanical or electrical stimulation of the cochlea. See, e.g., U.S. Pat. No. 5,993,376.

Commercially available auditory sensor devices suitable for the practice of the invention include: the HIRES 90K Bionic Ear Implant, HIRESOLUTION SOUND, CLARION CII Bionic Ear, and CLARION 1.2, from Advanced Bionics (Sylmar, California, a Boston Scientific Company, see advancedbionics.com); see also U.S. Pat. Nos. 6,778,858; 6,754,537; 6,735,474; 6,731,986; 6,658,302; 6,636,768; 6,631,296; 6,628,991; 6,498,954; 6,487,453; 6,473,651; 6,415,187; and 6,415,185; the NUCLEUS 3 cochlear implant from Cochlear (Lane Cove NSW, Australia, see cochlear.com); see also U.S. Pat. Nos. 6,810,289; 6,807,455; 6,788,790; 6,782,619; 6,751,505; 6,736,770; 6,700,982; 6,697,674; 6,678,564; 6,620,093; 6,575,894; 6,570,363; 6,565,503; 6,554,762; 6,537,200; 6,525,512; 6,496,734; 6,480,820; 6,421,569; 6,411,855; 6,394,947; 6,392,386; 6,377,075; 6,301,505; 6,289,246; 6,116,413; 5,720,099; 5,653,742; 5,645,585; and U.S. Patent Application Publication Nos. 2004/0172102A1 and 2002/0138115A1; the PULSAR CI 100 and COMBI 40+ cochlear implants from Med-EI (Austria, see medel.com); see also U.S. Patent Application 20040039245A1, U.S. Pat. Nos. 6,600,955; 6,594,525; 6,556,870; and 5,983,139; the ALLHEAR implants from AllHear, Inc. (Aurora, Oreg.; see allhear.com); see also WO 01/50816; EP 1 245 134; and the DIGISONIC CONVEX, DIGISONIC AUDITORY BRAINSTEM, and DIGISONIC MULTI-ARRAY implants from MXM (France; see mxmlab.com); see also U.S. Pat. Nos. 5,123,422; EP 0 219 380; WO 04/002193; EP 1 244 400 A1; U.S. Pat. No. 6,428,484; U.S. 20020095194A1; WO 01/50992.

Regardless of the specific design features of the auditory sensor, for accurate detection of sound, the device must be accurately positioned within the ear. If excessive scar tissue growth or extracellular matrix deposition occurs around the auditory sensor, the sensor may receive erroneous information that compromises its efficacy, or the scar tissue may block the flow of sound waves to the detector mechanism of the sensor. Auditory sensing devices that release a therapeutic agent able to reduce scarring can increase the efficiency of sound detection and increase the duration that these devices function clinically. In one aspect, the device includes implantable sensor devices that are coated with an anti-scarring agent or a composition that includes an anti-scarring agent. The fibrosis-inhibiting agent can also be incorporated into, and released from, the components (such as polymers) that are part of the structure of the implanted auditory sensor. As an alternative to this, or in addition to this, a composition that includes an anti-scarring agent can be infiltrated into the tissue surrounding where the device is, or will be, implanted.

f. Electrolyte and Metabolite Sensors

In another aspect, implantable sensors may be used to detect electrolytes and metabolites in the blood. For example, the implantable sensor may be a device to monitor constituent levels of metabolites or electrolytes in the blood by emitting a source of radiation directed towards blood such that it interacts with a plurality of detectors that provide an output signal. See, e.g., U.S. Pat. No. 6,122,536. The implantable sensor may be a biosensing transponder which is composed of a dye that has optical properties that change in response to changes in the environment, a photosensor to sense the optical changes, and a transponder for transmitting data to a remote reader. See, e.g., U.S. Pat. No. 5,833,603. The implantable sensor may be a monolithic bioelectronic device for detecting at least one analyte within the body of an animal. See, e.g., U.S. Pat. No. 6,673,596. Other sensors that measure chemical analytes are described in, e.g., U.S. Pat. Nos. 6,625,479 and 6,201,980.

If excessive scar tissue growth or extracellular matrix deposition occurs around the sensor, the sensor may receive erroneous information that compromises its efficacy, or the scar tissue may block the flow of metabolites or electrolytes to the detector mechanism of the sensor. For example, many metabolite/electrolyte sensing devices fail after initially successful implantation because encapsulation of the implant causes it to detect nonrelevant levels (i.e., the device detects conditions in the microenvironment of the capsule surrounding the implant, not blood levels). Sensing devices that release a therapeutic agent able to reduce scarring can increase the efficiency of metabolite/electrolyte detection and increase the duration that these devices function clinically. In one aspect, the device includes implantable sensor devices that are coated with an anti-scarring agent or a composition that includes an anti-scarring agent. The fibrosis-inhibiting agent can also be incorporated into, and released from, the components (such as polymers) that are part of the structure of the implanted sensor. As an alternative to this, or in addition to this, a composition that includes an anti-scarring agent can be infiltrated into the tissue surrounding where the device is, or will be, implanted.

Although numerous examples of implantable sensor devices have been described above, all possess similar design features and cause similar unwanted foreign body tissue reactions following implantation. It may be obvious to one of skill in the art that commercial sensor devices not specifically cited above as well as next-generation and/or subsequently-developed commercial sensor products are to be anticipated and are suitable for use under the present invention. The sensor device, particularly the sensing element, must be positioned in a very precise manner to ensure that detection is carried out at the correct anatomical location in the body. All, or parts, of a sensor device can migrate following surgery, or excessive scar tissue growth can occur around the implant, which can lead to a reduction in the performance of these devices. The formation of a fibrous capsule around the sensor can impede the flow of biological information to the detector and/or cause the device to detect levels that are not physiologically relevant (i.e., detect levels in the capsule instead of true physiological levels outside the capsule). Not only can this lead to incomplete or inaccurate readings, it can cause the physician or the patient to make incorrect therapeutic decisions based on the information generated. Implantable sensor devices that release a therapeutic agent for reducing scarring (or fibrosis) at the sensor-tissue interface can be used to increase the efficacy and/or the duration of activity of the implant. In one aspect, the present invention provides implantable sensor devices that include an anti-scarring agent or a composition that includes an anti-scarring agent. Numerous polymeric and non-polymeric delivery systems for use in implantable sensor devices will be described below. These compositions can further include one or more fibrosis-inhibiting agents such that the overgrowth of granulation, fibrous, or neointimal tissue is inhibited or reduced.

Methods for incorporating fibrosis-inhibiting compositions onto or into these sensor devices include: (a) directly affixing to the sensing device a fibrosis-inhibiting composition (e.g., by either a spraying process or dipping process as described below, with or without a carrier), (b) directly incorporating into the sensing device a fibrosis-inhibiting composition (e.g., by either a spraying process or dipping process as described below, with or without a carrier (c) by coating the sensing device with a substance such as a hydrogel which will in turn absorb the fibrosis-inhibiting composition, (d) by interweaving a fibrosis-inhibiting composition coated thread (or the polymer itself formed into a thread) into the sensing device, (e) by inserting the sensing device into a sleeve or mesh which is comprised of, or coated with, a fibrosis-inhibiting composition, (f) constructing the sensing device itself (or a portion of the device and/or the detector) with a fibrosis-inhibiting composition, or (g) by covalently binding the fibrosis-inhibiting agent directly to the sensing device surface or to a linker (small molecule or polymer) that is coated or attached to the device (or detector) surface. Each of these methods illustrates an approach for combining the sensor, detector or electrode with a fibrosis-inhibiting (also referred to herein as anti-scarring) agent according to the present invention.

For these sensors, detectors and electrodes, the coating process can be performed in such a manner as to: (a) coat a portion of the sensing device (such as the detector); or (b) coat the entire sensing device with the fibrosis-inhibiting composition. In addition to, or alternatively, the fibrosis-inhibiting agent can be mixed with the materials that are used to make the device such that the fibrosis-inhibiting agent is incorporated into the final product. In these manners, a medical device may be prepared which has a coating, where the coating is, e.g., uniform, non-uniform, continuous, discontinuous, or patterned.

In another aspect, an implantable sensor device may include a plurality of reservoirs within its structure, each reservoir configured to house and protect a therapeutic drug (i.e., one or more fibrosis-inhibiting agents). The reservoirs may be formed from divets in the device surface or micropores or channels in the device body. In one aspect, the reservoirs are formed from voids in the structure of the device. The reservoirs may house a single type of drug (e.g., fibrosis-inhibiting agent) or more than one type of drug (e.g., a fibrosis-inhibiting agent and an anti-infective agent). The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount and type of drug that is released from the substrate. The multi-layered carrier may further include a barrier layer that prevents release of the drug(s). The barrier layer can be used, for example, to control the direction that the drug elutes from the void. Thus, the coating of the medical device may directly contact the implantable sensor device, or it may indirectly contact the device when there is something, e.g., a polymer layer, that is interposed between the sensor device and the coating that contains the fibrosis-inhibiting agent.

In addition to, or as an alternative to, incorporating a fibrosis-inhibiting agent onto or into the implantable sensor device, the fibrosis-inhibiting agent can be applied directly or indirectly to the tissue adjacent to the sensor device (preferably near the sensor-tissue interface). This can be accomplished by applying the fibrosis-inhibiting agent, with or without a polymeric, non-polymeric, or secondary carrier: (a) to the sensor and/or detector surface (e.g., as an injectable, paste, gel or meSH) during the implantation procedure; (b) to the surface of the tissue (e.g., as an injectable, paste, gel, in situ forming gel or meSH) prior to, immediately prior to, or during, implantation of the sensor; (c) to the surface of the sensor and/or the tissue surrounding the implanted sensor and/or detector (e.g., as an injectable, paste, gel, in situ forming gel or meSH) immediately after the implantation of the sensor; (d) by topical application of the anti-fibrosis agent into the anatomical space where the implantable sensor will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the implantable sensor as a solution, as an infusate, or as a sustained release preparation; (f) by any combination of the aforementioned methods. Combination therapies (i.e., combinations of therapeutic agents and combinations with antithrombotic, antiplatelet, and/or anti-infective agents) can also be used.

It may be noted that certain polymeric carriers themselves can help prevent the formation of fibrous tissue on the sensor and/or fibrous encapsulation of the implanted sensor. These carriers (described below) are particularly useful for the practice of this embodiment, either alone, or in combination with a fibrosis-inhibiting composition. The following polymeric carriers can be infiltrated (as described in the previous paragraph) into the vicinity of the sensor-tissue interface and include: (a) sprayable collagen-containing formulations such as COSTASIS and crosslinked derivatized poly(ethylene glycol)-colagen compositions (described, e.g., in U.S. Pat. Nos. 5,874,500 and 5,565,519 and referred to herein as “CT3” (both from Angiotech Pharmaceuticals, Inc., Canada), either alone, or loaded with a fibrosis-inhibiting agent, applied to the implantation site (or the detector/sensor surface); (b) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Confluent Surgical, Inc., Boston, Mass.), either alone, or loaded with a fibrosis-inhibiting agent, applied to the implantation site (or the detector/sensor surface); (c) fibrinogen-containing formulations such as FLOSEAL or TISSEAL (both from Baxter Healthcare Corporation, Fremont, Calif.), either alone, or loaded with a fibrosis-inhibiting agent, applied to the implantation site (or the detector/sensor surface); (d) hyaluronic acid-containing formulations such as RESTYLANE or PERLANE (both from Q-Med AB, Sweden), HYLAFORM (Inamed Corporation, Santa Barbara, Calif.), SYNVISC (Biomatrix, Inc., Ridgefield, N.J.), SEPRAFILM or SEPRACOAT (both from Genzyme Corporation), loaded with a fibrosis-inhibiting agent applied to the implantation site (or the detector/sensor surface); (e) polymeric gels for surgical implantation such as REPEL (Life Medical Sciences, Inc., Princeton, N.J.) or FLOWGEL (Baxter Healthcare Corporation) alone, or loaded with a fibrosis-inhibiting agent, applied to the implantation site (or the detector/sensor surface); (f) orthopedic “cements” used to hold prostheses and tissues in place loaded with a fibrosis-inhibiting agent applied to the implantation site (or the detector/sensor surface), such as OSTEOBOND (Zimmer, Inc., Warsaw, Ind.), low viscosity cement (LVC) from Wright Medical Technology, Inc. (Arlington, Tenn.) SIMPLEX P (Stryker Corporation, Kalamazoo, Mich.), PALACOS (Smith & Nephew Corporation, United Kingdom), and ENDURANCE (Johnson & Johnson, Inc., New Brunswick, N.J.); (g) surgical adhesives containing cyanoacrylates such as DERMABOND (Johnson & Johnson, Inc., New Brunswick, N.J.), INDERMIL (U.S. Surgical Company, Norwalk, Conn.), GLUSTITCH (Blacklock Medical Products Inc., Canada), TISSUMEND (Veterinary Products Laboratories, Phoenix, Ariz.), VETBOND (3M Company, St. Paul, Minn.), HISTOACRYL BLUE (Davis & Geck, St. Louis, Mo.) and ORABASE SOOTHE-N-SEAL LIQUID PROTECTANT (Colgate-Palmolive Company, New York, N.Y.), either alone, or loaded with a fibrosis-inhibiting agent, applied to the implantation site (or the detector/sensor surface); (h) implants containing hydroxyapatite (or synthetic bone material such as calcium sulfate, VITOSS and CORTOSS (both available from Orthovita, Inc., Malvern, Pa.)) loaded with a fibrosis-inhibiting agent applied to the implantation site (or the detector/sensor surface); (i) other biocompatible tissue fillers alone, or loaded with a fibrosis-inhibiting agent, such as those made by BioCure, Inc. (Norcross, Ga.), 3M Company and Neomend, Inc. (Sunnyvale, Calif.), applied to the implantation site (or the detector/sensor surface); (j) polysaccharide gels such as the ADCON series of gels (available from Gliatech, Inc., Cleveland, Ohio) either alone, or loaded with a fibrosis-inhibiting agent, applied to the implantation site (or the detector/sensor surface); and/or (k) films, sponges or meshes such as INTERCEED (Gynecare Worldwide, a division of Ethicon, Inc., Somerville, N.J.), VICRYL mesh (Ethicon, Inc.), and GELFOAM (Pfizer, Inc., New York, N.Y.) alone, or loaded with a fibrosis-inhibiting agent applied to the implantation site (or the detector/sensor surface).

A preferred polymeric matrix which can be used to help prevent the formation of fibrous tissue on the sensor and/or fibrous encapsulation of the implanted sensor, either alone or in combination with a fibrosis inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue around the implanted sensor.

As should be apparent to one of skill in the art, potentially any anti-scarring agent described below may be utilized alone, or in combination, in the practice of this embodiment. As sensor devices are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the device (i.e., as a coating, incorporated into the structural components of the sensor, or infiltrated into the surrounding tissue), the fibrosis-inhibiting agents, used alone or in combination, may be administered under the following dosing guidelines:

Drugs and dosage: Therapeutic agents that may be used include but are not limited to: antimicrotubule agents including taxanes (e.g., paclitaxel and docetaxel), other microtubule stabilizing agents and anti-microtubule drugs, mycophenolic acid, sirolimus, tacrolimus, everolimus, ABT-578 and vinca alkaloids (e.g., vinblastine and vincristine sulfate) as well as analogues and derivatives thereof. Specific drugs and their corresponding dosages will be described in greater detail later, however, in general they are to be used at concentrations that range from several times more than a single systemic dose (e.g., the dose used in oral or i.v. administration) to a fraction of a single systemic dose (e.g., 50%, 10%, 5%, or even less than 1% of the concentration typically used in a single systemic dose application). In certain embodiments, the drug is released in effective concentrations for a period ranging from 1-90 days. Antimicrotubule agents including taxanes, such as paclitaxel and analogues and derivatives (e.g., docetaxel) thereof, and vinca alkaloids, including vinblastine and vincristine sulfate and analogues and derivatives thereof, should be used under the following parameters: total dose not to exceed 10 mg (range of 0.1 μg to 10 mg); preferred total dose 1 μg to 3 mg. Dose per unit area of the device of 0.05 μg-10 μg per mm 2; preferred dose/unit area of 0.20 μg/mm²-5 μg/mm². Minimum concentration of 10⁻⁹-10⁻⁴ M of drug is to be maintained on the device surface. Immunomodulators including sirolimus, ABT-578 and everolimus: sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁸-10⁻⁴ M of everolimus is to be maintained on the device surface. Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface.

2. Implantable Pumps

In another aspect, implantable pumps that include an anti-scarring agent are provided that can be used to deliver drugs to a desired location. Implantable drug delivery devices and pumps are a means to provide prolonged, site-specific release of a therapeutic agent for the management of a variety of medical conditions. Drug delivery implants and pumps are generally utilized when a localized pharmaceutical impact is desired (i.e., the condition affects only a specific region) or when systemic delivery of the agent is inefficient or ineffective (i.e., leads to toxicity or severe side effects, results in inactivation of the drug prior to reaching the target tissue, produces poor symptom/disease control, and/or leads to addiction to the medication). Implantable pumps can also deliver systemic drug levels in a constant, regulated manner for extended periods and help patients avoid the “peaks and valleys” of blood-level drug concentrations associated with intermittent systemic dosing. Another advantage of implantable pumps is improved patient compliance. Many patients forget to take their medications regularly (particularly the young, elderly, chronically ill, mentally handicapped), but with an implantable pump, this problem is alleviated. For many patients this can lead to better symptom control (the dosage can often be titrated to the severity of the symptoms), superior disease management (particularly for insulin delivery in diabetics), and lower drug requirements (particularly for pain medications).

Innumerable drug delivery implants and pumps have been used in a variety of clinical applications, including programmable insulin pumps for the treatment of diabetes, intrathecal (in the spine) pumps to administer narcotics (e.g., morphine, fentanyl) for the relief of pain (e.g., cancer, back problems, HIV, post-surgery), local and systemic delivery of chemotherapy for the treatment of cancer (e.g., hepatic artery 5-FU infusion for liver tumors), medications for the treatment of cardiac conditions (e.g., anti-arrhythmic drugs for cardiac rhythm abnormalities), intrathecal delivery of anti-spasmotic drugs (e.g., baclofen) for spasticity in neurological disorders (e.g., Multiple Sclerosis, spinal cord injuries, brain injury, cerebral palsy), or local/regional antibiotics for infection management (e.g., osteomyelitis, septic arthritis). Typically, drug delivery pumps are implanted subcutaneously and consist of a pump unit with a drug reservoir and a flexible catheter through which the drug is delivered to the target tissue. The pump stores and releases prescribed amounts of medication via the catheter to achieve therapeutic drug levels either locally or systemically (depending upon the application). The center of the pump has a self-sealing access port covered by a septum such that a needle can be inserted percutaneously (through both the skin and the septum) to refill the pump with medication as required. There are generally two types of implantable drug delivery pumps. Constant-rate pumps are usually powered by gas and are designed to dispense drugs under pressure as a continual dosage at a preprogrammed, constant rate. The amount and rate of drug flow and regulated by the length of the catheter used, temperature, and altitude and they are best when unchanging, long-term drug delivery is required. Programmable-rate pumps utilize a battery-powered pump and a constant pressure reservoir to deliver drugs on a periodic basis in a manner that can be programmed by the physician or the patient. For the programmable infusion device, the drug may be delivered in small, discrete doses based on a programmed regimen which can be altered according to an individual's clinical response.

In general, drug delivery pumps are implanted to deliver drug at a regulated dose and may, in certain applications, be used in conjunction with implantable sensors that collect information which is used to regulate drug delivery (often called a “closed loop” system). Implantable drug delivery pumps may function and deliver drug in a variety of ways, which include, but are not limited to: (a) delivering drugs only when changes in the body are detected (e.g., sensor stimulated); (b) delivering drugs as a continuous slow release (e.g., constant flow); (c) delivering drugs at prescribed dosages in a pulsatile manner (e.g., non-constant flow); (d) delivering drugs by programmable means; and (e) delivering drugs through a device that is designed for a specific anatomical site (e.g., intraocular, intrathecal, intraperitoneal, intra-arterial or intracardiac). In addition to delivering drugs in a specific way or to a specific location, drug delivery pumps may also be categorized based on their mechanical delivery technology (e.g., the driving force by which drug delivery occurs). For example, the mechanics for delivering drugs may include, without limitation, osmotic pumps, metering systems, peristaltic (roller) pumps, electronically driven pumps, ocular drug delivery pumps and implants, elastomeric pumps, spring-contraction pumps, gas-driven pumps (e.g., induced by electrolytic cell or chemical reaction), hydraulic pumps, piston-dependent pumps and non-piston-dependent pumps, dispensing chambers, infusion pumps, passive pumps, infusate pumps and osmotically-driven fluid dispensers.

The clinical function of an implantable drug delivery device or pump depends upon the device, particularly the catheter or drug-dispensing component(s), being able to effectively maintain intimate anatomical contact with the target tissue (e.g., the sudural space in the spinal cord, the arterial lumen, the peritoneum, the interstitial fluid) and not becoming encapsulated or obstructed by scar tissue. Unfortunately, in many instances when these devices are implanted in the body, they are subject to a “foreign body” response from the surrounding host tissues as described previously. For implantable pumps, the drug-delivery catheter lumen, catheter tip, dispensing components, or delivery membrane may become obstructed by scar tissue which may cause the flow of drug to slowdown or cease completely. Alternatively, the entire pump, the catheter and/or the dispensing components can become encapsulated by scar (i.e., the body “walls off” the device with fibrous tissue) so that the drug is incompletely delivered to the target tissue (i.e., the scar prevents proper drug movement and distribution from the implantable pump to the tissues on the other side of the capsule). Either of these developments may lead to inefficient or incomplete drug flow to the desired target tissues or organs (and loss of clinical benefit), while encapsulation can also lead to local drug accumulation (in the capsule) and additional clinical complications (e.g., local drug toxicity; drug sequestration followed by sudden “dumping” of large amounts of drug into the surrounding tissues). Additionally, the tissue surrounding the implantable pump can be inadvertently damaged from the inflammatory foreign body response leading to loss of function and/or tissue damage (e.g., scar tissue in the spinal canal causing pain or obstructing the flow of cerebrospinal fluid).

Implantable drug delivery pumps that release one or more therapeutic agents for reducing scarring at the device-tissue interface (particularly in and around the drug delivery catheter or drug dispensing components) may help prolong the clinical performance of these devices. Inhibition of fibrosis can make sure that the correct amount of drug is dispensed from the device at the appropriate rate and that potentially toxic drugs do not become sequestered in a fibrous capsule. For devices that include electrical or battery components, not only can fibrosis cause the device to function suboptimally or not at all, it can cause excessive drain on battery life as increased energy is required to overcome the increased resistance imposed by the intervening scar tissue.

Virtually any implantable pump may benefit from the present invention. In one aspect, the drug delivery pump may deliver drugs in a continuous, constant-flow, slow release manner. For example, the drug delivery pump may be a passive pump adapted to provide a constant flow of medication which may be regulated by a pressure sensing chamber and a valve chamber in which the constant flow rate may be changed to a new constant flow rate. See, e.g., U.S. Pat. No. 6,589,205. In another aspect, the drug delivery pump may deliver drugs at prescribed dosages in a non-constant flow or pulsatile manner. For example, the drug delivery pump may adapt a regular pump to generate a pulsatile fluid drug flow by continuously filling a chamber and then releasing a valve to provide a bolus pulse of the drug. See, e.g., U.S. Pat. No. 6,312,409. In another aspect, the drug delivery pump may be programmed to dispense drug in a very specific manner. For example, the drug delivery pump may be a programmable infusate pump composed of a variable volume infusate chamber, and variable volume control fluid pressure and displacement reservoirs, whereby a fluid flow is sampled by a microprocessor based on the programmed value and adjustments are made accordingly to maintain the programmed fluid flow. See, e.g., U.S. Pat. No. 4,443,218.

In another aspect, the drug delivery pump suitable for use in the present invention may be manufactured based on different mechanical technologies (e.g., driving forces) of delivering drugs. For example, the drug delivery pump may be an implant composed of a piston that divides two chambers in which one chamber contains a water-swellable agent and the other chamber contains a leuprolide formulation for delivery. See, e.g., U.S. Pat. No. 5,728,396. The drug delivery pump may be a non-cylindrical osmotic pump system that may not rely upon a piston to infuse drug and conforms to the anatomical implant site. See, e.g., U.S. Pat. No. 6,464,688. The drug delivery pump may be an osmotically driven fluid dispenser composed of a flexible inner bag that contains the drug composition and a port in which the composition can be delivered. See, e.g., U.S. Pat. No. 3,987,790. The drug delivery pump may be a fluid-imbibing delivery implant composed of a compartment with a composition permeable to the passage of fluid and has an extended rigid sleeve to resist transient mechanical forces. See, e.g., U.S. Pat. Nos. 5,234,692 and 5,234,693. The drug delivery pump may be a pump with an isolated hydraulic reservoir, metering device, displacement reservoir, drug reservoir, and drug infusion port that is all contained in a housing apparatus. See, e.g., U.S. Pat. No. 6,629,954. The drug delivery pump may be composed of a dispensing chamber that has a dispensing passage and valves that are under compressive force to enable drug to flow in a one-way direction. See, e.g., U.S. Pat. No. 6,283,949. The drug delivery pump may be spring-driven based on a spring regulating pressure difference with a variable volume drug chamber. See, e.g., U.S. Pat. No. 4,772,263. Other examples of drug delivery pumps are described in, e.g., U.S. Pat. Nos. 6,645,176; 6,471,688; 6,283,949; 5,137,727 and 5,112,614.

In addition, there are osmotically driven drug delivery pumps that are commercially available and suitable for the practice of the invention. These osmotic pumps include the DUROS Implant and ALZET Osmotic Pump from Alza Corporation (Mountain View, Calif.), which are used to delivery a wide variety of drugs and other therapeutics through the method of osmosis (see, e.g., U.S. Pat. Nos. 6,283,953; 6,270,787; 5,660,847; 5,112,614; 5,030,216 and 4,976,966).

As described above, the drug delivery pump can be combined with an agent that inhibits fibrosis to improve performance of the device. Fibrosis-inhibiting agents can also be incorporated into, and released from, the materials that are used to construct the device (e.g., the polymers that make up the delivery catheters, the semipermeable membranes etc.). Alternatively, or in addition, the fibrosis-inhibiting agent can be infiltrated into the region around the device-tissue interface. It may be obvious to one of skill in the art that commercial drug delivery pumps not specifically cited as well as next-generation and/or subsequently-developed commercial drug delivery products are to be anticipated and are suitable for use under the present invention.

Several specific drug delivery pumps and treatments will be described in greater detail including:

a. Implantable Insulin Pumps for Diabetes

In one aspect, the drug delivery pump may be an insulin pump. Insulin pumps are used for patients with diabetes to replace the need to control blood glucose levels by daily manual injections of insulin. Precise titration of the dosage and timing of insulin administration is a critical component in the effective management of diabetes. If the insulin dosage is too high, blood glucose levels drop precipitously, resulting in confusion and potentially even loss of consciousness. If insulin dosage is too low, blood glucose levels rise too high, leading to excessive thirst, urination, and changes in metabolism known as ketoacidosis. If the timing of insulin administration is incorrect, blood glucose levels can fluctuate wildly between the two extremes—a situation that is thought to contribute to some of the long-term complications of diabetes such as heart disease, kidney failure, nerve damage and blindness. Since in the extreme, all these conditions can be life threatening, the precise dosing and timing of insulin administration is essential to preventing the short and long-term complications of diabetes.

Implantable pumps automate the administration of insulin and eliminate human errors of dosage and timing that can have long-term health consequences. The pump has the capability to inject insulin regularly, multiple times a day and in small doses into the blood stream, peritoneal cavity or subcutaneous tissue. The pump is refilled with insulin once or twice a month by injection directly into the pump chamber. This reduces the number of externally administered injections the patient must undergo and also allows preprogrammed variable amounts of insulin to be released at different times into the blood stream; a situation which more closely resembles normal pancreas function and minimizes fluctuations in blood glucose levels. The insulin pump may be activated by an externally generated signal after the patient has withdrawn a drop of blood, subjected it to an analysis, and made a determination of the amount of insulin that needs to be delivered. However, the most widely pursued application of this technology is the production of a closed-loop “artificial pancreas” which can continuously detect blood glucose levels (through an implanted sensor) and provide feedback to an implantable pump to modulate the administration of insulin to a diabetic patient.

Numerous types of insulin pumps are suitable for use in the practice of the invention. For example, the drug delivery pump may include both an implantable sensor and a drug delivery pump by being composed of a mass of living cells and an electrical signal that regulates the delivery of glucose or glucagon or insulin. See, e.g., U.S. Pat. No. 5,474,552. The drug delivery pump may be composed of a single channel catheter with a sensor which is implanted in a vessel that transmits blood chemistry to a subcutaneously implanted infusion device which then dispenses medication through the catheter. See, e.g., U.S. Pat. No. 5,109,850.

Commercially available insulin pump devices suitable for the practice of the invention include the MINIMED 2007 Implantable Insulin Pump System from Medtronic MiniMed, Inc. (Northridge, Calif.). The MINIMED pump delivers insulin into the peritoneal cavity in short, frequent bursts to provide insulin to the body similar to that of the normal pancreas (see, e.g., U.S. Pat. Nos. 6,558,345 and 6,461,331). The MINIMED 2001 Implantable Insulin Pump System (Medtronic MiniMed Inc., Northridge, Calif.) delivers intraperitoneal insulin injections in a pulsatile manner from a negative pressure reservoir. Both these devices feature a long catheter that transports insulin from the subcutaneously implanted pump into the peritoneal cavity. As described above, the peritoneal drug-delivery catheter lumen or catheter tip may become partially or fully obstructed by scar tissue which may cause the flow of drug to slowdown or cease completely. In the present invention, the insulin delivery catheter can be combined with an agent that inhibits fibrosis to keep the delivery catheter lumen patent. Fibrosis-inhibiting agents can also be incorporated into, and released from, the materials that are used to construct the delivery catheters. Alternatively, or in addition, the fibrosis-inhibiting agent may be infiltrated into the region around the device-tissue interface.

It may be obvious to one of skill in the art that commercial drug delivery pumps not specifically cited as well as next-generation and/or subsequently-developed commercial drug delivery products are to be anticipated and are suitable for use under the present invention.

b. Intrathecal Drug Delivery Pumps

In another aspect, intrathecal drug delivery pumps combined with a fibrosis-inhibitor can be used to may used to deliver drugs into the spinal cord for pain management and movement disorders.

Chronic pain is one of the most important clinical problems in all of medicine. For example, it is estimated that over 5 million people in the United States are disabled by back pain. The economic cost of chronic back pain is enormous, resulting in over 100 million lost work days annually at an estimated cost of $50-100 billion. The cost of managing pain for oncology patients is thought to approach $12 billion. Chronic pain disables more people than cancer or heart disease and costs the American public more than both cancer and heart disease combined. In addition to the physical consequences, chronic pain has numerous other costs including loss of employment, marital discord, depression and prescription drug addiction. It goes without saying, therefore, that reducing the morbidity and costs associated with persistent pain remains a significant challenge for the healthcare system.

Intractable severe pain resulting from injury, illness, scoliosis, spinal disc degeneration, spinal cord injury, malignancy, arachnoiditis, chronic disease, pain syndromes (e.g., failed back syndrome, complex regional pain syndrome) and other causes is a debilitating and common medical problem. In many patients, the continued use of analgesics, particularly drugs like narcotics, are not a viable solution due to tolerance, loss of effectiveness, and addiction potential. In an effort to combat this, intrathecal drug delivery devices have been developed to treat severe intractable back pain that is resistant to other traditional treatment modalities such as drug therapy, invasive therapy (surgery), or behavioral/lifestyle changes.

Intrathecal drug delivery pumps are designed and used to reduce pain by delivering pain medication directly into the cerebrospinal fluid of the intrathecal space surrounding the spinal cord. Typically, since this therapy delivers pain medication topically to pain receptors contained in the spinal cord that transmit pain sensation directly to the brain, smaller doses of medication are needed to gain relief. Morphine and other narcotics (usually fentanyl and sufentanil) are the most commonly delivered agents and many patients receive superior relief with lower doses than can be achieved with systemic delivery. Intrathecal drug delivery also allows the administration of pain medications (such as Ziconotide; an N-type calcium channel blocker made by Elan Pharmaceuticals) that cannot cross the blood-brain barrier and are thus only effective when administered by this route.

Intrathecal pumps are also used in the management of neurological and movement disorders. Baclofen (marketed as Lioresal by Novartis) is an antispasmotic/muscle relaxant used to treat spasticity and improve mobility in patients with Multiple Sclerosis, cystic fibrosis and spinal injuries. This drug has been proven to be more effective and cause fewer side effects when administered into the CSF by an intrathecal drug delivery pump. Efforts are also underway to treat epilepsy, brain tumors, Alzheimer's disease, Parkinson's disease and Amyetropic Lateral Sclerosis (ALS—Lou Gehrig's disease) via intrathecal administration of agents that may be too toxic to deliver systemically or do not cross the blood-brain barrier. For example, trials of intrathecally administered recombinant brain-derived neurotrophic factor (r-BDNF made by Amgen) have been undertaken in ALS patients.

An intrathecal drug delivery system consists of an intrathecal drug infusion pump and an intraspinal catheter, both of which are fully implanted. The pump device is implanted under the skin in the abdominal area, just above or below the beltline and can be refilled by percutaneous injection of the drug into the reservoir. The catheter is tunneled under the skin and runs from the pump to the intrathecal space of the spine. When operational, the pump administers prescribed amounts of medication to the cerebrospinal fluid in either a continuous fashion or in a manner than can be controlled by the physician or the patient in response to symptoms.

Numerous types of implantable intrathecal pumps are suitable for use in combination with a fibrosis-inhibiting agent in the practice of the invention. For example, the implantable pump used to deliver medication may be composed of two osmotic pumps with semipermeable membranes configured to deliver up to two drug delivery regimens at different rates, and having a built-in backup drug delivery system whereby the delivery of drug may continue when the primary delivery system reaches the end of its useful life or fails unexpectedly. See, e.g., U.S. Pat. No. 6,471,688. The implantable pump may be may be composed of a battery-operated pump unit with a drug reservoir, catheter, and electrodes that are implanted in the epidural space of a patient for relief of pain by delivering a liquid pain-relieving agent through the catheter to the desired location. See, e.g., U.S. Pat. No. 5,458,631.

Similar drug-delivery pumps have been described for the infusion of agents into regions of the brain to locally affect the excitability of the neurons in the treatment of a variety of chronic neurogenerative diseases (such as those described above for intrathecal delivery). Implantable pumps may be implanted abdominally which then dispenses drug through a catheter that is tunneled from the abdominal implant site, through the neck to an entry site in the head, and then to the localized treatment site within the brain. Pumps that deliver drug to the brain may discharge the drug at a variety of locations, including, but not limited to, anterior thalamus, ventrolateral thalamus, internal segment of the globus pallidus, substantia nigra pars reticulate, subthalamic nucleus, external segment of globus pallidus, and neostriatum. For example, the drug delivery pump may be composed of an implantable pump portion coupled to a catheter for infusing dosages of drug to a predetermined location of the brain when a sensor detects a symptom, such that a neurological disorder (e.g., seizure) may be treated. See, e.g., U.S. Pat. No. 5,978,702. The implantable pump may be implanted adjacent to a predetermined infusion site in a brain such that a predetermined dosage of at least one drug capable of altering the level of excitation of neurons of the brain may be infused such that neurodegeneration is prevented and/or treated. See, e.g., U.S. Pat. No. 5,735,814. The implantable pump may include a reservoir for the therapeutic agent which is stored between the galea aponeurotica and cranium of a subject whereby drug is then dispensed via pumping action to the desired location. See, e.g., U.S. Pat. No. 6,726,678.

There are numerous commercially available implantable, intrathecal drug-delivery systems which are suitable for the practice of the invention. The SYNCHROMED EL Infusion System which is made by Medtronic, Inc. and is indicated for chronic Intrathecal Baclofen Therapy (ITB Therapy) (see, e.g., U.S. Pat. Nos. 6,743,204; 6,669,663; 6,635,048; 6,629,954; 6,626,867; 6,102,678; 5,978,702 and 5,820,589) The SYNCHROMED pump is a programmable, battery-operated device that stores and delivers medication based on the programmed dosing regimen. Medtronic, Inc. (Minneapolis, Minn.) also sells their ISOMED Constant-Flow Infusion System for use in delivering morphine sulfate directly into the intrathecal space as a treatment for chronic pain. Arrow International produces the Model 3000 infusion pump that provides constant-rate administration of agents such as morphine and baclofen into the intrathecal space. Tricumed Medizintechnik GmbH (Kiel, Germany) produces the Archimedes® constant flow implantable infusion pump for intrathecal administration of pain and antispasmotic drugs. Advanced Neuromodulation Systems (Piano, Tex.) produces the AccuRx® infusion pump for the treatment of pain and neuromuscular disorders. All these devices feature a long catheter that transports the active agent from a subcutaneously implanted pump into the intrathecal space in the spinal cord. As described above, the intrathecal drug-delivery catheter lumen or catheter tip may become partially or fully obstructed by scar tissue which may cause the flow of drug to slowdown or cease completely. Another potential complication with intrathecal drug delivery is the formation of fibrous tissue in the subdural space that can obstruct CSF flow and lead to serious complications (e.g., hydrocephalus, increased intracranial pressure). In the present invention, the drug delivery catheter can be combined with an agent that inhibits fibrosis to keep the delivery catheter lumen patent and/or prevents fibrosis in the surrounding tissue. Fibrosis-inhibiting agents can also be incorporated into, and released from, the materials that are used to construct the delivery catheters. Alternatively, or in addition, the fibrosis-inhibiting agent may be infiltrated into the region around the device-tissue interface. The adjuvant use of an anti-infective agent as a catheter coating and/or implant, with or without a fibrosis-inhibiting agent, may also be beneficial in the practice of this invention.

It may be obvious to one of skill in the art that commercial intrathecal drug delivery pumps not specifically cited as well as next-generation and/or subsequently-developed commercial drug delivery products are to be anticipated and are suitable for use under the present invention.

c. Implantable Drug Delivery Pumps for Chemotherapy

In another aspect, the drug delivery pump may be a pump that dispenses a chemotherapeutic drug for the treatment of cancer. Pumps for dispensing a drug for the treatment of cancer are used to deliver chemotherapeutic agents to a local area of the body. Although virtually any malignancy may potentially be treated in this manner (i.e., by infusing drug directly into a solid tumor or into the blood vessels that supply the tumor), current treatments revolve around the management of hepatic (liver) tumors. For example, FUDR (2′-deoxy 5-fluorouridine) is used in the palliative management of adenocarcinoma (Colon, breast, stomach) that has metastasized to the liver. In hepatic artery infusion therapy the drug is delivered via an implantable pump into the artery which provides blood supply to the liver. This allows for higher drug concentrations to reach the liver (the drug is not diluted in the blood as may occur in intravenous administration) and prevents clearance by the liver (the drug is metabolized by the liver and may be rapidly cleared from the bloodstream if administered i.v.); both of which allow higher concentrations of the drug to reach the tumor.

Numerous types of implantable pumps are suitable for delivering chemotherapeutic agents in the practice of the invention. For example, the implantable pump may have a dispensing chamber with a dispensing passage and actuator, reservoir housing with reservoir, and septum for refilling the reservoir. See, e.g., U.S. Pat. No. 6,283,949. Medtronic, Inc. sells their ISOMED Constant-Flow Infusion System which may be used to deliver chronic intravascular infusion of floxuridine in a fixed flow rate for the treatment of primary or metastatic cancer. Tricumed Medizintechnik GmbH (Kiel, Germany) sells their ARCHIMEDES DC implantable infusion pump specially adapted to deliver chemotherapy in a constant flow rate within the vicinity of a tumor (see, e.g., U.S. Pat. Nos. 5,908,414 and 5,769,823). Arrow International produces the Model 3000 infusion pump that provides constant-rate administration of chemotherapeutic agents into a tumor. All these devices feature a catheter that transports the chemotherapeutic agent from a subcutaneously implanted pump directly into the tumor or the artery that supplies a tumor. As described above, the drug-delivery catheter lumen or catheter tip may become partially or fully obstructed by scar tissue which may cause the flow of drug to slowdown or cease completely. If placed intravascularly, the drug-delivery catheter lumen or catheter tip may become partially or fully obstructed by neointimal tissue which may impair the flow of drug into the blood vessel. In the present invention, the drug delivery catheter can be combined with an agent that inhibits fibrosis to keep the delivery catheter lumen patent. Fibrosis-inhibiting agents can also be incorporated into, and released from, the materials that are used to construct the delivery catheters. Alternatively, or in addition, the fibrosis-inhibiting agent may be infiltrated into the region around the device-tissue interface. The adjuvant use of an anti-infective agent as a catheter coating and/or implant, with or without a fibrosis-inhibiting agent, may also be beneficial in the practice of this invention.

It may be obvious to one of skill in the art that commercial chemotherapy delivery pumps and implants not specifically cited as well as next-generation and/or subsequently-developed commercial chemotherapy delivery products are to be anticipated and are suitable for use in the present invention.

d. Drug Delivery Pumps for the Treatment of Heart Disease

In another aspect, the drug delivery pump may be a pump that dispenses a drug for the treatment of heart disease. Pumps for dispensing a drug for the treatment of heart disease may be used to treat conditions including, but not limited to atrial fibrillation and other cardiac rhythm disorders. Atrial fibrillation is a form of heart disease that afflicts millions of people. It is a condition in which the normal coordinated contraction of the heart is disrupted, primarily by abnormal and uncontrolled action of the atria of the heart. Normally, contractions occur in a controlled sequence with the contractions of the other chambers of the heart. When the right atrium fails to contract, contracts out of sequence, or contracts ineffectively, blood flow from the atria to the ventricles is disrupted. Atrial fibrillation can cause weakness, shortness of breath, angina, lightheadedness and other symptoms due to reduced ventricular filling and reduced cardiac output. Stroke can occur as a result of clot forming in a poorly contracting atria, breaking loose, and traveling via the bloodstream to the arteries of the brain where they become wedged and obstruct blood flow (which may lead to brain damage and death). Typically, atrial fibrillation is treated by medical or electrical conversion (defibrillation), however, complications may exist whereby the therapy causes substantial pain or has the potential to initiate a life threatening ventricular arrhythmia. The pain associated with the electrical shock is severe and unacceptable for many patients, since they are conscious and alert when the device delivers electrical therapy. Medical therapy involves the delivery of anti-arrhythmic drugs by injecting them intravenously, administering them orally or delivering them locally via a drug delivery pump.

Numerous types of implantable pumps are described for dispensing a drug for the treatment of heart disease and are suitable for use in the practice of the invention. For example, the drug delivery pump may be an implantable cardiac electrode which delivers stimulation energy and dispenses drug adjacent to the stimulation site. See, e.g., U.S. Pat. No. 5,496,360. The drug delivery pump may have a plurality of silicone septii to facilitate the filling of drug reservoirs within the pump which is subcutaneously implanted with a catheter which travels transvenously by way of the subclavian vein through the superior vena cava and into the right atrium for drug delivery. See, e.g., U.S. Pat. No. 6,296,630. As described above, the drug-delivery catheter lumen or catheter tip may become partially or fully obstructed by scar tissue which may cause the flow of drug to slowdown or cease completely. If placed intravascularly, the drug-delivery catheter lumen or catheter tip may become partially or fully obstructed by neointimal tissue which may impair the flow of drug into the blood vessel or the right atrium. In the present invention, the drug delivery catheter can be combined with an agent that inhibits fibrosis to keep the delivery catheter lumen patent. Fibrosis-inhibiting agents can also be incorporated into, and released from, the materials that are used to construct the delivery catheters. Alternatively, or in addition, the fibrosis-inhibiting agent may be infiltrated into the region around the device-tissue interface. The adjuvant use of an anti-infective agent as a catheter coating and/or implant, with or without a fibrosis-inhibiting agent, may also be beneficial in the practice of this invention.

It may be obvious to one of skill in the art that commercial cardiac drug delivery pumps not specifically cited as well as next-generation and/or subsequently-developed commercial cardiac drug delivery products are to be anticipated and are suitable for use under the present invention.

e. Other Drug Delivery Implants

Several other implantable pumps have been developed for continuous delivery of pharmaceutical agents.

For example, Debiotech S. A. (Switzerland) has developed the MIP device which is an implantable piezo-actuated silicon micropump for programmable drug delivery applications. This high-performance micropump is based on a MEMS (Micro-Electro-Mechanical) system which allows it to maintain a low flow rate. The DUROS sufentanil implant from Durect Corporation (Cupertino, Calif.) is a titanium cylinder that contains a drug reservoir, and a piston driven by an osmotic engine. The VIADUR (leuprolide acetate) implant available from Alza Corporation (Mountain View, Calif.) uses the same DUROS implant technology to deliver leuprolide over a 12 month period to reduces testosterone levels for the treatment prostate cancer (see, e.g., U.S. Pat. Nos. 6,283,953; 6,270,787; 5,660,847; 5,112,614; 5,030,216 and 4,976,966). Fibrous encapsulation of the device can cause failure in a number of ways including: obstructing the semipermeable membrane (which will impair functioning of the osmotic engine by preventing the flow of fluids into the engine), obstructing the exit port (which will impair drug flow out of the device) and/or complete encapsulation (which will create a microenvironment that prevents drug distribution). Many other drug delivery implants, osmotic pumps and the like suffer from similar problems—fibrous encapsulation prevents the appropriate release of drugs into the surrounding tissues. In the present invention, the drug delivery implant can be combined with an agent that inhibits fibrosis to prevent encapsulation, prevent obstruction of the semipermeable membrane and/or to keep the delivery port patent. Fibrosis-inhibiting agents can also be incorporated into, and released from, the materials that are used to construct the drug delivery implant. Alternatively, or in addition, the fibrosis-inhibiting agent may be infiltrated into the tissue around the drug delivery implant.

Although numerous implantable pumps have been described above, all possess similar design features and cause similar unwanted fibrous tissue reactions following implantation. The clinical function of an implantable drug delivery device or pump depends upon the device, particularly the catheter or drug-dispensing component(s), being able to effectively maintain intimate anatomical contact with the target tissue (e.g., the sudural space in the spinal cord, the arterial lumen, the peritoneum, the interstitial fluid) and not becoming encapsulated or obstructed by scar tissue. For implantable pumps, the drug-delivery catheter lumen, catheter tip, dispensing components, or delivery membrane may become obstructed by scar tissue which may cause the flow of drug to slowdown or cease completely. Alternatively, the entire pump, the catheter and/or the dispensing components can become encapsulated by scar (i.e., the body “walls off” the device with fibrous tissue) so that the drug is incompletely delivered to the target tissue (i.e., the scar prevents proper drug movement and distribution from the implantable pump to the tissues on the other side of the capsule). Either of these developments may lead to inefficient or incomplete drug flow to the desired target tissues or organs (and loss of clinical benefit), while encapsulation can also lead to local drug accumulation (in the capsule) and additional clinical complications (e.g., local drug toxicity; drug sequestration followed by sudden “dumping” of large amounts of drug into the surrounding tissues). For implantable pumps that include electrical or battery components, not only can fibrosis cause the device to function suboptimally or not at all, it can cause excessive drain on battery life as increased energy is required to overcome the increased resistance imposed by the intervening scar tissue.

Implantable pumps that release a therapeutic agent for reducing scarring at the device-tissue interface can be used to increase efficacy, prolong clinical performance, ensure that the correct amount of drug is dispensed from the device at the appropriate rate, and reduce the risk that potentially toxic drugs become sequestered in a fibrous capsule. In one aspect, the present invention provides implantable pumps that include a fibrosis-inhibiting agent or a composition that includes a fibrosis-inhibiting agent. Numerous polymeric and non-polymeric delivery systems for use in implantable pumps have been described above. These compositions can further include one or more fibrosis-inhibiting agents such that the overgrowth of granulation or fibrous tissue is inhibited or reduced.

Methods for incorporating fibrosis-inhibiting compositions onto or into implantable drug delivery pumps to reduce scarring at the device-tissue interface (particularly in and around the drug delivery catheter or drug dispensing components) include: (a) directly affixing to the implantable pump, catheter and/or drug dispensing components a fibrosis-inhibiting composition (e.g., by either a spraying process or dipping process as described below, with or without a carrier), (b) directly incorporating into the implantable pump, catheter and/or drug dispensing components a fibrosis-inhibiting composition (e.g., by either a spraying process or dipping process as described below, with or without a carrier (c) by coating the implantable pump, catheter and/or drug dispensing components with a substance such as a hydrogel which will in turn absorb the fibrosis-inhibiting composition, (d) by interweaving fibrosis-inhibiting composition coated thread (or the polymer itself formed into a thread) into the implantable pump, catheter and/or drug dispensing component structure, (e) by inserting the implantable pump, catheter and/or drug dispensing components into a sleeve or mesh which is comprised of, or coated with, a fibrosis-inhibiting composition, (f) constructing the implantable pump itself (or all, or a portion of the catheter and/or drug dispensing components) from a fibrosis-inhibiting composition, or (g) by covalently binding the fibrosis-inhibiting agent directly to the implantable pump, catheter and/or drug dispensing component surface, or to a linker (small molecule or polymer) that is coated or attached to the device surface. Each of these methods illustrates an approach for combining an implantable pump with a fibrosis-inhibiting (also referred to herein as anti-scarring) agent according to the present invention.

For implantable pump, the coating process can be performed in such a manner as to: (a) coat a portion of the device (such as the catheter, drug delivery port, semipermeable membrane); or (b) coat the entire device with the fibrosis-inhibiting composition. In addition to, or alternatively, the fibrosis-inhibiting agent can be mixed with the materials that are used to make the implantable pump such that the fibrosis-inhibiting agent is incorporated into the final product. In these manners, a medical device may be prepared which has a coating, where the coating is, e.g., uniform, non-uniform, continuous, discontinuous, or patterned.

In another aspect, an implantable drug delivery pump device may include a plurality of reservoirs within its structure, each reservoir configured to house and protect a therapeutic drug (i.e., one or more fibrosis-inhibiting agents). The reservoirs may be formed from divets in the device surface or micropores or channels in the device body. In one aspect, the reservoirs are formed from voids in the structure of the device. The reservoirs may house a single type of drug (e.g., fibrosis-inhibiting agent) or more than one type of drug (e.g., a fibrosis-inhibiting agent and an anti-infective agent). The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount and type of drug that is released from the substrate. The multi-layered carrier may further include a barrier layer that prevents release of the drug(s). The barrier layer can be used, for example, to control the direction that the drug elutes from the void. Thus, the coating of the medical device may directly contact the pump, or it may indirectly contact the pump when there is something, e.g., a polymer layer, that is interposed between the pump and the coating that contains the fibrosis-inhibiting agent.

In addition to (or as an alternative to) incorporating a fibrosis-inhibiting agent onto, or into, the implantable pump, catheter and/or drug dispensing components, the fibrosis-inhibiting agent can be applied directly or indirectly to the tissue adjacent to the implantable pump (preferably near in the tissue adjacent to where the drug is delivered from the device). This can be accomplished by applying the fibrosis-inhibiting agent, with or without a polymeric, non-polymeric, or secondary carrier: (a) to the implantable pump, catheter and/or drug dispensing component surface (e.g., as an injectable, paste, gel, or meSH) during the implantation procedure; (b) to the surface of the tissue (e.g., as an injectable, paste, gel, in situ forming gel, or meSH) prior to, immediately prior to, or during, implantation of the implantable pump, catheter and/or drug dispensing components; (c) to the surface of the implantable pump, catheter and/or drug dispensing components and/or to the tissue surrounding the implanted pump, catheter and/or drug dispensing components (e.g., as an injectable, paste, gel, in situ forming gel, or meSH) immediately after implantation; (d) by topical application of the anti-fibrosis agent into the anatomical space where the implantable pump, catheter and/or drug dispensing components will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the implantable pump, catheter and/or drug dispensing components will be inserted); (e) via percutaneous injection into the tissue surrounding the implantable pump, catheter and/or drug dispensing components as a solution, as an infusate, or as a sustained release preparation; (f) by any combination of the aforementioned methods. Combination therapies (i.e., combinations of therapeutic agents and combinations with antithrombotic, antiplatelet, and/or anti-infective agents) can also be used.

It may be noted that certain polymeric carriers themselves can help prevent the formation of fibrous tissue around the implanted pump, catheter and/or drug dispensing components. These carriers (described below) are particularly useful for the practice of this embodiment, either alone, or in combination with a fibrosis-inhibiting composition. The following polymeric carriers can be infiltrated (as described in the previous paragraph) into the vicinity of the interface between the implanted pump, catheter and/or drug dispensing components of the device and the tissue and include: (a) sprayable collagen-containing formulations such as COSTASIS and CT3, either alone, or loaded with a fibrosis-inhibiting agent, applied to the implantation site (or the pump, catheter and/or drug dispensing component surface); (b) sprayable PEG-containing formulations such as COSEAL, FOCALSEAL, SPRAYGEL or DURASEAL, either alone, or loaded with a fibrosis-inhibiting agent, applied to the implantation site (or the pump, catheter and/or drug dispensing component surface); (c) fibrinogen-containing formulations such as FLOSEAL or TISSEAL, either alone, or loaded with a fibrosis-inhibiting agent, applied to the implantation site (or the pump, catheter and/or drug dispensing component surface); (d) hyaluronic acid-containing formulations such as RESTYLANE, HYLAFORM, PERLANE, SYNVISC, SEPRAFILM, SEPRACOAT, loaded with a fibrosis-inhibiting agent applied to the implantation site (or the pump, catheter and/or drug dispensing component surface); (e) polymeric gels for surgical implantation such as REPEL or FLOWGEL loaded with a fibrosis-inhibiting agent applied to the implantation site (or the pump, catheter and/or drug dispensing component surface); (f) orthopedic “cements” used to hold prostheses and tissues in place loaded with a fibrosis-inhibiting agent applied to the implantation site (or the pump, catheter and/or drug dispensing component surface), such as OSTEOBOND, low viscosity cement (LVC), SIMPLEX P, PALACOS, and ENDURANCE; (g) surgical adhesives containing cyanoacrylates such as DERMABOND, INDERMIL, GLUSTITCH, TISSUMEND, VETBOND, HISTOACRYL BLUE and ORABASE SOOTHE-N-SEAL LIQUID PROTECTANT, either alone, or loaded with a fibrosis-inhibiting agent, applied to the implantation site (or the pump, catheter and/or drug dispensing component surface); (h) implants containing hydroxyapatite (or synthetic bone material such as calcium sulfate, VITOSS and CORTOSS) loaded with a fibrosis-inhibiting agent applied to the implantation site (or the pump, catheter and/or drug dispensing component surface); (i) other biocompatible tissue fillers loaded with a fibrosis-inhibiting agent, such as those made by BioCure, Inc., 3M Company and Neomend, Inc., applied to the implantation site (or the pump, catheter and/or drug dispensing component surface); (j) polysaccharide gels such as the ADCON series of gels either alone, or loaded with a fibrosis-inhibiting agent, applied to the implantation site (or the pump, catheter and/or drug dispensing component surface); and/or (k) films, sponges or meshes such as INTERCEED, VICRYL mesh, and GELFOAM loaded with a fibrosis-inhibiting agent applied to the implantation site (or the pump, catheter and/or drug dispensing component surface).

A preferred polymeric matrix which can be used to help prevent the formation of fibrous tissue around the implanted pump, catheter and/or drug dispensing components, either alone or in combination with a fibrosis inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue around the implanted pump, catheter and/or drug dispensing components.

It may be apparent to one of skill in the art that potentially any anti-scarring agent described below may be utilized alone, or in combination, in the practice of this embodiment. As implantable pumps and their drug delivery mechanisms (e.g., catheters, ports etc.) are made in a variety of configurations and sizes, the exact dose administered will vary with device size, surface area and design. However, certain principles can be applied in the application of this art. Drug dose can be calculated as a function of dose per unit area (of the portion of the device being coated), total drug dose administered can be measured, and appropriate surface concentrations of active drug can be determined. Regardless of the method of application of the drug to the device (i.e., as a coating or infiltrated into the surrounding tissue), the fibrosis-inhibiting agents, used alone or in combination, may be administered under the following dosing guidelines:

Drugs and dosage: Therapeutic agents that may be used include but are not limited to: antimicrotubule agents including taxanes (e.g., paclitaxel and docetaxel), other microtubule stabilizing and anti-microtubule agents, mycophenolic acid, sirolimus, tacrolimus, everolimus, ABT-578 and vinca alkaloids (e.g., vinblastine and vincristine sulfate) as well as analogues and derivatives thereof. Drugs are to be used at concentrations that range from several times more than a single systemic dose (e.g., the dose used in oral or i.v. administration) to a fraction of a single systemic dose (e.g., 50%, 10%, 5%, or even less than 1% of the concentration typically used in a single systemic dose application). Antimicrotubule agents including taxanes, such as paclitaxel and analogues and derivatives (e.g., docetaxel) thereof, and vinca alkaloids, including vinblastine and vincristine sulfate and analogues and derivatives thereof, should be used under the following parameters: total dose not to exceed 10 mg (range of 0.1 μg to 10 mg); preferred total dose 1 μg to 3 mg. Dose per unit area of the device of 0.05 μg-10 μg per mm²; preferred dose/unit area of 0.20 μg/mm²-5 μg/mm². Minimum concentration of 10⁻⁹-10⁻⁴ M of drug is to be maintained on the device surface. Immunomodulators including sirolimus, ABT-578 and everolimus. Sirolimus (i.e., rapamycin, RAPAMUNE): Total dose not to exceed 10 mg (range of 0.1 μg to 10 mg); preferred 10 μg to 1 mg. The dose per unit area of 0.1 μg-100 μg per mm²; preferred dose of 0.5 μg/mm²-10 μg/mm². Minimum concentration of 10⁻⁸-10⁻⁴ M is to be maintained on the device surface. Everolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface.

B. Therapeutic Agents for Use with Implantable Sensor and Drug Delivery Pump Devices

As described previously, numerous therapeutic agents are potentially suitable to inhibit fibrous tissue accumulation around the implantable sensor devices and drug-delivery pumps in the manner just described. The invention provides for medical devices that include an agent that inhibits this tissue accumulation in the vicinity of the device, i.e., between the medical device and the host into which the medical device is implanted. The agent is therefore effective for this goal, is present in an amount that is effective to achieve this goal, and is present at one or more locations that allow for this goal to be achieved, and the device is designed to allow the beneficial effects of the agent to occur. Also, these therapeutic agents can be used alone, or in combination, to prevent scar tissue build-up in the vicinity of the device-tissue interface in order to improve the clinical performance and longevity of these implants.

Suitable fibrosis agents may be readily identified based upon in vitro and in vivo (animal) models, such as those provided in Examples 34-47. Agents which inhibit fibrosis can also be identified through in vivo models including inhibition of intimal hyperplasia development in the rat balloon carotid artery model (Examples 39 and 47). The assays set forth in Examples 38 and 46 may be used to determine whether an agent is able to inhibit cell proliferation in fibroblasts and/or smooth muscle cells. In one aspect of the invention, the agent has an IC₅₀ for inhibition of cell proliferation within a range of about 10⁻⁶ to about 10⁻¹⁰ M. The assay set forth in Example 42 may be used to determine whether an agent may inhibit migration of fibroblasts and/or smooth muscle cells. In one aspect of the invention, the agent has an IC₅₀ for inhibition of cell migration within a range of about 10⁻⁶ to about 10⁻⁹M. Assays set forth herein may be used to determine whether an agent is able to inhibit inflammatory processes, including nitric oxide production in macrophages (Example 34), and/or TNF-alpha production by macrophages (Example 35), and/or IL-1 beta production by macrophages (Example 43), and/or IL-8 production by macrophages (Example 44), and/or inhibition of MCP-1 by macrophages (Example 45). In one aspect of the invention, the agent has an IC₅₀ for inhibition of any one of these inflammatory processes within a range of about 10⁻⁶ to about 10⁻¹⁰ M. The assay set forth in Example 40 may be used to determine whether an agent is able to inhibit MMP production. In one aspect of the invention, the agent has an IC₅₀ for inhibition of MMP production within a range of about 10⁻⁴ to about 10⁻⁸M. The assay set forth in Example 41 (also known as the CAM assay) may be used to determine whether an agent is able to inhibit angiogenesis. In one aspect of the invention, the agent has an IC₅₀ for inhibition of angiogenesis within a range of about 10⁻⁶ to about 10⁻¹⁰M. Agents which reduce the formation of surgical adhesions may be identified through in vivo models including the rabbit surgical adhesions model (Example 37) and the rat caecal sidewall model (Example 36). These pharmacologically active agents (described below) can then be delivered at appropriate dosages into to the tissue either alone, or via carriers (described herein), to treat the clinical problems described herein. Numerous therapeutic compounds have been identified that are of utility in the present invention including:

1. Angiogenesis Inhibitors

In one embodiment, the pharmacologically active compound is an angiogenesis inhibitor (e.g., 2-ME (NSC-659853), PI-88 (D-mannose, O-6-O-phosphono-alpha-D-mannopyranosyl-(1-3)-O-alpha-D-mannopyranosyl-(1-3)-O-alpha-D-mannopyranosyl-(1-3)-O-alpha-D-mannopyranosyl-(1-2)-hydrogen sulphate), thalidomide (1H-isoindole-1,3(2H)-dione, 2-(2,6-dioxo-3-piperidinyl)-), CDC-394, CC-5079, ENMD-0995 (S-3-amino-phthalidoglutarimide), AVE-8062A, vatalanib, SH-268, halofuginone hydrobromide, atiprimod dimaleate (2-azaspivo[4.5]decane-2-propanamine, N,N-diethyl-8,8-dipropyl, dimaleate), ATN-224, CHIR-258, combretastatin A-4 (phenol, 2-methoxy-5-[2-(3,4,5-trimethoxyphenyl)ethenyl]-, (Z)-), GCS-100LE, or an analogue or derivative thereof).

2. 5-Lipoxygenase Inhibitors and Antagonists

In another embodiment, the pharmacologically active compound is a 5-lipoxygenase inhibitor or antagonist (e.g., Wy-50295 (2-naphthaleneacetic acid, alpha-methyl-6-(2-quinolinylmethoxy)-, (S)-), ONO-LP-269 (2,11,14-eicosatrienamide, N-(4-hydroxy-2-(1H-tetrazol-5-yl)-8-quinolinyl)-, (E,Z,Z)-), licofelone (1H-pyrrolizine-5-acetic acid, 6-(4-chlorophenyl)-2,3-dihydro-2,2-dimethyl-7-phenyl-), CMI-568 (urea, N-butyl-N-hydroxy-N′-(4-(3-(methylsulfonyl)-2-propoxy-5-(tetrahydro-5-(3,4,5-trimethoxyphenyl)-2-furanyl)phenoxy)butyl)-,trans-), IP-751 ((3R,4R)-(delta 6)-THC-DMH-11-oic acid), PF-5901 (benzenemethanol, alpha-pentyl-3-(2-quinolinylmethoxy)-), LY-293111 (benzoic acid, 2-(3-(3-((5-ethyl-4′-fluoro-2-hydroxy(1,1′-biphenyl)-4-yl)oxy)propoxy)-2-propylphenoxy)-), RG-5901-A (benzenemethanol, alpha-pentyl-3-(2-quinolinylmethoxy)-, hydrochloride), rilopirox (2(1H)-pyridinone, 6-((4-(4-chlorophenoxy)phenoxy)methyl)-1-hydroxy-4-methyl-), L-674636 (acetic acid, ((4-(4-chlorophenyl)-1-(4-(2-quinolinylmethoxy)phenyl)butyl)thio)-AS)), 7-((3-(4-methoxy-tetrahydro-2H-pyran-4-yl)phenyl)methoxy)-4-phenylnaphtho(2,3-c)furan-1 (3H)-one, MK-886 (1H-indole-2-propanoic acid, 1-((4-chlorophenyl)methyl)-3-((1,1-dimethylethyl)thio)-alpha, alpha-dimethyl-5-(1-methylethyl)-), quiflapon (1H-indole-2-propanoic acid, 1-((4-chlorophenyl)methyl)-3-((1,1-dimethylethyl)thio)-alpha, alpha-dimethyl-5-(2-quinolinylmethoxy)-), quiflapon (1H-Indole-2-propanoic acid, 1-((4-chlorophenyl)methyl)-3-((1,1-dimethylethyl)thio)-alpha, alpha-dimethyl-5-(2-quinolinylmethoxy)-), docebenone (2,5-cyclohexadiene-1,4-dione, 2-(12-hydroxy-5,10-dodecadiynyl)-3,5,6-trimethyl-), zileuton (urea, N-(1-benzo(b)thien-2-ylethyl)-N-hydroxy-), or an analogue or derivative thereof).

3. Chemokine Receptor Antagonists CCR (1, 3, and 5)

In another embodiment, the pharmacologically active compound is a chemokine receptor antagonist which inhibits one or more subtypes of CCR (1, 3, and 5) (e.g., ONO-4128 (1,4,9-triazaspiro(5.5)undecane-2,5-dione, 1-butyl-3-(cyclohexylmethyl)-9-((2,3-dihydro-1,4-benzodioxin-6-yl)methyl-), L-381, CT-112 (L-arginine, L-threonyl-L-threonyl-L-seryl-L-glutaminyl-L-valyl-L-arginyl-L-prolyl-), AS-900004, SCH-C, ZK-811752, PD-172084, UK-427857, SB-380732, vMIP II, SB-265610, DPC-168, TAK-779 (N,N-dimethyl-N-(4-(2-(4-methylphenyl)-6,7-dihydro-5H-benzocyclohepten-8-ylcarboxamido)benyl)tetrahydro-2H-pyran-4-aminium chloride), TAK-220, KRH-1120), GSK766994, SSR-150106, or an analogue or derivative thereof). Other examples of chemokine receptor antagonists include a-Immunokine-NNS03, BX-471, CCX-282, Sch-350634; Sch-351125; Sch-417690; SCH-C, and analogues and derivatives thereof.

4. Cell Cycle Inhibitors

In another embodiment, the pharmacologically active compound is a cell cycle inhibitor. Representative examples of such agents include taxanes (e.g., paclitaxel (discussed in more detail below) and docetaxel) (Schiff et al., Nature 277: 665-667, 1979; Long and Fairchild, Cancer Research 54: 4355-4361, 1994; Ringel and Horwitz, J. Nat'l Cancer Inst. 83 (4): 288-291, 1991; Pazdur et al., Cancer Treat. Rev. 19 (40): 351-386, 1993), etanidazole, nimorazole (B. A. Chabner and D. L. Longo. Cancer Chemotherapy and Biotherapy—Principles and Practice. Lippincoft-Raven Publishers, New York, 1996, p. 554), perfluorochemicals with hyperbaric oxygen, transfusion, erythropoietin, BW12C, nicotinamide, hydralazine, BSO, WR-2721, ludR, DUdR, etanidazole, WR-2721, BSO, mono-substituted keto-aldehyde compounds (L. G. Egyud. Keto-aldehyde-amine addition products and method of making same. U.S. Pat. No. 4,066,650, Jan. 3, 1978), nitroimidazole (K. C. Agrawal and M. Sakaguchi. Nitroimidazole radiosensitizers for Hypoxic tumor cells and compositions thereof. U.S. Pat. No. 4,462,992, Jul. 31, 1984), 5-substituted-4-nitroimidazoles (Adams et al., Int. J. Radiat. Biol. Relat. Stud. Phys., Chem. Med. 40 (2): 153-61, 1981), SR-2508 (Brown et al., Int. J. Radiat. Oncol., Biol. Phys. 7 (6): 695-703, 1981), 2H-isoindolediones (J. A. Myers, 2H-Isoindolediones, the synthesis and use as radiosensitizers. U.S. Pat. No. 4,494,547, Jan. 22, 1985), chiral (((2-bromoethyl)-amino)methyl)-nitro-1H-imidazole-1-ethanol (V. G. Beylin, et al., Process for preparing chiral (((2-bromoethyl)-amino)methyl)nitro-1H-imidazole-1-ethanol and related compounds. U.S. Pat. No. 5,543,527, Aug. 6, 1996; U.S. Pat. No. 4,797,397; Jan. 10, 1989; U.S. Pat. No. 5,342,959, Aug. 30, 1994), nitroaniline derivatives (W. A. Denny, et al. Nitroaniline derivatives and the use as anti-tumor agents. U.S. Pat. No. 5,571,845, Nov. 5, 1996), DNA-affinic hypoxia selective cytotoxins (M. V. Papadopoulou-Rosenzweig. DNA-affinic hypoxia selective cytotoxins. U.S. Pat. No. 5,602,142, Feb. 11, 1997), halogenated DNA ligand (R. F. Martin. Halogenated DNA ligand radiosensitizers for cancer therapy. U.S. Pat. No. 5,641,764, Jun. 24, 1997), 1,2,4 benzotriazine oxides (W. W. Lee et al. 1,2,4-benzotriazine oxides as radiosensitizers and selective cytotoxic agents. U.S. Pat. No. 5,616,584, Apr. 1, 1997; U.S. Pat. No. 5,624,925, Apr. 29, 1997; Process for Preparing 1,2,4 Benzotriazine oxides. U.S. Pat. No. 5,175,287, Dec. 29, 1992), nitric oxide (J. B. Mitchell et al., Use of Nitric oxide releasing compounds as hypoxic cell radiation sensitizers. U.S. Pat. No. 5,650,442, Jul. 22, 1997), 2-nitroimidazole derivatives (M. J. Suto et al. 2-Nitroimidazole derivatives useful as radiosensitizers for hypoxic tumor cells. U.S. Pat. No. 4,797,397, Jan. 10, 1989; T. Suzuki. 2-Nitroimidazole derivative, production thereof, and radiosensitizer containing the same as active ingredient. U.S. Pat. No. 5,270,330, Dec. 14, 1993; T. Suzuki et al. 2-Nitroimidazole derivative, production thereof, and radiosensitizer containing the same as active ingredient. U.S. Pat. No. 5,270,330, Dec. 14, 1993; T. Suzuki. 2-Nitroimidazole derivative, production thereof and radiosensitizer containing the same as active ingredient; Patent EP 0 513 351 B1, Jan. 24, 1991), fluorine-containing nitroazole derivatives (T. Kagiya. Fluorine-containing nitroazole derivatives and radiosensitizer comprising the same. U.S. Pat. No. 4,927,941, May 22, 1990), copper (M. J. Abrams. Copper Radiosensitizers. U.S. Pat. No. 5,100,885, Mar. 31, 1992), combination modality cancer therapy (D. H. Picker et al. Combination modality cancer therapy. U.S. Pat. No. 4,681,091, Jul. 21, 1987). 5-CldC or (d)H₄U or 5-halo-2′-halo-2′-deoxy-cytidine or -uridine derivatives (S. B. Greer. Method and Materials for sensitizing neoplastic tissue to radiation. U.S. Pat. No. 4,894,364 Jan. 16, 1990), platinum complexes (K. A. Skov. Platinum Complexes with one radiosensitizing ligand. U.S. Pat. No. 4,921,963. May 1, 1990; K. A. Skov. Platinum Complexes with one radiosensitizing ligand. Patent EP 0 287 317 A3), fluorine-containing nitroazole (T. Kagiya, et al. Fluorine-containing nitroazole derivatives and radiosensitizer comprising the same. U.S. Pat. No. 4,927,941. May 22, 1990), benzamide (W. W. Lee. Substituted Benzamide Radiosensitizers. U.S. Pat. No. 5,032,617, Jul. 16, 1991), autobiotics (L. G. Egyud. Autobiotics and the use in eliminating nonself cells in vivo. U.S. Pat. No. 5,147,652. Sep. 15, 1992), benzamide and nicotinamide (W. W. Lee et al. Benzamide and Nictoinamide Radiosensitizers. U.S. Pat. No. 5,215,738, Jun. 1, 1993), acridine-intercalator (M. Papadopoulou-Rosenzweig. Acridine Intercalator based hypoxia selective cytotoxins. U.S. Pat. No. 5,294,715, Mar. 15, 1994), fluorine-containing nitroimidazole (T. Kagiya et al. Fluorine containing nitroimidazole compounds. U.S. Pat. No. 5,304,654, Apr. 19, 1994), hydroxylated texaphyrins (J. L. Sessler et al. Hydroxylated texaphrins. U.S. Pat. No. 5,457,183, Oct. 10, 1995), hydroxylated compound derivative (T. Suzuki et al. Heterocyclic compound derivative, production thereof and radiosensitizer and antiviral agent containing said derivative as active ingredient. Publication Number 011106775 A (Japan), Oct. 22, 1987; T. Suzuki et al. Heterocyclic compound derivative, production thereof and radiosensitizer, antiviral agent and anti cancer agent containing said derivative as active ingredient. Publication Number 01139596 A (Japan), Nov. 25, 1987; S. Sakaguchi et al. Heterocyclic compound derivative, its production and radiosensitizer containing said derivative as active ingredient; Publication Number 63170375 A (Japan), Jan. 7, 1987), fluorine containing 3-nitro-1,2,4-triazole (T. Kagitani et al. Novel fluorine-containing 3-nitro-1,2,4-triazole and radiosensitizer containing same compound. Publication Number 02076861 A (Japan), Mar. 31, 1988), 5-thiotretrazole derivative or its salt (E. Kano et al. Radiosensitizer for Hypoxic cell. Publication Number 61010511 A (Japan), Jun. 26, 1984), Nitrothiazole (T. Kagitani et al. Radiation-sensitizing agent. Publication Number 61167616 A (Japan) Jan. 22, 1985), imidazole derivatives (S. Inayma et al. Imidazole derivative. Publication Number 6203767 A (Japan) Aug. 1, 1985; Publication Number 62030768 A (Japan) Aug. 1, 1985; Publication Number 62030777 A (Japan) Aug. 1, 1985), 4-nitro-1,2,3-triazole (T. Kagitani et al. Radiosensitizer. Publication Number 62039525 A (Japan), Aug. 15, 1985), 3-nitro-1,2,4-triazole (T. Kagitani et al. Radiosensitizer. Publication Number 62138427 A (Japan), Dec. 12, 1985), Carcinostatic action regulator (H. Amagase. Carcinostatic action regulator. Publication Number 63099017 A (Japan), Nov. 21, 1986), 4,5-dinitroimidazole derivative (S. Inayama. 4,5-Dinitroimidazole derivative. Publication Number 63310873 A (Japan) Jun. 9, 1987), nitrotriazole Compound (T. Kagitanil Nitrotriazole Compound. Publication Number 07149737 A (Japan) Jun. 22, 1993), cisplatin, doxorubin, misonidazole, mitomycin, tiripazamine, nitrosourea, mercaptopurine, methotrexate, flurouracil, bleomycin, vincristine, carboplatin, epirubicin, doxorubicin, cyclophosphamide, vindesine, etoposide (I. F. Tannock. Review Article: Treatment of Cancer with Radiation and Drugs. Journal of Clinical Oncology 14 (12): 3156-3174, 1996), camptothecin (Ewend M. G. et al. Local delivery of chemotherapy and concurrent external beam radiotherapy prolongs survival in metastatic brain tumor models. Cancer Research 56 (22): 5217-5223, 1996) and paclitaxel (Tishler R. B. et al. Taxol: a novel radiation sensitizer. International Journal of Radiation Oncology and Biological Physics 22 (3): 613-617, 1992).

A number of the above-mentioned cell cycle inhibitors also have a wide variety of analogues and derivatives, including, but not limited to, cisplatin, cyclophosphamide, misonidazole, tiripazamine, nitrosourea, mercaptopurine, methotrexate, flurouracil, epirubicin, doxorubicin, vindesine and etoposide. Analogues and derivatives include (CPA)₂Pt(DOLYM) and (DACH)Pt(DOLYM) cisplatin (Choi et al., Arch. Pharmacal Res. 22 (2): 151-156, 1999), Cis-(PtCl₂(4,7-H-5-methyl-7-oxo)1,2,4(triazolo(1,5-a)pyrimidine)₂) (Navarro et al., J. Med. Chem. 41 (3): 332-338, 1998), (Pt(cis-1,4-DACH)(trans-Cl₂)(CBDCA)).½MeOH cisplatin (Shamsuddin et al., Inorg. Chem. 36 (25): 5969-5971, 1997), 4-pyridoxate diammine hydroxy platinum (Tokunaga et al., Pharm. Sci. 3 (7): 353-356, 1997), Pt(II). . . Pt(II) (Pt₂(NHCHN(C(CH₂)(CH₃)))₄) (Navarro et al., Inorg. Chem. 35 (26): 7829-7835, 1996), 254-S cisplatin analogue (Koga et al., Neurol. Res. 18 (3): 244-247, 1996), o-phenylenediamine ligand bearing cisplatin analogues (Koeckerbauer & Bednarski, J. Inorg. Biochem. 62 (4): 281-298, 1996), trans,cis-(Pt(OAc)₂I₂(en)) (Kratochwil et al., J. Med. Chem. 39 (13): 2499-2507, 1996), estrogenic 1,2-diarylethylenediamine ligand (with sulfur-containing amino acids and glutathione) bearing cisplatin analogues (Bednarski, J. Inorg. Biochem. 62 (1): 75, 1996), cis-1,4-diaminocyclohexane cisplatin analogues (Shamsuddin et al., J. Inorg. Biochem. 61 (4): 291-301, 1996), 5′ orientational isomer of cis-(Pt(NH₃)(4-aminoTEMP-O){d(GpG)}) (Dunham & Lippard, J. Am. Chem. Soc. 117 (43): 10702-12, 1995), chelating diamine-bearing cisplatin analogues (Koeckerbauer & Bednarski, J. Pharm. Sci. 84 (7): 819-23, 1995), 1,2-diarylethyleneamine ligand-bearing cisplatin analogues (Otto et al., J. Cancer Res. Clin. Oncol. 121 (1): 31-8, 1995), (ethylenediamine)platinum(II) complexes (Pasini et al., J. Chem. Soc., Dalton Trans. 4: 579-85, 1995), CI-973 cisplatin analogue (Yang et al., Int. J. Oncol. 5 (3): 597-602, 1994), cis-diamminedichloroplatinum(II) and its analogues cis-1,1-cyclobutanedicarbosylato(2R)-2-methyl-1,4-butanediammineplatinum(II) and cis-diammine(glycolato)platinum (Claycamp & Zimbrick, J. Inorg. Biochem., 26 (4): 257-67, 1986; Fan et al., Cancer Res. 48 (11): 3135-9, 1988; Heiger-Bernays et al., Biochemistry 29 (36): 8461-6, 1990; Kikkawa et al., J. Exp. Clin. Cancer Res. 12 (4): 233-40, 1993; Murray et al., Biochemistry 31(47): 11812-17, 1992; Takahashi et al., Cancer Chemother. Pharmacol. 33 (1): 31-5, 1993), cis-amine-cyclohexylamine-dichloroplatinum(II) (Yoshida et al., Biochem. Pharmacol. 48 (4): 793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35 (23): 4479-85, 1992), cisplatin analogues containing a tethered dansyl group (Hartwig et al., J. Am. Chem. Soc. 114 (21): 8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197 (2): 311-15, 1991), trans-diamminedichloroplatinum(II) and cis-(Pt(NH₃)₂(N₃-cytosine)Cl) (Bellon & Lippard, Biophys. Chem. 35 (2-3): 179-88, 1990), 3H-cis-1,2-diaminocyclohexanedichloroplatinum(II) and 3H-cis-1,2-diaminocyclohexanemalonatoplatinum(II) (Oswald et al., Res. Commun. Chem. Pathol. Pharmacol. 64 (1): 51-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25 (4): 349-57, 1988), aminoalkylaminoanthraquinone-derived cisplatin analogues (Kitov et al., Eur. J. Med. Chem. 23 (4): 381-3, 1988), spiroplatin, carboplatin, iproplatin and JM40 platinum analogues (Schroyen et al., Eur. J. Cancer Clin. Oncol. 24 (8): 1309-12, 1988), bidentate tertiary diamine-containing cisplatinum derivatives (Orbell et al., Inorg. Chim. Acta 152 (2): 125-34, 1988), platinum(II), platinum(IV) (Liu & Wang, Shandong Yike Daxue Xuebao 24 (1): 35-41, 1986), cis-diammine(1,1-cyclobutanedicarboxylato-)platinum(II) (carboplatin, JM8) and ethylenediamminemalonatoplatinum(II) (JM40) (Begg et al., Radiother. Oncol. 9 (2): 157-65, 1987), JM8 and JM9 cisplatin analogues (Harstrick et al., Int. J. Androl. 10 (1); 139-45, 1987), (NPr4)2((PtCL4).cis-(PtCl2-(NH2Me)2)) (Brammer et al., J. Chem. Soc., Chem. Commun. 6: 443-5, 1987), aliphatic tricarboxylic acid platinum complexes (EPA 185225), cis-dichloro(amino acid)(tert-butylamine)platinum(II) complexes (Pasini & Bersanetti, Inorg. Chim. Acta 107 (4): 259-67, 1985); 4-hydroperoxycylcophosphamide (Ballard et al., Cancer Chemother. Pharmacol. 26 (6): 397-402, 1990), acyclouridine cyclophosphamide derivatives (Zakerinia et al., Helv. Chim. Acta 73 (4): 912-15, 1990), 1,3,2-dioxa- and -oxazaphosphorinane cyclophosphamide analogues (Yang et al., Tetrahedron 44 (20): 6305-14, 1988), C5-substituted cyclophosphamide analogues (Spada, University of Rhode Island Dissertation, 1987), tetrahydrooxazine cyclophosphamide analogues (Valente, University of Rochester Dissertation, 1988), phenyl ketone cyclophosphamide analogues (Hales et al., Teratology 39 (1): 31-7, 1989), phenylketophosphamide cyclophosphamide analogues (Ludeman et al., J. Med. Chem. 29 (5): 716-27, 1986), ASTA Z-7557 cyclophosphamide analogues (Evans et al., Int. J. Cancer 34 (6): 883-90, 1984), 3-(1-oxy-2,2,6,6-tetramethyl-4-piperidinyl)cyclophosphamide (Tsui et al., J. Med. Chem. 25 (9): 1106-10, 1982), 2-oxobis(2-β-chloroethylamino)-4-,6-dimethyl-1,3,2-oxazaphosphorinane cyclophosphamide (Carpenter et al., Phosphorus Sulfur 12 (3): 287-93, 1982), 5-fluoro- and 5-chlorocyclophosphamide (Foster et al., J. Med. Chem. 24 (12): 1399-403, 1981), cis- and trans-4-phenylcyclophosphamide (Boyd et al., J. Med. Chem. 23 (4): 372-5, 1980), 5-bromocyclophosphamide, 3,5-dehydrocyclophosphamide (Ludeman et al., J. Med. Chem. 22 (2): 151-8, 1979), 4-ethoxycarbonyl cyclophosphamide analogues (Foster, J. Pharm. Sci. 67 (5): 709-10, 1978), arylaminotetrahydro-2H-1,3,2-oxazaphosphorine 2-oxide cyclophosphamide analogues (Hamacher, Arch. Pharm. (Weinheim, Ger.) 310 (5): J, 428-34, 1977), NSC-26271 cyclophosphamide analogues (Montgomery & Struck, Cancer Treat Rep. 60 (4): J381-93, 1976), benzo annulated cyclophosphamide analogues (Ludeman & Zon, J. Med. Chem. 18(12): J1251-3, 1975), 6-trifluoromethylcyclophosphamide (Farmer & Cox, J. Med. Chem. 18 (11): J1106-10, 1975), 4-methylcyclophosphamide and 6-methycyclophosphamide analogues (Cox et al., Biochem. Pharmacol. 24 (5): J599-606, 1975); FCE 23762 doxorubicin derivative (Quaglia et al., J. Liq. Chromatogr. 17 (18): 3911-3923, 1994), annamycin (Zou et al., J. Pharm. Sci. 82 (11): 1151-1154, 1993), ruboxyl (Rapoport et al., J. Controlled Release 58 (2): 153-162, 1999), anthracycline disaccharide doxorubicin analogue (Pratesi et al., Clin. Cancer Res. 4 (11): 2833-2839, 1998), N-(trifluoroacetyl)doxorubicin and 4′-O-acetyl-N-(trifluoroacetyl)doxorubicin (Berube & Lepage, Synth. Commun. 28 (6): 1109-1116, 1998), 2-pyrrolinodoxorubicin (Nagy et al., Proc. Nat'l Acad. Sci. U.S.A. 95 (4): 1794-1799, 1998), disaccharide doxorubicin analogues (Arcamone et al., J. Nat'Cancer Inst. 89 (16): 1217-1223, 1997), 4-demethoxy-7-O-(2,6-dideoxy-4-O-(2,3,6-trideoxy-3-amino-α-L-lyxo-hexopyranosyl)-α-L-lyxo-hexopyranosyl)-adriamicinone doxorubicin disaccharide analogue (Monteagudo et al., Carbohydr. Res. 300 (1): 11-16, 1997), 2-pyrrolinodoxorubicin (Nagy et al., Proc. Nat'l Acad. Sci. U.S.A. 94 (2): 652-656, 1997), morpholinyl doxorubicin analogues (Duran et al., Cancer Chemother. Pharmacol. 38 (3): 210-216, 1996), enaminomalonyl-α-alanine doxorubicin derivatives (Seitz et al., Tetrahedron Lett. 36 (9): 1413-16, 1995), cephalosporin doxorubicin derivatives (Vrudhula et al., J. Med. Chem. 38 (8): 1380-5, 1995), hydroxyrubicin (Solary et al., Int J. Cancer 58 (1): 85-94, 1994), methoxymorpholino doxorubicin derivative (Kuhl et al., Cancer Chemother. Pharmacol. 33 (1): 10-16, 1993), (6-maleimidocaproyl)hydrazone doxorubicin derivative (Willner et al., Bioconjugate Chem. 4 (6): 521-7, 1993), N-(5,5-diacetoxypent-1-yl) doxorubicin (Cherif & Farquhar, J. Med. Chem. 35 (17): 3208-14, 1992), FCE 23762 methoxymorpholinyl doxorubicin derivative (Ripamonti et al., Br. J. Cancer 65 (5): 703-7, 1992), N-hydroxysuccinimide ester doxorubicin derivatives (Demant et al., Biochim. Biophys. Acta 1118 (1): 83-90, 1991), polydeoxynucleotide doxorubicin derivatives (Ruggiero et al., Biochim. Biophys. Acta 1129 (3): 294-302, 1991), morpholinyl doxorubicin derivatives (EPA 434960), mitoxantrone doxorubicin analogue (Krapcho et al., J. Med. Chem. 34 (8): 2373-80. 1991), AD198 doxorubicin analogue (Traganos et al., Cancer Res. 51 (14): 3682-9, 1991), 4-demethoxy-3′-N-trifluoroacetyidoxorubicin (Horton et al., Drug Des. Delivery 6 (2): 123-9, 1990), 4′-epidoxorubicin (Drzewoski et al., Pol. J. Pharmacol. Pharm. 40 (2): 159-65, 1988; Weenen et al., Eur. J. Cancer Clin. Oncol. 20 (7): 919-26, 1984), alkylating cyanomorpholino doxorubicin derivative (Scudder et al., J. Nat'l Cancer Inst. 80 (16): 1294-8, 1988), deoxydihydroiodooxorubicin (EPA 275966), adriblastin (Kalishevskaya et al., Vestn. Mosk. Univ., 16 (Biol. 1): 21-7, 1988), 4′-deoxydoxorubicin (Schoelzel et al., Leuk. Res. 10 (12): 1455-9, 1986), 4-demethyoxy-4′-o-methyldoxorubicin (Giuliani et al., Proc. Int. Congr. Chemother. 16: 285-70-285-77, 1983), 3′-deamino-3′-hydroxydoxorubicin (Horton et al., J. Antibiot. 37 (8): 853-8, 1984), 4-demethyoxy doxorubicin analogues (Barbieri et al., Drugs Exp. Clin. Res. 10 (2): 85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-methyldoxorubicin (Giuliani et al., Int. J. Cancer 27 (1): 5-13, 1981), aglycone doxorubicin derivatives (Chan & Watson, J. Pharm. Sci. 67 (12): 1748-52, 1978), SM 5887 (Pharma Japan 1468: 20, 1995), MX-2 (Pharma Japan 1420: 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277); 4,5-dimethylmisonidazole (Born et al., Biochem. Pharmacol. 43 (6): 1337-44, 1992), azo and azoxy misonidazole derivatives (Gattavecchia & Tonelli, Int. J. Radiat. Biol. Relat. Stud. Phys., Chem. Med. 45 (5): 469-77, 1984); RB90740 (Wardman et al., Br. J. Cancer, 74 Suppl. (27): S70-S74, 1996); 6-bromo and 6-chloro-2,3-dihydro-1,4-benzothiazines nitrosourea derivatives (Rai et al., Heterocycl. Commun. 2 (6): 587-592, 1996), diamino acid nitrosourea derivatives (Dulude et al., Bioorg. Med. Chem. Lett. 4 (22): 2697-700, 1994; Dulude et al., Bioorg. Med. Chem. 3 (2): 151-60, 1995), amino acid nitrosourea derivatives (Zheleva et al., Pharmazie 50 (1): 25-6, 1995), 3′,4′-didemethoxy-3′,4′-dioxo-4-deoxypodophyllotoxin nitrosourea derivatives (Miyahara et al., Heterocycles 39 (1): 361-9, 1994), ACNU (Matsunaga et al., Immunopharmacology 23 (3): 199-204, 1992), tertiary phosphine oxide nitrosourea derivatives (Guguva et al., Pharmazie 46(8): 603, 1991), sulfamerizine and sulfamethizole nitrosourea derivatives (Chiang et al., Zhonghua Yaozue Zazhi 43 (5): 401-6, 1991), thymidine nitrosourea analogues (Zhang et al., Cancer Commun. 3 (4): 119-26, 1991), 1,3-bis(2-chloroethyl)-1-nitrosourea (August et al., Cancer Res. 51 (6): 1586-90, 1991), 2,2,6,6-tetramethyl-1-oxopiperidiunium nitrosourea derivatives (U.S.S.R. 1261253), 2- and 4-deoxy sugar nitrosourea derivatives (U.S. Pat. No. 4,902,791), nitroxyl nitrosourea derivatives (U.S.S.R. 1336489), fotemustine (Boutin et al., Eur. J. Cancer Clin. Oncol. 25 (9): 1311-16, 1989), pyrimidine(II) nitrosourea derivatives (Wei et al., Chung-hua Yao Hsuch Tsa Chih 41 (1): 19-26, 1989), CGP 6809 (Schieweck et al., Cancer Chemother. Pharmacol. 23 (6): 341-7, 1989), B-3839 (Prajda et al., In Vivo 2 (2): 151-4, 1988), 5-halogenocytosine nitrosourea derivatives (Chiang & Tseng, T'ai-wan Yao Hsuch Tsa Chih 38 (1): 37-43, 1986), 1-(2-chloroethyl)-3-isobutyl-3-(β-maltosyl)-1-nitrosourea (Fujimoto & Ogawa, J. Pharmacobio-Dyn. 10 (7): 341-5, 1987), sulfur-containing nitrosoureas (Tang et al., Yaoxue Xuebao 21 (7): 502-9, 1986), sucrose, 6-((((2-chloroethyl)nitrosoamino-)carbonyl)amino)-6-deoxysucrose (NS-1C) and 6′-((((2-chloroethyl)nitrosoamino)carbonyl)amino)-6′-deoxysucrose (NS-1D) nitrosourea derivatives (Tanoh et al., Chemotherapy (Tokyo) 33 (11): 969-77, 1985), CNCC, RFCNU and chlorozotocin (Mena et al., Chemotherapy (Basel) 32 (2): 131-7, 1986), CNUA (Edanami et al., Chemotherapy (Tokyo) 33 (5): 455-61, 1985), 1-(2-chloroethyl)-3-isobutyl-3-(β-maltosyl)-1-nitrosourea (Fujimoto & Ogawa, Jpn. J. Cancer Res. (Gann) 76 (7): 651-6, 1985), choline-like nitrosoalkylureas (Belyaev et al., Izv. Akad. NAUK SSSR, Ser. Khim. 3: 553-7, 1985), sucrose nitrosourea derivatives (JP 84219300), sulfa drug nitrosourea analogues (Chiang et al., Proc. Nat'l Sci. Counc., Repub. China, Part A 8(1): 18-22, 1984), DONU (Asanuma et al., J. Jpn. Soc. Cancer Ther. 17 (8): 2035-43, 1982), N,N′-bis (N-(2-chloroethyl)-N-nitrosocarbamoyl)cystamine (CNCC) (Blazsek et al., Toxicol. Appl. Pharmacol. 74 (2): 250-7, 1984), dimethylnitrosourea (Krutova et al., Izv. Akad. NAUK SSSR, Ser. Biol. 3: 439-45, 1984), GANU (Sava & Giraldi, Cancer Chemother. Pharmacol. 10 (3): 167-9, 1983), CCNU (Capelli et al., Med., Biol., Environ. 11 (1): 111-16, 1983), 5-aminomethyl-2′-deoxyuridine nitrosourea analogues (Shiau, Shih Ta Hsuch Pao (Taipei) 27: 681-9, 1982), TA-077 (Fujimoto & Ogawa, Cancer Chemother. Pharmacol. 9 (3): 134-9, 1982), gentianose nitrosourea derivatives (JP 82 80396), CNCC, RFCNU, RPCNU AND chlorozotocin (CZT) (Marzin et al., INSERM Symp., 19 (Nitrosoureas Cancer Treat.): 165-74, 1981), thiocolchicine nitrosourea analogues (George, Shih Ta Hsuch Pao (Taipei) 25: 355-62, 1980), 2-chloroethyl-nitrosourea (Zeller & Eisenbrand, Oncology 38 (1): 39-42, 1981), ACNU, (1-(4-amino-2-methyl-5-pyrimidinyl)methyl-3-(2-chloroethyl)-3-nitrosourea hydrochloride) (Shibuya et al., Gan To Kagaku Ryoho 7 (8): 1393-401, 1980), N-deacetylmethyl thiocolchicine nitrosourea analogues (Lin et al., J. Med. Chem. 23 (12): 1440-2, 1980), pyridine and piperidine nitrosourea derivatives (Crider et al., J. Med. Chem. 23 (8): 848-51, 1980), methyl-CCNU (Zimber & Perk, Refu. Vet. 35 (1): 28, 1978), phensuzimide nitrosourea derivatives (Crider et al., J. Med. Chem. 23 (3): 324-6, 1980), ergoline nitrosourea derivatives (Crider et al., J. Med. Chem. 22 (1): 32-5, 1979), glucopyranose nitrosourea derivatives (JP 78 95917), 1-(2-chloroethyl)-3-cyclohexyl-1-nitrosourea (Farmer et al., J. Med. Chem. 21 (6): 514-20, 1978), 4-(3-(2-chloroethyl)-3-nitrosoureid-o)-cis-cyclohexanecarboxylic acid (Drewinko et al., Cancer Treat. Rep. 61 (8): J1513-18, 1977), RPCNU (ICIG 1163) (Larnicol et al., Biomedicine 26 (3): J176-81, 1977), IOB-252 (Sorodoc et al., Rev. Roum. Med., Virol. 28 (1): J55-61, 1977), 1,3-bis(2-chloroethyl)-1-nitrosourea (BCNU) (Siebert & Eisenbrand, Mutat. Res. 42 (1): J45-50, 1977), 1-tetrahydroxycyclopentyl-3-nitroso-3-(2-chloroethyl)-urea (U.S. Pat. No. 4,039,578), d-1-1-(β-chloroethyl)-3-(2-oxo-3-hexahydroazepinyl)-1-nitrosourea (U.S. Pat. No. 3,859,277) and gentianose nitrosourea derivatives (JP 57080396); 6-S-aminoacyloxymethyl mercaptopurine derivatives (Harada et al., Chem. Pharm. Bull. 43 (10): 793-6, 1995), 6-mercaptopurine (6-MP) (Kashida et al., Biol. Pharm. Bull. 18 (11): 1492-7, 1995), 7,8-polymethyleneimidazo-1,3,2-diazaphosphorines (Nilov et al., Mendeleev Commun. 2: 67, 1995), azathioprine (Chifotides et al., J. Inorg. Biochem. 56 (4): 249-64, 1994), methyl-D-glucopyranoside mercaptopurine derivatives (Da Silva et al., Eur. J. Med. Chem. 29 (2): 149-52, 1994) and s-alkynyl mercaptopurine derivatives (Ratsino et al., Khim.-Farm. Zh. 15 (8): 65-7, 1981); indoline ring and a modified ornithine or glutamic acid-bearing methotrexate derivatives (Matsuoka et al., Chem. Pharm. Bull. 45 (7): 1146-1150, 1997), alkyl-substituted benzene ring C bearing methotrexate derivatives (Matsuoka et al., Chem. Pharm. Bull. 44 (12): 2287-2293, 1996), benzoxazine or benzothiazine moiety-bearing methotrexate derivatives (Matsuoka et al., J. Med. Chem. 40 (1): 105-111, 1997), 10-deazaminopterin analogues (DeGraw et al., J. Med. Chem. 40 (3): 370-376, 1997), 5-deazaminopterin and 5,10-dideazaminopterin methotrexate analogues (Piper et al., J. Med. Chem. 40 (3): 377-384, 1997), indoline moiety-bearing methotrexate derivatives (Matsuoka et al., Chem. Pharm. Bull. 44 (7): 1332-1337, 1996), lipophilic amide methotrexate derivatives (Pignatello et al., World Meet. Pharm., Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39 (1): 56-65, 1996), methotrexate tetrahydroquinazoline analogue (Gangjee, et al., J. Heterocycl. Chem. 32 (1): 243-8, 1995), N-(α-aminoacyl) methotrexate derivatives (Cheung et al., Pteridines 3 (1-2): 101-2, 1992), biotin methotrexate derivatives (Fan et al., Pteridines 3 (1-2): 131-2, 1992), D-glutamic acid or D-erythrou, threo-4-fluoroglutamic acid methotrexate analogues (McGuire et al., Biochem. Pharmacol. 42 (12): 2400-3, 1991), β,γ-methano methotrexate analogues (Rosowsky et al., Pteridines 2 (3): 133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28 (2): 751-8, 1989), meta and ortho isomers of aminopterin (Rosowsky et al., J. Med. Chem. 32 (12): 2582, 1989), hydroxymethylmethotrexate (DE 267495), γ-fluoromethotrexate (McGuire et al., Cancer Res. 49 (16): 4517-25, 1989), polyglutamyl methotrexate derivatives (Kumar et al., Cancer Res. 46 (10): 5020-3, 1986), gem-diphosphonate methotrexate analogues (WO 88/06158), α- and γ-substituted methotrexate analogues (Tsushima et al., Tetrahedron 44 (17): 5375-87, 1988), 5-methyl-5-deaza methotrexate analogues (U.S. Pat. No. 4,725,687), Nδ-acyl-Nα-(4-amino-4-deoxypteroyl)-L-ornithine derivatives (Rosowsky et al., J. Med. Chem. 31 (7): 1332-7, 1988), 8-deaza methotrexate analogues (Kuehl et al., Cancer Res. 48 (6): 1481-8, 1988), acivicin methotrexate analogue (Rosowsky et al., J. Med. Chem. 30 (8): 1463-9, 1987), polymeric platinol methotrexate derivative (Carraher et al., Polym. Sci. Technol. (Plenum), 35 (Adv. Biomed. Polym.): 311-24, 1987), methotrexate-γ-dimyristoylphophatidylethanolamine (Kinsky et al., Biochim. Biophys. Acta 917 (2): 211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62, 1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol. 35 (15): 2607-13, 1986), polyglutamate methotrexate derivatives (Kamen & Winick, Methods Enzymol. 122 (Vitam. Coenzymes, Pt. G): 339-46, 1986), 5-methyl-5-deaza analogues (Piper et al., J. Med. Chem. 29 (6): 1080-7, 1986), quinazoline methotrexate analogue (Mastropaolo et al., J. Med. Chem. 29 (1): 155-8, 1986), pyrazine methotrexate analogue (Lever & Vestal, J. Heterocycl. Chem. 22 (1): 5-6, 1985), cysteic acid and homocysteic acid methotrexate analogues (U.S. Pat. No. 4,490,529), γ-tert-butyl methotrexate esters (Rosowsky et al., J. Med. Chem. 28 (5): 660-7, 1985), fluorinated methotrexate analogues (Tsushima et al., Heterocycles 23 (1): 45-9, 1985), folate methotrexate analogue (Trombe, J. Bacteriol. 160 (3): 849-53, 1984), phosphonoglutamic acid analogues (Sturtz & Guillamot, Eur. J. Med. Chem.—Chim. Ther. 19 (3): 267-73, 1984), poly (L-lysine) methotrexate conjugates (Rosowsky et al., J. Med. Chem. 27 (7): 888-93, 1984), dilysine and trilysine methotrexate derivates (Forsch & Rosowsky, J. Org. Chem. 49 (7): 1305-9, 1984), 7-hydroxymethotrexate (Fabre et al., Cancer Res. 43 (10): 4648-52, 1983), poly-γ-glutamyl methotrexate analogues (Piper & Montgomery, Adv. Exp. Med. Biol., 163 (Folyl Antifolyl Polyglutamates): 95-100, 1983), 3′,5′-dichloromethotrexate (Rosowsky & Yu, J. Med. Chem. 26 (10): 1448-52, 1983), diazoketone and chloromethylketone methotrexate analogues (Gangjee et al., J. Pharm. Sci. 71 (6): 717-19, 1982), 10-propargylaminopterin and alkyl methotrexate homologs (Piper et al., J. Med. Chem. 25 (7): 877-80, 1982), lectin derivatives of methotrexate (Lin et al., JNCI 66 (3): 523-8, 1981), polyglutamate methotrexate derivatives (Galivan, Mol. Pharmacol. 17 (1): 105-10, 1980), halogentated methotrexate derivatives (Fox, JNCI 58 (4): J955-8, 1977), 8-alkyl-7,8-dihydro analogues (Chaykovsky et al., J. Med. Chem. 20 (10): J1323-7, 1977), 7-methyl methotrexate derivatives and dichloromethotrexate (Rosowsky & Chen, J. Med. Chem. 17 (12): J1308-11, 1974), lipophilic methotrexate derivatives and 3′,5′-dichloromethotrexate (Rosowsky, J. Med. Chem. 16 (10): J1190-3, 1973), deaza amethopterin analogues (Montgomery et al., Ann. N.Y. Acad. Sci. 186: J227-34, 1971), MX068 (Pharma Japan, 1658: 18, 1999) and cysteic acid and homocysteic acid methotrexate analogues (EPA 0142220); N3-alkylated analogues of 5-fluorouracil (Kozai et al., J. Chem. Soc., Perkin Trans. 1 (19): 3145-3146, 1998), 5-fluorouracil derivatives with 1,4-oxaheteroepane moieties (Gomez et al., Tetrahedron 54 (43): 13295-13312, 1998), 5-fluorouracil and nucleoside analogues (Li, Anticancer Res. 17 (1A): 21-27, 1997), cis- and trans-5-fluoro-5,6-dihydro-6-alkoxyuracil (Van der Wilt et al., Br. J. Cancer 68 (4): 702-7, 1993), cyclopentane 5-fluorouracil analogues (Hronowski & Szarek, Can. J. Chem. 70 (4): 1162-9, 1992), A-OT-fluorouracil (Zhang et al., Zongguo Yiyao Gongye Zazhi 20 (11): 513-15, 1989), N4-trimethoxybenzoyl-5′-deoxy-5-fluorocytidine and 5′-deoxy-5-fluorouridine (Miwa et al., Chem. Pharm. Bull. 38 (4): 998-1003, 1990), 1-hexylcarbamoyl-5-fluorouracil (Hoshi et al., J. Pharmacobio-Dun. 3 (9): 478-81, 1980; Maehara et al., Chemotherapy (Basel) 34 (6): 484-9, 1988), B-3839 (Prajda et al., In Vivo 2 (2): 151-4, 1988), uracil-1-(2-tetrahydrofuryl)-5-fluorouracil (Anai et al., Oncology 45 (3): 144-7, 1988), 1-(2′-deoxy-2′-fluoro-β-D-arabinofuranosyl)-5-fluorouracil (Suzuko et al., Mol. Pharmacol. 31 (3): 301-6, 1987), doxifluridine (Matuura et al., Oyo Yakuri 29 (5): 803-31, 1985), 5′-deoxy-5-fluorouridine (Bollag & Hartmann, Eur. J. Cancer 16 (4): 427-32, 1980), 1-acetyl-3-O-toluyl-5-fluorouracil (Okada, Hiroshima J. Med. Sci. 28 (1): 49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680); 4′-epidoxorubicin (Lanius, Adv. Chemother. Gastrointest. Cancer, (Int. Symp.), 159-67, 1984); N-substituted deacetylvinblastine amide (vindesine) sulfates (Conrad et al., J. Med. Chem. 22 (4): 391-400, 1979); and Cu(II)-VP-16 (etoposide) complex (Tawa et al., Bioorg. Med. Chem. 6 (7): 1003-1008, 1998), pyrrolecarboxamidino-bearing etoposide analogues (Ji et al., Bioorg. Med. Chem. Lett. 7 (5): 607-612, 1997), γ-amino etoposide analogues (Hu, University of North Carolina Dissertation, 1992), γ-lactone ring-modified arylamino etoposide analogues (Zhou et al., J. Med. Chem. 37 (2): 287-92, 1994), N-glucosyl etoposide analogue (Allevi et al., Tetrahedron Lett. 34 (45): 7313-16, 1993), etoposide A-ring analogues (Kadow et al., Bioorg. Med. Chem. Lett. 2 (1): 17-22, 1992), 4′-deshydroxy-4′-methyl etoposide (Saulnier et al., Bioorg. Med. Chem. Lett. 2 (10): 1213-18, 1992), pendulum ring etoposide analogues (Sinha et al., Eur. J. Cancer 26 (5): 590-3, 1990) and E-ring desoxy etoposide analogues (Saulnier et al., J. Med. Chem. 32 (7): 1418-20, 1989).

Within one preferred embodiment of the invention, the cell cycle inhibitor is paclitaxel, a compound which disrupts mitosis (M-phase) by binding to tubulin to form abnormal mitotic spindles or an analogue or derivative thereof. Briefly, paclitaxel is a highly derivatized diterpenoid (Wani et al., J. Am. Chem. Soc. 93: 2325, 1971) which has been obtained from the harvested and dried bark of Taxus brevifolia (Pacific Yew) and Taxomyces Andreanae and Endophytic Fungus of the Pacific Yew (Stierle et al., Science 60: 214-216, 1993). “Paclitaxel” (which may be understood herein to include formulations, prodrugs, analogues and derivatives such as, for example, TAXOL (Bristol Myers Squibb, New York, N.Y., TAXOTERE (Aventis Pharmaceuticals, France), docetaxel, 10-desacetyl analogues of paclitaxel and 3'N-desbenzoyl-3'N-t-butoxy carbonyl analogues of paclitaxel) may be readily prepared utilizing techniques known to those skilled in the art (see, e.g., Schiff et al., Nature 277: 665-667, 1979; Long and Fairchild, Cancer Research 54: 4355-4361, 1994; Ringel and Horwitz, J. Nat'l Cancer Inst. 83 (4): 288-291, 1991; Pazdur et al., Cancer Treat. Rev. 19 (4): 351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35 (52): 9709-9712, 1994; J. Med. Chem. 35: 4230-4237, 1992; J. Med. Chem. 34: 992-998, 1991; J. Natural Prod. 57 (10): 1404-1410, 1994; J. Natural Prod. 57 (11): 1580-1583, 1994; J. Am. Chem. Soc. 110: 6558-6560, 1988), or obtained from a variety of commercial sources, including for example, Sigma Chemical Co., St. Louis, Mo. (T7402—from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′- and/or 7-O-ester derivatives), (2′-and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG(5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, taxol analogues with modified phenylisoserine side chains, TAXOTERE, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the cell cycle inhibitor is a taxane having the formula (C1): 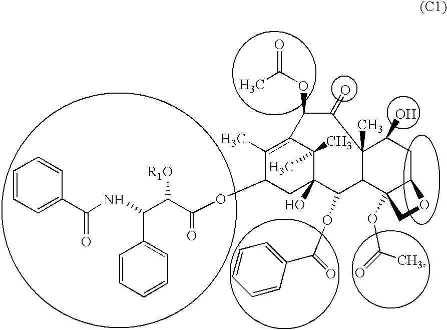
where the gray-highlighted portions may be substituted and the non-highlighted portion is the taxane core. A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE, Merck Index entry 3458), and 3′-desphenyl-3′-(4-ntirophenyl)-N-debenzoyl-N-(t-butoxycarbonyl)-10-deacetyltaxol.

In one aspect, suitable taxanes such as paclitaxel and its analogues and derivatives are disclosed in U.S. Pat. No. 5,440,056 as having the structure (C2): 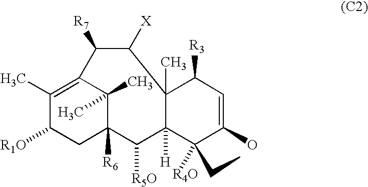
wherein X may be oxygen (paclitaxel), hydrogen (9-deoxy derivatives), thioacyl, or dihydroxyl precursors; R₁ is selected from paclitaxel or TAXOTERE side chains or alkanoyl of the formula (C3) 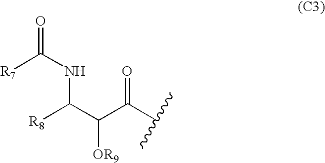
wherein R₇ is selected from hydrogen, alkyl, phenyl, alkoxy, amino, phenoxy (substituted or unsubstituted); R₈ is selected from hydrogen, alkyl, hydroxyalkyl, alkoxyalkyl, aminoalkyl, phenyl (substituted or unsubstituted), alpha or beta-naphthyl; and R₉ is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO₃H, and/or may refer to groups containing such substitutions; R₂ is selected from hydrogen or oxygen-containing groups, such as hydrogen, hydroxyl, alkoyl, alkanoyloxy, aminoalkanoyloxy, and peptidyalkanoyloxy; R₃ is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R₄ is selected from acyl, alkyl, alkanoyl, aminoalkanoyl, peptidylalkanoyl and aroyl; R₅ is selected from acyl, alkyl, alkanoyl, aminoalkanoyl, peptidylalkanoyl and aroyl; R₆ is selected from hydrogen or oxygen-containing groups, such as hydrogen, hydroxyl alkoyl, alkanoyloxy, aminoalkanoyloxy, and peptidyalkanoyloxy.

In one aspect, the paclitaxel analogues and derivatives useful as cell cycle inhibitors are disclosed in PCT International Patent Application No. WO 93/10076. As disclosed in this publication, the analogue or derivative may have a side chain attached to the taxane nucleus at C₁₃, as shown in the structure below (formula C4), in order to confer antitumor activity to the taxane.

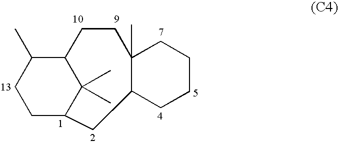

WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In one aspect, the taxane-based cell cycle inhibitor useful in the present invention is disclosed in U.S. Pat. No. 5,440,056, which discloses 9-deoxo taxanes. These are compounds lacking an oxo group at the carbon labeled 9 in the taxane structure shown above (formula C4). The taxane ring may be substituted at the carbons labeled 1, 7 and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (C3) may be substituted at R₇ and R₈ (independently) with phenyl rings, substituted phenyl rings, linear alkanes/alkenes, and groups containing H, O or N. R₉ may be substituted with H, or a substituted or unsubstituted alkanoyl group.

Taxanes in general, and paclitaxel is particular, is considered to function as a cell cycle inhibitor by acting as an anti-microtubule agent, and more specifically as a stabilizer. These compounds have been shown useful in the treatment of proliferative disorders, including: non-small cell (NSC) lung; small cell lung; breast; prostate; cervical; endometrial; head and neck cancers.

In another aspect, the anti-microtuble agent (microtubule inhibitor) is albendazole (carbamic acid, [5-(propylthio)-1H-benzimidazol-2-yl]-, methyl ester), LY-355703 (1,4-dioxa-8,11-diazacyclohexadec-13-ene-2,5,9,12-tetrone, 10-[(3-chloro-4-methoxyphenyl)methyl]-6,6-dimethyl-3-(2-methylpropyl)-16-[(1S)-1-[(2S,3R)-3-phenyloxiranyl]ethyl]-, (3S,10R,13E,16S)-), vindesine (vincaleukoblastine, 3-(aminocarbonyl)-04-deacetyl-3-de(methoxycarbonyl)-), or WAY-174286.

In another aspect, the cell cycle inhibitor is a vinca alkaloid. Vinca alkaloids have the following general structure. They are indole-dihydroindole dimers.

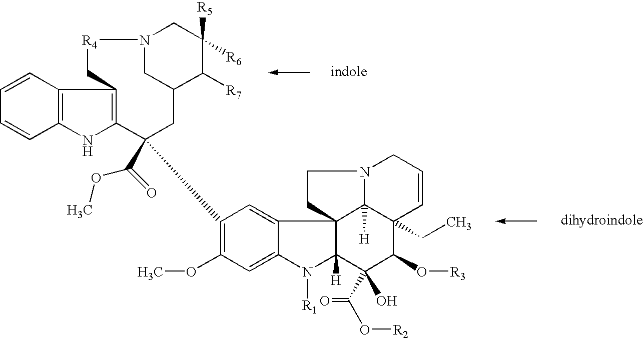

As disclosed in U.S. Pat. Nos. 4,841,045 and 5,030,620, R₁ can be a formyl or methyl group or alternately H. R₁ can also be an alkyl group or an aldehyde-substituted alkyl (e.g., CH₂CHO). R₂ is typically a CH₃ or NH₂ group. However it can be alternately substituted with a lower alkyl ester or the ester linking to the dihydroindole core may be substituted with C(O)—R where R is NH₂, an amino acid ester or a peptide ester. R₃ is typically C(O)CH₃, CH₃ or H. Alternately, a protein fragment may be linked by a bifunctional group, such as maleoyl amino acid. R₃ can also be substituted to form an alkyl ester which may be further substituted. R₄ may be —CH₂— or a single bond. R₅ and R₆ may be H, OH or a lower alkyl, typically —CH₂CH₃. Alternatively R₆ and R₇ may together form an oxetane ring. R₇ may alternately be H. Further substitutions include molecules wherein methyl groups are substituted with other alkyl groups, and whereby unsaturated rings may be derivatized by the addition of a side group such as an alkane, alkene, alkyne, halogen, ester, amide or amino group.

Exemplary vinca alkaloids are vinblastine, vincristine, vincristine sulfate, vindesine, and vinorelbine, having the structures:

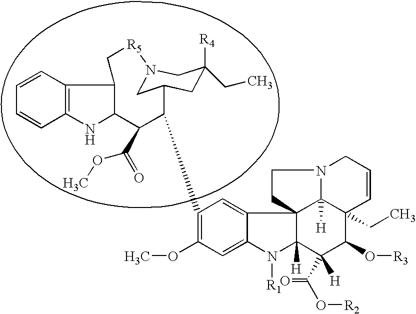

| | R₁ | R₂ | R₃ | R₄ | R₅ |
|---|---|---|---|---|---|
| Vinblastine: | CH3 | CH₃ | C(O)CH₃ | OH | CH₂ |
| Vincristine: | CH₂O | CH₃ | C(O)CH₃ | OH | CH₂ |
| Vindesine: | CH₃ | NH₂ | H | OH | CH₂ |
| Vinorelbine: | CH₃ | CH₃ | CH₃ | H | single bond |

Analogues typically require the side group (shaded area) in order to have activity. These compounds are thought to act as cell cycle inhibitors by functioning as anti-microtubule agents, and more specifically to inhibit polymerization. These compounds have been shown useful in treating proliferative disorders, including NSC lung; small cell lung; breast; prostate; brain; head and neck; retinoblastoma; bladder; and penile cancers; and soft tissue sarcoma.

In another aspect, the cell cycle inhibitor is a camptothecin, or an anolog or derivative thereof. Camptothecins have the following general structure.

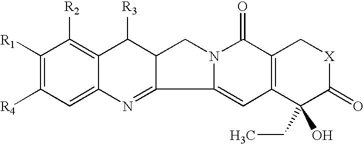

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R₁.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

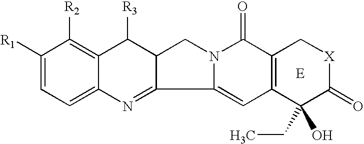

| | R₁ | R₂ | R₃ |
|---|---|---|---|
| Camptothecin: | H | H | H |
| Topotecen: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity. These compounds are useful to as cell cycle inhibitors, where they can function as topoisomerase I inhibitors and/or DNA cleavage agents. They have been shown useful in the treatment of proliferative disorders, including, for example, NSC lung; small cell lung; and cervical cancers.

In another aspect, the cell cycle inhibitor is a podophyllotoxin, or a derivative or an analogue thereof. Exemplary compounds of this type are etoposide or teniposide, which have the following structures:

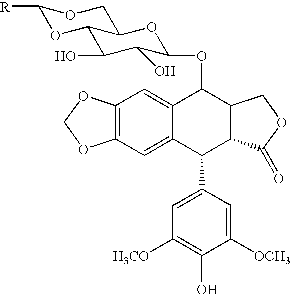

| | R |
|---|---|
| Etoposide | CH₃ |
| Teniposide | 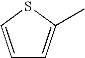 |

These compounds are thought to function as cell cycle inhibitors by being topoisomerase II inhibitors and/or by DNA cleaving agents. They have been shown useful as antiproliferative agents in, e.g., small cell lung, prostate, and brain cancers, and in retinoblastoma.

Another example of a DNA topoisomerase inhibitor is lurtotecan dihydrochloride (11H-1,4-dioxino[2,3-g]pyrano[3′,4′:6,7]indolizino[1,2-b]quinoline-9,12(8H,14H)-dione, 8-ethyl-2,3-dihydro-8-hydroxy-15-[(4-methyl-1-piperazinyl)methyl]-, dihydrochloride, (S)-).

In another aspect, the cell cycle inhibitor is an anthracycline. Anthracyclines have the following general structure, where the R groups may be a variety of organic groups:

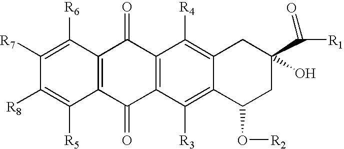

According to U.S. Pat. No. 5,594,158, suitable R groups are: R₁ is CH₃ or CH₂OH; R₂ is daunosamine or H; R₃ and R₄ are independently one of OH, NO₂, NH₂, F, Cl, Br, I, CN, H or groups derived from these; R₅₋₇ are all H or R₅ and R₆ are H and R₇ and R₈ are alkyl or halogen, or vice versa: R₇ and R₈ are H and R₅ and R₆ are alkyl or halogen.

According to U.S. Pat. No. 5,843,903, R₂ may be a conjugated peptide. According to U.S. Pat. Nos. 4,215,062 and 4,296,105, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 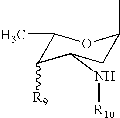
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁, is H, or forms a C₃₄ membered alkylene with R₁₂. R₁₂ may be H, alkyl, aminoalkyl, amino, hydroxy, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

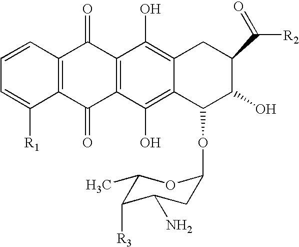

| | R₁ | R₂ | R₃ |
|---|---|---|---|
| Doxorubicin: | OCH₃ | CH₂OH | OH out of ring plane |
| Epirubicin: | OCH₃ | CH₂OH | OH in ring plane |
| (4′ spimer | | | |
| of doxorubicin) | | | |
| Daunorubicin: | OCH₃ | CH₃ | OH out of ring plane |
| Idarubicin: | H | CH₃ | OH out of ring plane |
| Pirarubicin | OCH₃ | OH | A |
| Zorubicin | OCH₃ | ═N—NHC(O)C₆H₅ | B |
| Carubicin | OH | CH₃ | B |

| | |
|---|---|
| A: | 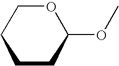 |
| B: | 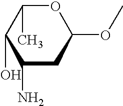 |

Other suitable anthracyclines are anthramycin, mitoxantrone, menogaril, nogalamycin, aclacinomycin A, olivomycin A, chromomycin A₃, and plicamycin having the structures:

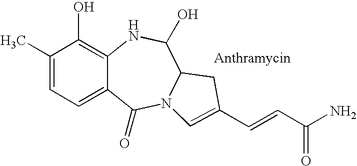

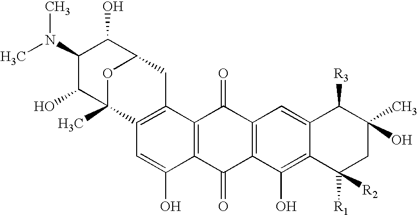

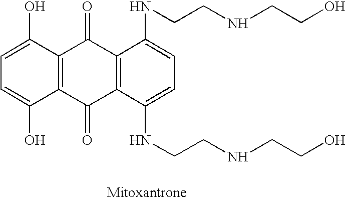

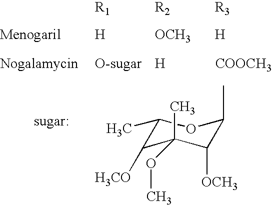

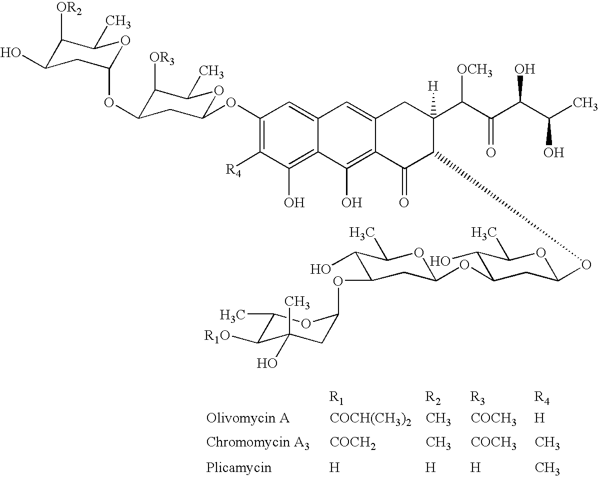

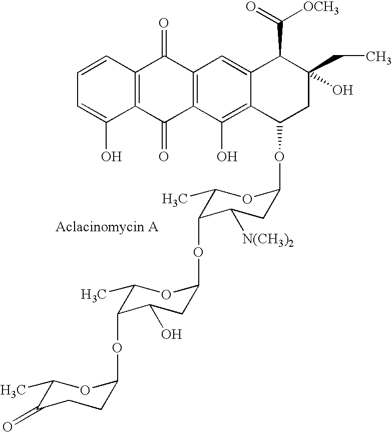

These compounds are thought to function as cell cycle inhibitors by being topoisomerase inhibitors and/or by DNA cleaving agents. They have been shown useful in the treatment of proliferative disorders, including small cell lung; breast; endometrial; head and neck; retinoblastoma; liver; bile duct; islet cell; and bladder cancers; and soft tissue sarcoma.

In another aspect, the cell cycle inhibitor is a platinum compound. In general, suitable platinum complexes may be of Pt(II) or Pt(IV) and have this basic structure: 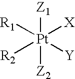
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

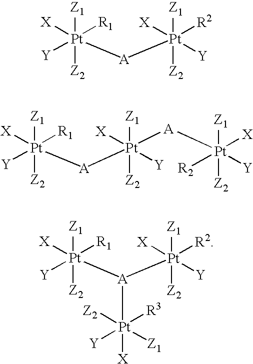

Exemplary platinum compounds are cisplatin, carboplatin, oxaliplatin, and miboplatin having the structures:

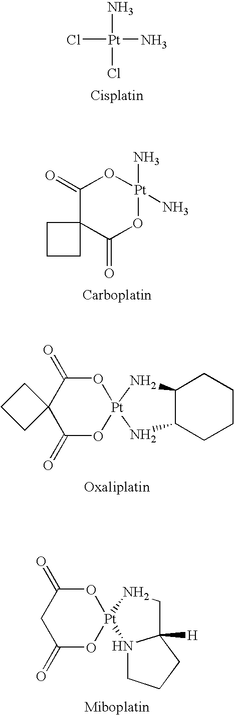

These compounds are thought to function as cell cycle inhibitors by binding to DNA, i.e., acting as alkylating agents of DNA. These compounds have been shown useful in the treatment of cell proliferative disorders, including, e.g., NSC lung; small cell lung; breast; cervical; brain; head and neck; esophageal; retinoblastom; liver; bile duct; bladder; penile; and vulvar cancers; and soft tissue sarcoma.

In another aspect, the cell cycle inhibitor is a nitrosourea. Nitrosourease have the following general structure (C5), where typical R groups are shown below.

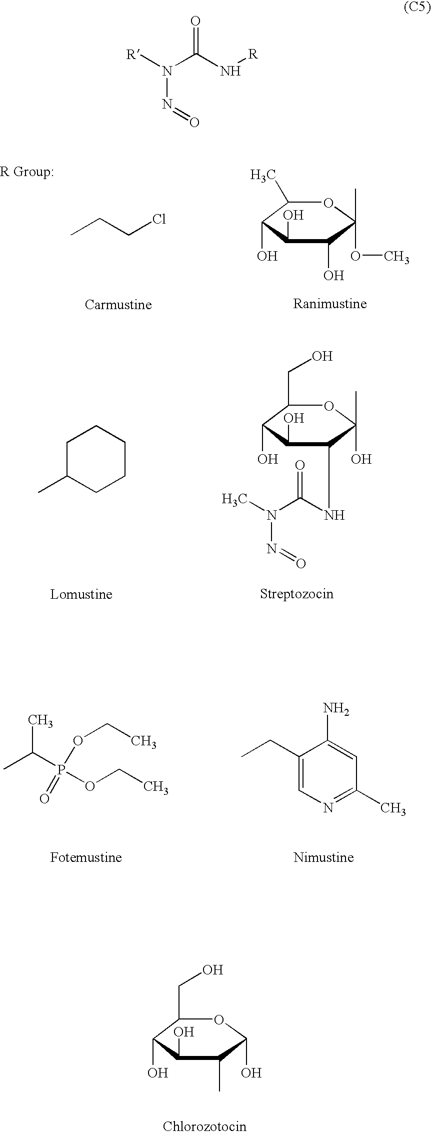

Other suitable R groups include cyclic alkanes, alkanes, halogen substituted groups, sugars, aryl and heteroaryl groups, phosphonyl and sulfonyl groups. As disclosed in U.S. Pat. No. 4,367,239, R may suitably be CH₂—C(X)(Y)(Z), wherein X and Y may be the same or different members of the following groups: phenyl, cyclyhexyl, or a phenyl or cyclohexyl group substituted with groups such as halogen, lower alkyl (C₁₋₄), trifluore methyl, cyano, phenyl, cyclohexyl, lower alkyloxy (C₁₋₄). Z has the following structure: -alkylene-N—R₁R₂, where R₁ and R₂ may be the same or different members of the following group: lower alkyl (C₁₋₄) and benzyl, or together R₁ and R₂ may form a saturated 5 or 6 membered heterocyclic such as pyrrolidine, piperidine, morfoline, thiomorfoline, N-lower alkyl piperazine, where the heterocyclic may be optionally substituted with lower alkyl groups.

As disclosed in U.S. Pat. No. 6,096,923, R and R′ of formula (C5) may be the same or different, where each may be a substituted or unsubstituted hydrocarbon having 1-10 carbons. Substitutions may include hydrocarbyl, halo, ester, amide, carboxylic acid, ether, thioether and alcohol groups. As disclosed in U.S. Pat. No. 4,472,379, R of formula (C5) may be an amide bond and a pyranose structure (e.g., methyl 2′-(N-(N-(2-chloroethyl)-N-nitroso-carbamoyl)-glycyl)amino-2′-deoxy-α-D-glucopyranoside). As disclosed in U.S. Pat. No. 4,150,146, R of formula (C5) may be an alkyl group of 2 to 6 carbons and may be substituted with an ester, sulfonyl, or hydroxyl group. It may also be substituted with a carboxylic acid or CONH₂ group.

Exemplary nitrosoureas are BCNU (carmustine), methyl-CCNU (semustine), CCNU (lomustine), ranimustine, nimustine, chlorozotocin, fotemustine, and streptozocin, having the structures:

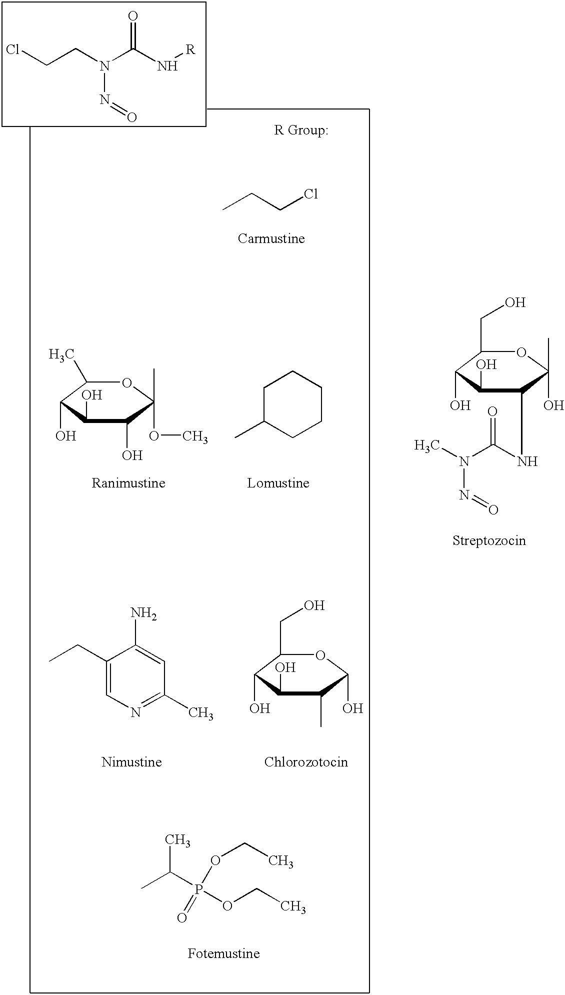

These nitrosourea compounds are thought to function as cell cycle inhibitors by binding to DNA, that is, by functioning as DNA alkylating agents. These cell cycle inhibitors have been shown useful in treating cell proliferative disorders such as, for example, islet cell; small cell lung; melanoma; and brain cancers.

In another aspect, the cell cycle inhibitor is a nitroimidazole, where exemplary nitroimidazoles are metronidazole, benznidazole, etanidazole, and misonidazole, having the structures:

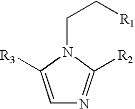

| | R₁ | R₂ | R₃ |
|---|---|---|---|
| Metronidazole | OH | CH₃ | NO₂ |
| Benznidazole | C(O)NHCH₂-benzyl | NO₂ | H |
| Etanidazole | CONHCH₂CH₂OH | NO₂ | H |

Suitable nitroimidazole compounds are disclosed in, e.g., U.S. Pat. Nos. 4,371,540 and 4,462,992.

In another aspect, the cell cycle inhibitor is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

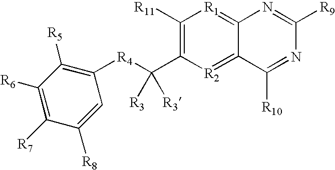

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 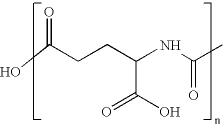
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

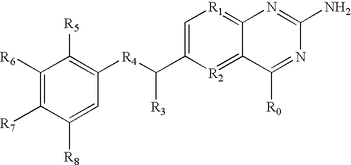

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A(n = 1) | H |
| Edatrexate | NH₂ | N | N | H | N(CH₂CH₃) | H | H | A(n = 1) | H |
| Trimetrexate | NH₂ | N | C(CH₃) | H | NH | H | OCH₃ | OCH₃ | OCH₃ |
| Pteropterin | NH₂ | N | N | H | N(CH₃) | H | H | A(n = 3) | H |
| Denopterin | OH | N | N | CH₃ | N(CH₃) | H | H | A(n = 1) | H |
| Piritrexim | NH₂ | N | C(CH₃)H | single | OCH₃ | H | H | OCH₃ | H |
| | | | | bond | | | | | |

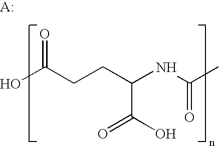

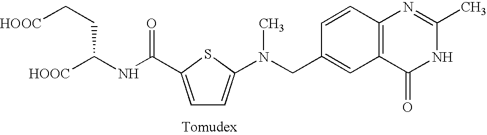

These compounds are thought to function as cell cycle inhibitors by serving as antimetabolites of folic acid. They have been shown useful in the treatment of cell proliferative disorders including, for example, soft tissue sarcoma, small cell lung, breast, brain, head and neck, bladder, and penile cancers.

In another aspect, the cell cycle inhibitor is a cytidine analogue, such as cytarabine or derivatives or analogues thereof, including enocitabine, FMdC ((E(−2′-deoxy-2′-(fluoromethylene)cytidine), gemcitabine, 5-azacitidine, ancitabine, and 6-azauridine. Exemplary compounds have the structures:

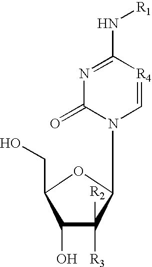

| | R₁ | R₂ | R₃ | R₄ |
|---|---|---|---|---|
| Cytarabine | H | OH | H | CH |
| Enocitabine | C(O)(CH₂)₂₀CH₃ | OH | H | CH |
| Gemcitabine | H | F | F | CH |
| Azacitidine | H | H | OH | N |
| FMdC | H | CH₂F | H | CH |

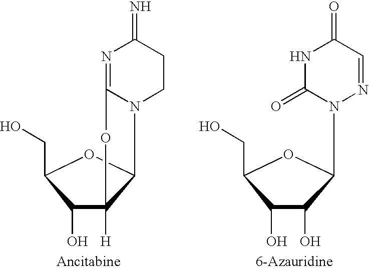

These compounds are thought to function as cell cycle inhibitors as acting as antimetabolites of pyrimidine. These compounds have been shown useful in the treatment of cell proliferative disorders including, for example, pancreatic, breast, cervical, NSC lung, and bile duct cancers.

In another aspect, the cell cycle inhibitor is a pyrimidine analogue. In one aspect, the pyrimidine analogues have the general structure: 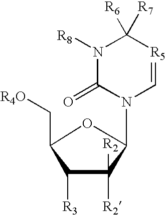
wherein positions 2′, 3′ and 5′ on the sugar ring (R₂, R₃ and R₄, respectively) can be H, hydroxyl, phosphoryl (see, e.g., U.S. Pat. No. 4,086,417) or ester (see, e.g., U.S. Pat. No. 3,894,000). Esters can be of alkyl, cycloalkyl, aryl or heterocyclo/aryl types. The 2′ carbon can be hydroxylated at either R₂ or R₂′, the other group is H. Alternately, the 2′ carbon can be substituted with halogens e.g., fluoro or difluoro cytidines such as Gemcytabine. Alternately, the sugar can be substituted for another heterocyclic group such as a furyl group or for an alkane, an alkyl ether or an amide linked alkane such as C(O)NH(CH₂)₅CH₃. The 2° amine can be substituted with an aliphatic acyl (R₁) linked with an amide (see, e.g., U.S. Pat. No. 3,991,045) or urethane (see, e.g., U.S. Pat. No. 3,894,000) bond. It can also be further substituted to form a quaternary ammonium salt. R₅ in the pyrimidine ring may be N or CR, where R is H, halogen containing groups, or alkyl (see, e.g., U.S. Pat. No. 4,086,417). R₆ and R₇ can together can form an oxo group or R₆═—NH—R, and R₇═H. R₈ is H or R₇ and R₈ together can form a double bond or R₈ can be X, where X is:

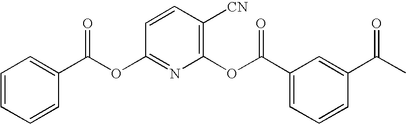

Specific pyrimidine analogues are disclosed in U.S. Pat. No. 3,894,000 (see, e.g., 2′-O-palmityl-ara-cytidine, 3′-O-benzoyl-ara-cytidine, and more than 10 other examples); U.S. Pat. No. 3,991,045 (see, e.g., N4-acyl-1-β-D-arabinofuranosylcytosine, and numerous acyl groups derivatives as listed therein, such as palmitoyl.

In another aspect, the cell cycle inhibitor is a fluoropyrimidine analogue, such as 5-fluorouracil, or an analogue or derivative thereof, including carmofur, doxifluridine, emitefur, tegafur, and floxuridine. Exemplary compounds have the structures:

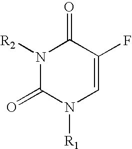

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxundine | A₂ | H |
| Emitefur | CH₂OCH₂CH₃ | B |
| Tegafur | | H |

| | |
|---|---|
| A₁ | 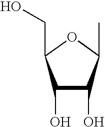 |
| A₂ | 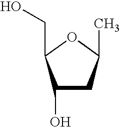 |
| B | 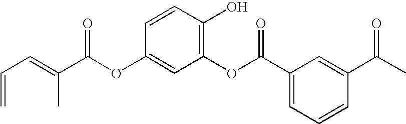 |
| C | 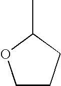 |

Other suitable fluoropyrimidine analogues include 5-FudR (5-fluorodeoxyuridine), or an analogue or derivative thereof, including 5-iododeoxyuridine (5-ludR), 5-bromodeoxyuridine (5-BudR), fluorouridine triphosphate (5-FUTP), and fluorodeoxyuridine monophosphate (5-dFUMP). Exemplary compounds have the structures:

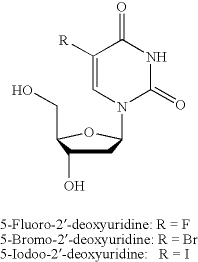

These compounds are thought to function as cell cycle inhibitors by serving as antimetabolites of pyrimidine. These compounds have been shown useful in the treatment of cell proliferative disorders such as breast, cervical, non-melanoma skin, head and neck, esophageal, bile duct, pancreatic, islet cell, penile, and vulvar cancers.

In another aspect, the cell cycle inhibitor is a purine analogue. Purine analogues have the following general structure. 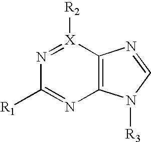
wherein X is typically carbon; R₁ is H, halogen, amine or a substituted phenyl; R₂ is H, a primary, secondary or tertiary amine, a sulfur containing group, typically —SH, an alkane, a cyclic alkane, a heterocyclic or a sugar; R₃ is H, a sugar (typically a furanose or pyranose structure), a substituted sugar or a cyclic or heterocyclic alkane or aryl group. See, e.g., U.S. Pat. No. 5,602,140 for compounds of this type.

In the case of pentostatin, X—R2 is —CH₂CH(OH)—. In this case a second carbon atom is inserted in the ring between X and the adjacent nitrogen atom. The X—N double bond becomes a single bond.

U.S. Pat. No. 5,446,139 describes suitable purine analogues of the type shown in the formula. 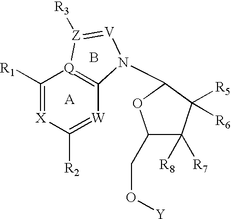
wherein N signifies nitrogen and V, W, X, Z can be either carbon or nitrogen with the following provisos. Ring A may have 0 to 3 nitrogen atoms in its structure. If two nitrogens are present in ring A, one must be in the W position. If only one is present, it must not be in the Q position. V and Q must not be simultaneously nitrogen. Z and Q must not be simultaneously nitrogen. If Z is nitrogen, R₃ is not present. Furthermore, R₁₋₃ are independently one of H, halogen, C₁₋₇ alkyl, C₁₋₇ alkenyl, hydroxyl, mercapto, C₁₋₇ alkylthio, C₁₋₇ alkoxy, C₂₋₇ alkenyloxy, aryl oxy, nitro, primary, secondary or tertiary amine containing group. R 8 are H or up to two of the positions may contain independently one of OH, halogen, cyano, azido, substituted amino, R₅ and R₇ can together form a double bond. Y is H, a C₁₋₇ alkylcarbonyl, or a mono- di or tri phosphate.

Exemplary suitable purine analogues include 6-mercaptopurine, thiguanosine, thiamiprine, cladribine, fludaribine, tubercidin, puromycin, pentoxyfilline; where these compounds may optionally be phosphorylated. Exemplary compounds have the structures:

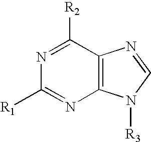

| | R₁ | R₂ | R₃ |
|---|---|---|---|
| 6-Mercaptopurine | H | SH | H |
| Thioguanosine | NH₂ | SH | B₁ |
| Thiamiprine | NH₂ | A | H |
| Cladribine | Cl | NH₂ | B₂ |
| Fludarabine | F | NH₂ | B₂ |
| Puromycin | H | N(CH₃)₂ | B₄ |
| Tubercidin | H | NH₂ | B₁ |

| | |
|---|---|
| A: | 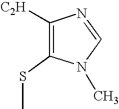 |
| B₁: | 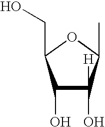 |
| B₂: | 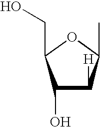 |
| B₃: | 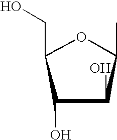 |
| B₄: | 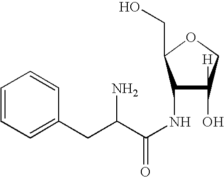 |

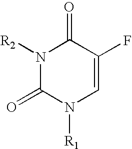

These compounds are thought to function as cell cycle inhibitors by serving as antimetabolites of purine.

In another aspect, the cell cycle inhibitor is a nitrogen mustard. Many suitable nitrogen mustards are known and are suitably used as a cell cycle inhibitor in the present invention. Suitable nitrogen mustards are also known as cyclophosphamides.

A preferred nitrogen mustard has the general structure: 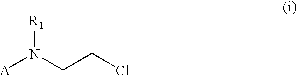
Where A is: 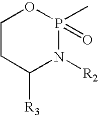
or —CH₃ or other alkane, or chloronated alkane, typically CH₂CH(CH₃)Cl, or a polycyclic group such as B, or a substituted phenyl such as C or a heterocyclic group such as D.

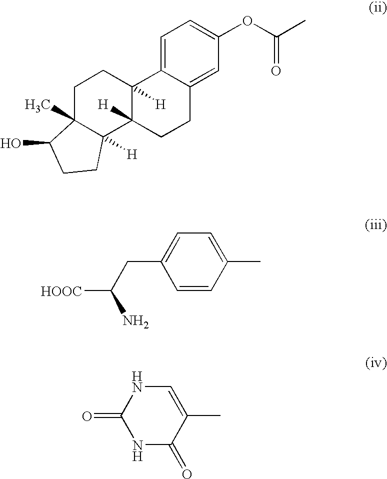

Examples of suitable nitrogen mustards are disclosed in U.S. Pat. No. 3,808,297, wherein A is:

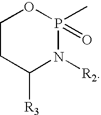

R₁₋₂ are H or CH₂CH₂Cl; R₃ is H or oxygen-containing groups such as hydroperoxy; and R₄ can be alkyl, aryl, heterocyclic.

The cyclic moiety need not be intact. See, e.g., U.S. Pat. Nos. 5,472,956, 4,908,356, 4,841,085 that describe the following type of structure: 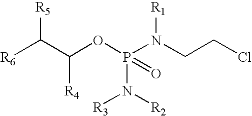
wherein R₁ is H or CH₂CH₂Cl, and R₂₋₆ are various substituent groups.

Exemplary nitrogen mustards include methylchloroethamine, and analogues or derivatives thereof, including methylchloroethamine oxide hydrohchloride, novembichin, and mannomustine (a halogenated sugar). Exemplary compounds have the structures:

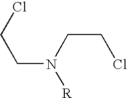

| | R |
|---|---|
| Mechlorethanime | CH₃ |
| Novembichin | CH₂CH(CH₃)Cl |

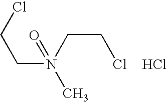

Mechlorethanime Oxide HCl

The nitrogen mustard may be cyclophosphamide, ifosfamide, perfosfamide, or torofosfamide, where these compounds have the structures:

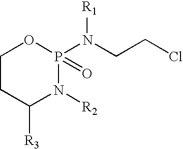

| | R₁ | R₂ | R₃ |
|---|---|---|---|
| Cyclophosphamide | H | CH₂CH₂Cl | H |
| Ifosfamide | CH₂CH₂Cl | H | H |
| Perfosfamide | CH₂CH₂Cl | H | OOH |
| Torofosfamide | CH₂CH₂Cl | CH₂CH₂Cl | H |

The nitrogen mustard may be estramustine, or an analogue or derivative thereof, including phenesterine, prednimustine, and estramustine PO₄. Thus, suitable nitrogen mustard type cell cycle inhibitors of the present invention have the structures:

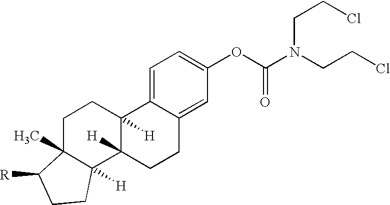

| | R |
|---|---|
| Estramustine | OH |
| Phenesterine | C(CH₃)(CH₂)₃CH(CH₃)₂ |

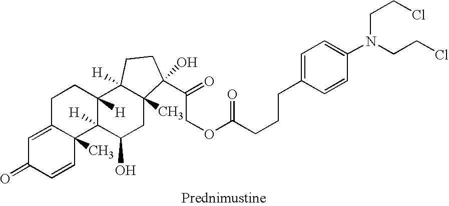

The nitrogen mustard may be chlorambucil, or an analogue or derivative thereof, including melphalan and chlormaphazine. Thus, suitable nitrogen mustard type cell cycle inhibitors of the present invention have the structures:

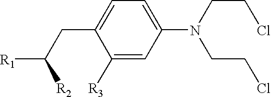

| | R₁ | R₂ | R₃ |
|---|---|---|---|
| Chlorambucil | CH₂COOH | H | H |
| Melphalan | COOH | NH₂ | H |

| | | |
|---|---|---|
| Chlornaphazine | H | together forms a |
| | | benzene ring |

The nitrogen mustard may be uracil mustard, which has the structure:

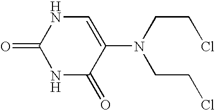

The nitrogen mustards are thought to function as cell cycle inhibitors by serving as alkylating agents for DNA. Nitrogen mustards have been shown useful in the treatment of cell proliferative disorders including, for example, small cell lung, breast, cervical, head and neck, prostate, retinoblastoma, and soft tissue sarcoma.

The cell cycle inhibitor of the present invention may be a hydroxyurea. Hydroxyureas have the following general structure:

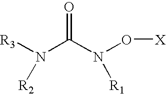

Suitable hydroxyureas are disclosed in, for example, U.S. Pat. No. 6,080,874, wherein R₁ is: 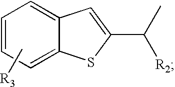
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-(3-(5-(4-fluorophenylthio)-furyl)-2-cyclopenten-1-yl)N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with on or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 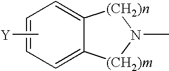
wherein m is 1 or 2, n is 0-2 and Y is an alkyl group.

In one aspect, the hydroxy urea has the structure:

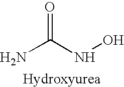

Hydroxyureas are thought to function as cell cycle inhibitors by serving to inhibit DNA synthesis.

In another aspect, the cell cycle inhibitor is a mytomicin, such as mitomycin C, or an analogue or derivative thereof, such as porphyromycin. Exemplary compounds have the structures:

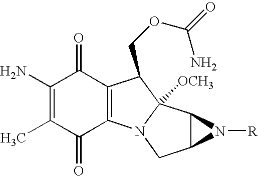

| | R |
|---|---|
| Mitomycin C | H |
| Porphyromycin | CH₃ |
| (N-methyl Mitomycin C) | |

These compounds are thought to function as cell cycle inhibitors by serving as DNA alkylating agents. Mitomycins have been shown useful in the treatment of cell proliferative disorders such as, for example, esophageal, liver, bladder, and breast cancers.

In another aspect, the cell cycle inhibitor is an alkyl sulfonate, such as busulfan, or an analogue or derivative thereof, such as treosulfan, improsulfan, piposulfan, and pipobroman. Exemplary compounds have the structures:

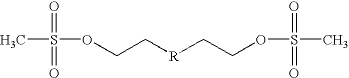

| | R |
|---|---|
| Busulfan | single band |
| Improsulfan | —CH₂—NH—CH₂— |
| Piposulfan | 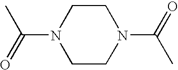 |

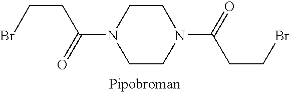

These compounds are thought to function as cell cycle inhibitors by serving as DNA alkylating agents.

In another aspect, the cell cycle inhibitor is a benzamide. In yet another aspect, the cell cycle inhibitor is a nicotinamide. These compounds have the basic structure: 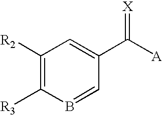
wherein X is either O or S; A is commonly NH₂ or it can be OH or an alkoxy group; B is N or C—R₄, where R₄ is H or an ether-linked hydroxylated alkane such as OCH₂CH₂OH, the alkane may be linear or branched and may contain one or more hydroxyl groups. Alternately, B may be N—R₅ in which case the double bond in the ring involving B is a single bond. R₅ may be H, and alkyl or an aryl group (see, e.g., U.S. Pat. No. 4,258,052); R₂ is H, OR₆, SR₆ or NHR₆, where R₆ is an alkyl group; and R₃ is H, a lower alkyl, an ether linked lower alkyl such as —O-Me or —O-ethyl (see, e.g., U.S. Pat. No. 5,215,738).

Suitable benzamide compounds have the structures: 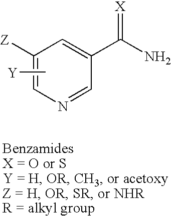
where additional compounds are disclosed in U.S. Pat. No. 5,215,738, (listing some 32 compounds).

Suitable nicotinamide compounds have the structures:

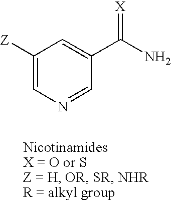

where additional compounds are disclosed in U.S. Pat. No. 5,215,738,

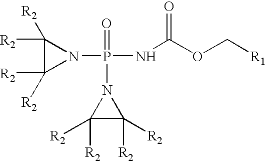

| | R₁ | R₂ |
|---|---|---|
| Benzodepa | phenyl | H |
| Meturedepa | CH₃ | CH₃ |
| Uredepa | CH₃ | H |

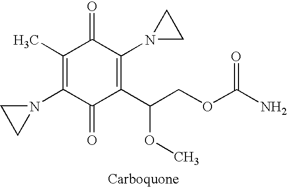

In another aspect, the cell cycle inhibitor is a halogenated sugar, such as mitolactol, or an analogue or derivative thereof, including mitobronitol and mannomustine. Examplary compounds have the structures:

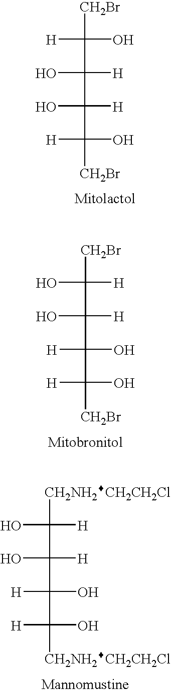

In another aspect, the cell cycle inhibitor is a diazo compound, such as azaserine, or an analogue or derivative thereof, including 6-diazo-5-oxo-L-norleucine and 5-diazouracil (also a pyrimidine analog). Examplary compounds have the structures:

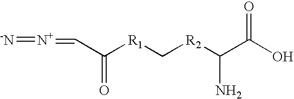

| | R¹ | R² |
|---|---|---|
| Azaserine | O | single bond |
| 6-diazo-5-oxo-L-norleucine | single bond | CH₂ |

Other compounds that may serve as cell cycle inhibitors according to the present invention are pazelliptine; wortmannin; metoclopramide; RSU; buthionine sulfoxime; tumeric; curcumin; AG337, a thymidylate synthase inhibitor; levamisole; lentinan, a polysaccharide; razoxane, an EDTA analogue; indomethacin; chlorpromazine; α and β interferon; MnBOPP; gadolinium texaphyrin; 4-amino-1,8-naphthalimide; staurosporine derivative of CGP; and SR-2508.

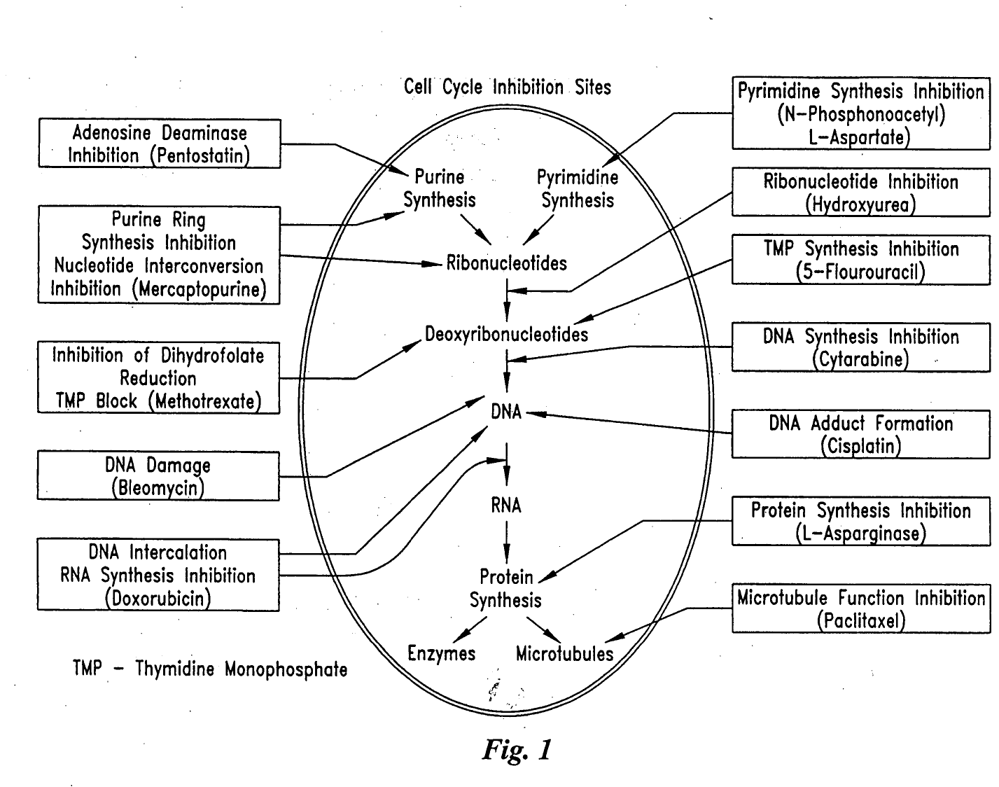

Thus, in one aspect, the cell cycle inhibitor is a DNA alylating agent. In another aspect, the cell cycle inhibitor is an anti-microtubule agent. In another aspect, the cell cycle inhibitor is a topoisomerase inhibitor. In another aspect, the cell cycle inhibitor is a DNA cleaving agent. In another aspect, the cell cycle inhibitor is an antimetabolite. In another aspect, the cell cycle inhibitor functions by inhibiting adenosine deaminase (e.g., as a purine analogue). In another aspect, the cell cycle inhibitor functions by inhibiting purine ring synthesis and/or as a nucleotide interconversion inhibitor (e.g., as a purine analogue such as mercaptopurine). In another aspect, the cell cycle inhibitor functions by inhibiting dihydrofolate reduction and/or as a thymidine monophosphate block (e.g., methotrexate). In another aspect, the cell cycle inhibitor functions by causing DNA damage (e.g., bleomycin). In another aspect, the cell cycle inhibitor functions as a DNA intercalation agent and/or RNA synthesis inhibition (e.g., doxorubicin, aclarubicin, or detorubicin (acetic acid, diethoxy-, 2-[4-[(3-amino-2,3,6-trideoxy-alpha-L-lyxo-hexopyranosyl)oxy]-1,2,3,4,6,11-hexahydro-2,5,12-trihydroxy-7-methoxy-6,11-dioxo-2-naphthacenyl]-2-oxoethyl ester, (2S-cis)-)). In another aspect, the cell cycle inhibitor functions by inhibiting pyrimidine synthesis (e.g., N-phosphonoacetyl-L-aspartate). In another aspect, the cell cycle inhibitor functions by inhibiting ribonucleotides (e.g., hydroxyurea). In another aspect, the cell cycle inhibitor functions by inhibiting thymidine monophosphate (e.g., 5-fluorouracil). In another aspect, the cell cycle inhibitor functions by inhibiting DNA synthesis (e.g., cytarabine). In another aspect, the cell cycle inhibitor functions by causing DNA adduct formation (e.g., platinum compounds). In another aspect, the cell cycle inhibitor functions by inhibiting protein synthesis (e.g., L-asparginase). In another aspect, the cell cycle inhibitor functions by inhibiting microtubule function (e.g., taxanes). In another aspect, the cell cycle inhibitor acts at one or more of the steps in the biological pathway shown in FIG. 1.

Additional cell cycle inhibitor s useful in the present invention, as well as a discussion of the mechanisms of action, may be found in Hardman J. G., Limbird L. E. Molinoff R. B., Ruddon R W., Gilman A. G. editors, Chemotherapy of Neoplastic Diseases in Goodman and Gilman's The Pharmacological Basis of Therapeutics Ninth Edition, McGraw-Hill Health Professions Division, New York, 1996, pages 1225-1287. See also U.S. Pat. Nos. 3,387,001; 3,808,297; 3,894,000; 3,991,045; 4,012,390; 4,057,548; 4,086,417; 4,144,237; 4,150,146; 4,210,584; 4,215,062; 4,250,189; 4,258,052; 4,259,242; 4,296,105; 4,299,778; 4,367,239; 4,374,414; 4,375,432; 4,472,379; 4,588,831; 4,639,456; 4,767,855; 4,828,831; 4,841,045; 4,841,085; 4,908,356; 4,923,876; 5,030,620; 5,034,320; 5,047,528; 5,066,658; 5,166,149; 5,190,929; 5,215,738; 5,292,731; 5,380,897; 5,382,582; 5,409,915; 5,440,056; 5,446,139; 5,472,956; 5,527,905; 5,552,156; 5,594,158; 5,602,140; 5,665,768; 5,843,903; 6,080,874; 6,096,923; and RE030561.

In another embodiment, the cell-cycle inhibitor is camptothecin, mitoxantrone, etoposide, 5-fluorouracil, doxorubicin, methotrexate, peloruside A, mitomycin C, or a CDK-2 inhibitor or an analogue or derivative of any member of the class of listed compounds.

In another embodiment, the cell-cycle inhibitor is HTI-286, plicamycin; or mithramycin, or an analogue or derivative thereof.

Other examples of cell cycle inhibitors also include, e.g., 7-hexanoyltaxol (QP-2), cytochalasin A, lantrunculin D, actinomycin-D, Ro-31-7453 (3-(6-nitro-1-methyl-3-indolyl)-4-(1-methyl-3-indolyl)pyrrole-2,5-dione), PNU-151807, brostallicin, C2-ceramide, cytarabine ocfosfate (2(1H)-pyrimidinone, 4-amino-1-(5-O-(hydroxy(octadecyloxy)phosphinyl)-β-D-arabinofuranosyl)-, monosodium salt), paclitaxel (5β,20-epoxy-1,2 alpha,4,7β,10β,13 alpha-hexahydroxytax-11-en-9-one-4,10-diacetate-2-benzoate-13-(alpha-phenylhippurate)), doxorubicin (5,12-naphthacenedione, 10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-, (8S)-cis-), daunorubicin (5,12-naphthacenedione, 8-acetyl-10-((3-amino-2,3,6-trideoxy-alpha-L-lyxo-hexopyranosyl)oxy)-7,8,9,10-tetrahydro-6,8,11-trihydroxy-1-methoxy-, (8S-cis)-), gemcitabine hydrochloride (cytidine, 2′-deoxy-2′,2′-difluoro-,monohydrochloride), nitacrine (1,3-propanediamine, N,N-dimethyl-N′-(1-nitro-9-acridinyl)-), carboplatin (platinum, diammine(1,1-cyclobutanedicarboxylato(2-))-, (SP-4-2)-), altretamine (1,3,5-triazine-2,4,6-triamine, N,N,N′,N′,N″,N″-hexamethyl-), teniposide (furo(3′,4′:6,7)naphtho(2,3-d)-1,3-dioxol-6(5aH)-one, 5,8,8a,9-tetrahydro-5-(4-hydroxy-3,5-dimethoxyphenyl)-9-((4,6-O-(2-thienylmethylene)-β-D-glucopyranosyl)oxy)-, (5R-(5alpha,5aβ,8aAlpha,9β(R*)))-), eptaplatin (platinum, ((4R,5R)-2-(1-methylethyl)-1,3-dioxolane-4,5-dimethanamine-kappa N4,kappa N5)(propanedioato(2-)-kappa O1, kappa O3)-, (SP-4-2)-), amrubicin hydrochloride (5,12-naphthacenedione, 9-acetyl-9-amino-7-((2-deoxy-β-D-erythro-pentopyranosyl)oxy)-7,8,9,10-tetrahydro-6,11-dihydroxy-, hydrochloride, (7S-cis)-), ifosfamide (2H-1,3,2-oxazaphosphorin-2-amine, N,3-bis(2-chloroethyl)tetrahydro-,2-oxide), cladribine (adenosine, 2-chloro-2′-deoxy-), mitobronitol (D-mannitol, 1,6-dibromo-1,6-dideoxy-), fludaribine phosphate (9H-purin-6-amine, 2-fluoro-9-(5-O-phosphono-β-D-arabinofuranosyl)-), enocitabine (docosanamide, N-(1-β-D-arabinofuranosyl-1,2-dihydro-2-oxo-4-pyrimidinyl)-), vindesine (vincaleukoblastine, 3-(aminocarbonyl)-O4-deacetyl-3-de(methoxycarbonyl)-), idarubicin (5,12-naphthacenedione, 9-acetyl-7-((3-amino-2,3,6-trideoxy-alpha-L-lyxo-hexopyranosyl)oxy)-7,8,9,10-tetrahydro-6,9,11-trihydroxy-, (7S-cis)-), zinostatin (neocarzinostatin), vincristine (vincaleukoblastine, 22-oxo-), tegafur (2,4(1H,3H)-pyrimidinedione, 5-fluoro-1-(tetrahydro-2-furanyl)-), razoxane (2,6-piperazinedione, 4,4′-(1-methyl-1,2-ethanediyl)bis-), methotrexate (L-glutamic acid, N-(4-(((2,4-diamino-6-pteridinyl)methyl)methylamino)benzoyl)-), raltitrexed (L-glutamic acid, N-((5-(((1,4-dihydro-2-methyl-4-oxo-6-quinazolinyl)methyl)methylamino)-2-thienyl)carbonyl)-), oxaliplatin (platinum, (1,2-cyclohexanediamine-N,N′)(ethanedioato(2-)-O,O′)-, (SP-4-2-(1R-trans))-), doxifluridine (uridine, 5′-deoxy-5-fluoro-), mitolactol (galactitol, 1,6-dibromo-1,6-dideoxy-), piraubicin (5,12-naphthacenedione, 10-((3-amino-2,3,6-trideoxy-4-O-(tetrahydro-2H-pyran-2-yl)-alpha-L-lyxo-hexopyranosyl)oxy)-7,8,9,10-tetrahydro-6,8,11-trihydroxy-8-(hydroxyacetyl)-1-methoxy-, (8S-(8 alpha, 10 alpha(S*)))-), docetaxel ((2R,3S)-N-carboxy-3-phenylisoserine, N-tert-butyl ester, 13-ester with 5β,20-epoxy-1,2 alpha,4,7β,10β,13 alpha-hexahydroxytax-11-en-9-one 4-acetate 2-benzoate-), capecitabine (cytidine, 5-deoxy-5-fluoro-N-((pentyloxy)carbonyl)-), cytarabine (2(1H)-pyrimidone, 4-amino-1-β-D-arabino furanosyl-), valrubicin (pentanoic acid, 2-(1,2,3,4,6,11-hexahydro-2,5,12-trihydroxy-7-methoxy-6,11-dioxo-4-((2,3,6-trideoxy-3-((trifluoroacetyl)amino)-alpha-L-lyxo-hexopyranosyl)oxy)-2-naphthacenyl)-2-oxoethyl ester (2S-cis)-), trofosfamide (3-2-(chloroethyl)-2-(bis(2-chloroethyl)amino)tetrahydro-2H-1,3,2-oxazaphosphorin 2-oxide), prednimustine (pregna-1,4-diene-3,20-dione; 21-(4-(4-(bis(2-chloroethyl)amino)phenyl)-1-oxobutoxy)-11,17-dihydroxy-, (11β)-), lomustine (Urea, N-(2-chloroethyl)-N′-cyclohexyl-N-nitroso-), epirubicin (5,12-naphthacenedione, 10-((3-amino-2,3,6-trideoxy-alpha-L-arabino-hexopyranosyl)oxy)-7,8,9,10-tetrahydro-6,8,11-trihydroxy-8-(hydroxyacetyl)-1-methoxy-, (8S-cis)-), or an analogue or derivative thereof).

5. Cyclin Dependent Protein Kinase Inhibitors

In another embodiment, the pharmacologically active compound is a cyclin dependent protein kinase inhibitor (e.g., R-roscovitine, CYC-101, CYC-103, CYC-400, MX-7065, alvocidib (4H-1-Benzopyran-4-one, 2-(2-chlorophenyl)-5,7-dihydroxy-8-(3-hydroxy-1-methyl-4-piperidinyl)-, cis-(−)-), SU-9516, AG-12275, PD-0166285, CGP-79807, fascaplysin, GW-8510 (benzenesulfonamide, 4-(((Z)-(6,7-dihydro-7-oxo-8H-pyrrolo(2,3-g)benzothiazol-8-ylidene)methyl)amino)-N-(3-hydroxy-2,2-dimethylpropyl)-), GW-491619, Indirubin 3′ monoxime, GW8510, AZD-5438, ZK-CDK or an analogue or derivative thereof).

6. EGF (Epidermal Growth Factor) Receptor Kinase Inhibitors

In another embodiment, the pharmacologically active compound is an EGF (epidermal growth factor) kinase inhibitor (e.g., erlotinib (4-quinazolinamine, N-(3-ethynylphenyl)-6,7-bis(2-methoxyethoxy)-, monohydrochloride), erbstatin, BIBX-1382, gefitinib (4-quinazolinamine, N-(3-chloro-4-fluorophenyl)-7-methoxy-6-(3-(4-morpholinyl)propoxy)), or an analogue or derivative thereof).

7. Elastase Inhibitors

In another embodiment, the pharmacologically active compound is an elastase inhibitor (e.g., ONO-6818, sivelestat sodium hydrate (glycine, N-(2-(((4-(2,2-dimethyl-1-oxopropoxy)phenyl)sulfonyl)amino)benzoyl)-), erdosteine (acetic acid, ((2-oxo-2-((tetrahydro-2-oxo-3-thienyl)amino)ethyl)thio)-), MDL-100948A, MDL-104238 (N-(4-(4-morpholinylcarbonyl)benzoyl)-L-valyl-N′-(3,3,4,4,4-pentafluoro-1-(1-methylethyl)-2-oxobutyl)-L-2-azetamide), MDL-27324 (L-prolinamide, N-((5-(dimethylamino)-1-naphthalenyl)sulfonyl)-L-alanyl-L-ala nyl-N-(3,3,3-trifluoro-1-(1-methylethyl)-2-oxopropyl)-, (S)-), S R-26831 (thieno(3,2-c)pyridinium, 5-((2-chlorophenyl)methyl)-2-(2,2-dimethyl-1-oxopropoxy)-4,5,6,7-tetrahydro-5-hydroxy-), Win-68794, Win-63110, SSR-69071 (2-(9(2-piperidinoethoxy)-4-oxo-4H-pyrido(1,2-a)pyrimidin-2-yloxymethyl)-4-(1-methylethyl)-6-methyoxy-1,2-benzisothiazol-3(2H)-one-1,1-dioxide), (N(Alpha)-(1-adamantylsulfonyl)N(epsilon)-succinyl-L-lysyl-L-prolyl-L-valinal), Ro-31-3537 (N alpha-(1-adamantanesulphonyl)-N-(4-carboxybenzoyl)-L-lysyl-alanyl-L-valinal), R-665, FCE-28204, ((6R,7R)-2-(benzoyloxy)-7-methoxy-3-methyl-4-pivaloyl-3-cephem 1,1-dioxide), 1,2-benzisothiazol-3(2H)-one, 2-(2,4-dinitrophenyl)-, 1,1-dioxide, L-658758 (L-proline, 1-((3-((acetyloxy)methyl)-7-methoxy-8-oxo-5-thia-1-azabicyclo(4.2.0)oct-2-en-2-yl)carbonyl)-, S,S-dioxide, (6R-cis)-), L-659286 (pyrrolidine, 1-((7-methoxy-8-oxo-3-(((1,2,5,6-tetrahydro-2-methyl-5,6-dioxo-1,2,4-triazin-3-yl)thio)methyl)-5-thia-1-azabicyclo(4.2.0)oct-2-en-2-yl)carbonyl)-, S,S-dioxide, (6R-cis)-), L-680833 (benzeneacetic acid, 4-((3,3-diethyl-1-(((1-(4-methylphenyl)butyl)amino)carbonyl)-4-oxo-2-azetidinyl)oxy)-, (S-(R*,S*))-), FK-706 (L-prolinamide, N-[4-[[(carboxymethyl)amino]carbonyl]benzoyl]-L-valyl-N-[3,3,3-trifluoro-1-(1-methylethyl)-2-oxopropyl]-, monosodium salt), Roche R-665, or an analogue or derivative thereof).

8. Factor Xa Inhibitors

In another embodiment, the pharmacologically active compound is a factor Xa inhibitor (e.g., CY-222, fondaparinux sodium (alpha-D-glucopyranoside, methyl O-2-deoxy-6-O-sulfo-2-(sulfoamino)-alpha-D-glucopyranosyl-(1-4)-O-β-D-glucopyranuronosyl-(1-4)-O-2-deoxy-3,6-di-O-sulfo-2-(sulfoamino)-alpha-D-glucopyranosyl-(1-4)-O-2-O-sulfo-alpha-L-idopyranuronosyl-(1-4)-2-deoxy-2-(sulfoamino)-, 6-(hydrogen sulfate)), danaparoid sodium, or an analogue or derivative thereof).

9. Farnesyltransferase Inhibitors

In another embodiment, the pharmacologically active compound is a farnesyltransferase inhibitor (e.g., dichlorobenzoprim (2,4-diamino-5-(4-(3,4-dichlorobenzylamino)-3-nitrophenyl)-6-ethylpyrimidine), B-581, B-956 (N-(8(R)-amino-2(S)-benzyl-5(S)-isopropyl-9-sulfanyl-3(Z),6(E)-nonadienoyl)-L-methionine), OSI-754, perillyl alcohol (1-cyclohexene-1-methanol, 4-(1-methylethenyl)-, RPR-114334, lonafarnib (1-piperidinecarboxamide, 4-(2-(4-((1R)-3,10-dibromo-8-chloro-6,11-dihydro-5H-benzo(5,6)cyclohepta(1,2-b)pyridin-11-yl)-1-piperidinyl)-2-oxoethyl)-), Sch-48755, Sch-226374, (7,8-dichloro-5H-dibenzo(b,e)(1,4)diazepin-11-yl)-pyridin-3-ylmethylamine, J-104126, L-639749, L-731734 (pentanamide, 2-((2-((2-amino-3-mercaptopropyl)amino)-3-methylpentyl)amino)-3-methyl-N-(tetrahydro-2-oxo-3-furanyl)-, (3S-(3R*(2R*(2R*(S*),3S*),3R*)))-), L-744832 (butanoic acid, 2-((2-((2-((2-amino-3-mercaptopropyl)amino)-3-methylpentyl)oxy)-1-oxo-3-phenylpropyl)amino)-4-(methylsulfonyl)-, 1-methylethyl ester, (2S-(1(R*(R*)),2R*(S*),3R*))-), L-745631 (1-piperazinepropanethiol, β-amino-2-(2-methoxyethyl)-4-(1-naphthalenylcarbonyl)-, (1R,2S)-), N-acetyl-N-naphthylmethyl-2(S)-((1-(4-cyanobenzyl)-1H-imidazol-5-yl)acetyl)amino-3(S)-methylpentamine, (2alpha)-2-hydroxy-24,25-dihydroxylanost-8-en-3-one, BMS-316810, UCF-1-C (2,4-decadienamide, N-(5-hydroxy-5-(7-((2-hydroxy-5-oxo-1-cyclopenten-1-yl)amino-oxo-1,3,5-heptatrienyl)-2-oxo-7-oxabicyclo(4.1.0)hept-3-en-3-yl)-2,4,6-trimethyl-, (1S-(1 alpha,3(2E,4E,6S*),5 alpha, 5(1E,3E,5E), 6 alpha))-), UCF-116-B, ARGLABIN (3H-oxireno[8,8a]azuleno[4,5-b]furan-8(4aH)-one, 5,6,6a,7,9a,9b-hexahydro-1,4a-dimethyl-7-methylene-, (3aR,4aS,6aS,9aS,9bR)-) from ARGLABIN—Paracure, Inc. (Virginia Beach, Va.), or an analogue or derivative thereof).

10. Fibrinogen Antagonists

In another embodiment, the pharmacologically active compound is a fibrinogen antagonist (e.g., 2(S)-((p-toluenesulfonyl)amino)-3-(((5,6,7,8,-tetrahydro-4-oxo-5-(2-(piperidin-4-yl)ethyl)-4H-pyrazolo-(1,5-a)(1,4)diazepin-2-yl)carbonyl)amino)propionic acid, streptokinase (kinase (enzyme-activating), strepto-), urokinase (kinase (enzyme-activating), uro-), plasminogen activator, pamiteplase, monteplase, heberkinase, anistreplase, alteplase, pro-urokinase, picotamide (1,3-benzenedicarboxamide, 4-methoxy-N,N′-bis(3-pyridinylmethyl)-), or an analogue or derivative thereof).

11. Guanylate Cyclase Stimulants

In another embodiment, the pharmacologically active compound is a guanylate cyclase stimulant (e.g., isosorbide-5-mononitrate (D-glucitol, 1,4:3,6-dianhydro-, 5-nitrate), or an analogue or derivative thereof).

12. Heat Shock Protein 90 Antagonists

In another embodiment, the pharmacologically active compound is a heat shock protein 90 antagonist (e.g., geldanamycin; NSC-33050 (17-allylaminogeldanamycin), rifabutin (rifamycin XIV, 1′,4-didehydro-1-deoxy-1,4-dihydro-5′-(2-methylpropyl)-1-oxo-), 17AAG, or an analogue or derivative thereof).

13. HMGCoA Reductase Inhibitors

In another embodiment, the pharmacologically active compound is an HMGCoA reductase inhibitor (e.g., BCP-671, BB-476, fluvastatin (6-heptenoic acid, 7-(3-(4-fluorophenyl)-1-(1-methylethyl)-1H-indol-2-yl)-3,5-dihydroxy-, monosodium salt, (R*,S*-(E))-(+)-), dalvastatin (2H-pyran-2-one, 6-(2-(2-(2-(4-fluoro-3-methylphenyl)-4,4,6,6-tetramethyl-1-cyclohexen-1-yl)ethenyl)tetrahydro)-4-hydroxy-, (4alpha,6β(E))-(+/−)-), glenvastatin (2H-pyran-2-one, 6-(2-(4-(4-fluorophenyl)-2-(1-methylethyl)-6-phenyl-3-pyridinyl)ethenyl)tetrahydro-4-hydroxy-, (4R-(4alpha,6β(E)))-), S-2468, N-(1-oxododecyl)-4Alpha,10-dimethyl-8-aza-trans-decal-3β-ol, atorvastatin calcium (1H-Pyrrole-1-heptanoic acid, 2-(4-fluorophenyl)-β,delta-dihydroxy-5-(1-methylethyl)-3-phenyl-4-((phenylamino)carbonyl)-, calcium salt (R-(R*,R*))-), CP-83101 (6,8-nonadienoic acid, 3,5-dihydroxy-9,9-diphenyl-, methyl ester, (R*,S*-(E))-(+/−)-), pravastatin (1-naphthaleneheptanoic acid, 1,2,6,7,8,8a-hexahydro-β,delta,6-trihydroxy-2-methyl-8-(2-methyl-1-oxobutoxy)-, monosodium salt, (1S-(1 alpha(βS*,deltaS*),2 alpha,6 alpha,8β(R*),8a alpha))-), U-20685, pitavastatin (6-heptenoic acid, 7-(2-cyclopropyl-4-(4-fluorophenyl)-3-quinolinyl)-3,5-dihydroxy-, calcium salt (2:1), (S-(R*,S*-(E)))-), N-((1-methylpropyl)carbonyl)-8-(2-(tetrahydro-4-hydroxy-6-oxo-2H-pyran-2-yl)ethyl)-perhydro-isoquinoline, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha, 4a alpha,7β,8β(2S*,4S*),8aβ))-), HBS-107, dihydromevinolin (butanoic acid, 2-methyl-, 1,2,3,4,4a,7,8,8a-octahydro-3,7-dimethyl-8-(2-(tetrahydro-4-hydroxy-6-oxo-2H-pyran-2-yl)ethyl)-1-naphthalenyl ester(1 alpha(R*), 3 alpha,4a alpha,7β,8β(2S*,4S*),8aβ))-), L-669262 (butanoic acid, 2,2-dimethyl-, 1,2,6,7,8,8a-hexahydro-3,7-dimethyl-6-oxo-8-(2-(tetrahydro-4-hydroxy-6-oxo-2H-pyran-2-yl)ethyl)-1-naphthalenyl(1S-(1 Alpha,7β,8β(2S*,4S*),8aβ))-), simvastatin (butanoic acid, 2,2-dimethyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1alpha, 3alpha,7β,8β(2S*,4S*),8aβ))-), rosuvastatin calcium (6-heptenoic acid, 7-(4-(4-fluorophenyl)-6-(1-methylethyl)-2-(methyl(methylsulfonyl)amino)-5-pyrimdinyl)-3,5-dihydroxy-calcium salt (2:1) (S-(R*,S*-(E)))), meglutol (2-hydroxy-2-methyl-1,3-propandicarboxylic acid), lovastatin (butanoic acid, 2-methyl-, 1,2,3,7,8,8a-hexahydro-3,7-dimethyl-8-(2-(tetrahydro-4-hydroxy-6-oxo-2H-pyran-2-yl)ethyl)-1-naphthalenyl ester, (1S-(1 alpha.(R*),3 alpha,7β,8β(2S*,4S*),8β))-), or an analogue or derivative thereof).

14. Hydroorotate Dehydrogenase Inhibitors

In another embodiment, the pharmacologically active compound is a hydroorotate dehydrogenase inhibitor (e.g., leflunomide (4-isoxazolecarboxamide, 5-methyl-N-(4-(trifluoromethyl)phenyl)-), laflunimus (2-propenamide, 2-cyano-3-cyclopropyl-3-hydroxy-N-(3-methyl-4(trifluoromethyl)phenyl)-, (Z)-), or atovaquone (1,4-naphthalenedione, 2-[4-(4-chlorophenyl)cyclohexyl]-3-hydroxy-, trans-, or an analogue or derivative thereof).

15. IKK2 Inhibitors

In another embodiment, the pharmacologically active compound is an IKK2 inhibitor (e.g., MLN-120B, SPC-839, or an analogue or derivative thereof).

16. IL-1, ICE and IRAK Antagonists

In another embodiment, the pharmacologically active compound is an IL-1, ICE or an IRAK antagonist (e.g., E-5090 (2-propenoic acid, 3-(5-ethyl-4-hydroxy-3-methoxy-1-naphthalenyl)-2-methyl-, (Z)-), CH-164, CH-172, CH-490, AMG-719, iguratimod (N-(3-(formylamino)-4-oxo-6-phenoxy-4H-chromen-7-yl) methanesulfonamide), AV94-88, pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), (2S-cis)-5-(benzyloxycarbonylamino-1,2,4,5,6,7-hexahydro-4-(oxoazepino(3,2,1-hi)indole-2-carbonyl)-amino)-4-oxobutanoic acid, AVE-9488, esonarimod (benzenebutanoic acid, alpha-((acetylthio)methyl)-4-methyl-gamma-oxo-), pralnacasan (6H-pyridazino(1,2-a)(1,2)diazepine-1-carboxamide, N-((2R,3S)-2-ethoxytetrahydro-5-oxo-3-furanyl)octahydro-9-((1-isoquinolinylcarbonyl)amino)-6,10-dioxo-, (1S,9S)-), tranexamic acid (cyclohexanecarboxylic acid, 4-(aminomethyl)-, trans-), Win-72052, romazarit (Ro-31-3948) (propanoic acid, 2-((2-(4-chlorophenyl)-4-methyl-5-oxazolyl)methoxy)-2-methyl-), PD-163594, SDZ-224-015 (L-alaninamide N-((phenylmethoxy)carbonyl)-L-valyl-N-((1S)-3-((2,6-dichlorobenzoyl)oxy)-1-(2-ethoxy-2-oxoethyl)-2-oxopropyl)-), L-709049 (L-alaninamide, N-acetyl-L-tyrosyl-L-valyl-N-(2-carboxy-1-formylethyl)-, (S)-), TA-383 (1H-imidazole, 2-(4-chlorophenyl)-4,5-dihydro-4,5-diphenyl-, monohydrochloride, cis-), EI-1507-1 (6a,12a-epoxybenz(a)anthracen-1,12(2H,7H)-dione, 3,4-dihydro-3,7-dihydroxy-8-methoxy-3-methyl-), ethyl 4-(3,4-dimethoxyphenyl)-6,7-dimethoxy-2-(1,2,4-triazol-1-yl methyl)quinoline-3-carboxylate, EI-1941-1, TJ-114, anakinra (interleukin 1 receptor antagonist (human isoform x reduced), N2-L-methionyl-), IX-207-887 (acetic acid, (10-methoxy-4H-benzo[4,5]cyclohepta[1,2-b]thien-4-ylidene)-), K-832, or an analogue or derivative thereof).

17. IL-4 Agonists

In another embodiment, the pharmacologically active compound is an IL-4 agonist (e.g., glatiramir acetate (L-glutamic acid, polymer with L-alanine, L-lysine and L-tyrosine, acetate (salt)), or an analogue or derivative thereof).

18. Immunomodulatory Agents

In another embodiment, the pharmacologically active compound is an immunomodulatory agent (e.g., biolimus, ABT-578, methylsulfamic acid 3-(2-methoxyphenoxy)-2-(((methylamino)sulfonyl)oxy)propyl ester, sirolimus (also referred to as rapamycin or RAPAMUNE (American Home Products, Inc., Madison, N.J.)), CCI-779 (rapamycin 42-(3-hydroxy-2-(hydroxymethyl)-2-methylpropanoate)), LF-15-0195, NPC15669 (L-leucine, N-(((2,7-dimethyl-9H-fluoren-9-yl)methoxy)carbonyl)-), NPC-15670 (L-leucine, N-(((4,5-dimethyl-9H-fluoren-9-yl)methoxy)carbonyl)-), NPC-16570 (4-(2-(fluoren-9-yl)ethyloxycarbonyl)aminobenzoic acid), sufosfamide (ethanol, 2-((3-(2-chloroethyl)tetrahydro-2H-1,3,2-oxazaphosphorin-2-yl)amino)-, methanesulfonate (ester), P-oxide), tresperimus (2-(N-(4-(3-aminopropylamino)butyl)carbamoyloxy)-N-(6-guanidinohexyl)acetamide), 4-(2-(fluoren-9-yl)ethoxycarbonylamino)-benzo-hydroxamic acid, iaquinimod, PBI-1411, azathioprine (6-((1-Methyl-4-nitro-1H-imidazol-5-yl)thio)-1H-purine), PBI0032, beclometasone, MDL-28842 (9H-purin-6-amine, 9-(5-deoxy-5-fluoro-β-D-threo-pent-4-enofuranosyl)-, (Z)-), FK-788, AVE-1726, ZK-90695, ZK-90695, Ro-54864, didemnin-B, Illinois (didemnin A, N-(1-(2-hydroxy-1-oxopropyl)-L-prolyl)-, (S)-), SDZ-62-826 (ethanaminium, 2-((hydroxy((1-((octadecyloxy)carbonyl)-3-piperidinyl)methoxy)phosphinyl)oxy)-N,N,N-trimethyl-, inner salt), argyrin B ((4S,7S,13R,22R)-13-Ethyl-4-(1H-indol-3-ylmethyl)-7-(4-methoxy-1H-indol-3-ylmethyl)18,22-dimethyl-16-methyl-ene-24-thia-3,6,9,12,15,18,21,26-octaazabicyclo(21.2.1)-hexacosa-1(25),23(26)-diene-2,5,8,11,14,17,20-heptaone), everolimus (rapamycin, 42-O-(2-hydroxyethyl)-), SAR-943, L-687795, 6-((4-chlorophenyl)sulfinyl)-2,3-dihydro-2-(4-methoxyphenyl)-5-methyl-3-oxo-4-pyridazinecarbonitrile, 91Y78 (1H-imidazo[4,5-c)pyridin-4-amine, 1-β-D-ribofuranosyl-), auranofin (gold, (1-thio-β-D-glucopyranose 2,3,4,6-tetraacetato-S)(triethylphosphine)-), 27-O-demethylrapamycin, tipredane (androsta-1,4-dien-3-one, 17-(ethylthio)-9-fluoro-11-hydroxy-17-(methylthio)-, (11R,17 alpha)-), AI-402, LY-178002 (4-thiazolidinone, 5-((3,5-bis(1,1-dimethylethyl)-4-hydroxyphenyl)methylene)-), SM-8849 (2-thiazolamine, 4-(1-(2-fluoro(1,1′-biphenyl)-4-yl)ethyl)-N-methyl-), piceatannol, resveratrol, triamcinolone acetonide (pregna-1,4-diene-3,20-dione, 9-fluoro-11,21-dihydroxy-16,17-((1-methylethylidene)bis(oxy))-, (11β,16 alpha)-), ciclosporin (cyclosporin A), tacrolimus (15,19-epoxy-3H-pyrido(2,1-c)(1,4)oxaazacyclotricosine-1,7,20,21(4H,23H)-tetrone, 5,6,8,11,12,13,14,15,16,17,18,19,24,25,26,26a-hexadecahydro-5,19-dihydroxy-3-(2-(4-hydroxy-3-methoxycyclohexyl)-1-methylethenyl)-14,16-dimethoxy-4,10,12,18-tetramethyl-8-(2-propenyl)-, (3S-(3R*(E(1S*,3S*,4S*)),4S*,5R*,8S*,9E,12R*,14R*,15S*,16R*,18S*,19S*,26aR*))-), gusperimus (heptanamide, 7-((aminoiminomethyl)amino)-N-(2-((4-((3-aminopropyl)amino)butyl)amino)-1-hydroxy-2-oxoethyl)-, (+/−)-), tixocortol pivalate (pregn-4-ene-3,20-dione, 21-((2,2-dimethyl-1-oxopropyl)thio)-11,17-dihydroxy-, (11β)-), alefacept (1-92 LFA-3 (antigen) (human) fusion protein with immunoglobulin G1 (human hinge-CH2-CH3 gamma1-chain), dimer), halobetasol propionate (pregna-1,4-diene-3,20-dione, 21-chloro-6,9-difluoro-11-hydroxy-16-methyl-17-(1-oxopropoxy)-, (6Alpha,11β,16β)-), iloprost trometamol (pentanoic acid, 5-(hexahydro-5-hydroxy-4-(3-hydroxy-4-methyl-1-octen-6-ynyl)-2(1H)-pentalenylidene)-), beraprost (1H-cyclopenta(b)benzofuran-5-butanoic acid, 2,3,3a,8b-tetrahydro-2-hydroxy-1-(3-hydroxy-4-methyl-1-octen-6-ynyl)-), rimexolone (androsta-1,4-dien-3-one,11-hydroxy-16,17-dimethyl-17-(1-oxopropyl)-, (11β,16Alpha,17β)-), dexamethasone (pregna-1,4-diene-3,20-dione,9-fluoro-11,17,21-trihydroxy-16-methyl-, (11β,16alpha)-), sulindac (cis-5-fluoro-2-methyl-1-((p-methylsulfinyl)benzylidene)indene-3-acetic acid), proglumetacin (1H-Indole-3-acetic acid, 1-(4-chlorobenzoyl)-5-methoxy-2-methyl-, 2-(4-(3-((4-(benzoylamino)-5-(dipropylamino)-1,5-dioxopentyl)oxy)propyl)-1-piperazinyl)ethylester, (+/−)-), alclometasone dipropionate (pregna-1,4-diene-3,20-dione, 7-chloro-11-hydroxy-16-methyl-17,21-bis(1-oxopropoxy)-, (7alpha,11β,16alpha)-), pimecrolimus (15,19-epoxy-3H-pyrido(2,1-c)(1,4)oxaazacyclotricosine-1,7,20,21 (4H,23H)-tetrone, 3-(2-(4-chloro-3-methoxycyclohexyl)-1-methyletheny)-8-ethyl-5,6,8,11,12,13,14,15,16,17,18,19,24,25,26,26a-hexadecahydro-5,19-dihydroxy-14,16-dimethoxy-4,10,12,18-tetramethyl-, (3S-(3R*(E(1S*,3S*,4R*)),4S*,5R*,8S*,9E,12R*,14R*,15S*,16R*,18S*,19S*,26aR*))-), hydrocortisone-17-butyrate (pregn-4-ene-3,20-dione, 11,21-dihydroxy-17-(1-oxobutoxy)-, (11β)-), mitoxantrone (9,10-anthracenedione, 1,4-dihydroxy-5,8-bis((2-((2-hydroxyethyl)amino)ethyl)amino)-), mizoribine (1H-imidazole-4-carboxamide, 5-hydroxy-1-β-D-ribofuranosyl-), prednicarbate (pregna-1,4-diene-3,20-dione, 17-((ethoxycarbonyl)oxy)-11-hydroxy-21-(1-oxopropoxy)-, (11β)-), iobenzarit (benzoic acid, 2-((2-carboxyphenyl)amino)-4-chloro-), glucametacin (D-glucose, 2-(((1-(4-chlorobenzoyl)-5-methoxy-2-methyl-1H-indol-3-yl)acetyl)amino)-2-deoxy-), fluocortolone monohydrate ((6 alpha)-fluoro-16alpha-methylpregna-1,4-dien-11β,21-diol-3,20-dione), fluocortin butyl (pregna-1,4-dien-21-oic acid, 6-fluoro-11-hydroxy-16-methyl-3,20-dioxo-, butyl ester, (6alpha,11β,16alpha)-), difluprednate (pregna-1,4-diene-3,20-dione, 21-(acetyloxy)-6,9-difluoro-11-hydroxy-17-(1-oxobutoxy)-, (6 alpha,11β)-), diflorasone diacetate (pregna-1,4-diene-3,20-dione, 17,21-bis(acetyloxy)-6,9-difluoro-11-hydroxy-16-methyl-, (6Alpha,11β,16β)-), dexamethasone valerate (pregna-1,4-diene-3,20-dione, 9-fluoro-11,21-dihydroxy-16-methyl-17-((1-oxopentyl)oxy)-, (11β,16Alpha)-), methylprednisolone, deprodone propionate (pregna-1,4-diene-3,20-dione, 11-hydroxy-17-(1-oxopropoxy)-, (11.beta.)-), bucillamine (L-cysteine, N-(2-mercapto-2-methyl-1-oxopropyl)-), amcinonide (benzeneacetic acid, 2-amino-3-benzoyl-, monosodium salt, monohydrate), acemetacin (1H-indole-3-acetic acid, 1-(4-chlorobenzoyl)-5-methoxy-2-methyl-, carboxymethyl ester), or an analogue or derivative thereof).

Further, analogues of rapamycin include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives can be found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

The structures of sirolimus, everolimus, and tacrolimus are provided below:

| Name | Code Name | Company | Structure |
|---|---|---|---|
| Everolimus | SAR-943 | Novartis | See below |
| Sirolimus | AY-22989 | Wyeth | See below |
| RAPAMUNE | NSC-226080 | | |
| Rapamycin | | | |
| Tacrolimus | FK506 | Fujusawa | See below |

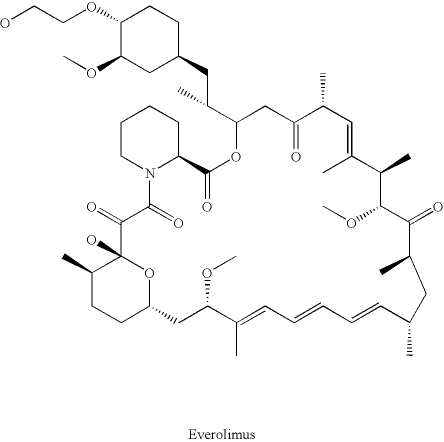

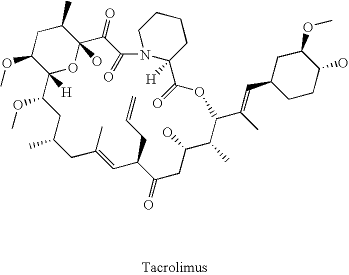

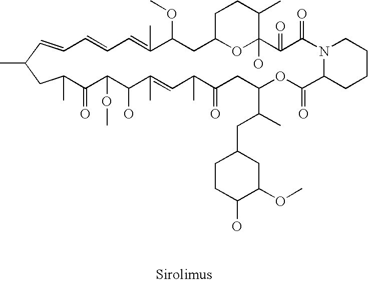

Further sirolimus analogues and derivatives include tacrolimus and derivatives thereof (e.g., EP0184162B1 and U.S. Pat. No. 6,258,823) everolimus and derivatives thereof (e.g., U.S. Pat. No. 5,665,772). Further representative examples of sirolimus analogues and derivatives include ABT-578 and others may be found in PCT Publication Nos. WO 97/10502, WO 96/41807, WO 96/35423, WO 96/03430, WO 9600282, WO 95/16691, WO 9515328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179. Representative U.S. patents include U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241, 5,200,411; 5,198,421; 5,147,877; 5,140,018; 5,116,756; 5,109,112; 5,093,338; and 5,091,389.

In one aspect, the fibrosis-inhibiting agent may be, e.g., rapamycin (sirolimus), everolimus, biolimus, tresperimus, auranofin, 27-O-demethylrapamycin, tacrolimus, gusperimus, pimecrolimus, or ABT-578.

19. Inosine Monophosphate Dehydrogenase Inhibitors

In another embodiment, the pharmacologically active compound is an inosine monophosphate dehydrogenase (IMPDH) inhibitor (e.g., mycophenolic acid, mycophenolate mofetil (4-hexenoic acid, 6-(1,3-dihydro-4-hydroxy-6-methoxy-7-methyl-3-oxo-5-isobenzofuranyl)-4-methyl-, 2-(4-morpholinyl)ethyl ester, (E)-), ribavirin (1H-1,2,4-triazole-3-carboxamide, 1-β-D-ribofuranosyl-), tiazofurin (4-thiazolecarboxamide, 2-β-D-ribofuranosyl-), viramidine, aminothiadiazole, thiophenfurin, tiazofurin) or an analogue or derivative thereof. Additional representative examples are included in U.S. Pat. Nos. 5,536,747, 5,807,876, 5,932,600, 6,054,472, 6,128,582, 6,344,465, 6,395,763, 6,399,773, 6,420,403, 6,479,628, 6,498,178, 6,514,979, 6,518,291, 6,541,496, 6,596,747, 6,617,323, 6,624,184, Patent Application Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491 A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, 2003/0195202A1, and PCT Publication Nos. WO 0024725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 2051814A1, WO 2057287A2, WO2057425A2, WO 2060875A1, WO 2060896A1, WO 2060898A1, WO 2068058A2, WO 3020298A1, WO 3037349A1, WO 3039548A1, WO 3045901A2, WO 3047512A2, WO 3053958A1, WO 3055447A2, WO 3059269A2, WO 3063573A2, WO 3087071A1, WO 90/01545A1, WO 97/40028A1, WO 97/41211 A1, WO 98/40381 A1, and WO 99/55663A1).

20. Leukotriene Inhibitors

In another embodiment, the pharmacologically active compound is a leukotreine inhibitor (e.g., ONO-4057(benzenepropanoic acid, 2-(4-carboxybutoxy)-6-((6-(4-methoxyphenyl)-5-hexenyl)oxy)-, (E)-), ONO-LB-448, pirodomast 1,8-naphthyridin-2(1H)-one, 4-hydroxy-1-phenyl-3-(1-pyrrolidinyl)-, Sch-40120 (benzo(b)(1,8)naphthyridin-5(7H)-one, 10-(3-chlorophenyl)-6,8,9,10-tetrahydro-), L-656224 (4-benzofuranol, 7-chloro-2-((4-methoxyphenyl)methyl)-3-methyl-5-propyl-), MAFP (methyl arachidonyl fluorophosphonate), ontazolast (2-benzoxazolamine, N-(2-cyclohexyl-1-(2-pyridinyl)ethyl)-5-methyl-, (S)-), amelubant (carbamic acid, ((4-((3-((4-(1-(4-hydroxyphenyl)-1-methylethyl)phenoxy)methyl)phenyl)methoxy)phenyl)iminomethyl)-ethyl ester), SB-201993 (benzoic acid, 3-((((6-((1E)-2-carboxyethenyl)-5-((8-(4-methoxyphenyl)octyl)oxy)-2-pyridinyl)methyl)thio)methyl)-), LY-203647 (ethanone, 1-(2-hydroxy-3-propyl-4-(4-(2-(4-(1H-tetrazol-5-yl)butyl)-2H-tetrazol-5-yl)butoxy)phenyl)-), LY-210073, LY-223982 (benzenepropanoic acid, 5-(3-carboxybenzoyl)-2-((6-(4-methoxyphenyl)-5-hexenyl)oxy)-, (E)-), LY-293111 (benzoic acid, 2-(3-(3-((5-ethyl-4′-fluoro-2-hydroxy(1,1′-biphenyl)-4-yl)oxy)propoxy)-2-propylphenoxy)-), SM-9064 (pyrrolidine, 1-(4,11-dihydroxy-13-(4-methoxyphenyl)-1-oxo-5,7,9-tridecatrienyl)-, (E, E, E)-), T-0757 (2,6-octadienamide, N-(4-hydroxy-3,5-dimethylphenyl)-3,7-dimethyl-, (2E)-), or an analogue or derivative thereof).

21. MCP-1 Antagonists

In another embodiment, the pharmacologically active compound is a MCP-1 antagonist (e.g., nitronaproxen (2-napthaleneacetic acid, 6-methoxy-alpha-methyl 4-(nitrooxy)butyl ester (alpha S)-), bindarit (2-(1-benzylindazol-3-ylmethoxy)-2-methylpropanoic acid), 1-alpha-25 dihydroxy vitamin D₃, or an analogue or derivative thereof).

22. MMP Inhibitors

In another embodiment, the pharmacologically active compound is a matrix metalloproteinase (MMP) inhibitor (e.g., D-9120, doxycycline (2-naphthacenecarboxamide, 4-(dimethylamino)-1,4,4a,5,5a,6,11,12a-octahydro-3,5,10,12,12a-pentahydroxy-6-methyl-1,11-dioxo-(4S-(4 alpha, 4a alpha, 5 lpha, 5a alpha, 6 alpha, 12a alpha))-), BB-2827, BB-1101 (2S-allyl-N-1-hydroxy-3R-isobutyl-N-4-(1S-methylcarbamoyl-2-phenylethyl)-succinamide), BB-2983, solimastat (N′-(2,2-dimethyl-1 (S)-(N-(2-pyridyl)carbamoyl)propyl)-N-4-hydroxy-2(R)-isobutyl-3(S)-methoxysuccinamide), batimastat (butanediamide, N4-hydroxy-N-1-(2-(methylamino)-2-oxo-1-(phenylmethyl)ethyl)-2-(2-methylpropyl)-3-((2-thienylthio)methyl)-, (2R-(1(S*),2R*,3S*))-), CH-138, CH-5902, D-1927, D-5410, EF-13 (gamma-linolenic acid lithium salt),CMT-3 (2-naphthacenecarboxamide, 1,4,4a,5,5a,6,11,12a-octahydro-3,10,12,12a-tetrahydroxy-1,11-dioxo-, (4aS,5aR,12aS)-), marimastat (N-(2,2-dimethyl-1 (S)-(N-methylcarbamoyl)propyl)-N,3(S)-dihydroxy-2(R)-isobutylsuccinamide), TIMP'S, ONO-4817, rebimastat (L-Valinamide, N-((2S)-2-mercapto-1-oxo-4-(3,4,4-trimethyl-2,5-dioxo-1-imidazolidinyl)butyl)-L-leucyl-N,3-dimethyl-), PS-508, CH-715, nimesulide (methanesulfonamide, N-(4-nitro-2-phenoxyphenyl)-), hexahydro-2-(2(R)-(1(RS)-(hydroxycarbamoyl)-4-phenylbutyl)nonanoyl)-N-(2,2,6,6-etramethyl-4-piperidinyl)-3(S)-pyridazine carboxamide, Rs-113-080, Ro-1130830, cipemastat (1-piperidinebutanamide, β-(cyclopentylmethyl)-N-hydroxy-gamma-oxo-alpha-((3,4,4-trimethyl-2,5-dioxo-1-imidazolidinyl)methyl)-,(alpha R,βR)-), 5-(4′-biphenyl)-5-(N-(4-nitrophenyl)piperazinyl)barbituric acid, 6-methoxy-1,2,3,4-tetrahydro-norharman-1-carboxylic acid, Ro-31-4724 (L-alanine, N-(2-(2-(hydroxyamino)-2-oxoethyl)-4-methyl-1-oxopentyl)-L-leucyl-, ethyl ester), prinomastat (3-thiomorpholinecarboxamide, N-hydroxy-2,2-dimethyl-4-((4-(4-pyridinyloxy)phenyl)sulfonyl)-, (3R)-), AG-3433 (1H-pyrrole-3-propanic acid, 1-(4′-cyano(1,1′-biphenyl)-4-yl)-b-((((3S)-tetrahydro-4,4-dimethyl-2-oxo-3-furanyl)amino)carbonyl)-, phenylmethyl ester, (bS)-), PNU-142769 (2H-Isoindole-2-butanamide, 1,3-dihydro-N-hydroxy-alpha-((3S)-3-(2-methylpropyl)-2-oxo-1-(2-phenylethyl)-3-pyrrolidinyl)-1,3-dioxo-, (alpha R)-), (S)-1-(2-((((4,5-dihydro-5-thioxo-1,3,4-thiadiazol-2-yl)amino)-carbonyl)amino)-1-oxo-3-(pentafluorophenyl)propyl)-4-(2-pyridinyl)piperazine, SU-5402 (1H-pyrrole-3-propanoic acid, 2-((1,2-dihydro-2-oxo-3H-indol-3-ylidene)methyl)-4-methyl-), SC-77964, PNU-171829, CGS-27023A, N-hydroxy-2(R)-((4-methoxybenzene-sulfonyl)(4-picolyl)amino)-2-(2-tetrahydrofuranyl)-acetamide, L-758354 ((1,1′-biphenyl)-4-hexanoic acid, alpha-butyl-gamma-(((2,2-dimethyl-1-((methylamino)carbonyl)propyl)amino)carbonyl)-4′-fluoro-, (alpha S-(alpha R*,gammaS*(R*)))-, GI-155704A, CPA-926, TMI-005, XL-784, or an analogue or derivative thereof). Additional representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,691,381; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359.

23. NF Kappa B Inhibitors

In another embodiment, the pharmacologically active compound is a NF kappa B (NFKB) inhibitor (e.g., AVE-0545, Oxi-104 (benzamide, 4-amino-3-chloro-N-(2-(diethylamino)ethyl)-), dexlipotam, R-flurbiprofen ((1,1′-biphenyl)-4-acetic acid, 2-fluoro-alpha-methyl), SP100030 (2-chloro-N-(3,5-di(trifluoromethyl)phenyl)-4-(trifluoromethyl)pyrimidine-5-carboxamide), AVE-0545, Viatris, AVE-0547, Bay 11-7082, Bay 11-7085, 15 deoxy-prostaylandin J2, bortezomib (boronic acid, ((1R)-3-methyl-1-(((2S)-1-oxo-3-phenyl-2-((pyrazinylcarbonyl)amino)propyl)amino)butyl)-, benzamide an d nicotinamide derivatives that inhibit NF-kappaB, such as those described in U.S. Pat. Nos. 5,561,161 and 5,340,565 (OxiGene), PG490-88Na, or an analogue or derivative thereof).

24. NO Antagonists

In another embodiment, the pharmacologically active compound is a NO antagonist (e.g., NCX-4016 (benzoic acid, 2-(acetyloxy)-, 3-((nitrooxy)methyl)phenyl ester, NCX-2216, L-arginine or an analogue or derivative thereof).

25. P38 MAP Kinase Inhibitors

In another embodiment, the pharmacologically active compound is a p38 MAP kinase inhibitor (e.g., GW-2286, CGP-52411, BIRB-798, SB220025, RO-320-1195, RWJ-67657, RWJ-68354, SCIO-469, SCIO-323, AMG-548, CMC-146, SD-31145, CC-8866, Ro-320-1195, PD-98059 (4H-1-benzopyran-4-one, 2-(2-amino-3-methoxyphenyl)-), CGH-2466, doramapimod, SB-203580 (pyridine, 4-(5-(4-fluorophenyl)-2-(4-(methylsulfinyl)phenyl)-1H-imidazol-4-yl)-), SB-220025 ((5-(2-amino-4-pyrimidinyl)-4-(4-fluorophenyl)-1-(4-piperidinyl)imidazole), SB-281832, PD169316, SB202190, GSK-681323, EO-1606, GSK-681323, or an analogue or derivative thereof). Additional representative examples are included in U.S. Pat. Nos. 6,300,347; 6,316,464; 6,316,466; 6,376,527; 6,444,696; 6,479,507; 6,509,361; 6,579,874; 6,630,485, U.S. Patent Application Publication Nos. 2001/0044538A1; 2002/0013354A1; 2002/0049220A1; 2002/0103245A1; 2002/0151491A1; 2002/0156114A1; 2003/0018051A1; 2003/0073832A1; 2003/0130257A1; 2003/0130273A1; 2003/0130319A1; 2003/0139388A1; 20030139462A1; 2003/0149031A1; 2003/0166647A1; 2003/0181411A1; and PCT Publication Nos. WO 00/63204A2; WO 01/21591A1; WO 01/35959A1; WO 01/74811A2; WO 02/18379A2; WO 2064594A2; WO 2083622A2; WO 2094842A2; WO 2096426A1; WO 2101015A2; WO 2103000A2; WO 3008413A1; WO 3016248A2; WO 3020715A1; WO 3024899A2; WO 3031431A1; WO3040103A1; WO 3053940A1; WO 3053941A2; WO 3063799A2; WO 3079986A2; WO 3080024A2; WO 3082287A1; WO 97/44467A1; WO 99/01449A1; and WO 99/58523A1.

26. Phosphodiesterase Inhibitors

In another embodiment, the pharmacologically active compound is a phosphodiesterase inhibitor (e.g., CDP-840 (pyridine, 4-((2R)-2-(3-(cyclopentyloxy)-4-methoxyphenyl)-2-phenylethyl)-), CH-3697, CT-2820, D-22888 (imidazo[1,5-a)pyrido(3,2-e)pyrazin-6(5H)-one, 9-ethyl-2-methoxy-7-methyl-5-propyl-), D-4418 (8-methoxyquinoline-5-(N-(2,5-dichloropyridin-3-yl))carboxamide), 1-(3-cyclopentyloxy-4-methoxyphenyl)-2-(2,6-dichloro-4-pyridyl) ethanone oxime, D-4396, ONO-6126, CDC-998, CDC-801, V-11294A (3-(3-(cyclopentyloxy)-4-methoxybenzyl)-6-(ethylamino)-8-isopropyl-3H-purine hydrochloride), S,S′-methylene-bis(2-(8-cyclopropyl-3-propyl-6-(4-pyridylmethylamino)-2-thio-3H-purine))tetrahyrochloride, rolipram (2-pyrrolidinone, 4-(3-(cyclopentyloxy)-4-methoxyphenyl)-), CP-293121, CP-353164 (5-(3-cyclopentyloxy-4-methoxyphenyl)pyridine-2-carboxamide), oxagrelate (6-phthalazinecarboxylic acid, 3,4-dihydro-1-(hydroxymethyl)-5,7-dimethyl-4-oxo-, ethyl ester), PD-168787, ibudilast (1-propanone, 2-methyl-1-(2-(1-methylethyl)pyrazolo(1,5-a)pyridin-3-yl)-), oxagrelate (6-phthalazinecarboxylic acid, 3,4-dihydro-1-(hydroxymethyl)-5,7-dimethyl-4-oxo-, ethyl ester), griseolic acid (alpha-L-talo-oct-4-enofuranuronic acid, 1-(6-amino-9H-purin-9-yl)-3,6-anhydro-6-C-carboxy-1,5-dideoxy-), KW-4490, KS-506, T-440, roflumilast (benzamide, 3-(cyclopropylmethoxy)-N-(3,5-dichloro-4-pyridinyl)-4-(difluoromethoxy)-), rolipram, milrinone, triflusinal (benzoic acid, 2-(acetyloxy)-4-(trifluoromethyl)-), anagrelide hydrochloride (imidazo[2,1-b)quinazolin-2(3H)-one, 6,7-dichloro-1,5-dihydro-, monohydrochloride), cilostazol (2(1H)-quinolinone, 6-(4-(1-cyclohexyl-1H-tetrazol-5-yl)butoxy)-3,4-dihydro-), propentofylline (1H-purine-2,6-dione, 3,7-dihydro-3-methyl-1-(5-oxohexyl)-7-propyl-), sildenafil citrate (piperazine, 1-((3-(4,7-dihydro-1-methyl-7-oxo-3-propyl-1H-pyrazolo(4,3-d)pyrimidin-5-yl)-4-ethoxyphenyl)sulfonyl)-4-methyl, 2-hydroxy-1,2,3-propanetricarboxylate-(1:1)), tadalafil (pyrazino(1′,2′:1,6)pyrido(3,4-b)indole1,4-dione, 6-(1,3-benzodioxol-5-yl)-2,3,6,7,12,12a-hexahydro-2-methyl-, (6R-trans)), vardenafil (piperazine, 1-(3-(1,4-dihydro-5-methyl(-4-oxo-7-propylimidazo(5,1-f)(1,2,4)-triazin-2-yl)-4-ethoxyphenyl)sulfonyl)-4-ethyl-), milrinone ((3,4′-bipyridine)-5-carbonitrile, 1,6-dihydro-2-methyl-6-oxo-), enoximone (2H-imidazol-2-one, 1,3-dihydro-4-methyl-5-(4-(methylthio)benzoyl)-), theophylline (1H-purine-2,6-dione, 3,7-dihydro-1,3-dimethyl-), ibudilast (1-propanone, 2-methyl-1-(2-(1-methylethyl)pyrazolo(1,5-a)pyridin-3-yl)-), aminophylline (1H-purine-2,6-dione, 3,7-dihydro-1,3-dimethyl-, compound with 1,2-ethanediamine (2:1)-), acebrophylline (7H-purine-7-acetic acid, 1,2,3,6-tetrahydro-1,3-dimethyl-2,6-dioxo-, compd. with trans-4-(((2-amino-3,5-dibromophenyl)methyl)amino)cyclohexanol (1:1)), plafibride (propanamide, 2-(4-chlorophenoxy)-2-methyl-N-(((4-morpholinylmethyl)amino)carbonyl)-), ioprinone hydrochloride (3-pyridinecarbonitrile, 1,2-dihydro-5-imidazo(1,2-a)pyridin-6-yl-6-methyl-2-oxo-, monohydrochloride-), fosfosal (benzoic acid, 2-(phosphonooxy)-), amrinone ((3,4′-bipyridin)-6(1H)-one, 5-amino-, or an analogue or derivative thereof).

Other examples of phosphodiesterase inhibitors include denbufylline (1H-purine-2,6-dione, 1,3-dibutyl-3,7-dihydro-7-(2-oxopropyl)-), propentofylline (1H-purine-2,6-dione, 3,7-dihydro-3-methyl-1-(5-oxohexyl)-7-propyl-) and pelrinone (5-pyrimidinecarbonitrile, 1,4-dihydro-2-methyl-4-oxo-6-[(3-pyridinylmethyl)amino]-).

Other examples of phosphodiesterase III inhibitors include enoximone (2H-imidazol-2-one, 1,3-dihydro-4-methyl-5-[4-(methylthio)benzoyl]-), and saterinone (3-pyridinecarbonitrile, 1,2-dihydro-5-[4-[2-hydroxy-3-[4-(2-methoxyphenyl)-1-piperazinyl]propoxy]phenyl]-6-methyl-2-oxo-).

Other examples of phosphodiesterase IV inhibitors include AWD-12-281, 3-auinolinecarboxylic acid, 1-ethyl-6-fluoro-1,4-dihydro-7-(4-methyl-1-piperazinyl)-4-oxo-), tadalafil (pyrazino(1′,2′:1,6)pyrido(3,4-b)indole1,4-dione, 6-(1,3-benzodioxol-5-yl)-2,3,6,7,12,12a-hexahydro-2-methyl-, (6R-trans)), and filaminast (ethanone, 1-[3-(cyclopentyloxy)-4-methoxyphenyl]-, O-(aminocarbonyl)oxime,(1E)-).

Another example of a phosphodiesterase V inhibitor is vardenafil (piperazine, 1-(3-(1,4-dihydro-5-methyl(−4-oxo-7-propylimidazo(5,1-f)(1,2,4)-triazin-2-yl)-4-ethoxyphenyl)sulfonyl)-4-ethyl-).

27. TGF beta Inhibitors

In another embodiment, the pharmacologically active compound is a TGF beta Inhibitor (e.g., mannose-6-phosphate, LF-984, tamoxifen (ethanamine, 2-(4-(1,2-diphenyl-1-butenyl)phenoxy)-N,N-dimethyl-, (Z)-), tranilast, or an analogue or derivative thereof).

28. Thromboxane A2 Antagonists

In another embodiment, the pharmacologically active compound is a thromboxane A2 antagonist (e.g., CGS-22652 (3-pyridineheptanoic acid, γ-(4-(((4-chlorophenyl)sulfonyl)amino)butyl)-, (.+-.)-), ozagrel (2-propenoic acid, 3-(4-(1H-imidazol-1-ylmethyl)phenyl)-, (E)-), argatroban (2-piperidinecarboxylic acid, 1-(5-((aminoiminomethyl)amino)-1-oxo-2-(((1,2,3,4-tetrahydro-3-methyl-8-quinolinyl)sulfonyl)amino)pentyl)-4-methyl-), ramatroban (9H-carbazole-9-propanoic acid, 3-(((4-fluorophenyl)sulfonyl)amino)-1,2,3,4-tetrahydro-, (R)-), torasemide (3-pyridinesulfona mide, N-(((1-methylethyl)amino)carbonyl)-4-((3-methylphenyl)amino)-), gamma linoleic acid ((Z,Z,Z)-6,9,12-octadecatrienoic acid), seratrodast (benzeneheptanoic acid, zeta-(2,4,5-trimethyl-3,6-dioxo-1,4-cyclohexadien-1-yl)-, (+/−)-, or an analogue or derivative thereof).

29. TNF Alpha Antagonists and TACE Inhibitors

In another embodiment, the pharmacologically active compound is a TNF alpha antagonist or TACE inhibitor (e.g., E-5531 (2-deoxy-6-O-(2-deoxy-3-O-(3(R)-(5(Z)-dodecenoyloxy)-decyl)-6-O-methyl-2-(3-oxotetradecanamido)-4-O-phosphono-β-D-glucopyranosyl)-3-O-(3(R)-hydroxydecyl)-2-(3-oxotetradecanamido)-alpha-D-glucopyranose-1-O-phosphate), AZD-4717, glycophosphopeptical, UR-12715 (B=benzoic acid, 2-hydroxy-5-((4-(3-(4-(2-methyl-1H-imidazol(4,5-c)pyridin-1-yl)methyl)-1-piperidinyl)-3-oxo-1-phenyl-1-propenyl)phenyl)azo) (Z)), PMS-601, AM-87, xyloadenosine (9H-purin-6-amine, 9-β-D-xylofuranosyl-), RDP-58, RDP-59, BB2275, benzydamine, E-3330 (undecanoic acid, 2-((4,5-dimethoxy-2-methyl-3,6-dioxo-1,4-cyclohexadien-1-yl)methylene)-, (E)-), N-(D, L-2-(hydroxyaminocarbonyl)methyl-4-methylpentanoyl)-L-3-(2′-naphthyl)alanyl-L-alanine, 2-aminoethyl amide, CP-564959, MLN-608, SPC-839, ENMD-0997, Sch-23863 ((2-(10,11-dihydro-5-ethoxy-5H-dibenzo (a,d) cyclohepten-S-yl)-N,N-dimethyl-ethanamine), SH-636, PKF-241-466, PKF-242-484, TNF-484A, cilomilast (cis-4-cyano-4-(3-(cyclopentyloxy)-4-methoxyphenyl)cyclohexane-1-carboxylic acid), GW-3333, GW-4459, BMS-561392, AM-87, cloricromene (acetic acid, ((8-chloro-3-(2-(diethylamino)ethyl)-4-methyl-2-oxo-2H-1-benzopyran-7-yl)oxy)-, ethyl ester), thalidomide (1H-Isoindole-1,3(2H)-dione, 2-(2,6-dioxo-3-piperidinyl)-), vesnarinone (piperazine, 1-(3,4-dimethoxybenzoyl)-4-(1,2,3,4-tetrahydro-2-oxo-6-quinolinyl)-), infliximab, lentinan, etanercept (1-235-tumor necrosis factor receptor (human) fusion protein with 236-467-immunoglobulin G1 (human gamma1-chain Fc fragment)), diacerein (2-anthracenecarboxylic acid, 4,5-bis(acetyloxy)-9,10-dihydro-9,10-dioxo-, or an analogue or derivative thereof).

30. Tyrosine Kinase Inhibitors

In another embodiment, the pharmacologically active compound is a tyrosine kinase inhibitor (e.g., SKI-606, ER-068224, SD-208, N-(6-benzothiazolyl)-4-(2-(1-piperazinyl)pyrid-5-yl)-2-pyrimidineamine, celastrol (24,25,26-trinoroleana-1(10),3,5,7-tetraen-29-oic acid, 3-hydroxy-9,13-dimethyl-2-oxo-, (9 beta., 13alpha,14β,20 alpha)-), CP-127374 (geldanamycin, 17-demethoxy-17-(2-propenylamino)-), CP-564959, PD-171026, CGP-52411 (1H-Isoindole-1,3(2H)-dione, 4,5-bis(phenylamino)-), CGP-53716 (benzamide, N-(4-methyl-3-((4-(3-pyridinyl)-2-pyrimidinyl)amino)phenyl)-), imatinib (4-((methyl-1-piperazinyl)methyl)-N-(4-methyl-3-((4-(3-pyridinyl)-2-pyrimidinyl)amino)-phenyl)benzamide methanesulfonate), NVP-AAK980-NX, KF-250706 (13-chloro,5(R),6(S)-epoxy-14,16-dihydroxy-11-(hydroyimino)-3(R)-methyl-3,4,5,6,11,12-hexahydro-1H-2-benzoxacyclotetradecin-1-one), 5-(3-(3-methoxy-4-(2-((E)-2-phenylethenyl)-4-oxazolylmethoxy)phenyl)propyl)-3-(2-((E)-2-phenylethenyl)-4-oxazolylmethyl)-2,4-oxazolidinedione, genistein, NV-06, or an analogue or derivative thereof).

31. Vitronectin Inhibitors

In another embodiment, the pharmacologically active compound is a vitronectin inhibitor (e.g., O-(9,10-dimethoxy-1,2,3,4,5,6-hexahydro-4-((1,4,5,6-tetrahydro-2-pyrimidinyl)hydrazono)-8-benz(e)azulenyl)-N-((phenylmethoxy)carbonyl)-DL-homoserine 2,3-dihydroxypropyl ester, (2S)-benzoylcarbonylamino-3-(2-((4S)-(3-(4,5-dihydro-1H-imidazol-2-ylamino)-propyl)-2,5-dioxo-imidazolidin-1-yl)-acetylamino)-propionate, Sch-221153, S-836, SC-68448 (β-((2-2-(((3-((aminoiminomethyl)amino)phenyl)carbonyl)amino)acetyl)amino)-3,5-dichlorobenzenepropanoic acid), SD-7784, S-247, or an analogue or derivative thereof).

32. Fibroblast Growth Factor Inhibitors

In another embodiment, the pharmacologically active compound is a fibroblast growth factor inhibitor (e.g., CT-052923 (((2H-benzo(d)1,3-dioxalan-5-methyl)amino)(4-(6,7-dimethoxyquinazolin-4-yl)piperazinyl)methane-1-thione), or an analogue or derivative thereof).

33. Protein Kinase Inhibitors

In another embodiment, the pharmacologically active compound is a protein kinase inhibitor (e.g., KP-0201448, NPC15437 (hexanamide, 2,6-diamino-N-((1-(1-oxotridecyl)-2-piperidinyl)methyl)-), fasudil (1H-1,4-diazepine, hexahydro-1-(5-isoquinolinylsulfonyl)-), midostaurin (benzamide, N-(2,3,10,11,12,13-hexahydro-10-methoxy-9-methyl-1-oxo-9,13-epoxy-1H,9H-diindolo(1,2,3-gh:3′,2′,1′-lm)pyrrolo(3,4-j)(1,7)benzodiazonin-11-yl)-N-methyl-, (9Alpha,10β,11β,13Alpha)-),fasudil (1H-1,4-diazepine, hexahydro-1-(5-isoquinolinylsulfonyl)-, dexniguldipine (3,5-pyridinedicarboxylic acid, 1,4-dihydro-2,6-dimethyl-4-(3-nitrophenyl)-, 3-(4,4-diphenyl-1-piperidinyl)propyl methyl ester, monohydrochloride, (R)-), LY-317615 (1H-pyrole-2,5-dione, 3-(1-methyl-1H-indol-3-yl)-4-[1-[1-(2-pyridinylmethyl)-4-piperidinyl]-1H-indol-3-yl]-, monohydrochloride), perifosine (piperid inium, 4-[[hydroxy(octadecyloxy)phosphinyl]oxy]-1,1-dimethyl-, inner salt), LY-333531 (9H,18H-5,21:12,17-dimethenodibenzo(e,k)pyrrolo(3,4-h)(1,4,13)oxadiazacyclohexadecine-18,20(19H)-dione,9-((dimethylamino)methyl)-6,7,10,11-tetrahydro-, (S)-), Kynac; SPC-100270 (1,3-octadecanediol, 2-amino-, [S-(R*,R*)]-), Kynacyte, or an analogue or derivative thereof).

34. PDGF Receptor Kinase Inhibitors

In another embodiment, the pharmacologically active compound is a PDGF receptor kinase inhibitor (e.g., RPR-127963E, or an analogue or derivative thereof).

35. Endothelial Growth Factor Receptor Kinase Inhibitors

In another embodiment, the pharmacologically active compound is an endothelial growth factor receptor kinase inhibitor (e.g., CEP-7055, SU-0879 ((E)-3-(3,5-di-tert-butyl-4-hydroxyphenyl)-2-(aminothiocarbonyl)acrylonitrile), BIBF-1000, AG-013736 (CP-868596), AMG-706, AVE-0005, NM-3 (3-(2-methylcarboxymethyl)-6-methoxy-8-hydroxy-isocoumarin), Bay-43-9006, SU-011248, or an analogue or derivative thereof).

36. Retinoic Acid Receptor Antagonists

In another embodiment, the pharmacologically active compound is a retinoic acid receptor antagonist (e.g., etarotene (Ro-15-1570) (naphthalene, 6-(2-(4-(ethylsulfonyl)phenyl)-1-methylethenyl)-1,2,3,4-tetrahydro-1,1,4,4-tetramethyl-, (E)-), (2E,4E)-3-methyl-5-(2-((E)-2-(2,6,6-trimethyl-1-cyclohexen-1-yl)ethenyl)-1-cyclohexen-1-yl)-2,4-pentadienoic acid, tocoretinate (retinoic acid, 3,4-dihydro-2,5,7,8-tetramethyl-2-(4,8,12-trimethyltridecyl)-2H-1-benzopyran-6-yl ester, (2R*(4R*,8R*))-(+)-), aliretinoin (retinoic acid, cis-9, trans-13-), bexarotene (benzoic acid, 4-(1-(5,6,7,8-tetrahydro-3,5,5,8,8-pentamethyl-2-naphthalenyl)ethenyl)-), tocoretinate (retinoic acid, 3,4-dihydro-2,5,7,8-tetramethyl-2-(4,8,12-trimethyltridecyl)-2H-1-benzopyran-6-yl ester, [2R*(4R*,8R*)]-(+)-, or an analogue or derivative thereof).

37. Platelet Derived Growth Factor Receptor Kinase Inhibitors

In another embodiment, the pharmacologically active compound is a platelet derived growth factor receptor kinase inhibitor (e.g., leflunomide (4-isoxazolecarboxamide, 5-methyl-N-(4-(trifluoromethyl)phenyl)-, or an analogue or derivative thereof).

38. Fibronogin Antagonists

In another embodiment, the pharmacologically active compound is a fibrinogin antagonist (e.g., picotamide (1,3-benzenedicarboxamide, 4-methoxy-N,N′-bis(3-pyridinylmethyl)-, or an analogue or derivative thereof).

39. Antimycotic Agents

In another embodiment, the pharmacologically active compound is an antimycotic agent (e.g., miconazole, sulconizole, parthenolide, rosconitine, nystatin, isoconazole, fluconazole, ketoconasole, imidazole, itraconazole, terpinafine, elonazole, bifonazole, clotrimazole, conazole, terconazole (piperazine, 1-(4-((2-(2,4-dichlorophenyl)-2-(1H-1,2,4-triazol-1-ylmethyl)-1,3-dioxolan-4-yl)methoxy)phenyl)-4-(1-methylethyl)-, cis-), isoconazole (1-(2-(2-6-dichlorobenzyloxy)-2-(2-,4-dichlorophenyl)ethyl)), griseofulvin (spiro(benzofuran-2(3H),1′-(2)cyclohexane)-3,4′-dione, 7-chloro-2′,4,6-trimeth-oxy-6′methyl-, (1′S-trans)-), bifonazole (1H-imidazole, 1-((1,1′-biphenyl)-4-ylphenylmethyl)-), econazole nitrate (1-(2-((4-chlorophenyl)methoxy)-2-(2,4-dichlorophenyl)ethyl)-1H-imidazole nitrate), croconazole (1H-imidazole, 1-(1-(2-((3-chlorophenyl)methoxy)phenyl)ethenyl)-), sertaconazole (1H-Imidazole, 1-(2-((7-chlorobenzo(b)thien-3-yl)methoxy)-2-(2,4-dichlorophenyl)ethyl)-), omoconazole (1H-imidazole, 1-(2-(2-(4-chlorophenoxy)ethoxy)-2-(2,4-dichlorophenyl)-1-methylethenyl)-, (Z)-), flutrimazole (1H-imidazole, 1-((2-fluorophenyl)(4-fluorophenyl)phenylmethyl)-), fluconazole (1H-1,2,4-triazole-1-ethanol, alpha-(2,4-difluorophenyl)-alpha-(1H-1,2,4-triazol-1-ylmethyl)-), neticonazole (1H-Imidazole, 1-(2-(methylthio)-1-(2-(pentyloxy)phenyl)ethenyl)-, monohydrochloride, (E)-), butoconazole (1H-imidazole, 1-(4-(4-chlorophenyl)-2-((2,6-dichlorophenyl)thio)butyl)-, (+/−)-), clotrimazole (1-((2-chlorophenyl)diphenylmethyl)-1H-imidazole, or an analogue or derivative thereof).

40. Bisphosphonates

In another embodiment, the pharmacologically active compound is a bisphosphonate (e.g., clodronate, alendronate, pamidronate, zoledronate, or an analogue or derivative thereof).

41. Phospholipase A1 Inhibitors

In another embodiment, the pharmacologically active compound is a phospholipase A1 inhibitor (e.g., ioteprednol etabonate (androsta-1,4-diene-17-carboxylic acid, 17-((ethoxycarbonyl)oxy)-11-hydroxy-3-oxo-, chloromethyl ester, (11β,17 alpha)-, or an analogue or derivative thereof).

42. Histamine H1/H2/H3 Receptor Antagonists

In another embodiment, the pharmacologically active compound is a histamine H1, H2, or H3 receptor antagonist (e.g., ranitidine (1,1-ethenediamine, N-(2-(((5-((dimethylamino)methyl)-2-furanyl)methyl)thio)ethyl)-N′-methyl-2-nitro-), niperotidine (N-(2-((5-((dimethylamino)methyl)furfuryl)thio)ethyl)-2-nitro-N′-piperonyl-1,1-ethenediamine), famotidine (propanimidamide, 3-(((2-((aminoiminomethyl)amino)-4-thiazolyl)methyl)thio)-N-(aminosulfonyl)-), roxitadine acetate HCl (acetamide, 2-(acetyloxy)-N-(3-(3-(1-piperidinylmethyl)phenoxy)propyl)-, monohydrochloride), lafutidine (acetamide, 2-((2-furanylmethyl)sulfinyl)-N-(4-((4-(1-piperidinylmethyl)-2-pyridinyl)oxy)-2-butenyl)-(Z)-), nizatadine (1,1-ethenediamine, N-(2-(((2-((dimethylamino)methyl)-4-thiazolyl)methyl)thio)ethyl)-N′-methyl-2-nitro-), ebrotidine (benzenesulfonamide, N-(((2-(((2-((aminoiminomethyl)amino)-4-thiazoly)methyl)thio)ethyl)amino)methylene)-4-bromo-), rupatadine (5H-benzo(5,6)cyclohepta(1,2-b)pyridine, 8-chloro-6,11-dihydro-11-(1-((5-methyl-3-pyridinyl)methyl)-4-piperidinylidene)-, trihydrochloride-), fexofenadine HCl (benzeneacetic acid, 4-(1-hydroxy-4-(4(hydroxydiphenylmethyl)-1-piperidinyl)butyl)-alpha, alpha-dimethyl-, hydrochloride, or an analogue or derivative thereof).

43. Macrolide Antibiotics

In another embodiment, the pharmacologically active compound is a macrolide antibiotic (e.g., dirithromycin (erythromycin, 9-deoxo-11-deoxy-9,11-(imino(2-(2-methoxyethoxy)ethylidene)oxy)-, (9S(R))-), flurithromycin ethylsuccinate (erythromycin, 8-fluoro-mono(ethyl butanedioate) (ester)-), erythromycin stinoprate (erythromycin, 2′-propanoate, compound with N-acetyl-L-cysteine (1:1)), clarithromycin (erythromycin, 6-O-methyl-), azithromycin (9-deoxo-9a-aza-9a-methyl-9a-homoerythromycin-A), telithromycin (3-de((2,6-dideoxy-3-C-methyl-3-O-methyl-alpha-L-ribo-hexopyranosyl)oxy)-11,12-dideoxy-6-O-methyl-3-oxo-12,11-(oxycarbonyl((4-(4-(3-pyridinyl)-1H-imidazol-1-yl)butyl)imino))-), roxithromycin (erythromycin, 9-(O-((2-methoxyethoxy)methyl)oxime)), rokitamycin (leucomycin V, 4B-butanoate 3B-propanoate), RV-11 (erythromycin monopropionate mercaptosuccinate), midecamycin acetate (leucomycin V, 3B,9-diacetate 3,4B-dipropanoate), midecamycin (leucomycin V, 3,4B-dipropanoate), josamycin (leucomycin V, 3-acetate 4B-(3-methylbutanoate), or an analogue or derivative thereof).

44. GPIIb IIIa Receptor Antagonists

In another embodiment, the pharmacologically active compound is a GPIIb IIIa receptor antagonist (e.g., tirofiban hydrochloride (L-tyrosine, N-(butylsulfonyl)-O-(4-(4-piperidinyl)butyl)-, monohydrochloride-), eptifibatide (L-cysteinamide, N6-(aminoiminomethyl)-N-2-(3-mercapto-1-oxopropyl)-L-lysylglycyl-L-alpha-aspartyl-L-tryptophyl-L-prolyl-, cyclic(1->6)-disulfide), xemiofiban hydrochloride, or an analogue or derivative thereof).

45. Endothelin Receptor Antagonists

In another embodiment, the pharmacologically active compound is an endothelin receptor antagonist (e.g., bosentan (benzenesulfonamide, 4-(1,1-dimethylethyl]-N-(6-(2-hydroxyethoxy)-5-(2-methoxyphenoxy)(2,2′-bipyrimidin)-4-yl)-, or an analogue or derivative thereof).

46. Peroxisome Proliferator-Activated Receptor Agonists

In another embodiment, the pharmacologically active compound is a peroxisome proliferator-activated receptor agonist (e.g., gemfibrozil (pentanoic acid, 5-(2,5-dimethylphenoxy)-2,2-dimethyl-), fenofibrate (propanoic acid, 2-(4-(4-chlorobenzoyl)phenoxy)-2-methyl-, 1-methylethyl ester), ciprofibrate (propanoic acid, 2-(4-(2,2-dichlorocyclopropyl)phenoxy)-2-methyl-), rosiglitazone maleate (2,4-thiazolidinedione, 5-((4-(2-(methyl-2-pyridinylamino)ethoxy)phenyl)methyl)-, (Z)-2-butenedioate (1:1)), pioglitazone hydrochloride (2,4-thiazolidinedione, 5-((4-(2-(5-ethyl-2-pyridinyl)ethoxy)phenyl)methyl)-, monohydrochloride (+/−)-), etofylline clofibrate (propanoic acid, 2-(4-chlorophenoxy)-2-methyl-, 2-(1,2,3,6-tetrahydro-1,3-dimethyl-2,6-dioxo-7H-purin-7-yl)ethyl ester), etofibrate (3-pyridinecarboxylic acid, 2-(2-(4-chlorophenoxy)-2-methyl-1-oxopropoxy)ethyl ester), clinofibrate (butanoic acid, 2,2′-(cyclohexylidenebis(4,1-phenyleneoxy))bis(2-methyl-)), bezafibrate (propanoic acid, 2-(4-(2-((4-chlorobenzoyl)amino)ethyl)phenoxy)-2-methyl-), binifibrate (3-pyridinecarboxylic acid, 2-(2-(4-chlorophenoxy)-2-methyl-1-oxopropoxy)-1,3-propanediyl ester), or an analogue or derivative thereof).

In one aspect, the pharmacologically active compound is a peroxisome proliferator-activated receptor alpha agonist, such as GW-590735, GSK-677954, GSK501516, pioglitazone hydrochloride (2,4-thiazolidinedione, 5-[[4-[2-(5-ethyl-2-pyrid inyl)ethoxy]phenyl]methyl]-, monohydrochloride (+/−)-, or an analogue or derivative thereof).

47. Estrogen Receptor Agents

In another embodiment, the pharmacologically active compound is an estrogen receptor agent (e.g., estradiol, 17-β-estradiol, or an analogue or derivative thereof).

48. Somatostatin Analogues

In another embodiment, the pharmacologically active compound is a somatostatin analogue (e.g., angiopeptin, or an analogue or derivative thereof).

49. Neurokinin 1 Antagonists

In another embodiment, the pharmacologically active compound is a neurokinin 1 antagonist (e.g., GW-597599, lanepitant ((1,4′-bipiperidine)-1′-acetamide, N-(2-(acetyl((2-methoxyphenyl)methyl)amino)-1-(1H-indol-3-ylmethyl)ethyl)-(R)-), nolpitantium chloride (1-azoniabicyclo[2.2.2]octane, 1-[2-[3-(3,4-dichlorophenyl)-1-[[3-(1-methylethoxy)phenyl]acetyl]-3-piperidinyl]ethyl]-4-phenyl-, chloride, (S)-), or saredutant (benzamide, N-[4-[4-(acetylamino)-4-phenyl-1-piperidinyl]-2-(3,4-dichlorophenyl)butyl]-N-methyl-, (S)-), or vofopitant (3-piperidinamine, N-[[2-methoxy-5-[5-(trifluoromethyl)-1H-tetrazol-1-yl]phenyl]methyl]-2-phenyl-, (2S,3S)-, or an analogue or derivative thereof).

50. Neurokinin 3 Antagonist

In another embodiment, the pharmacologically active compound is a neurokinin 3 antagonist (e.g., talnetant (4-quinolinecarboxamide, 3-hydroxy-2-phenyl-N-[(1S)-1-phenylpropyl]-, or an analogue or derivative thereof).

51. Neurokinin Antagonist

In another embodiment, the pharmacologically active compound is a neurokinin antagonist (e.g., GSK-679769, GSK-823296, SR-489686 (benzamide, N-[4-[4-(acetylamino)-4-phenyl-1-piperidinyl]-2-(3,4-dichlorophenyl)butyl]-N-methyl-(S)-), SB-223412; SB-235375 (4-quinolinecarboxamide, 3-hydroxy-2-phenyl-N-[(1S)-1-phenylpropyl]-), UK-226471, or an analogue or derivative thereof).

52. VLA-4 Antagonist

In another embodiment, the pharmacologically active compound is a VLA-4 antagonist (e.g., GSK683699, or an analogue or derivative thereof).

53. Osteoclast Inhibitor

In another embodiment, the pharmacologically active compound is a osteoclast inhibitor (e.g., ibandronic acid (phosphonic acid, [1-hydroxy-3-(methylpentylamino)propylidene]bis-), alendronate sodium, or an analogue or derivative thereof).

54. DNA topoisomerase ATP Hydrolysing Inhibitor

In another embodiment, the pharmacologically active compound is a DNA topoisomerase ATP hydrolysing inhibitor (e.g., enoxacin (1,8-naphthyridine-3-carboxylic acid, 1-ethyl-6-fluoro-1,4-dihydro-4-oxo-7-(1-piperazinyl)-), levofloxacin (7H-Pyrido[1,2,3-de]-1,4-benzoxazine-6-carboxylic acid, 9-fluoro-2,3-dihydro-3-methyl-10-(4-methyl-1-piperazinyl)-7-oxo-, (S)-), ofloxacin (7H-pyrido[1,2,3-de]-1,4-benzoxazine-6-carboxylic acid, 9-fluoro-2,3-dihydro-3-methyl-10-(4-methyl-1-piperazinyl)-7-oxo-, (+/−)-), pefloxacin (3-quinolinecarboxylic acid, 1-ethyl-6-fluoro-1,4-dihydro-7-(4-methyl-1-piperazinyl)-4-oxo-), pipemidic acid (pyrido[2,3-d]pyrimidine-6-carboxylic acid, 8-ethyl-5,8-dihydro-5-oxo-2-(1-piperazinyl)-), pirarubicin (5,12-naphthacenedione, 10-[β-amino-2,3,6-trideoxy-4-O-(tetrahydro-2H-pyran-2-yl)-alpha-L-Iyxo-hexopyranosyl]oxy]-7,8,9,10-tetrahydro-6,8,11-trihydroxy-8-(hydroxyacetyl)-1-methoxy-, [8S-[8 alpha,10 alpha(S*)]]-), sparfloxacin (3-quinolinecarboxylic acid, 5-amino-1-cyclopropyl-7-(3,5-dimethyl-1-piperazinyl)-6,8-difluoro-1,4-dihydro-4-oxo-, cis-), AVE-6971, cinoxacin ([1,3]dioxolo[4,5-g]cinnoline-3-carboxylic acid, 1-ethyl-1,4-dihydro-4-oxo-), or an analogue or derivative thereof).

55. Angiotensin I Converting Enzyme Inhibitor

In another embodiment, the pharmacologically active compound is an angiotensin I converting enzyme inhibitor (e.g., ramipril (cyclopenta[b]pyrrole-2-carboxylic acid, 1-[2-[[1-(ethoxycarbonyl)-3-phenylpropyl]amino]-1-oxopropyl]octahydro-, [2S-[1 [R*(R*)],2 alpha,3aβ,6aβ]]-), trandolapril (1H-indole-2-carboxylic acid, 1-[2-[(1-carboxy-3-phenylpropyl)amino]-1-oxopropyl]octahydro-, [2S-[1[R*(R*)],2 alpha,3a alpha,7aβ]]-), fasidotril (L-alanine, N-[(2S)-3-(acetylthio)-2-(1,3-benzodioxol-5-ylmethyl)-1-oxopropyl]-, phenylmethyl ester), cilazapril (6H-pyridazino[1,2-a][1,2]diazepine-1-carboxylic acid, 9-[[1-(ethoxycarbonyl)-3-phenylpropyl]amino]octahydro-10-oxo-, [1S-[1 alpha, 9 alpha(R*)]]-), ramipril (cyclopenta[b]pyrrole-2-carboxylic acid, 1-[2-[[1-(ethoxycarbonyl)-3-phenylpropyl]amino]-1-oxopropyl]octahydro-, [2S-[1[R*(R*)], 2 alpha,3aβ,6aβ]]-, or an analogue or derivative thereof).

56. Angiotensin II Antagonist

In another embodiment, the pharmacologically active compound is an angiotensin II antagonist (e.g., HR-720 (1H-imidazole-5-carboxylic acid, 2-butyl-4-(methylthio)-1-[[2′-[[[(propylamino)carbonyl]amino]sulfonyl][1,1′-biphenyl]-4-yl]methyl]-, dipotassium salt, or an analogue or derivative thereof).

57. Enkephalinase Inhibitor

In another embodiment, the pharmacologically active compound is an enkephalinase inhibitor (e.g., Aventis 100240 (pyrido[2,1-a][2]benzazepine-4-carboxylic acid, 7-[[2-(acetylthio)-1-oxo-3-phenylpropyl]amino]-1,2,3,4,6,7,8,12b-octahydro-6-oxo-, [4S-[4 alpha, 7 alpha(R*),12bβ]]-), AVE-7688, or an analogue or derivative thereof).

58. Peroxisome Proliferator-Activated Receptor Gamma Agonist Insulin Sensitizer

In another embodiment, the pharmacologically active compound is peroxisome proliferator-activated receptor gamma agonist insulin sensitizer (e.g., rosiglitazone maleate (2,4-thiazolidinedione, 5-((4-(2-(methyl-2-pyridinylamino)ethoxy)phenyl)methyl)-, (Z)-2-butenedioate (1:1), farglitazar (GI-262570, GW-2570, GW-3995, GW-5393, GW-9765), LY-929, LY-519818, LY-674, or LSN-862), or an analogue or derivative thereof).

59. Protein Kinase C Inhibitor

In another embodiment, the pharmacologically active compound is a protein kinase C inhibitor, such as ruboxistaurin mesylate (9H,18H-5,21:12,17-dimethenodibenzo(e,k)pyrrolo(3,4-h)(1,4,13)oxadiazacyclohexadecine-18,20(19H)-dione, 9-((dimethylamino)methyl)-6,7,10,11-tetrahydro-, (S)-), safingol (1,3-octadecanediol, 2-amino-, [S-(R*,R*)]-), or enzastaurin hydrochloride (1H-pyrole-2,5-dione, 3-(1-methyl-1H-indol-3-yl)-4-[1-[1-(2-pyridinylmethyl)-4-piperidinyl]-1H-indol-3-yl]-, monohydrochloride), or an analogue or derivative thereof.

60. ROCK (Rho-Associated Kinase) Inhibitors

In another embodiment, the pharmacologically active compound is a ROCK (rho-associated kinase) inhibitor, such as Y-27632, HA-1077, H-1152 and 4-1-(aminoalkyl)-N-(4-pyridyl) cyclohexanecarboxamide or an analogue or derivative thereof.

61. CXCR3 Inhibitors

In another embodiment, the pharmacologically active compound is a CXCR3 inhibitor such as T-487, T0906487 or analogue or derivative thereof.

62. Itk Inhibitors

In another embodiment, the pharmacologically active compound is an Itk inhibitor such as BMS-509744 or an analogue or derivative thereof.

63. Cytosolic phospholipase A₂-Alpha Inhibitors

In another embodiment, the pharmacologically active compound is a cytosolic phospholipase A₂-alpha inhibitor such as efipladib (PLA-902) or analogue or derivative thereof.

64. PPAR Agonist

In another embodiment, the pharmacologically active compound is a PPAR Agonist (e.g., Metabolex ((−)-benzeneacetic acid, 4-chloro-alpha-[3-(trifluoromethyl)-phenoxy]-, 2-(acetylamino)ethyl ester), balaglitazone (5-(4-(3-methyl-4-oxo-3,4-dihydro-quinazolin-2-yl-methoxy)-benzyl)-thiazolidine-2,4-dione), ciglitazone (2,4-thiazolidinedione, 5-[[4-[(1-methylcyclohexyl)methoxy]phenyl]methyl]-), DRF-10945, farglitazar, GSK-677954, GW-409544, GW-501516, GW-590735, GW-590735, K-111, KRP-101, LSN-862, LY-519818, LY-674, LY-929, muraglitazar; BMS-298585 (Glycine, N-[(4-methoxyphenoxy)carbonyl]-N-[[4-[2-(5-methyl-2-phenyl-4-oxazolyl)ethoxy]phenyl]methyl]-), netoglitazone; isaglitazone (2,4-thiazolidinedione, 5-[[6-[(2-fluorophenyl)methoxy]-2-naphthalenyl]methyl]-), Actos AD-4833; U-72107A (2,4-thiazolidinedione, 5-[[4-[2-(5-ethyl-2-pyridinyl)ethoxy]phenyl]methyl]-, monohydrochloride (+/−)-), JTT-501; PNU-182716 (3,5-Isoxazolidinedione, 4-[[4-[2-(5-methyl-2-phenyl-4-oxazolyl)ethoxy]phenyl]methyl]-), AVANDIA (from SB Pharmco Puerto Rico, Inc. (Puerto Rico); BRL-48482; BRL-49653; BRL-49653c; NYRACTA and Venvia (both from (SmithKline Beecham (United Kingdom)); tesaglitazar ((2S)-2-ethoxy-3-[4-[2-[4-[(methylsulfonyl)oxy]phenyl]ethoxy]phenyl] propanoic acid), troglitazone (2,4-Thiazolidinedione, 5-[[4-[(3,4-dihydro-6-hydroxy-2,5,7,8-tetra methyl-2H-1-benzopyran-2-yl)methoxy]phenyl]methyl]-), and analogues and derivatives thereof).

65. Immunosuppressants

In another embodiment, the pharmacologically active compound is an immunosuppressant (e.g., batebulast (cyclohexanecarboxylic acid, 4-[[(aminoiminomethyl)amino]methyl]-, 4-(1,1-dimethylethyl)phenyl ester, trans-), cyclomunine, exalamide (benzamide, 2-(hexyloxy)-), LYN-001, CCI-779 (rapamycin 42-(3-hydroxy-2-(hydroxymethyl)-2-methylpropanoate)), 1726; 1726-D; AVE-1726, or an analogue or derivative thereof).

66. Erb Inhibitor

In another embodiment, the pharmacologically active compound is an Erb inhibitor (e.g., canertinib dihydrochloride (N-[4-(3-(chloro-4-fluorophenylamino)-7-(3-morpholin-4-yl-propoxy)-quinazolin-6-yl]-acrylamide dihydrochloride), CP-724714, or an analogue or derivative thereof).

67. Apoptosis Agonist

In another embodiment, the pharmacologically active compound is an apoptosis agonist (e.g., CEFLATONIN (CGX-635) (from Chemgenex Therapeutics, Inc., Menlo Park, Calif.), CHML, LBH-589, metoclopramide (benzamide, 4-amino-5-chloro-N-[2-(diethylamino)ethyl]-2-methoxy-), patupilone (4,17-dioxabicyclo(14.1.0)heptadecane-5,9-dione, 7,11-dihydroxy-8,8,10,12,16-pentamethyl-3-(1-methyl-2-(2-methyl-4-thiazolyl)ethenyl, (1R,3S,7S,10R,11S,12S,16R)), AN-9; pivanex (butanoic acid, (2,2-dimethyl-1-oxopropoxy)methyl ester), SL-100; SL-102; SL-11093; SL-11098; SL-11099; SL-93; SL-98; SL-99, or an analogue or derivative thereof).

68. Lipocortin Agonist

In another embodiment, the pharmacologically active compound is an lipocortin agonist (e.g., CGP-13774 (9Alpha-chloro-6Alpha-fluoro-11β,17alpha-dihydroxy-16Alpha-methyl-3-oxo-1,4-androstadiene-17β-carboxylic acid-methylester-17-propionate), or analogue or derivative thereof).

69. VCAM-1 Antagonist

In another embodiment, the pharmacologically active compound is a VCAM-1 antagonist (e.g., DW-908e, or an analogue or derivative thereof).

70. Collagen Antagonist

In another embodiment, the pharmacologically active compound is a collagen antagonist (e.g., E-5050 (Benzenepropanamide, 4-(2,6-dimethylheptyl)-N-(2-hydroxyethyl)-β-methyl-), lufironil (2,4-Pyridinedicarboxamide, N,N′-bis(2-methoxyethyl)-), or an analogue or derivative thereof).

71. Alpha 2 Integrin Antagonist

In another embodiment, the pharmacologically active compound is an alpha 2 integrin antagonist (e.g., E-7820, or an analogue or derivative thereof).

72. TNF Alpha Inhibitor

In another embodiment, the pharmacologically active compound is a TNF alpha inhibitor (e.g., ethyl pyruvate, Genz-29155, lentinan (Ajinomoto Co., Inc. (Japan)), linomide (3-quinolinecarboxamide, 1,2-dihydro-4-hydroxy-N,1-dimethyl-2-oxo-N-phenyl-), UR-1505, or an analogue or derivative thereof).

73. Nitric Oxide Inhibitor

In another embodiment, the pharmacologically active compound is a nitric oxide inhibitor (e.g., guanidioethyldisulfide, or an analogue or derivative thereof).

74. Cathepsin Inhibitor

In another embodiment, the pharmacologically active compound is a cathepsin inhibitor (e.g., SB-462795 or an analogue or derivative therof).

Combination Therapies

In addition to incorporation of a fibrosis-inhibiting agent, one or more other pharmaceutically active agents can be incorporated into the present compositions to improve or enhance efficacy. In one aspect, the composition may further include a compound which acts to have an inhibitory effect on pathological processes in or around the treatment site. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

In one aspect, the present invention also provides for the combination of an implantable pump or implantable sensor device (as well as compositions and methods for making implantable pump and sensor devices) that includes an anti-fibrosing agent and an anti-infective agent, which reduces the likelihood of infections.

Infection is a common complication of the implantation of foreign bodies such as, for example, medical devices. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases.

The present invention provides agents (e.g., chemotherapeutic agents) that can be released from a composition, and which have potent antimicrobial activity at extremely low doses. A wide variety of anti-infective agents can be utilized in combination with the present compositions. Suitable anti-infective agents may be readily determined based the assays provided in Example 52. Discussed in more detail below are several representative examples of agents that can be used: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

(A) Anthracyclines

Anthracyclines have the following general structure, where the R groups may be a variety of organic groups:

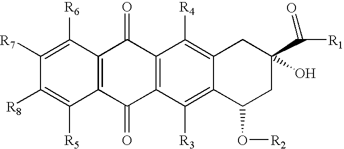

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 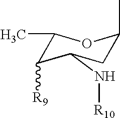
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Alternately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

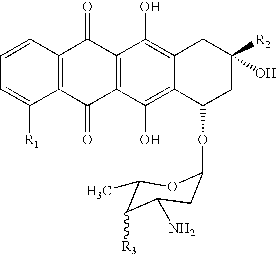

| | R₁ | R₂ | R₃ |
|---|---|---|---|
| Doxorubicin: | OCH₃ | C(O)CH₂OH | OH out of ring |
| | | | plane |
| Epirubicin: | OCH₃ | C(O)CH₂OH | OH in ring |
| (4′ epimer | | | plane |
| of doxorubicin) | | | |
| Daunorubicin: | OCH₃ | C(O)CH₃ | OH out of ring |
| | | | plane |
| Idarubicin: | H | C(O)CH₃ | OH out of ring |
| | | | plane |
| Pirarubicin: | OCH₃ | C(O)CH₂OH | 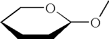 |
| Zorubicin: | OCH₃ | C(CH₃)(=N)NHC(O)C₆H₅ | OH |
| Carubicin: | OH | C(O)CH₃ | OH out of ring |
| | | | plane |

Other suitable anthracyclines are anthramycin, mitoxantrone, menogaril, nogalamycin, aclacinomycin A, olivomycin A, chromomycin A₃, and plicamycin having the structures:

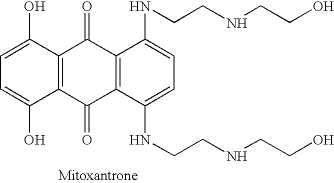

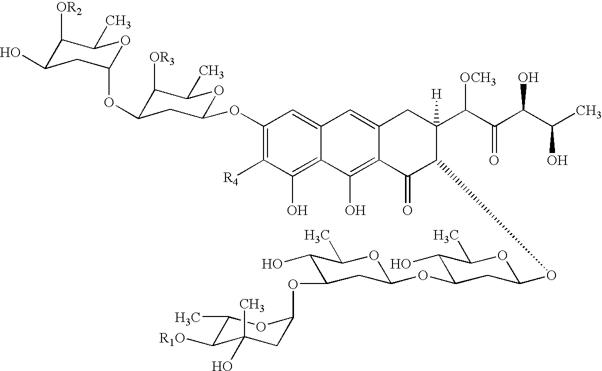

| | R₁ | R₂ | R₃ | R₄ |
|---|---|---|---|---|
| Olivomycin A | COCH(CH₃)₂ | CH₃ | COCH₃ | H |
| Chromomycin A₃ | COCH₃ | CH₃ | COCH₃ | CH₃ |
| Plicamycin | H | H | H | CH₃ |

| | R₁ | R₂ | R₃ |
|---|---|---|---|
| Menogaril | H | OCH₃ | H |
| Nogalamycin | O-sugar | H | COOCH₃ |

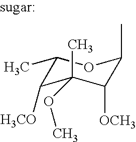

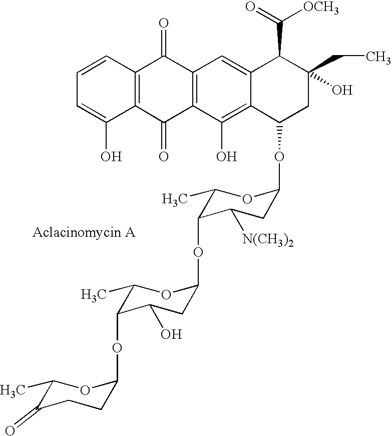

Other representative anthracyclines include, FCE 23762, a doxorubicin derivative (Quaglia et al., J. Liq. Chromatogr. 17 (18): 3911-3923, 1994), annamycin (Zou et al., J. Pharm. Sci. 82 (11): 1151-1154, 1993), ruboxyl (Rapoport et al., J. Controlled Release 58 (2): 153-162, 1999), anthracycline disaccharide doxorubicin analogue (Pratesi et al., Clin. Cancer Res. 4 (11): 2833-2839, 1998), N-(trifluoroacetyl)doxorubicin and 4′-O-acetyl-N-(trifluoroacetyl)doxorubicin (Berube & Lepage, Synth. Commun. 28 (6): 1109-1116, 1998), 2-pyrrolinodoxorubicin (Nagy et al., Proc. Nat'l Acad. Sci. U.S.A. 95 (4): 1794-1799, 1998), disaccharide doxorubicin analogues (Arcamone et al., J. Nat'l Cancer Inst. 89 (16): 1217-1223, 1997), 4-demethoxy-7-O-[2,6-dideoxy-4-O-(2,3,6-trideoxy-3-amino-α-L-lyxo-hexopyranosyl)-α-L-lyxo-hexopyranosyl]-adriamicinone doxorubicin disaccharide analogue (Monteagudo et al., Carbohydr. Res. 300 (1): 11-16, 1997), 2-pyrrolinodoxorubicin (Nagy et al., Proc. Nat'l Acad. Sci. U.S.A. 94 (2): 652-656, 1997), morpholinyl doxorubicin analogues (Duran et al., Cancer Chemother. Pharmacol. 38 (3): 210-216, 1996), enaminomalonyl-α-alanine doxorubicin derivatives (Seitz et al., Tetrahedron Lett. 36 (9): 1413-16, 1995), cephalosporin doxorubicin derivatives (Vrudhula et al., J. Med. Chem. 38 (8): 1380-5, 1995), hydroxyrubicin (Solary et al., Int. J. Cancer 58 (1): 85-94, 1994), methoxymorpholino doxorubicin derivative (Kuhl et al., Cancer Chemother. Pharmacol. 33 (1): 10-16, 1993), (6-maleimidocaproyl)hydrazone doxorubicin derivative (Willner et al., Bioconjugate Chem. 4 (6): 521-7, 1993), N-(5,5-diacetoxypent-1-yl) doxorubicin (Cherif & Farquhar, J. Med. Chem. 35 (17): 3208-14, 1992), FCE 23762 methoxymorpholinyl doxorubicin derivative (Ripamonti et al., Br. J. Cancer 65 (5): 703-7, 1992), N-hydroxysuccinimide ester doxorubicin derivatives (Demant et al., Biochim. Biophys. Acta 1118 (1): 83-90, 1991), polydeoxynucleotide doxorubicin derivatives (Ruggiero et al., Biochim. Biophys. Acta 1129 (3): 294-302, 1991), morpholinyl doxorubicin derivatives (EPA 434960), mitoxantrone doxorubicin analogue (Krapcho et al., J. Med. Chem. 34 (8): 2373-80. 1991), AD198 doxorubicin analogue (Traganos et al., Cancer Res. 51 (14): 3682-9, 1991), 4-demethoxy-3′-N-trifluoroacetyidoxorubicin (Horton et al., Drug Des. Delivery 6 (2): 123-9, 1990), 4′-epidoxorubicin (Drzewoski et al., Pol. J. Pharmacol. Pharm. 40 (2): 159-65, 1988; Weenen et al., Eur. J. Cancer Clin. Oncol. 20 (7): 919-26, 1984), alkylating cyanomorpholino doxorubicin derivative (Scudder et al., J. Nat'l Cancer Inst. 80 (16): 1294-8, 1988), deoxydihydroiodooxorubicin (EPA 275966), adriblastin (Kalishevskaya et al., Vestn. Mosk. Univ., 16 (Biol. 1): 21-7, 1988), 4′-deoxydoxorubicin (Schoelzel et al., Leuk. Res. 10 (12): 1455-9, 1986), 4-demethyoxy-4′-o-methyldoxorubicin (Giuliani et al., Proc. Int. Congr. Chemother. 16: 285-70-285-77, 1983), 3′-deamino-3′-hydroxydoxorubicin (Horton et al., J. Antibiot. 37 (8): 853-8, 1984), 4-demethyoxy doxorubicin analogues (Barbieri et al., Drugs Exp. Clin. Res. 10 (2): 85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27 (1): 5-13, 1981), aglycone doxorubicin derivatives (Chan & Watson, J. Pharm. Sci. 67 (12): 1748-52, 1978), SM 5887 (Pharma Japan 1468: 20, 1995), MX-2 (Pharma Japan 1420: 19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

(B) Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

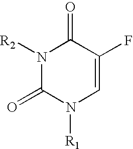

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

B

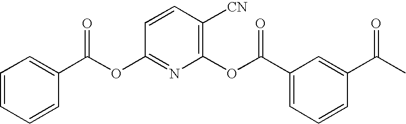

C

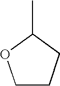

Other suitable fluoropyrimidine analogues include 5-FudR (5-fluorodeoxyuridine), or an analogue or derivative thereof, including 5-iododeoxyuridine (5-ludR), 5-bromodeoxyuridine (5-BudR), fluorouridine triphosphate (5-FUTP), and fluorodeoxyuridine monophosphate (5-dFUMP). Exemplary compounds have the structures:

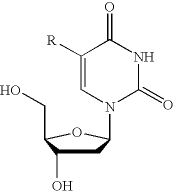

Other representative examples of fluoropyrimidine analogues include N3-alkylated analogues of 5-fluorouracil (Kozai et al., J. Chem. Soc., Perkin Trans. 1 (19): 3145-3146, 1998), 5-fluorouracil derivatives with 1,4-oxaheteroepane moieties (Gomez et al., Tetrahedron 54 (43): 13295-13312, 1998), 5-fluorouracil and nucleoside analogues (Li, Anticancer Res. 17 (1A): 21-27, 1997), cis- and trans-5-fluoro-5,6-dihydro-6-alkoxyuracil (Van der Wilt et al., Br. J. Cancer 68 (4): 702-7, 1993), cyclopentane 5-fluorouracil analogues (Hronowski & Szarek, Can. J. Chem. 70 (4): 1162-9, 1992), A-OT-fluorouracil (Zhang et al., Zongguo Yiyao Gongye Zazhi 20 (11): 513-15, 1989), N4-trimethoxybenzoyl-5′-deoxy-5-fluorocytidine and 5′-deoxy-5-fluorouridine (Miwa et al., Chem. Pharm. Bull. 38 (4): 998-1003, 1990), 1-hexylcarbamoyl-5-fluorouracil (Hoshi et al., J. Pharmacobio-Dun. 3 (9): 478-81, 1980; Maehara et al., Chemotherapy (Basel) 34 (6): 484-9, 1988), B-3839 (Prajda et al., In Vivo 2 (2): 151-4, 1988), uracil-1-(2-tetrahydrofuryl)-5-fluorouracil (Anai et al., Oncology 45 (3): 144-7, 1988), 1-(2′-deoxy-2′-fluoro-β-D-arabinofuranosyl)-5-fluorouracil (Suzuko et al., Mol. Pharmacol. 31 (3): 301-6, 1987), doxifluridine (Matuura et al., Oyo Yakuri 29 (5): 803-31, 1985), 5′-deoxy-5-fluorouridine (Bollag & Hartmann, Eur. J. Cancer 16 (4): 427-32, 1980), 1-acetyl-3-O-toluyl-5-fluorouracil (Okada, Hiroshima J. Med. Sci. 28 (1): 49-66, 1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

(C) Folic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure: 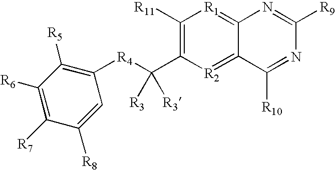
The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 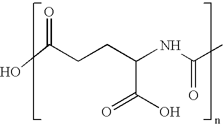
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

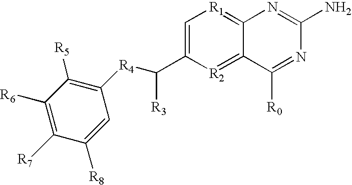

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A(n = 1) | H |
| Edatrexate | NH₂ | N | N | H | CH(CH₂CH₃) | H | H | A(n = 1) | H |
| Trimetrexate | NH₂ | CH | C(CH₃) | H | NH | H | OCH₃ | OCH₃ | OCH₃ |
| Pteropterin | OH | N | N | H | NH | H | H | A(n = 3) | H |
| Denopterin | OH | N | N | CH₃ | N(CH₃) | H | H | A(n = 1) | H |
| Peritrexim | NH₂ | N | C(CH₃) | H | single bond | OCH₃ | H | H | OCH₃ |

A:

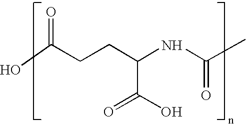

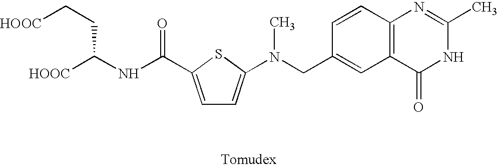

Other representative examples include 6-S-aminoacyloxymethyl mercaptopurine derivatives (Harada et al., Chem. Pharm. Bull. 43 (10): 793-6, 1995), 6-mercaptopurine (6-MP) (Kashida et al., Biol. Pharm. Bull. 18 (11): 1492-7, 1995), 7,8-polymethyleneimidazo-1,3,2-diazaphosphorines (Nilov et al., Mendeleev Commun. 2: 67, 1995), azathioprine (Chifotides et al., J. Inorg. Biochem. 56 (4): 249-64, 1994), methyl-D-glucopyranoside mercaptopurine derivatives (Da Silva et al., Eur. J. Med. Chem. 29 (2): 149-52, 1994) and s-alkynyl mercaptopurine derivatives (Ratsino et al., Khim.-Farm. Zh. 15 (8): 65-7, 1981); indoline ring and a modified ornithine or glutamic acid-bearing methotrexate derivatives (Matsuoka et al., Chem. Pharm. Bull. 45 (7): 1146-1150, 1997), alkyl-substituted benzene ring C bearing methotrexate derivatives (Matsuoka et al., Chem. Pharm. Bull. 44 (12): 2287-2293, 1996), benzoxazine or benzothiazine moiety-bearing methotrexate derivatives (Matsuoka et al., J. Med. Chem. 40 (1): 105-111, 1997), 10-deazaminopterin analogues (DeGraw et al., J. Med. Chem. 40 (3): 370-376, 1997), 5-deazaminopterin and 5,10-dideazaminopterin methotrexate analogues (Piper et al., J. Med. Chem. 40 (3): 377-384, 1997), indoline moiety-bearing methotrexate derivatives (Matsuoka et al., Chem. Pharm. Bull. 44 (7): 1332-1337, 1996), lipophilic amide methotrexate derivatives (Pignatello et al., World Meet Pharm. Biopharm. Pharm. Technol., 563-4, 1995), L-threo-(2S,4S)-4-fluoroglutamic acid and DL-3,3-difluoroglutamic acid-containing methotrexate analogues (Hart et al., J. Med. Chem. 39 (1): 56-65, 1996), methotrexate tetrahydroquinazoline analogue (Gangjee, et al., J. Heterocycl. Chem. 32 (1): 243-8, 1995), N-α-aminoacyl) methotrexate derivatives (Cheung et al., Pteridines 3 (1-2): 101-2, 1992), biotin methotrexate derivatives (Fan et al., Pteridines 3 (1-2): 131-2, 1992), D-glutamic acid or D-erythrou, threo-4-fluoroglutamic acid methotrexate analogues (McGuire et al., Biochem. Pharmacol. 42 (12): 2400-3, 1991), β,γ-methano methotrexate analogues (Rosowsky et al., Pteridines 2 (3): 133-9, 1991), 10-deazaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28 (2): 751-8, 1989), meta and ortho isomers of aminopterin (Rosowsky et al., J. Med. Chem. 32(12): 2582, 1989), hydroxymethylmethotrexate (DE 267495), γ-fluoromethotrexate (McGuire et al., Cancer Res. 49 (16): 4517-25, 1989), polyglutamyl methotrexate derivatives (Kumar et al., Cancer Res. 46 (10): 5020-3, 1986), gem-diphosphonate methotrexate analogues (WO 88/06158), α- and γ-substituted methotrexate analogues (Tsushima et al., Tetrahedron 44 (17): 5375-87, 1988), 5-methyl-5-deaza methotrexate analogues (U.S. Pat. No. 4,725,687), N6-acyl-Nα-(4-amino-4-deoxypteroyl)-L-ornithine derivatives (Rosowsky et al., J. Med. Chem. 31 (7): 1332-7, 1988), 8-deaza methotrexate analogues (Kuehl et al., Cancer Res. 48 (6): 1481-8, 1988), acivicin methotrexate analogue (Rosowsky et al., J. Med. Chem. 30 (8): 1463-9, 1987), polymeric platinol methotrexate derivative (Carraher et al., Polym. Sci. Technol. (Plenum), 35 (Adv. Biomed. Polym.): 311-24, 1987), methotrexate-γ-dimyristoylphophatidylethanolamine (Kinsky et al., Biochim. Biophys. Acta 917 (2): 211-18, 1987), methotrexate polyglutamate analogues (Rosowsky et al., Chem. Biol. Pteridines, Pteridines Folic Acid Deriv., Proc. Int. Symp. Pteridines Folic Acid Deriv.: Chem., Biol. Clin. Aspects: 985-8, 1986), poly-γ-glutamyl methotrexate derivatives (Kisliuk et al., Chem. Biol. Pteridines, Pteridines Folic Acid Deriv., Proc. Int. Symp. Pteridines Folic Acid Deriv.: Chem., Biol. Clin. Aspects: 989-92, 1986), deoxyuridylate methotrexate derivatives (Webber et al., Chem. Biol. Pteridines, Pteridines Folic Acid Deriv., Proc. Int. Symp. Pteridines Folic Acid Deriv.: Chem., Biol. Clin. Aspects: 659-62, 1986), iodoacetyl lysine methotrexate analogue (Delcamp et al., Chem. Biol. Pteridines, Pteridines Folic Acid Deriv., Proc. Int. Symp. Pteridines Folic Acid Deriv.: Chem., Biol. Clin. Aspects: 807-9, 1986), 2,.omega.-diaminoalkanoid acid-containing methotrexate analogues (McGuire et al., Biochem. Pharmacol. 35 (15): 2607-13, 1986), polyglutamate methotrexate derivatives (Kamen & Winick, Methods Enzymol. 122 (Vitam. Coenzymes, Pt. G): 339-46, 1986), 5-methyl-5-deaza analogues (Piper et al., J. Med. Chem. 29 (6): 1080-7, 1986), quinazoline methotrexate analogue (Mastropaolo et al., J. Med. Chem. 29 (1): 155-8, 1986), pyrazine methotrexate analogue (Lever & Vestal, J. Heterocycl. Chem. 22 (1): 5-6, 1985), cysteic acid and homocysteic acid methotrexate analogues (U.S. Pat. No. 4,490,529), γ-tert-butyl methotrexate esters (Rosowsky et al., J. Med. Chem. 28 (5): 660-7, 1985), fluorinated methotrexate analogues (Tsushima et al., Heterocycles 23 (1): 45-9, 1985), folate methotrexate analogue (Trombe, J. Bacteriol. 160 (3): 849-53, 1984), phosphonoglutamic acid analogues (Sturtz & Guillamot, Eur. J. Med. Chem.—Chim. Ther. 19 (3): 267-73, 1984), poly (L-lysine) methotrexate conjugates (Rosowsky et al., J. Med. Chem. 27 (7): 888-93, 1984), dilysine and trilysine methotrexate derivates (Forsch & Rosowsky, J. Org. Chem. 49 (7): 1305-9, 1984), 7-hydroxymethotrexate (Fabre et al., Cancer Res. 43 (10): 4648-52, 1983), poly-γ-glutamyl methotrexate analogues (Piper & Montgomery, Adv. Exp. Med. Biol., 163 (Folyl Antifolyl Polyglutamates): 95-100, 1983), 3′,5′-dichloromethotrexate (Rosowsky & Yu, J. Med. Chem. 26 (10): 1448-52, 1983), diazoketone and chloromethylketone methotrexate analogues (Gangjee et al., J. Pharm. Sci. 71 (6): 717-19, 1982), 10-propargylaminopterin and alkyl methotrexate homologs (Piper et al., J. Med. Chem. 25 (7): 877-80, 1982), lectin derivatives of methotrexate (Lin et al., JNCI 66 (3): 523-8, 1981), polyglutamate methotrexate derivatives (Galivan, Mol. Pharmacol. 17 (1): 105-10, 1980), halogentated methotrexate derivatives (Fox, JNCI 58 (4): J955-8, 1977), 8-alkyl-7,8-dihydro analogues (Chaykovsky et al., J. Med. Chem. 20 (10): J1323-7, 1977), 7-methyl methotrexate derivatives and dichloromethotrexate (Rosowsky & Chen, J. Med. Chem. 17 (12): J1308-11, 1974), lipophilic methotrexate derivatives and 3′,5′-dichloromethotrexate (Rosowsky, J. Med. Chem. 16 (10): J1190-3, 1973), deaza amethopterin analogues (Montgomery et al., Ann. N.Y. Acad. Sci. 186: J227-34, 1971), MX068 (Pharma Japan, 1658: 18, 1999) and cysteic acid and homocysteic acid methotrexate analogues (EPA 0142220);

These compounds are believed to act as antimetabolites of folic acid.

(D) Podophyllotoxins

In another aspect, the therapeutic agent is a podophyllotoxin, or a derivative or an analogue thereof. Exemplary compounds of this type are etoposide or teniposide, which have the following structures:

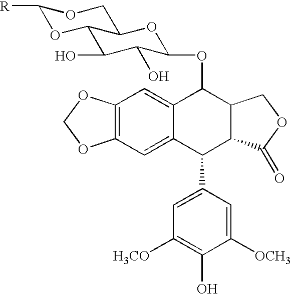

| | R |
|---|---|
| Etoposide | CH₃ |
| Teniposide | 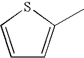 |

Other representative examples of podophyllotoxins include Cu(II)-VP-16 (etoposide) complex (Tawa et al., Bioorg. Med. Chem. 6 (7): 1003-1008, 1998), pyrrolecarboxamidino-bearing etoposide analogues (Ji et al., Bioorg. Med. Chem. Lett. 7 (5): 607-612, 1997), 4β-amino etoposide analogues (Hu, University of North Carolina Dissertation, 1992), γ-lactone ring-modified arylamino etoposide analogues (Zhou et al., J. Med. Chem. 37 (2): 287-92, 1994), N-glucosyl etoposide analogue (Allevi et al., Tetrahedron Lett. 34 (45): 7313-16, 1993), etoposide A-ring analogues (Kadow et al., Bioorg. Med. Chem. Lett 2 (1): 17-22, 1992), 4′-deshydroxy-4′-methyl etoposide (Saulnier et al., Bioorg. Med. Chem. Lett. 2 (10): 1213-18, 1992), pendulum ring etoposide analogues (Sinha et al., Eur. J. Cancer 26 (5): 590-3, 1990) and E-ring desoxy etoposide analogues (Saulnier et al., J. Med. Chem. 32 (7): 1418-20, 1989).

These compounds are believed to act as topoisomerase II inhibitors and/or DNA cleaving agents.

(E) Camptothecins

In another aspect, the therapeutic agent is camptothecin, or an analogue or derivative thereof. Camptothecins have the following general structure.

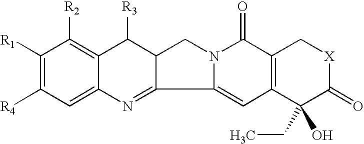

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R.

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

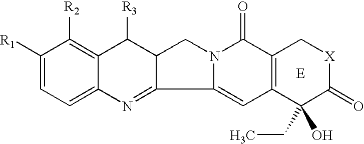

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

(F) Hydroxyureas

The therapeutic agent of the present invention may be a hydroxyurea. Hydroxyureas have the following general structure:

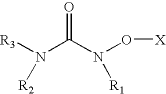

Suitable hydroxyureas are disclosed in, for example, U.S. Pat. No. 6,080,874, wherein R₁ is: 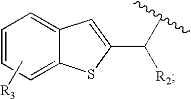
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 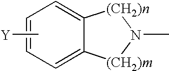
wherein m is 1 or 2, n is 0-2 and Y is an alkyl group.

In one aspect, the hydroxyurea has the structure:

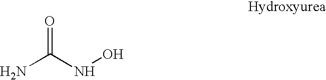

These compounds are thought to function by inhibiting DNA synthesis.

(G) Platinum Complexes

In another aspect, the therapeutic agent is a platinum compound. In general, suitable platinum complexes may be of Pt(II) or Pt(IV) and have this basic structure: 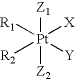
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxy, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

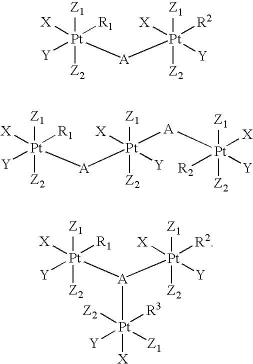

Exemplary platinum compounds are cisplatin, carboplatin, oxaliplatin, and miboplatin having the structures:

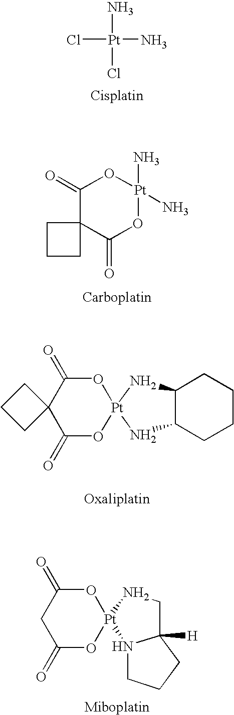

Other representative platinum compounds include (CPA)₂Pt[DOLYM] and (DACH)Pt[DOLYM] cisplatin (Choi et al., Arch. Pharmacal Res. 22 (2): 151-156, 1999), Cis-[PtCl₂(4,7-H-5-methyl-7-oxo]1,2,4[triazolo[1,5-a]pyrimidine)₂] (Navarro et al., J. Med. Chem. 41 (3): 332-338, 1998), [Pt(cis-1,4-DACH)(trans-Cl₂)(CBDCA)].½MeOH cisplatin (Shamsuddin et al., Inorg. Chem. 36 (25): 5969-5971, 1997), 4-pyridoxate diammine hydroxy platinum (Tokunaga et al., Pharm. Sci. 3 (7): 353-356, 1997), Pt(II) . . . Pt(II) (Pt₂[NHCHN(C(CH₂)(CH₃))]₄) (Navarro et al., Inorg. Chem. 35 (26): 7829-7835, 1996), 254-S cisplatin analogue (Koga et al., Neurol. Res. 18 (3): 244-247, 1996), o-phenylenediamine ligand bearing cisplatin analogues (Koeckerbauer & Bednarski, J. Inorg. Biochem. 62 (4): 281-298, 1996), trans, cis-[Pt(OAc)₂I₂(en)] (Kratochwil et al., J. Med. Chem. 39 (13): 2499-2507, 1996), estrogenic 1,2-diarylethylenediamine ligand (with sulfur-containing amino acids and glutathione) bearing cisplatin analogues (Bednarski, J. Inorg. Biochem. 62(1): 75, 1996), cis-1,4-diaminocyclohexane cisplatin analogues (Shamsuddin et al., J. Inorg. Biochem. 61 (4): 291-301, 1996), 5′ orientational isomer of cis-[Pt(NH₃)(4-aminoTEMP-O){d(GpG)}] (Dunham & Lippard, J. Am. Chem. Soc. 117 (43): 10702-12, 1995), chelating diamine-bearing cisplatin analogues (Koeckerbauer & Bednarski, J. Pharm. Sci. 84 (7): 819-23, 1995), 1,2-diarylethyleneamine ligand-bearing cisplatin analogues (Otto et al., J. Cancer Res. Clin. Oncol. 121 (1): 31-8, 1995), (ethylenediamine)platinum(II) complexes (Pasini et al., J. Chem. Soc., Dalton Trans. 4: 579-85, 1995), CI-973 cisplatin analogue (Yang et al., Int. J. Oncol. 5 (3): 597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26 (4): 257-67, 1986; Fan et al., Cancer Res. 48 (11): 3135-9, 1988; Heiger-Bernays et al., Biochemistry 29 (36): 8461-6, 1990; Kikkawa et al., J. Exp. Clin. Cancer Res. 12 (4): 233-40, 1993; Murray et al., Biochemistry 31 (47): 11812-17, 1992; Takahashi et al., Cancer Chemother. Pharmacol. 33 (1): 31-5, 1993), cis-amine-cyclohexylamine-dichloroplatinum(II) (Yoshida et al., Biochem. Pharmacol. 48 (4): 793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35 (23): 4479-85, 1992), cisplatin analogues containing a tethered dansyl group (Hartwig et al., J. Am. Chem. Soc. 114 (21): 8292-3, 1992), platinum(II) polyamines (Siegmann et al., Inorg. Met.-Containing Polym. Mater., (Proc. Am. Chem. Soc. Int. Symp.), 335-61, 1990), cis-(3H)dichloro(ethylenediamine)platinum(II) (Eastman, Anal. Biochem. 197 (2): 311-15, 1991), trans-diamminedichloroplatinum(II) and cis-(Pt(NH₃)₂(N₃-cytosine)Cl) (Bellon & Lippard, Biophys. Chem. 35 (2-3): 179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64 (1): 41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25 (4): 349-57, 1988), aminoalkylaminoanthraquinone-derived cisplatin analogues (Kitov et al., Eur. J. Med. Chem. 23 (4): 381-3, 1988), spiroplatin, carboplatin, iproplatin and JM40 platinum analogues (Schroyen et al., Eur. J. Cancer Clin. Oncol. 24 (8): 1309-12, 1988), bidentate tertiary diamine-containing cisplatinum derivatives (Orbell et al., Inorg. Chim. Acta 152 (2): 125-34, 1988), platinum(II), platinum(IV) (Liu & Wang, Shandong Yike Daxue Xuebao 24 (1): 35-41, 1986), cis-diammine(1,1-cyclobutanedicarboxylato-)platinum(II) (carboplatin, JM8) and ethylenediamminemalonatoplatinum(II) (JM40) (Begg et al., Radiother. Oncol. 9 (2): 157-65, 1987), JM8 and JM9 cisplatin analogues (Harstrick et al., Int. J. Androl. 10 (1); 139-45, 1987), (NPr4)2((PtCL4).cis-(PtCl2-(NH2Me)2)) (Brammer et al., J. Chem. Soc., Chem. Commun. 6: 443-5, 1987), aliphatic tricarboxylic acid platinum complexes (EPA 185225), and cis-dichloro(amino acid)(tert-butylamine)platinum(II) complexes (Pasini & Bersanetti, Inorg. Chim. Acta 107 (4): 259-67, 1985). These compounds are thought to function by binding to DNA, i.e., acting as alkylating agents of DNA.

As medical implants are made in a variety of configurations and sizes, the exact dose administered may vary with device size, surface area, design and portions of the implant coated. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the cardiac implant, the preferred anticancer agents, used alone or in combination, may be administered under the following dosing guidelines:

(a) Anthracyclines. Utilizing the anthracycline doxorubicin as an example, whether applied as a polymer coating, incorporated into the polymers which make up the implant components, or applied without a carrier polymer, the total dose of doxorubicin applied to the implant should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, doxorubicin should be applied to the implant surface at a dose of 0.1 μg/mm²-10 μg/mm². As different polymer and non-polymer coatings may release doxorubicin at differing rates, the above dosing parameters should be utilized in combination with the release rate of the drug from the implant surface such that a minimum concentration of 10⁻⁸-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the implant, or applied without a carrier polymer, the total dose of mitoxantrone applied should not exceed 5 mg (range of 0.01 μg to 5 mg). In a particularly preferred embodiment, the total amount of drug applied should be in the range of 0.1 μg to 3 mg. The dose per unit area (i.e., the amount of drug as a function of the surface area of the portion of the implant to which drug is applied and/or incorporated) should fall within the range of 0.01 μg-20 μg per mm² of surface area. In a particularly preferred embodiment, mitoxantrone should be applied to the implant surface at a dose of 0.05 μg/mm²-5 μg/mm². As different polymer and non-polymer coatings will release mitoxantrone at differing rates, the above dosing parameters should be utilized in combination with the release rate of the drug from the implant surface such that a minimum concentration of 10⁻⁴-10⁻⁸ M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.05 μg-200 μg per mm² of surface area. In a particularly preferred embodiment, 5-fluorouracil should be applied to the implant surface at a dose of 0.5 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the cardiac implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁴-10⁻⁷ M of etoposide is maintained. It is necessary to insure that surface drug concentrations exceed concentrations of etoposide known to be lethal to a variety of bacteria and fungi (i.e., are in excess of 10⁻⁵ M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

It may be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the composition.

In another aspect, an anti-infective agent (e.g., anthracyclines (e.g., doxorubicin or mitoxantrone), fluoropyrimidines (e.g., 5-fluorouracil), folic acid antagonists (e.g., methotrexate and/or podophylotoxins (e.g., etoposide)) can be combined with traditional antibiotic and/or antifungal agents to enhance efficacy. The anti-infective agent may be further combined with anti-thrombotic and/or antiplatelet agents (for example, heparin, dextran sulphate, danaparoid, lepirudin, hirudin, AMP, adenosine, 2-chloroadenosine, aspirin, phenylbutazone, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

In addition to incorporation of the above-mentioned therapeutic agents (i.e., anti-infective agents or fibrosis-inhibiting agents), one or more other pharmaceutically active agents can be incorporated into the present compositions and devices to improve or enhance efficacy. Representative examples of additional therapeutically active agents include, by way of example and not limitation, anti-thrombotic agents, anti-proliferative agents, anti-inflammatory agents, neoplastic agents, enzymes, receptor antagonists or agonists, hormones, antibiotics, antimicrobial agents, antibodies, cytokine inhibitors, IMPDH (inosine monophosplate dehydrogenase) inhibitors tyrosine kinase inhibitors, MMP inhibitors, p38 MAP kinase inhibitors, immunosuppressants, apoptosis antagonists, caspase inhibitors, and JNK inhibitors.

Implantable implantable pump and sensor devices and compositions for use with implantable pump and sensor devices may further include an anti-thrombotic agent and/or antiplatelet agent and/or a thrombolytic agent, which reduces the likelihood of thrombotic events upon implantation of a medical implant. Within various embodiments of the invention, a device is coated on one aspect with a composition which inhibits fibrosis (and/or restenosis), as well as being coated with a composition or compound which prevents thrombosis on another aspect of the device. Representative examples of anti-thrombotic and/or antiplatelet and/or thrombolytic agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, acetylsalicylic acid, phenylbutazone, indomethacin, meclofenamate, hydrochloroquine, dipyridamole, iloprost, streptokinase, factor Xa inhibitors, such as DX9065a, magnesium, and tissue plasminogen activator. Further examples include plasminogen, lys-plasminogen, alpha-2-antiplasmin, urokinase, aminocaproic acid, ticlopidine, clopidogrel, trapidil (triazolopyrimidine), naftidrofuryl, auriritricarboxylic acid and glycoprotein IIb/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone.

Compositions for use with implantable pump and sensor devices may be or include a hydrophilic polymer gel that itself has anti-thrombogenic properties. For example, the composition can be in the form of a coating that can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87), chain extended PLURONIC polymers, various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Implantable pump and sensor devices and compositions for use with implantable pump and sensor devices may further include a compound which acts to have an inhibitory effect on pathological processes in or around the treatment site. In certain aspects, the agent may be selected from one of the following classes of compounds: anti-inflammatory agents (e.g., dexamethasone, cortisone, fludrocortisone, prednisone, prednisolone, 6α-methylprednisolone, triamcinolone, betamethasone, and aspirin); MMP inhibitors (e.g., batimistat, marimistat, TIMP's representative examples of which are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, 1α-hydroxy vitamin D₃), IMPDH (inosine monophosplate dehydrogenase) inhibitors (e.g., mycophenolic acid, ribaviran, aminothiadiazole, thiophenfurin, tiazofurin, viramidine) (Representative examples are included in U.S. Pat. Nos. 5,536,747; 5,807,876; 5,932,600; 6,054,472; 6,128,582;, 6,344,465; 6,395,763; 6,399,773; 6,420,403; 6,479,628; 6,498,178; 6,514,979; 6,518,291; 6,541,496; 6,596,747; 6,617,323; and 6,624,184, U.S. Patent Application Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071 A1, WO 99/001545A1, WO 97/40028A1, WO 97/41211A1, WO 98/40381A1, and WO 99/55663A1), p38 MAP kinase inhibitors (MAPK) (e.g., GW-2286, CGP-52411, BIRB-798, SB220025, RO-320-1195, RWJ-67657, RWJ-68354, SCIO-469) (Representative examples are included in U.S. Pat. Nos. 6,300,347; 6,316,464; 6,316,466; 6,376,527; 6,444,696; 6,479,507; 6,509,361; 6,579,874, and 6,630,485, and U.S. Patent Application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031 A1, 2003/0166647A1, and 2003/0181411 A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2, WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1), and immunomodulatory agents (rapamycin, everolimus, ABT-578, azathioprine azithromycin, analogues of rapamycin, including tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389.

Other examples of biologically active agents which may be combined with implantable pump and sensor devices according to the invention include tyrosine kinase inhibitors, such as imantinib, ZK-222584, CGP-52411, CGP-53716, NVP-AAK980-NX, CP-127374, CP-564959, PD-171026, PD-173956, PD-180970, SU-0879, and SKI-606; MMP inhibitors such as nimesulide, PKF-241-466, PKF-242-484, CGS-27023A, SAR-943, primomastat, SC-77964, PNU-171829, AG-3433, PNU-142769, SU-5402, and dexlipotam; p38 MAP kinase inhibitors such as include CGH-2466 and PD-98-59; immunosuppressants such as argyrin B, macrocyclic lactone, ADZ-62-826, CCI-779, tilomisole, amcinonide, FK-778, AVE-1726, and MDL-28842; cytokine inhibitors such as TNF-484A, PD-172084, CP-293121, CP-353164, and PD-168787; NFKB inhibitors, such as, AVE-0547, AVE-0545, and IPL-576092; HMGCoA reductase inhibitors, such as, pravestatin, atorvastatin, fluvastatin, dalvastatin, glenvastatin, pitavastatin, CP-83101, U-20685; apoptosis antagonist (e.g., troloxamine, TCH-346 (N-methyl-N-propargyl-10-aminomethyl-dibenzo(b,f)oxepin); and caspase inhibitors (e.g., PF-5901 (benzenemethanol, alpha-pentyl-3-(2-quinolinylmethoxy)-), and JNK inhibitor (e.g., AS-602801).

In another aspect, the implantable pump and sensor devices may further include an antibiotic (e.g., amoxicillin, trimethoprim-sulfamethoxazole, azithromycin, clarithromycin, amoxicillin-clavulanate, cefprozil, cefuroxime, cefpodoxime, or cefdinir).

In certain aspects, a polymeric composition comprising a fibrosis-inhibiting agent is combined with an agent that can modify metabolism of the agent in vivo to enhance efficacy of the fibrosis-inhibiting agent. One class of therapeutic agents that can be used to alter drug metabolism includes agents capable of inhibiting oxidation of the anti-scarring agent by cytochrome P450 (CYP). In one embodiment, compositions are provided that include a fibrosis-inhibiting agent (e.g., paclitaxel, rapamycin, everolimus) and a CYP inhibitor, which may be combined (e.g., coated) with any of the devices described herein. Representative examples of CYP inhibitors include flavones, azole antifungals, macrolide antibiotics, HIV protease inhibitors, and anti-sense oligomers. Devices comprising a combination of a fibrosis-inhibiting agent and a CYP inhibitor may be used to treat a variety of proliferative conditions that can lead to undesired scarring of tissue, including intimal hyperplasia, surgical adhesions, and tumor growth.

Within various embodiments of the invention, a device incorporates or is coated on one aspect, portion or surface, portion or surface with a composition which inhibits fibrosis (and/or restenosis), as well as with a composition or compound which promotes or stimulates fibrosis on another aspect, portion or surface, portion or surface of the device. Compounds that promote or stimulate fibrosis can be identified by, for example, the in vivo (animal) models provided in Examples 48-51. Representative examples of agents that promote fibrosis include silk and other irritants (e.g., talc, wool (including animal wool, wood wool, and synthetic wool), talcum powder, copper, metallic beryllium (or its oxides), quartz dust, silica, crystalline silicates), polymers (e.g., polylysine, polyurethanes, poly(ethylene terephthalate), PTFE, poly(alkylcyanoacrylates), and poly(ethylene-co-vinylacetate); vinyl chloride and polymers of vinyl chloride; peptides with high lysine content; growth factors and inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-β (TGF-β-1, TGF-β-2, TGF-β-3, platelet-derived growth factor (PDGF), fibroblast growth factor (acidic—aFGF; and basic—bFGF), fibroblast stimulating factor-1, activins, vascular endothelial growth factor (including VEGF-2, VEGF-3, VEGF-A, VEGF-B, VEGF-C, placental growth factor-PIGF), angiopoietins, insulin-like growth factors (IGF), hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, and IL-6), tumor necrosis factor-α (TNFα), nerve growth factor (NGF), interferon-α, interferon-β, histamine, endothelin-1, angiotensin II, growth hormone (GH), and synthetic peptides, analogues or derivatives of these factors are also suitable for release from specific implants and devices to be described later. Other examples include CTGF (Connective tissue growth factor); inflammatory microcrystals (e.g., crystalline minerals such as crystalline silicates); bromocriptine, methylsergide, methotrexate, chitosan, N-carboxybutyl chitosan, carbon tetrachloride, thioacetamide, fibrosin, ethanol, bleomycin, naturally occurring or synthetic peptides containing the Arg-Gly-Asp (RGD) sequence, generally at one or both termini (see, e.g., U.S. Pat. No. 5,997,895), and tissue adhesives, such as cyanoacrylate and crosslinked poly(ethylene glycol)-methylated collagen compositions. Other examples of fibrosis-inducing agents include bone morphogenic proteins (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242 (4885); 1528-1534.

Other representative examples of fibrosis-inducing agents include components of extracellular matrix (e.g., fibronectin, fibrin, fibrinogen, collagen (e.g., bovine collagen), including fibrillar and non-fibrillar collagen, adhesive glycoproteins, proteoglycans (e.g., heparin sulfate, chondroitin sulfate, dermatan sulfate), hyaluronan, secreted protein acidic and rich in cysteine (SPARC), thrombospondins, tenacin, and cell adhesion molecules (including integrins, vitronectin, fibronectin, laminin, hyaluronic acid, elastin, bitronectin), proteins found in basement membranes, and fibrosin) and inhibitors of matrix metalloproteinases, such as TIMPs (tissue inhibitors of matrix metalloproteinases) and synthetic TIMPs, such as, e.g., marimistat, batimistat, doxycycline, tetracycline, minocycline, TROCADE, Ro-1130830, CGS 27023A, and BMS-275291 and analogues and derivatives thereof.

Although the above therapeutic agents have been provided for the purposes of illustration, it may be understood that the present invention is not so limited. For example, although agents are specifically referred to above, the present invention may be understood to include analogues, derivatives and conjugates of such agents. As an illustration, paclitaxel may be understood to refer to not only the common chemically available form of paclitaxel, but analogues (e.g., TAXOTERE, as noted above) and paclitaxel conjugates (e.g., paclitaxel-PEG, paclitaxel-dextran, or paclitaxel-xylos). In addition, as will be evident to one of skill in the art, although the agents set forth above may be noted within the context of one class, many of the agents listed in fact have multiple biological activities. Further, more than one therapeutic agent may be utilized at a time (i.e., in combination), or delivered sequentially.

C. Dosages

Since implantable sensor and implantable pumps (and their drug delivery catheters or ports) are made in a variety of configurations and sizes, the exact dose administered will vary with device size, surface area and design. However, as described above, certain principles can be applied in the application of this art. Drug dose can be calculated as a function of dose (i.e., amount) per unit area of the portion of the device being coated. Surface area can be measured or determined by methods known to one of ordinary skill in the art. Total drug dose administered can be measured and appropriate surface concentrations of active drug can be determined. Drugs are to be used at concentrations that range from several times more than to 10%, 5%, or even less than 1% of the concentration typically used in a single systemic dose application. In certain embodiments, the drug is released in effective concentrations for a period ranging from 1-90 days. Regardless of the method of application of the drug to the device, the fibrosis-inhibiting agents, used alone or in combination, may be administered under the following dosing guidelines:

As described above, implantable sensors and pumps may be used in combination with a composition that includes an anti-scarring agent. The total amount (dose) of anti-scarring agent in or on the device may be in the range of about 0.01 μg-10 μg, or 10 μg-10 mg, or 10 mg-250 mg, or 250 mg-1000 mg, or 1000 mg-2500 mg. The dose (amount) of anti-scarring agent per unit area of device surface to which the agent is applied may be in the range of about 0.01 μg/mm²-1 μg/mm², or 1 μg/mm²-10 μg/mm², or 10 μg/mm²-250 μg/mm², 250 g/mm²-1000 μg/mm², or 1000 μg/mm²-2500 μg/mm².

It may be apparent to one of skill in the art that potentially any anti-fibrosis agent described above may be utilized alone, or in combination, in the practice of this embodiment.

In various aspects, the present invention provides implantable sensors and pumps containing an angiogenesis inhibitor in a dosage as set forth above. In various aspects, the present invention provides implantable sensors and pumps containing a 5-lipoxygenase inhibitor or antagonist in a dosage as set forth above. In various aspects, the present invention provides implantable sensors and pumps containing a chemokine receptor antagonist in a dosage as set forth above. In various aspects, the present invention provides implantable sensors and pumps containing a cell cycle inhibitor in a dosage as set forth above. In various aspects, the present invention provides implantable sensors and pumps containing an anthracycline (e.g., doxorubicin and mitoxantrone) in a dosage as set forth above. In various aspects, the present invention provides implantable sensors and pumps containing a taxane (e.g., paclitaxel or an analogue or derivative of paclitaxel) in a dosage as set forth above. In various aspects, the present invention provides implantable sensors and pumps containing a podophyllotoxin (e.g., etoposide) in a dosage as set forth above. In various aspects, the present invention provides implantable sensors and pumps containing a vinca alkaloid in a dosage as set forth above. In various aspects, the present invention provides implantable sensors and pumps containing a camptothecin or an analogue or derivative thereof in a dosage as set forth above. In various aspects, the present invention provides implantable sensors and pumps containing a platinum compound in a dosage as set forth above. In various aspects, the present invention provides implantable sensors and pumps containing a nitrosourea in a dosage as set forth above. In various aspects, the present invention provides implantable sensors and pumps containing a nitroimidazole in a dosage as set forth above. In various aspects, the present invention provides implantable sensors and pumps containing a folic acid antagonist in a dosage as set forth above. In various aspects, the present invention provides implantable sensors and pumps containing a cytidine analogue in a dosage as set forth above. In various aspects, the present invention provides implantable sensors and pumps containing a pyrimidine analogue in a dosage as set forth above. In various aspects, the present invention provides implantable sensors and pumps containing a fluoropyrimidine analogue in a dosage as set forth above. In various aspects, the present invention provides implantable sensors and pumps containing a purine analogue in a dosage as set forth above. In various aspects, the present invention provides implantable sensors and pumps containing a nitrogen mustard in a dosage as set forth above. In various aspects, the present invention provides implantable sensors and pumps containing a hydroxyurea in a dosage as set forth above. In various aspects, the present invention provides implantable sensors and pumps containing a mytomicin in a dosage as set forth above. In various aspects, the present invention provides implantable sensors and pumps containing an alkyl sulfonate in a dosage as set forth above. In various aspects, the present invention provides implantable sensors and pumps containing a benzamide in a dosage as set forth above. In various aspects, the present invention provides implantable sensors and pumps containing a nicotinamide in a dosage as set forth above. In various aspects, the present invention provides implantable sensors and pumps containing a halogenated sugar in a dosage as set forth above. In various aspects, the present invention provides implantable sensors and pumps containing a DNA alkylating agent in a dosage as set forth above. In various aspects, the present invention provides implantable sensors and pumps containing an anti-microtubule agent in a dosage as set forth above. In various aspects, the present invention provides implantable sensors and pumps containing a topoisomerase inhibitor in a dosage as set forth above. In various aspects, the present invention provides implantable sensors and pumps containing a DNA cleaving agent in a dosage as set forth above. In various aspects, the present invention provides implantable sensors and pumps containing an antimetabolite in a dosage as set forth above. In various aspects, the present invention provides implantable sensors and pumps containing an agent that inhibits adenosine deaminase in a dosage as set forth above. In various aspects, the present invention provides implantable sensors and pumps containing an agent that inhibits purine ring synthesis in a dosage as set forth above. In various aspects, the present invention provides implantable sensors and pumps containing a nucleotide interconversion inhibitor in a dosage as set forth above. In various aspects, the present invention provides implantable sensors and pumps containing an agent that inhibits dihydrofolate reduction in a dosage as set forth above. In various aspects, the present invention provides implantable sensors and pumps containing an agent that blocks thymidine monophosphate function in a dosage as set forth above. In various aspects, the present invention provides implantable sensors and pumps containing an agent that causes DNA damage in a dosage as set forth above. In various aspects, the present invention provides implantable sensors and pumps containing a DNA intercalation agent in a dosage as set forth above. In various aspects, the present invention provides implantable sensors and pumps containing an agent that is a RNA synthesis inhibitor in a dosage as set forth above. In various aspects, the present invention provides implantable sensors and pumps containing an agent that is a pyrimidine synthesis inhibitor in a dosage as set forth above. In various aspects, the present invention provides implantable sensors and pumps containing an agent that inhibits ribonucleotide synthesis in a dosage as set forth above. In various aspects, the present invention provides implantable sensors and pumps containing an agent that inhibits thymidine monophosphate synthesis in a dosage as set forth above. In various aspects, the present invention provides implantable sensors and pumps containing an agent that inhibits DNA synthesis in a dosage as set forth above. In various aspects, the present invention provides implantable sensors and pumps containing an agent that causes DNA adduct formation in a dosage as set forth above. In various aspects, the present invention provides implantable sensors and pumps containing an agent that inhibits protein synthesis in a dosage as set forth above. In various aspects, the present invention provides implantable sensors and pumps containing an agent that inhibits microtubule function in a dosage as set forth above. In various aspects, the present invention provides implantable sensors and pumps containing an immunomodulatory agent (e.g., sirolimus, everolimus, tacrolimus, or an analogue or derivative thereof) in a dosage as set forth above. In various aspects, the present invention provides implantable sensors and pumps containing a heat shock protein 90 antagonist (e.g., geldanamycin) in a dosage as set forth above. In various aspects, the present invention provides implantable sensors and pumps containing an HMGCoA reductase inhibitor (e.g., simvastatin) in a dosage as set forth above. In various aspects, the present invention provides implantable sensors and pumps containing an inosine monophosphate dehydrogenase inhibitor (e.g., mycophenolic acid, 1-alpha-25 dihydroxy vitamin D₃) in a dosage as set forth above. In various aspects, the present invention provides implantable sensors and pumps containing an NF kappa B inhibitor (e.g., Bay 11-7082) in a dosage as set forth above. In various aspects, the present invention provides implantable sensors and pumps containing an antimycotic agent (e.g., sulconizole) in a dosage as set forth above. In various aspects, the present invention provides implantable sensors and pumps containing a p38 MAP kinase inhibitor (e.g., SB202190) in a dosage as set forth above. In various aspects, the present invention provides implantable sensors and pumps containing a cyclin dependent protein kinase inhibitor in a dosage as set forth above. In various aspects, the present invention provides implantable sensors and pumps containing an epidermal growth factor kinase inhibitor in a dosage as set forth above. In various aspects, the present invention provides implantable sensors and pumps containing an elastase inhibitor in a dosage as set forth above. In various aspects, the present invention provides implantable sensors and pumps containing a factor Xa inhibitor in a dosage as set forth above. In various aspects, the present invention provides implantable sensors and pumps containing a farnesyltransferase inhibitor in a dosage as set forth above. In various aspects, the present invention provides implantable sensors and pumps containing a fibrinogen antagonist in a dosage as set forth above. In various aspects, the present invention provides implantable sensors and pumps containing a guanylate cyclase stimulant in a dosage as set forth above. In various aspects, the present invention provides implantable sensors and pumps containing a hydroorotate dehydrogenase inhibitor in a dosage as set forth above. In various aspects, the present invention provides implantable sensors and pumps containing an IKK2 inhibitor in a dosage as set forth above. In various aspects, the present invention provides implantable sensors and pumps containing an IL-1 antagonist in a dosage as set forth above. In various aspects, the present invention provides implantable sensors and pumps containing an ICE antagonist in a dosage as set forth above. In various aspects, the present invention provides implantable sensors and pumps containing an IRAK antagonist in a dosage as set forth above. In various aspects, the present invention provides implantable sensors and pumps containing an IL-4 agonist in a dosage as set forth above. In various aspects, the present invention provides implantable sensors and pumps containing a leukotriene inhibitor in a dosage as set forth above. In various aspects, the present invention provides implantable sensors and pumps containing an MCP-1 antagonist in a dosage as set forth above. In various aspects, the present invention provides implantable sensors and pumps containing a MMP inhibitor in a dosage as set forth above. In various aspects, the present invention provides implantable sensors and pumps containing an NO antagonist in a dosage as set forth above. In various aspects, the present invention provides implantable sensors and pumps containing a phosphodiesterase inhibitor in a dosage as set forth above. In various aspects, the present invention provides implantable sensors and pumps containing a TGF beta inhibitor in a dosage as set forth above. In various aspects, the present invention provides implantable sensors and pumps containing a thromboxane A2 antagonist in a dosage as set forth above. In various aspects, the present invention provides implantable sensors and pumps containing a TNF alpha antagonist in a dosage as set forth above. In various aspects, the present invention provides implantable sensors and pumps containing a TACE inhibitor in a dosage as set forth above. In various aspects, the present invention provides implantable sensors and pumps containing a tyrosine kinase inhibitor in a dosage as set forth above. In various aspects, the present invention provides implantable sensors and pumps containing a vitronectin inhibitor in a dosage as set forth above. In various aspects, the present invention provides implantable sensors and pumps containing a fibroblast growth factor inhibitor in a dosage as set forth above. In various aspects, the present invention provides implantable sensors and pumps containing a protein kinase inhibitor in a dosage as set forth above. In various aspects, the present invention provides implantable sensors and pumps containing a PDGF receptor kinase inhibitor in a dosage as set forth above. In various aspects, the present invention provides implantable sensors and pumps containing an endothelial growth factor receptor kinase inhibitor in a dosage as set forth above. In various aspects, the present invention provides implantable sensors and pumps containing a retinoic acid receptor antagonist in a dosage as set forth above. In various aspects, the present invention provides implantable sensors and pumps containing a platelet derived growth factor receptor kinase inhibitor in a dosage as set forth above. In various aspects, the present invention provides implantable sensors and pumps containing a fibrinogen antagonist in a dosage as set forth above. In various aspects, the present invention provides implantable sensors and pumps containing a bisphosphonate in a dosage as set forth above. In various aspects, the present invention provides implantable sensors and pumps containing a phospholipase A1 inhibitor in a dosage as set forth above. In various aspects, the present invention provides implantable sensors and pumps containing a histamine H1/H2/H3 receptor antagonist in a dosage as set forth above. In various aspects, the present invention provides implantable sensors and pumps containing a macrolide antibiotic in a dosage as set forth above. In various aspects, the present invention provides implantable sensors and pumps containing a GPIIb IIIa receptor antagonist in a dosage as set forth above. In various aspects, the present invention provides implantable sensors and pumps containing an endothelin receptor antagonist in a dosage as set forth above. In various aspects, the present invention provides implantable sensors and pumps containing a peroxisome proliferator-activated receptor agonist in a dosage as set forth above. In various aspects, the present invention provides implantable sensors and pumps containing an estrogen receptor agent in a dosage as set forth above. In various aspects, the present invention provides implantable sensors and pumps containing a somastostatin analogue in a dosage as set forth above. In various aspects, the present invention provides implantable sensors and pumps containing a neurokinin 1 antagonist in a dosage as set forth above. In various aspects, the present invention provides implantable sensors and pumps containing a neurokinin 3 antagonist in a dosage as set forth above. In various aspects, the present invention provides implantable sensors and pumps containing a VLA-4 antagonist in a dosage as set forth above. In various aspects, the present invention provides implantable sensors and pumps containing an osteoclast inhibitor in a dosage as set forth above. In various aspects, the present invention provides implantable sensors and pumps containing a DNA topoisomerase ATP hydrolyzing inhibitor in a dosage as set forth above. In various aspects, the present invention provides implantable sensors and pumps containing an angiotensin I converting enzyme inhibitor in a dosage as set forth above. In various aspects, the present invention provides implantable sensors and pumps containing an angiotensin II antagonist in a dosage as set forth above. In various aspects; the present invention provides implantable sensors and pumps containing an enkephalinase inhibitor in a dosage as set forth above. In various aspects, the present invention provides implantable sensors and pumps containing a peroxisome proliferator-activated receptor gamma agonist insulin sensitizer in a dosage as set forth above. In various aspects, the present invention provides implantable sensors and pumps containing a protein kinase C inhibitor in a dosage as set forth above. In various aspects, the present invention provides implantable sensors and pumps containing a ROCK (rho-associated kinase) inhibitor in a dosage as set forth above. In various aspects, the present invention provides implantable sensors and pumps containing a CXCR3 inhibitor in a dosage as set forth above. In various aspects, the present invention provides implantable sensors and pumps containing a Itk inhibitor in a dosage as set forth above. In various aspects, the present invention provides implantable sensors and pumps containing a cytosolic phospholipase A₂-alpha inhibitor in a dosage as set forth above. In various aspects, the present invention provides implantable sensors and pumps containing a PPAR agonist in a dosage as set forth above. In various aspects, the present invention provides implantable sensors and pumps containing an Immunosuppressant in a dosage as set forth above. In various aspects, the present invention provides implantable sensors and pumps containing an Erb inhibitor in a dosage as set forth above. In various aspects, the present invention provides implantable sensors and pumps containing an apoptosis agonist in a dosage as set forth above. In various aspects, the present invention provides implantable sensors and pumps containing a lipocortin agonist in a dosage as set forth above. In various aspects, the present invention provides implantable sensors and pumps containing a VCAM-1 antagonist in a dosage as set forth above. In various aspects, the present invention provides implantable sensors and pumps containing a collagen antagonist in a dosage as set forth above. In various aspects, the present invention provides implantable sensors and pumps containing an alpha 2 integrin antagonist in a dosage as set forth above. In various aspects, the present invention provides implantable sensors and pumps containing a TNF alpha inhibitor in a dosage as set forth above. In various aspects, the present invention provides implantable sensors and pumps containing a nitric oxide inhibitor in a dosage as set forth above. In various aspects, the present invention provides implantable sensors and pumps containing a cathepsin inhibitor in a dosage as set forth above.

Provided below are exemplary dosage ranges for a variety of anti-fibrosis agents which can be used in conjunction with implantable sensors and pumps in accordance with the invention. A) Cell cycle inhibitors including doxorubicin and mitoxantrone. Doxorubicin analogues and derivatives thereof: total dose not to exceed 25 mg (range of 0.1 μg to 25 mg); preferred 1 μg to 5 mg. The dose per unit area of 0.01 μg-100 μg per mm²; preferred dose of 0.1 μg/mm²-10 μg/mm². Minimum concentration of 10⁻⁸-10⁻⁴ M of doxorubicin is to be maintained on the device surface. Mitoxantrone and analogues and derivatives thereof: total dose not to exceed 5 mg (range of 0.01 μg to 5 mg); preferred 0.1 μg to 1 mg. The dose per unit area of the device of 0.01 μg-20 μg per mm²; preferred dose of 0.05 μg/mm²-3 μg/mm². Minimum concentration of 10⁻⁸-10⁻⁴ M of mitoxantrone is to be maintained on the device surface. B) Cell cycle inhibitors including paclitaxel and analogues and derivatives (e.g., docetaxel) thereof: total dose not to exceed 10 mg (range of 0.1 μg to 10 mg); preferred 1 μg to 3 mg. The dose per unit area of the device of 0.05 μg-10 μg per mm²; preferred dose of 0.2 μg/mm²-5 μg/mm². Minimum concentration of 10⁻⁹-10⁻⁴ M of paclitaxel is to be maintained on the device surface. (C) Cell cycle inhibitors such as podophyllotoxins (e.g., etoposide): total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of etoposide is to be maintained on the device surface. (D) Immunomodulators including sirolimus and everolimus. Sirolimus (i.e., Rapamycin, RAPAMUNE): Total dose not to exceed 10 mg (range of 0.1 μg to 10 mg); preferred 10 μg to 1 mg. The dose per unit area of 0.1 μg-100 μg per mm 2; preferred dose of 0.5 μg/mm²-10 μg/mm². Minimum concentration of 10⁻⁸-10⁻⁴ M is to be maintained on the device surface. Everolimus and derivatives and analogues thereof: Total dose may not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (E) Heat shock protein 90 antagonists (e.g., geldanamycin) and analogues and derivatives thereof: total dose not to exceed 20 mg (range of 0.1 μg to 20 mg); preferred 1 μg to 5 mg. The dose per unit area of the device of 0.1 μg-10 μg per mm²; preferred dose of 0.25 μg/mm²-5 μg/mm². Minimum concentration of 10⁻⁸-10⁻⁴ M of paclitaxel is to be maintained on the device surface. (F) HMGCoA reductase inhibitors (e.g., simvastatin) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of simvastatin is to be maintained on the device surface. (G) Inosine monophosphate dehydrogenase inhibitors (e.g., mycophenolic acid, 1-alpha-25 dihydroxy vitamin D₃) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of mycophenolic acid is to be maintained on the device surface. (H)NF kappa B inhibitors (e.g., Bay 11-7082) and analogues and derivatives thereof: total dose not to exceed 200 mg (range of 1.0 μg to 200 mg); preferred 1 μg to 50 mg. The dose per unit area of the device of 1.0 μg-100 μg per mm²; preferred dose of 2.5 μg/mm²-50 μg/mm². Minimum concentration of 10⁻⁸-10⁻⁴ M of Bay 11-7082 is to be maintained on the device surface. (I) Antimycotic agents (e.g., sulconizole) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of sulconizole is to be maintained on the device surface. (J) P38 MAP Kinase inhibitors (e.g., SB202190) and analogues and derivatives thereof: total dose not to exceed 2000 mg (range of 10.0 μg to 2000 mg); preferred 10 μg to 300 mg. The dose per unit area of the device of 1.0 μg-1000 μg per mm²; preferred dose of 2.5 μg/mm²-500 μg/mm². Minimum concentration of 10⁻⁸-10⁻³ M of SB202190 is to be maintained on the device surface. (K) Anti-angiogenic agents (e.g., halofuginone bromide) and analogues and derivatives thereof: total dose not to exceed 10 mg (range of 0.1 μg to 10 mg); preferred 1 μg to 3 mg. The dose per unit area of the device of 0.1 μg-10 μg per mm²; preferred dose of 0.25 μg/mm²-5 μg/mm². Minimum concentration of 10⁻⁸-10⁻⁴ M of halofuginone bromide is to be maintained on the device surface.

In addition to those described above (e.g., sirolimus, everolimus, and tacrolimus), several other examples of immunomodulators and appropriate dosage ranges for use with implantable pump and sensor devices include the following: (A) Biolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of everolimus is to be maintained on the device surface. (B) Tresperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of tresperimus is to be maintained on the device surface. (C) Auranofin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of auranofin is to be maintained on the device surface. (D) 27-O-Demethylrapamycin and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10-810⁻⁴ M of 27-O-Demethylrapamycin is to be maintained on the device surface. (E) Gusperimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of gusperimus is to be maintained on the device surface. (F) Pimecrolimus and derivatives and analogues thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of pimecrolimus is to be maintained on the device surface and (G) ABT-578 and analogues and derivatives thereof: Total dose should not exceed 10 mg (range of 0.1 μg to 10 mg); preferred 10 μg to 1 mg. The dose per unit area of 0.1 μg-100 μg per mm² of surface area; preferred dose of 0.3 μg/mm²-10 μg/mm². Minimum concentration of 10⁻⁸-10⁻⁴ M of ABT-578 is to be maintained on the device surface.

In addition to those described above (e.g., paclitaxel, TAXOTERE, and docetaxel), several other examples of anti-microtubule agents and appropriate dosage ranges for use with ear ventilation devices include vinca alkaloids such as vinblastine and vincristine sulfate and analogues and derivatives thereof: total dose not to exceed 10 mg (range of 0.1 μg to 10 mg); preferred 1 μg to 3 mg. Dose per unit area of the device of 0.1 μg-10 μg per mm²; preferred dose of 0.25 μg/mm²-5 μg/mm². Minimum concentration of 10⁻⁸-10⁻⁴ M of drug is to be maintained on the device surface.

D. Methods for Generating Implantable Sensors and Drug Delivery Pumps Which Include and Release a Fibrosis-Inhibiting Agent

In the practice of this invention, drug-coated or drug-impregnated implants and medical devices are provided which inhibit fibrosis in and around the implantable sensor or implantable pump. Within various embodiments, fibrosis is inhibited by local, regional or systemic release of specific pharmacological agents that become localized to the tissue adjacent to the device or implant. There are numerous implantable sensors or implantable pumps where the occurrence of a fibrotic reaction will adversely affect the functioning of the device or the biological problem for which the device was implanted or used. Typically, fibrotic encapsulation of the device (or the growth of fibrous tissue between the device and the target tissue) slows, impairs, or interrupts detection (sensors) or drug delivery (pumps) to/from the device to/from the tissue. This can cause the device to function suboptimally or not at all, negatively affect disease management, and/or shorten the lifespan of the device. There are numerous methods available for optimizing delivery of the fibrosis-inhibiting agent to the site of the intervention and several of these are described below.

1. Devices and Implants that Release Fibrosis-Inhibiting Agents

Medical devices or implants of the present invention are coated with, or otherwise adapted to release an agent which inhibits fibrosis on the surface of, or around, the implantable sensor and/or implantable pump. In one aspect, the present invention provides implantable sensors and implantable pumps that include an anti-scarring agent or a composition that includes an anti-scarring agent such that the overgrowth of fibrous or granulation tissue is inhibited or reduced.

Methods for incorporating fibrosis-inhibiting compositions onto or into implantable sensors and implantable pumps include: (a) directly affixing to the device a fibrosis-inhibiting composition (e.g., by either a spraying process or dipping process as described above, with or without a carrier), (b) directly incorporating into the device a fibrosis-inhibiting composition (e.g., by either a spraying process or dipping process as described above, with or without a carrier (c) by coating the device with a substance such as a hydrogel which will in turn absorb the fibrosis-inhibiting composition, (d) by interweaving fibrosis-inhibiting composition coated thread (or the polymer itself formed into a thread) into the device structure, (e) by inserting the device into a sleeve or mesh which is comprised of, or coated with, a fibrosis-inhibiting composition, (f) constructing the device itself (or a portion of the device such as the detector, drug delivery catheter or port) with a fibrosis-inhibiting composition, or (g) by covalently binding the fibrosis-inhibiting agent directly to the device surface or to a linker (small molecule or polymer) that is coated or attached to the device surface. Each of these methods illustrates an approach for combining an implantable sensor or an implantable pump with a fibrosis-inhibiting (also referred to herein as anti-scarring) agent according to the present invention.

For these devices, the coating process can be performed in such a manner as to coat all or parts (such as the sensor or the drug delivery catheter/port) of the entire device with the fibrosis-inhibiting composition. In addition to, or alternatively, the fibrosis-inhibiting agent can be mixed with the materials that are used to make the implantable sensor or implantable pump such that the fibrosis-inhibiting agent is incorporated into the final product. In these manners, a medical device may be prepared which has a coating, where the coating is, e.g., uniform, non-uniform, continuous, discontinuous, or patterned.

In another aspect, an implantable sensor or drug delivery/catheter/port device may include a plurality of reservoirs within its structure, each reservoir configured to house and protect a therapeutic drug (i.e., one or more fibrosis-inhibiting agents). The reservoirs may be formed from divets in the device surface or micropores or channels in the device body. In one aspect, the reservoirs are formed from voids in the structure of the device. The reservoirs may house a single type of drug (e.g., fibrosis-inhibiting agent) or more than one type of drug (e.g., a fibrosis-inhibiting agent and an anti-infective agent). The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount and type of drug that is released from the substrate. The multi-layered carrier may further include a barrier layer that prevents release of the drug(s). The barrier layer can be used, for example, to control the direction that the drug elutes from the void. Thus, the coating of the medical device may directly contact the implantable device, or it may indirectly contact the device when there is something, e.g., a polymer layer, that is interposed between the device and the coating that contains the fibrosis-inhibiting agent.

In addition to, or as an alternative to incorporating a fibrosis-inhibiting agent onto or into the implantable sensors and implantable pump, the fibrosis-inhibiting agent can be applied directly or indirectly to the tissue adjacent to the implantable sensors and implantable pump (preferably near the interface of the tissue and the detector, drug delivery catheter and/or drug delivery port). This can be accomplished by applying the fibrosis-inhibiting agent, with or without a polymeric, non-polymeric, or secondary carrier: (a) to the device surface (e.g., as an injectable, paste, gel or meSH) during the implantation procedure; (b) to the surface of the tissue (e.g., as an injectable, paste, gel, in situ forming gel or meSH) prior to, immediately prior to, or during, implantation of the implantable sensors and implantable pump; (c) to the surface of the device and/or the tissue surrounding the implanted pump or sensor (e.g., as an injectable, paste, gel, in situ forming gel or meSH) immediately after implantation; (d) by topical application of the anti-fibrosis agent into the anatomical space where the implantable sensors and implantable pump will be placed (particularly useful for this embodiment is the use of polymeric carriers which release the fibrosis-inhibiting agent over a period ranging from several hours to several weeks—fluids, suspensions, emulsions, microemulsions, microspheres, pastes, gels, microparticulates, sprays, aerosols, solid implants and other formulations which release the agent can be delivered into the region where the device will be inserted); (e) via percutaneous injection into the tissue surrounding the implantable sensor or implantable pump as a solution, as an infusate, or as a sustained release preparation; (f) by any combination of the aforementioned methods. Combination therapies (i.e., combinations of therapeutic agents and combinations with antithrombotic, antiplatelet and/or anti-infective agents) can also be used.

2. Systemic, Regional and Local Delivery of Fibrosis-Inhibiting Agents

A variety of drug-delivery technologies are available for systemic, regional and local delivery of fibrosis-inhibiting therapeutic agents. Several of these techniques may be suitable to achieve preferentially elevated levels of fibrosis-inhibiting agents in the vicinity of the implantable sensors and implantable pump, including: (a) using drug-delivery catheters for local, regional or systemic delivery of fibrosis-inhibiting agents to the tissue surrounding the device or implant. Typically, drug delivery catheters are advanced through the circulation or inserted directly into tissues under radiological guidance until they reach the desired anatomical location. The fibrosis-inhibiting agent can then be released from the catheter lumen in high local concentrations in order to deliver therapeutic doses of the drug to the tissue surrounding the device or implant; (b) drug localization techniques such as magnetic, ultrasonic or MRI-guided drug delivery; (c) chemical modification of the fibrosis-inhibiting drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the fibrosis-inhibiting drug or formulation designed to localize the drug to areas of bleeding or disrupted vasculature; and/or (e) direct injection or administration of the fibrosis-inhibiting agent, for example, under endoscopic vision.

3. Infiltration of Fibrosis-Inhibiting Agents into the Tissue Surrounding a Device or Implant

Alternatively, the tissue surrounding the implantable sensor or implantable pump can be treated with a fibrosis-inhibiting agent prior to, during, or after the implantation procedure. A fibrosis-inhibiting agent or a composition comprising a fibrosis-inhibiting agent may be infiltrated around the device or implant, for example, by applying the composition directly and/or indirectly into and/or onto (a) tissue adjacent to the medical device; (b) the vicinity of the medical device-tissue interface; (c) the region around the medical device; and (d) tissue surrounding the medical device. It may be noted that certain polymeric carriers themselves can help prevent the formation of fibrous tissue around the implantable sensors and implantable pumps. The following exemplary polymer compositions may be used for the practice of this embodiment, either alone, or in combination with a fibrosis inhibiting composition. The following polymeric carriers can be infiltrated (as described in the previous paragraph) into the vicinity of the device-tissue interface and include: (a) sprayable collagen-containing formulations such as COSTASIS and CT3, either alone, or loaded with a fibrosis-inhibiting agent, applied to the implantation site (or the device, detector, semipermeable membrane, drug delivery catheter, and/or drug delivery port surface); (b) sprayable PEG-containing formulations such as COSEAL, FOCALSEAL, SPRAYGEL or DURASEAL, either alone, or loaded with a fibrosis-inhibiting agent, applied to the implantation site (or the device, detector, semipermeable membrane, drug delivery catheter, and/or drug delivery port surface); (c) fibrinogen-containing formulations such as FLOSEAL or TISSEAL, either alone, or loaded with a fibrosis-inhibiting agent, applied to the implantation site (or the device, detector, semipermeable membrane, drug delivery catheter, and/or drug delivery port surface); (d) hyaluronic acid-containing formulations such as RESTYLANE, HYLAFORM, PERLANE, SYNVISC, SEPRAFILM, SEPRACOAT, loaded with a fibrosis-inhibiting agent applied to the implantation site (or the device, detector, semipermeable membrane, drug delivery catheter, and/or drug delivery port surface); (e) polymeric gels for surgical implantation such as REPEL or FLOWGEL loaded with a fibrosis-inhibiting agent applied to the implantation site (or the device, detector, semipermeable membrane, drug delivery catheter, and/or drug delivery port surface); (f) orthopedic “cements” used to hold prostheses and tissues in place loaded with a fibrosis-inhibiting agent applied to the implantation site (or the device, detector, semipermeable membrane, drug delivery catheter, and/or drug delivery port surface), such as OSTEOBOND, low viscosity cement (LVC), SIMPLEX P, PALACOS, and ENDURANCE; (g) surgical adhesives containing cyanoacrylates such as DERMABOND, INDERMIL, GLUSTITCH, TISSUMEND, VETBOND, HISTOACRYL BLUE and ORABASE® SOOTHE-N-SEAL LIQUID PROTECTANT, either alone, or loaded with a fibrosis-inhibiting agent, applied to the implantation site (or the device, detector, semipermeable membrane, drug delivery catheter, and/or drug delivery port surface); (h) implants containing hydroxyapatite (or synthetic bone material such as calcium sulfate, VITOSS and CORTOSS) loaded with a fibrosis-inhibiting agent applied to the implantation site (or the device, detector, semipermeable membrane, drug delivery catheter, and/or drug delivery port surface); (i) other biocompatible tissue fillers loaded with a fibrosis-inhibiting agent, such as those made by BioCure, Inc., 3M Company and Neomend, Inc., applied to the implantation site (or the device, detector, semipermeable membrane, drug delivery catheter, and/or drug delivery port surface); 0) polysaccharide gels such as the ADCON series of gels either alone, or loaded with a fibrosis-inhibiting agent, applied to the implantation site (or the device, detector, semipermeable membrane, drug delivery catheter, and/or drug delivery port surface); and/or (k) films, sponges or meshes such as INTERCEED, VICRYL mesh, and GELFOAM loaded with a fibrosis-inhibiting agent applied to the implantation site (or the device, detector, semipermeable membrane, drug delivery catheter, and/or drug delivery port surface).

A preferred polymeric matrix which can be used to help prevent the formation of fibrous tissue around the implantable sensor or implantable pump, either alone or in combination with a fibrosis (or gliosis) inhibiting agent/composition, is formed from reactants comprising either one or both of pentaerythritol poly(ethylene glycol)ether tetra-sulfhydryl] (4-armed thiol PEG, which includes structures having a linking group(s) between a sulfhydryl group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Another preferred composition comprises either one or both of pentaerythritol poly(ethylene glycol)ether tetra-amino] (4-armed amino PEG, which includes structures having a linking group(s) between an amino group(s) and the terminus of the polyethylene glycol backbone) and pentaerythritol poly(ethylene glycol)ether tetra-succinimidyl glutarate] (4-armed NHS PEG, which again includes structures having a linking group(s) between a NHS group(s) and the terminus of the polyethylene glycol backbone) as reactive reagents. Chemical structures for these reactants are shown in, e.g., U.S. Pat. No. 5,874,500. Optionally, collagen or a collagen derivative (e.g., methylated collagen) is added to the poly(ethylene glycol)-containing reactant(s) to form a preferred crosslinked matrix that can serve as a polymeric carrier for a therapeutic agent or a stand-alone composition to help prevent the formation of fibrous tissue around the implantable sensor or implantable pump.

4. Sustained-Release Preparations of Fibrosis-Inhibiting Agents

As described previously, desired fibrosis-inhibiting agents may be admixed with, blended with, conjugated to, or, otherwise modified to contain a polymer composition (which may be either biodegradable or non-biodegradable), or a non-polymeric composition, in order to release the therapeutic agent over a prolonged period of time. For many of the aforementioned embodiments, localized delivery as well as localized sustained delivery of the fibrosis-inhibiting agent may be required. For example, a desired fibrosis-inhibiting agent may be admixed with, blended with, conjugated to, or otherwise modified to contain a polymeric composition (which may be either biodegradable or non-biodegradable), or non-polymeric composition, in order to release the fibrosis-inhibiting agent over a period of time. In certain aspects, the polymer composition may include a bioerodable or biodegradable polymer. Representative examples of biodegradable polymer compositions suitable for the delivery of fibrosis-inhibiting agents include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, poly(ethylene terephthalate), poly(malic acid), poly(tartronic acid), poly(acrylamides), polyanhydrides, polyphosphazenes, poly(amino acids), poly(alkylene oxide)-poly(ester) block copolymers (e.g., X-Y, X-Y-X or Y-X-Y, where X is a polyalkylene oxide and Y is a polyester (e.g., PLGA, PLA, PCL, polydioxanone and copolymers thereof) and their copolymers as well as blends thereof. (see generally, Illum, L., Davids, S. S. (eds.) “Polymers in Controlled Drug Delivery” Wright, Bristol, 1987; Arshady, J. Controlled Release 17: 1-22, 1991; Pitt, Int. J. Phar. 59: 173-196, 1990; Holland et al., J. Controlled Release 4: 155-0180, 1986).

Representative examples of non-degradable polymers suitable for the delivery of fibrosis-inhibiting agents include poly(ethylene-co-vinyl acetate) (“EVA”) copolymers, silicone rubber, acrylic polymers (polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), polyethylene, polypropylene, polyamides (nylon 6,6), polyurethane, poly(ester urethanes), poly(ether urethanes), poly(ester-urea), polyethers (poly(ethylene oxide), poly(propylene oxide), block copolymers based on ethylene oxide and propylene oxide (i.e., copolymers of ethylene oxide and propylene oxide polymers), such as the family of PLURONIC polymers available from BASF Corporation (Mount Olive, N.J.), and poly(tetramethylene glycol)), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends thereof (see generally, Dunn et al., J. Applied Polymer Sci. 50: 353-365, 1993; Cascone et al., J. Materials Sci.: Materials in Medicine 5: 770-774, 1994; Shiraishi et al., Biol. Pharm. Bull. 16 (11): 1164-1168, 1993; Thacharodi and Rao, Int'l J. Pharm. 120: 115-118, 1995; Miyazaki et al., Int'l J. Pharm. 118: 257-263, 1995).

Particularly preferred polymeric carriers include poly(ethylene-co-vinyl acetate), polyurethanes, poly (D,L-lactic acid) oligomers and polymers, poly (L-lactic acid) oligomers and polymers, poly (glycolic acid), copolymers of lactic acid and glycolic acid, poly (caprolactone), poly (valerolactone); polyanhydrides, copolymers of poly (caprolactone) or poly (lactic acid) with a polyethylene glycol (e.g., MePEG), silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery of fibrosis-inhibiting agents include carboxylic polymers, polyacetates, polyacrylamides, polycarbonates, polyethers, polyesters, polyethylenes, polyvinylbutyrals, polysilanes, polyureas, polyurethanes, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxy, melamine, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, polyethers, polysaccharides, hydrophilic polyurethane, polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, polyurethane, polyacrylate, natural and synthetic elastomers, rubber, acetal, nylon, polyester, styrene polybutadiene, acrylic resin, polyvinylidene chloride, polycarbonate, homopolymers and copolymers of vinyl compounds, polyvinylchloride, polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as their corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052 and U.S. Patent Application Publication Nos. 2003/0068377, 2002/0192286, 2002/0076441, and 2002/0090398.

It may be obvious to one of skill in the art that the polymers as described herein can also be blended or copolymerized in various compositions as required to deliver therapeutic doses of fibrosis-inhibiting agents.

Polymeric carriers for fibrosis-inhibiting agents can be fashioned in a variety of forms, with desired release characteristics and/or with specific properties depending upon the device, composition or implant being utilized. For example, polymeric carriers may be fashioned to release a fibrosis-inhibiting agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48: 343-354, 1993; Dong et al., J. Controlled Release 19: 171-178, 1992; Dong and Hoffman, J. Controlled Release 15: 141-152, 1991; Kim et al., J. Controlled Release 28: 143-152, 1994; Cornejo-Bravo et al., J. Controlled Release 33: 223-229, 1995; Wu and Lee, Pharm. Res. 10 (10): 1544-1547, 1993; Serres et al., Pharm. Res. 13 (2): 196-201, 1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and/or acrylate or acrylamide lmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inhibiting agents can be delivered via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact. Mater. 22: 167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact Mater. 22: 111-112, Controlled Release Society, Inc., 1995; Johnston et al., Pharm. Res. 9 (3): 425-433, 1992; Tung, Int'l J. Pharm. 107: 85-90, 1994; Harsh and Gehrke, J. Controlled Release 17: 175-186, 1991; Bae et al., Pharm. Res. 8 (4): 531-537, 1991; Dinarvand and D'Emanuele, J. Controlled Release 36: 221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9 (3): 283-290, 1992; Bae et al., Pharm. Res. 8 (5): 624-628, 1991; Kono et al., J. Controlled Release 30: 69-75, 1994; Yoshida et al., J. Controlled Release 32: 97-102, 1994; Okano et al., J. Controlled Release 36: 125-133, 1995; Chun and Kim, J. Controlled Release 38: 39-47, 1996; D'Emanuele and Dinarvand, Int'l J. Pharm. 118: 237-242, 1995; Katono et al., J. Controlled Release 16: 215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22: 95-104, 1992; Palasis and Gehrke, J. Controlled Release 18: 1-12, 1992; Paavola et al., Pharm. Res. 12 (12): 1997-2002, 1995).

Representative examples of thermogelling polymers, and their gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof, such as methylacrylic acid, acrylate monomers and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X-Y, Y-X-Y and X-Y-X where X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and their preparation include U.S. Pat. Nos. 6,451,346; 6,201,072; 6,117,949; 6,004,573; 5,702,717; and 5,484,610 and PCT Publication Nos. WO 99/07343; WO 99/18142; WO 03/17972; WO 01/82970; WO 00/18821; WO 97/15287; WO 01/41735; WO 00/00222 and WO 00/38651.

Fibrosis-inhibiting agents may be linked by occlusion in the matrices of the polymer, bound by covalent linkages, or encapsulated in microcapsules. Within certain embodiments of the invention, therapeutic compositions are provided in non-capsular formulations such as microspheres (ranging from nanometers to micrometers in size), pastes, threads of various size, films and sprays.

Within certain aspects of the present invention, therapeutic compositions may be fashioned into particles having any size ranging from 50 nm to 500 μm, depending upon the particular use. These compositions can be in the form of microspheres, microparticles and/or nanoparticles. These compositions can be formed by spray-drying methods, milling methods, coacervation methods, W/O emulsion methods, W/O/W emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of paste or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosis-inhibiting agents are particularly useful for application to the surface of tissues that will be in contact with the implant or device.

Within yet other aspects of the invention, the therapeutic compositions of the present invention may be formed as a film or tube. These films or tubes can be porous or non-porous. Such films or tubes are generally less than 5, 4, 3, 2, or 1 mm thick, or less than 0.75 mm, or less than 0.5 mm, or less than 0.25 mm, or, less than 0.10 mm thick. Films or tubes can also be generated of thicknesses less than 50 μm, 25 μm or 10 μm. Such films may be flexible with a good tensile strength (e.g., greater than 50, or greater than 100, or greater than 150 or 200 N/cm²), good adhesive properties (i.e., adheres to moist or wet surfaces), and have controlled permeability. Fibrosis-inhibiting agents contained in polymeric films are particularly useful for application to the surface of a device or implant as well as to the surface of tissue, cavity or an organ.

Within further aspects of the present invention, polymeric carriers are provided which are adapted to contain and release a hydrophobic fibrosis-inhibiting compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic fibrosis-inhibiting compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan, hyaluronic acid, proteins or polypeptides such as albumin, collagen and gelatin. Within alternative embodiments, hydrophobic compounds may be contained within a hydrophobic core, and this core contained within a hydrophilic shell.

Other carriers that may likewise be utilized to contain and deliver fibrosis-inhibiting agents described herein include: hydroxypropyl cyclodextrin (Cserhati and Hollo, Int. J. Pharm. 108: 69-75, 1994), liposomes (see, e.g., Sharma et al., Cancer Res. 53: 5877-5881, 1993; Sharma and Straubinger, Pharm. Res. 11 (60): 889-896, 1994; WO 93/18751; U.S. Pat. No. 5,242,073), liposome/gel (WO 94/26254), nanocapsules (Bartoli et al., J. Microencapsulation 7 (2): 191-197, 1990), micelles (Alkan-Onyuksel et al., Pharm. Res. 11 (2): 206-212, 1994), implants (Jampel et al., Invest. Ophthalm. Vis. Science 34 (11): 3076-3083, 1993; Walter et al., Cancer Res. 54: 22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12 (2): 192-195; Kwon et al., Pharm Res. 10 (7): 970-974; Yokoyama et al., J. Contr. Rel. 32: 269-277, 1994; Gref et al., Science 263: 1600-1603, 1994; Bazile et al., J. Pharm. Sci. 84: 493-498, 1994) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible light, UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in WO 01/44307, WO 01/68720, WO 02/072166, WO 03/043552, WO 93/17669, WO 00/64977, U.S. Pat. Nos. 5,900,245, 6,051,248, 6,083,524, 6,177,095, 6,201,065, 6,217,894, 6,639,014, 6,352,710, 6,410,645, 6,531,147, 5,567,435, 5,986,043, 6,602,975, and U.S. Patent Application Publication Nos. 2002/012796A1, 2002/0127266A1, 2002/0151650A1, 2003/0104032A1, 2002/0091229A1, and 2003/0059906A1.

In another embodiment, the reagents can undergo an electrophilic-nucleophilic reaction to produce a crosslinked matrix. For example, a 4-armed thiol derivatized polyethylene glycol can be reacted with a 4 armed NHS-derivatized polyethylene glycol under basic conditions (pH>about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; U.S. Patent Application Publication No. 2003/0077272; and PCT Application Publication Nos. WO 04/060405 and WO 04/060346. Other examples of in situ forming materials that can be used include those based on the crosslinking of proteins (described in U.S. Patent Nos. RE38158; 4,839,345; 5,514,379, 5,583,114; 6,458,147; 6,371,975; U.S. Patent Application Publication Nos 2002/0161399; 2001/0018598 and PCT Publication Nos. WO 03/090683; WO 01/45761; WO 99/66964 and WO 96/03159).

The following further and additionally describes polymeric crosslinked matrices, and polymeric carriers, that may be used to assist in the prevention of the formation or growth of fibrous connective tissue. The composition may contain and deliver fibrosis-inhibiting agents in the vicinity of the medical device. The following compositions are particularly useful when it is desired to infiltrate around the device, with or without a fibrosis-inhibiting agent. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a device in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

First and Second Synthetic Polymers

In one embodiment, crosslinked polymer compositions (in other words, crosslinked matrices) are prepared by reacting a first synthetic polymer containing two or more nucleophilic groups with a second synthetic polymer containing two or more electrophilic groups, where the electrophilic groups are capable of covalently binding with the nucleophilic groups. In one embodiment, the first and second polymers are each non-immunogenic. In another embodiment, the matrices are not susceptible to enzymatic cleavage by, e.g., a matrix metalloproteinase (e.g., collagenase) and are therefore expected to have greater long-term persistence in vivo than collagen-based compositions.

As used herein, the term “polymer” refers inter alia to polyalkyls, polyamino acids, polyalkyleneoxides and polysaccharides. Additionally, for external or oral use, the polymer may be polyacrylic acid or carbopol. As used herein, the term “synthetic polymer” refers to polymers that are not naturally occurring and that are produced via chemical synthesis. As such, naturally occurring proteins such as collagen and naturally occurring polysaccharides such as hyaluronic acid are specifically excluded. Synthetic collagen, and synthetic hyaluronic acid, and their derivatives, are included. Synthetic polymers containing either nucleophilic or electrophilic groups are also referred to herein as “multifunctionally activated synthetic polymers.” The term “multifunctionally activated” (or, simply, “activated”) refers to synthetic polymers which have, or have been chemically modified to have, two or more nucleophilic or electrophilic groups which are capable of reacting with one another (i.e., the nucleophilic groups react with the electrophilic groups) to form covalent bonds. Types of multifunctionally activated synthetic polymers include difunctionally activated, tetrafunctionally activated, and star-branched polymers.

Multifunctionally activated synthetic polymers for use in the present invention must contain at least two, more preferably, at least three, functional groups in order to form a three-dimensional crosslinked network with synthetic polymers containing multiple nucleophilic groups (i.e., “multi-nucleophilic polymers”). In other words, they must be at least difunctionally activated, and are more preferably trifunctionally or tetrafunctionally activated. If the first synthetic polymer is a difunctionally activated synthetic polymer, the second synthetic polymer must contain three or more functional groups in order to obtain a three-dimensional crosslinked network. Most preferably, both the first and the second synthetic polymer contain at least three functional groups.

Synthetic polymers containing multiple nucleophilic groups are also referred to generically herein as “multi-nucleophilic polymers.” For use in the present invention, multi-nucleophilic polymers must contain at least two, more preferably, at least three, nucleophilic groups. If a synthetic polymer containing only two nucleophilic groups is used, a synthetic polymer containing three or more electrophilic groups must be used in order to obtain a three-dimensional crosslinked network.

Preferred multi-nucleophilic polymers for use in the compositions and methods of the present invention include synthetic polymers that contain, or have been modified to contain, multiple nucleophilic groups such as primary amino groups and thiol groups. Preferred multi-nucleophilic polymers include: (i) synthetic polypeptides that have been synthesized to contain two or more primary amino groups or thiol groups; and (II) polyethylene glycols that have been modified to contain two or more primary amino groups or thiol groups. In general, reaction of a thiol group with an electrophilic group tends to proceed more slowly than reaction of a primary amino group with an electrophilic group.

In one embodiment, the multi-nucleophilic polypeptide is a synthetic polypeptide that has been synthesized to incorporate amino acid residues containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000.

Poly(lysine)s for use in the present invention preferably have a molecular weight within the range of about 1,000 to about 300,000; more preferably, within the range of about 5,000 to about 100,000; most preferably, within the range of about 8,000 to about 15,000. Poly(lysine)s of varying molecular weights are commercially available from Peninsula Laboratories, Inc. (Belmont, Calif.) and Aldrich Chemical (Milwaukee, Wis.).

Polyethylene glycol can be chemically modified to contain multiple primary amino or thiol groups according to methods set forth, for example, in Chapter 22 of Poly(ethylene Glycol) Chemistry: Biotechnical and Biomedical Applications, J. Milton Harris, ed., Plenum Press, N.Y. (1992). Polyethylene glycols which have been modified to contain two or more primary amino groups are referred to herein as “multi-amino PEGs.” Polyethylene glycols which have been modified to contain two or more thiol groups are referred to herein as “multi-thiol PEGs.” As used herein, the term “polyethylene glycol(s)” includes modified and or derivatized polyethylene glycol(s).

Various forms of multi-amino PEG are commercially available from Shearwater Polymers (Huntsville, Ala.) and from Huntsman Chemical Company (Utah) under the name “Jeffamine.” Multi-amino PEGs useful in the present invention include Huntsman's Jeffamine diamines (“D” series) and triamines (“T” series), which contain two and three primary amino groups per molecule, respectively.

Polyamines such as ethylenediamine (H₂N—CH₂—CH₂—NH₂), tetramethylenediamine (H₂N—(CH₂)₄—NH₂), pentamethylenediamine (cadaverine) (H₂N—(CH₂)₅—NH₂), hexamethylenediamine (H₂N—(CH₂)₆-NH₂), di(2-aminoethyl)amine (HN—(CH₂—CH₂-NH₂)₂), and tris(2-aminoethyl)amine (N—(CH₂—CH₂-NH₂)₃) may also be used as the synthetic polymer containing multiple nucleophilic groups.

Synthetic polymers containing multiple electrophilic groups are also referred to herein as “multi-electrophilic polymers.” For use in the present invention, the multifunctionally activated synthetic polymers must contain at least two, more preferably, at least three, electrophilic groups in order to form a three-dimensional crosslinked network with multi-nucleophilic polymers. Preferred multi-electrophilic polymers for use in the compositions of the invention are polymers which contain two or more succinimidyl groups capable of forming covalent bonds with nucleophilic groups on other molecules. Succinimidyl groups are highly reactive with materials containing primary amino (NH₂) groups, such as multi-amino PEG, poly(lysine), or collagen. Succinimidyl groups are slightly less reactive with materials containing thiol (SH) groups, such as multi-thiol PEG or synthetic polypeptides containing multiple cysteine residues.

As used herein, the term “containing two or more succinimidyl groups” is meant to encompass polymers which are preferably commercially available containing two or more succinimidyl groups, as well as those that must be chemically derivatized to contain two or more succinimidyl groups. As used herein, the term “succinimidyl group” is intended to encompass sulfosuccinimidyl groups and other such variations of the “generic” succinimidyl group. The presence of the sodium sulfite moiety on the sulfosuccinimidyl group serves to increase the solubility of the polymer.

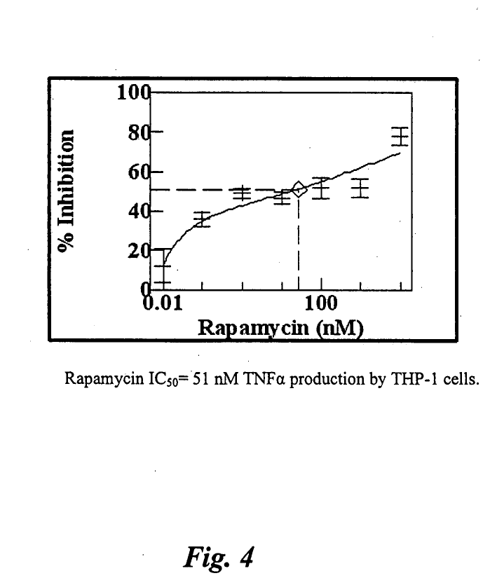

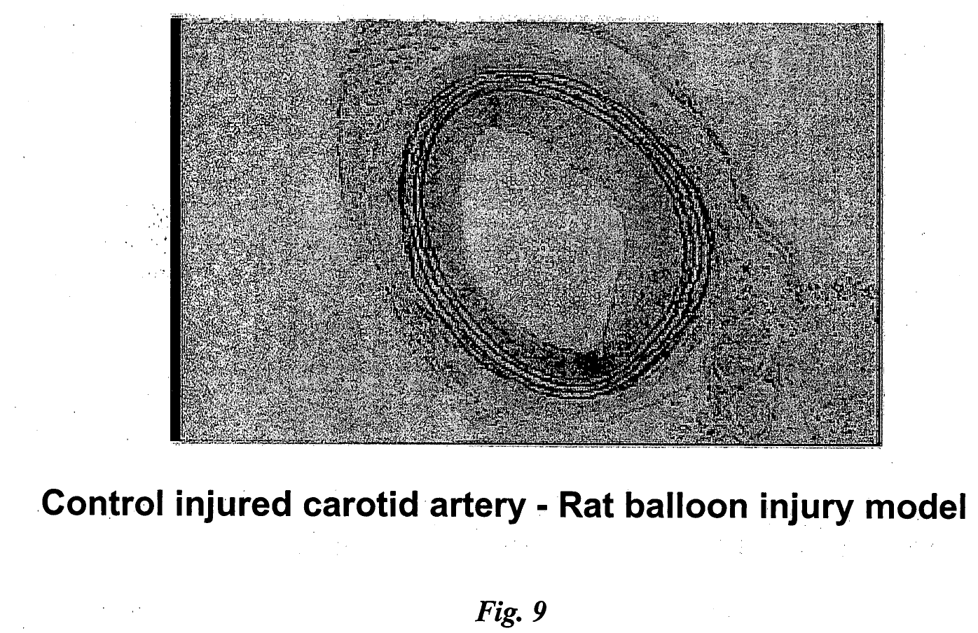

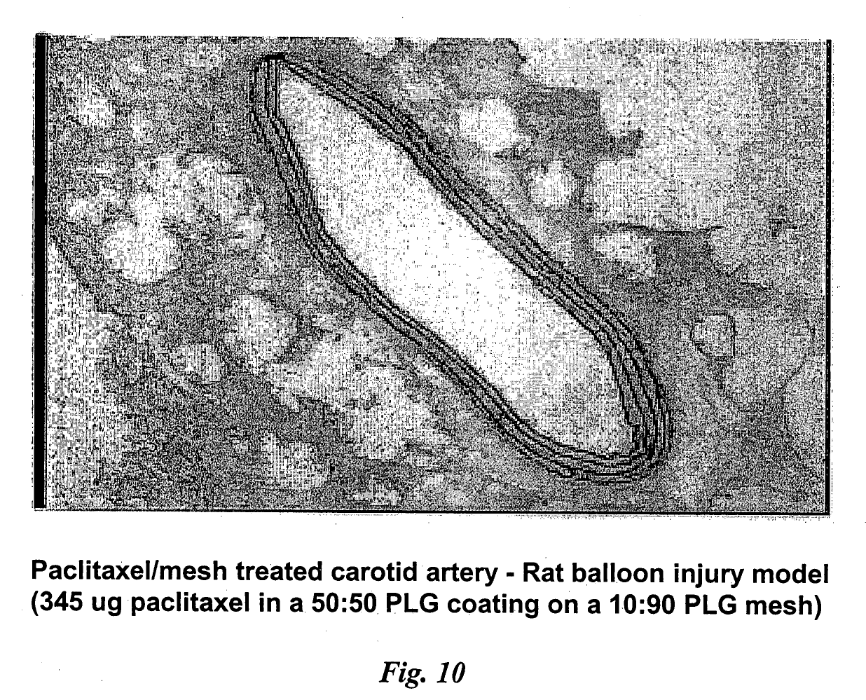

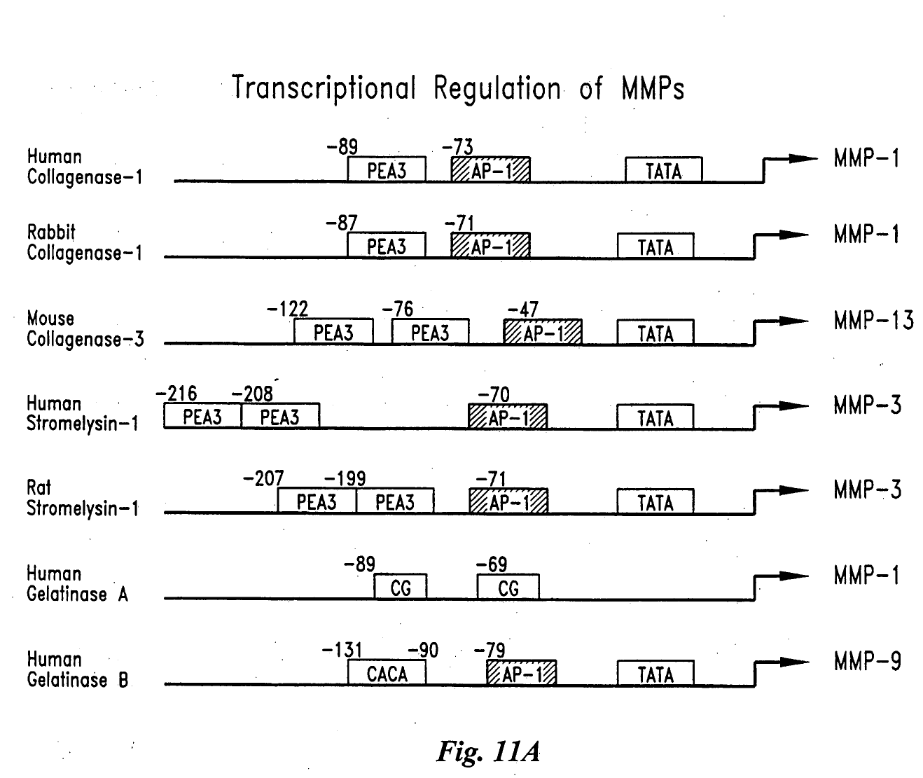

Hydrophilic polymers and, in particular, various derivatized polyethylene glycols, are preferred for use in the compositions of the present invention. As used herein, the term “PEG” refers to polymers having the repeating structure (OCH₂—CH₂)ₙ. Structures for some specific, tetrafunctionally activated forms of PEG are shown in FIGS. 4 to 13 of U.S. Pat. No. 5,874,500, incorporated herein by reference. Examples of suitable PEGS include PEG succinimidyl propionate (SE-PEG), PEG succinimidyl succinamide (SSA-PEG), and PEG succinimidyl carbonate (SC-PEG). In one aspect of the invention, the crosslinked matrix is formed in situ by reacting pentaerythritol poly(ethylene glycol)ether tetra-sulfhydryl] (4-armed thiol PEG) and pentaerythritol poly(ethylene glycol)ether tetra-succinimidyl glutarate] (4-armed NHS PEG) as reactive reagents. Structures for these reactants are shown in U.S. Pat. No. 5,874,500. Each of these materials has a core with a structure that may be seen by adding ethylene oxide-derived residues to each of the hydroxyl groups in pentaerythritol, and then derivatizing the terminal hydroxyl groups (derived from the ethylene oxide) to contain either thiol groups (so as to form 4-armed thiol PEG) or N-hydroxysuccinimydyl groups (so as to form 4-armed NHS PEG), optionally with a linker group present between the ethylene oxide derived backbone and the reactive functional group, where this product is commercially available as COSEAL from Angiotech Pharmaceuticals Inc. Optionally, a group “D” may be present in one or both of these molecules, as discussed in more detail below.

As discussed above, preferred activated polyethylene glycol derivatives for use in the invention contain succinimidyl groups as the reactive group. However, different activating groups can be attached at sites along the length of the PEG molecule. For example, PEG can be derivatized to form functionally activated PEG propionaldehyde (A-PEG), or functionally activated PEG glycidyl ether (E-PEG), or functionally activated PEG-isocyanate (1-PEG), or functionally activated PEG-vinylsulfone (V-PEG).

Hydrophobic polymers can also be used to prepare the compositions of the present invention. Hydrophobic polymers for use in the present invention preferably contain, or can be derivatized to contain, two or more electrophilic groups, such as succinimidyl groups, most preferably, two, three, or four electrophilic groups. As used herein, the term “hydrophobic polymer” refers to polymers which contain a relatively small proportion of oxygen or nitrogen atoms.

Hydrophobic polymers which already contain two or more succinimidyl groups include, without limitation, disuccinimidyl suberate (DSS), bis(sulfosuccinimidyl) suberate (BS3), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The above-referenced polymers are commercially available from Pierce (Rockford, Ill.), under catalog Nos. 21555, 21579, 22585, 21554, and 21577, respectively.

Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

Certain polymers, such as polyacids, can be derivatized to contain two or more functional groups, such as succinimidyl groups. Polyacids for use in the present invention include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid). Many of these polyacids are commercially available from DuPont Chemical Company (Wilmington, Del.). According to a general method, polyacids can be chemically derivatized to contain two or more succinimidyl groups by reaction with an appropriate molar amount of N-hydroxysuccinimide (NHS) in the presence of N,N′-dicyclohexylcarbodiimide (DCC).

Polyalcohols such as trimethylolpropane and di(trimethylol propane) can be converted to carboxylic acid form using various methods, then further derivatized by reaction with NHS in the presence of DCC to produce trifunctionally and tetrafunctionally activated polymers, respectively, as described in U.S. application Ser. No. 08/403,358. Polyacids such as heptanedioic acid (HOOC—(CH₂)₅—COOH), octanedioic acid (HOOC—(CH₂)₆—COOH), and hexadecanedioic acid (HOOC—(CH₂)₁₄—COOH) are derivatized by the addition of succinimidyl groups to produce difunctionally activated polymers.

Polyamines such as ethylenediamine, tetramethylenediamine, pentamethylenediamine (cadaverine), hexamethylenediamine, bis (2-aminoethyl)amine, and tris(2-aminoethyl)amine can be chemically derivatized to polyacids, which can then be derivatized to contain two or more succinimidyl groups by reacting with the appropriate molar amounts of N-hydroxysuccinimide in the presence of DCC, as described in U.S. application Ser. No. 08/403,358. Many of these polyamines are commercially available from DuPont Chemical Company.

In a preferred embodiment, the first synthetic polymer will contain multiple nucleophilic groups (represented below as “X”) and it will react with the second synthetic polymer containing multiple electrophilic groups (represented below as “Y”), resulting in a covalently bound polymer network, as follows:
Polymer-Xₘ+Polymer-Yₙ→Polymer-Z-Polymer
wherein m≦2, n≦2, and m+n≦5;
where exemplary X groups include —NH₂, —SH, —OH, —PH₂, CO—NH—NH₂, etc., where the X groups may be the same or different in polymer-Xₘ;
where exemplary Y groups include —CO₂—N(COCH₂)₂, —CO₂H, —CHO, —CHOCH₂ (epoxide), —N═C═O, —SO₂—CH═CH₂, —N(COCH)₂ (i.e., a five-membered heterocyclic ring with a double bond present between the two CH groups), —S—S—(C₅H₄N), etc., where the Y groups may be the same or different in polymer-Yₙ; and
where Z is the functional group resulting from the union of a nucleophilic group (X) and an electrophilic group (Y).

As noted above, it is also contemplated by the present invention that X and Y may be the same or different, i.e., a synthetic polymer may have two different electrophilic groups, or two different nucleophilic groups, such as with glutathione.

In one embodiment, the backbone of at least one of the synthetic polymers comprises alkylene oxide residues, e.g., residues from ethylene oxide, propylene oxide, and mixtures thereof. The term ‘backbone’ refers to a significant portion of the polymer.

For example, the synthetic polymer containing alkylene oxide residues may be described by the formula X-polymer-X or Y-polymer-Y, wherein X and Y are as defined above, and the term “polymer” represents —(CH₂CH₂O)ₙ— or —(CH(CH₃)CH₂O)ₙ— or —(CH₂—CH₂—O)ₙ—(CH(CH₃)CH₂—O)ₙ—. In these cases the synthetic polymer may be difunctional.

The required functional group X or Y is commonly coupled to the polymer backbone by a linking group (represented below as “Q”), many of which are known or possible. There are many ways to prepare the various functionalized polymers, some of which are listed below:
Polymer-Q₁-X+Polymer-Q₂→Y Polymer-Q₁-Z-Q₂-Polymer

Exemplary Q groups include —O—(CH₂)ₙ—; —S—(CH₂)ₙ—; —NH—(CH₂)ₙ—; —O₂C—NH—(CH₂)ₙ—; —O₂C—(CH₂)ₙ—; —O₂C—(CR¹H)ᵣ—; and —O—R₂—CO—NH—, which provide synthetic polymers of the partial structures: polymer-O—(CH₂)ₙ—(X or Y); polymer-S—(CH₂)ₙ—(X or Y); polymer-NH—(CH₂)ₙ—(X or Y); polymer-O₂C—NH—(CH₂)ₙ—(X or Y); polymer-O₂C—(CH₂)ᵣ—(X or Y); polymer-O₂C—(CR¹H)ₙ—(X or Y); and polymer-O—R₂—CO—NH—(X or Y), respectively. In these structures, n=1-10, R¹═H or alkyl (i.e., CH₃, C₂H₅, etc.); R²═CH₂, or CO—NH—CH₂CH₂; and Q₁ and Q₂ may be the same or different.

For example, when Q₂=OCH₂CH₂ (there is no Q₁ in this case); Y=—CO₂—N(COCH₂)₂; and X≡NH₂, —SH, or —OH, the resulting reactions and Z groups may be as follows:
Polymer-NH₂+Polymer-O—CH₂—CH₂—CO₂—N(COCH₂)₂→Polymer-NH—CO—CH₂—CH₂—O-Polymer;
Polymer-SH+Polymer-O—CH₂—CH₂—CO₂—N(COCH₂)₂→Polymer-S—COCH₂CH₂—O-Polymer; and
Polymer-OH+Polymer-O—CH₂—CH₂—CO₂—N(COCH₂)₂→Polymer-O—COCH₂CH₂—O-Polymer.

An additional group, represented below as “D”, can be inserted between the polymer and the linking group, if present. One purpose of such a D group is to affect the degradation rate of the crosslinked polymer composition in vivo, for example, to increase the degradation rate, or to decrease the degradation rate. This may be useful in many instances, for example, when drug has been incorporated into the matrix, and it is desired to increase or decrease polymer degradation rate so as to influence a drug delivery profile in the desired direction. An illustration of a crosslinking reaction involving first and second synthetic polymers each having D and Q groups is shown below.
Polymer-D-Q-X+Polymer-D-Q-Y→Polymer-D-Q-Z-Q-D-Polymer

Some useful biodegradable groups “D” include polymers formed from one or more α-hydroxy acids, e.g., lactic acid, glycolic acid, and the cyclization products thereof (e.g., lactide, glycolide), ε-caprolactone, and amino acids. The polymers may be referred to as polylactide, polyglycolide, poly(co-lactide-glycolide); poly-ε-caprolactone, polypeptide (also known as poly amino acid, for example, various di- or tri-peptides) and poly(anhydride)s.

In a general method for preparing the crosslinked polymer compositions used in the context of the present invention, a first synthetic polymer containing multiple nucleophilic groups is mixed with a second synthetic polymer containing multiple electrophilic groups. Formation of a three-dimensional crosslinked network occurs as a result of the reaction between the nucleophilic groups on the first synthetic polymer and the electrophilic groups on the second synthetic polymer.

The concentrations of the first synthetic polymer and the second synthetic polymer used to prepare the compositions of the present invention will vary depending upon a number of factors, including the types and molecular weights of the particular synthetic polymers used and the desired end use application. In general, when using multi-amino PEG as the first synthetic polymer, it is preferably used at a concentration in the range of about 0.5 to about 20 percent by weight of the final composition, while the second synthetic polymer is used at a concentration in the range of about 0.5 to about 20 percent by weight of the final composition. For example, a final composition having a total weight of 1 gram (1000 milligrams) may contain between about 5 to about 200 milligrams of multi-amino PEG, and between about 5 to about 200 milligrams of the second synthetic polymer.

Use of higher concentrations of both first and second synthetic polymers will result in the formation of a more tightly crosslinked network, producing a stiffer, more robust gel. Compositions intended for use in tissue augmentation will generally employ concentrations of first and second synthetic polymer that fall toward the higher end of the preferred concentration range. Compositions intended for use as bioadhesives or in adhesion prevention do not need to be as firm and may therefore contain lower polymer concentrations.

Because polymers containing multiple electrophilic groups will also react with water, the second synthetic polymer is generally stored and used in sterile, dry form to prevent the loss of crosslinking ability due to hydrolysis which typically occurs upon exposure of such electrophilic groups to aqueous media. Processes for preparing synthetic hydrophilic polymers containing multiple electrophilic groups in sterile, dry form are set forth in U.S. Pat. No. 5,643,464. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates. In contrast, polymers containing multiple nucleophilic groups are generally not water-reactive and can therefore be stored in aqueous solution.

In certain embodiments, one or both of the electrophilic- or nucleophilic-terminated polymers described above can be combined with a synthetic or naturally occurring polymer. The presence of the synthetic or naturally occurring polymer may enhance the mechanical and/or adhesive properties of the in situ forming compositions. Naturally occurring polymers, and polymers derived from naturally occurring polymer that may be included in in situ forming materials include naturally occurring proteins, such as collagen, collagen derivatives (such as methylated collagen), fibrinogen, thrombin, albumin, fibrin, and derivatives of and naturally occurring polysaccharides, such as glycosaminoglycans, including deacetylated and desulfated glycosaminoglycan derivatives.

In one aspect, a composition comprising naturally-occurring protein and both of the first and second synthetic polymer as described above is used to form the crosslinked matrix according to the present invention. In one aspect, a composition comprising collagen and both of the first and second synthetic polymer as described above is used to form the crosslinked matrix according to the present invention. In one aspect, a composition comprising methylated collagen and both of the first and second synthetic polymer as described above is used to form the crosslinked matrix according to the present invention. In one aspect, a composition comprising fibrinogen and both of the first and second synthetic polymer as described above is used to form the crosslinked matrix according to the present invention. In one aspect, a composition comprising thrombin and both of the first and second synthetic polymer as described above is used to form the crosslinked matrix according to the present invention. In one aspect, a composition comprising albumin and both of the first and second synthetic polymer as described above is used to form the crosslinked matrix according to the present invention. In one aspect, a composition comprising fibrin and both of the first and second synthetic polymer as described above is used to form the crosslinked matrix according to the present invention. In one aspect, a composition comprising naturally occurring polysaccharide and both of the first and second synthetic polymer as described above is used to form the crosslinked matrix according to the present invention. In one aspect, a composition comprising glycosaminoglycan and both of the first and second synthetic polymer as described above is used to form the crosslinked matrix according to the present invention. In one aspect, a composition comprising deacetylated glycosaminoglycan and both of the first and second synthetic polymer as described above is used to form the crosslinked matrix according to the present invention. In one aspect, a composition comprising desulfated glycosaminoglycan and both of the first and second synthetic polymer as described above is used to form the crosslinked matrix according to the present invention.

In one aspect, a composition comprising naturally-occurring protein and the first synthetic polymer as described above is used to form the crosslinked matrix according to the present invention. In one aspect, a composition comprising collagen and the first synthetic polymer as described above is used to form the crosslinked matrix according to the present invention. In one aspect, a composition comprising methylated collagen and the first synthetic polymer as described above is used to form the crosslinked matrix according to the present invention. In one aspect, a composition comprising fibrinogen and the first synthetic polymer as described above is used to form the crosslinked matrix according to the present invention. In one aspect, a composition comprising thrombin and the first synthetic polymer as described above is used to form the crosslinked matrix according to the present invention. In one aspect, a composition comprising albumin and the first synthetic polymer as described above is used to form the crosslinked matrix according to the present invention. In one aspect, a composition comprising fibrin and the first synthetic polymer as described above is used to form the crosslinked matrix according to the present invention. In one aspect, a composition comprising naturally occurring polysaccharide and the first synthetic polymer as described above is used to form the crosslinked matrix according to the present invention. In one aspect, a composition comprising glycosaminoglycan and the first synthetic polymer as described above is used to form the crosslinked matrix according to the present invention. In one aspect, a composition comprising deacetylated glycosaminoglycan and the first synthetic polymer as described above is used to form the crosslinked matrix according to the present invention. In one aspect, a composition comprising desulfated glycosaminoglycan and the first synthetic polymer as described above is used to form the crosslinked matrix according to the present invention.

In one aspect, a composition comprising naturally-occurring protein and the second synthetic polymer as described above is used to form the crosslinked matrix according to the present invention. In one aspect, a composition comprising collagen and the second synthetic polymer as described above is used to form the crosslinked matrix according to the present invention. In one aspect, a composition comprising methylated collagen and the second synthetic polymer as described above is used to form the crosslinked matrix according to the present invention. In one aspect, a composition comprising fibrinogen and the second synthetic polymer as described above is used to form the crosslinked matrix according to the present invention. In one aspect, a composition comprising thrombin and the second synthetic polymer as described above is used to form the crosslinked matrix according to the present invention. In one aspect, a composition comprising albumin and the second synthetic polymer as described above is used to form the crosslinked matrix according to the present invention. In one aspect, a composition comprising fibrin and the second synthetic polymer as described above is used to form the crosslinked matrix according to the present invention. In one aspect, a composition comprising naturally occurring polysaccharide and the second synthetic polymer as described above is used to form the crosslinked matrix according to the present invention. In one aspect, a composition comprising glycosaminoglycan and the second synthetic polymer as described above is used to form the crosslinked matrix according to the present invention. In one aspect, a composition comprising deacetylated glycosaminoglycan and the second synthetic polymer as described above is used to form the crosslinked matrix according to the present invention. In one aspect, a composition comprising desulfated glycosaminoglycan and the second synthetic polymer as described above is used to form the crosslinked matrix according to the present invention.

The presence of protein or polysaccharide components which contain functional groups that can react with the functional groups on multiple activated synthetic polymers can result in formation of a crosslinked synthetic polymer-naturally occurring polymer matrix upon mixing and/or crosslinking of the synthetic polymer(s). In particular, when the naturally occurring polymer (protein or polysaccharide) also contains nucleophilic groups such as primary amino groups, the electrophilic groups on the second synthetic polymer will react with the primary amino groups on these components, as well as the nucleophilic groups on the first synthetic polymer, to cause these other components to become part of the polymer matrix. For example, lysine-rich proteins such as collagen may be especially reactive with electrophilic groups on synthetic polymers.

In one aspect, the naturally occurring protein is polymer may be collagen. As used herein, the term “collagen” or “collagen material” refers to all forms of collagen, including those which have been processed or otherwise modified and is intended to encompass collagen of any type, from any source, including, but not limited to, collagen extracted from tissue or produced recombinantly, collagen analogues, collagen derivatives, modified collagens, and denatured collagens, such as gelatin.

In general, collagen from any source may be included in the compositions of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. U.S. Pat. No. 5,428,022 discloses methods of extracting and purifying collagen from the human placenta. U.S. Pat. No. 5,667,839, discloses methods of producing recombinant human collagen in the milk of transgenic animals, including transgenic cows. Collagen of any type, including, but not limited to, types I, II, III, IV, or any combination thereof, may be used in the compositions of the invention, although type I is generally preferred. Either atelopeptide or telopeptide-containing collagen may be used; however, when collagen from a xenogeneic source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from Inamed Aesthetics (Santa Barbara, Calif.) at collagen concentrations of 35 mg/ml and 65 mg/ml under the trademarks ZYDERM I Collagen and ZYDERM II Collagen, respectively. Glutaraldehyde crosslinked atelopeptide fibrillar collagen is commercially available from Inamed Corporation (Santa Barbara, Calif.) at a collagen concentration of 35 mg/ml under the trademark ZYPLAST Collagen.

Collagens for use in the present invention are generally in aqueous suspension at a concentration between about 20 mg/ml to about 120 mg/ml; preferably, between about 30 mg/ml to about 90 mg/ml.

Because of its tacky consistency, nonfibrillar collagen may be preferred for use in compositions that are intended for use as bioadhesives. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559, issued Aug. 14, 1979, to Miyata et al., which is hereby incorporated by reference in its entirety. Due to its inherent tackiness, methylated collagen is particularly preferred for use in bioadhesive compositions, as disclosed in U.S. application Ser. No. 08/476,825.

Collagens for use in the crosslinked polymer compositions of the present invention may start out in fibrillar form, then be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids (e.g., arginine), inorganic salts (e.g., sodium chloride and potassium chloride), and carbohydrates (e.g., various sugars including sucrose).

In one aspect, the polymer may be collagen or a collagen derivative, for example methylated collagen. An example of an in situ forming composition uses pentaerythritol poly(ethylene glycol)ether tetra-sulfhydryl] (4-armed thiol PEG), pentaerythritol poly(ethylene glycol)ether tetra-succinimidyl glutarate] (4-armed NHS PEG) and methylated collagen as the reactive reagents. This composition, when mixed with the appropriate buffers can produce a crosslinked hydrogel. (See, e.g., U.S. Pat. Nos. 5,874,500; 6,051,648; 6,166,130; 5,565,519 and 6,312,725).

In another aspect, the naturally occurring polymer may be a glycosaminoglycan. Glycosaminoglycans, e.g., hyaluronic acid, contain both anionic and cationic functional groups along each polymeric chain, which can form intramolecular and/or intermolecular ionic crosslinks, and are responsible for the thixotropic (or shear thinning) nature of hyaluronic acid.

In certain aspects, the glycosaminoglycan may be derivatized. For example, glycosaminoglycans can be chemically derivatized by, e.g., deacetylation, desulfation, or both in order to contain primary amino groups available for reaction with electrophilic groups on synthetic polymer molecules. Glycosaminoglycans that can be derivatized according to either or both of the aforementioned methods include the following: hyaluronic acid, chondroitin sulfate A, chondroitin sulfate B (dermatan sulfate), chondroitin sulfate C, chitin (can be derivatized to chitosan), keratan sulfate, keratosulfate, and heparin. Derivatization of glycosaminoglycans by deacetylation and/or desulfation and covalent binding of the resulting glycosaminoglycan derivatives with synthetic hydrophilic polymers is described in further detail in commonly assigned, allowed U.S. patent application Ser. No. 08/146,843, filed Nov. 3, 1993.

In general, the collagen is added to the first synthetic polymer, then the collagen and first synthetic polymer are mixed thoroughly to achieve a homogeneous composition. The second synthetic polymer is then added and mixed into the collagen/first synthetic polymer mixture, where it will covalently bind to primary amino groups or thiol groups on the first synthetic polymer and primary amino groups on the collagen, resulting in the formation of a homogeneous crosslinked network. Various deacetylated and/or desulfated glycosaminoglycan derivatives can be incorporated into the composition in a similar manner as that described above for collagen. In addition, the introduction of hydrocolloids such as carboxymethylcellulose may promote tissue adhesion and/or swellability.

Administration of the Crosslinked Synthetic Polymer Compositions

The compositions of the present invention having two synthetic polymers may be administered before, during or after crosslinking of the first and second synthetic polymer. Certain uses, which are discussed in greater detail below, such as tissue augmentation, may require the compositions to be crosslinked before administration, whereas other applications, such as tissue adhesion, require the compositions to be administered before crosslinking has reached “equilibrium.” The point at which crosslinking has reached equilibrium is defined herein as the point at which the composition no longer feels tacky or sticky to the touch.

In order to administer the composition prior to crosslinking, the first synthetic polymer and second synthetic polymer may be contained within separate barrels of a dual-compartment syringe. In this case, the two synthetic polymers do not actually mix until the point at which the two polymers are extruded from the tip of the syringe needle into the patient's tissue. This allows the vast majority of the crosslinking reaction to occur in situ, avoiding the problem of needle blockage which commonly occurs if the two synthetic polymers are mixed too early and crosslinking between the two components is already too advanced prior to delivery from the syringe needle. The use of a dual-compartment syringe, as described above, allows for the use of smaller diameter needles, which is advantageous when performing procedures in delicate tissue, such as that surrounding the eyes.

Alternatively, the first synthetic polymer and second synthetic polymer may be mixed according to the methods described above prior to delivery to the tissue site, then injected to the desired tissue site immediately (preferably, within about 60 seconds) following mixing.

In another embodiment of the invention, the first synthetic polymer and second synthetic polymer are mixed, then extruded and allowed to crosslink into a sheet or other solid form. The crosslinked solid is then dehydrated to remove substantially all unbound water. The resulting dried solid may be ground or comminuted into particulates, then suspended in a nonaqueous fluid carrier, including, without limitation, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid. The suspension of particulates can be injected through a small-gauge needle to a tissue site. Once inside the tissue, the crosslinked polymer particulates will rehydrate and swell in size at least five-fold.

Hydrophilic Polymer+Plurality of Crosslinkable Components

As mentioned above, the first and/or second synthetic polymers may be combined with a hydrophilic polymer, e.g., collagen or methylated collagen, to form a composition useful in the present invention. In one general embodiment, the compositions useful in the present invention include a hydrophilic polymer in combination with two or more crosslinkable components. This embodiment is described in further detail in this section.

The Hydrophilic Polymer Component:

The hydrophilic polymer component may be a synthetic or naturally occurring hydrophilic polymer. Naturally occurring hydrophilic polymers include, but are not limited to: proteins such as collagen and derivatives therof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. See, e.g., U.S. Pat. No. 5,428,022, to Palefsky et al., which discloses methods of extracting and purifying collagen from the human placenta. See also U.S. Pat. No. 5,667,839, to Berg, which discloses methods of producing recombinant human collagen in the milk of transgenic animals, including transgenic cows. Unless otherwise specified, the term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from McGhan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in U.S. Pat. No. 5,614,587 to Rhee et al.

Collagens for use in the crosslinkable compositions of the present invention may start out in fibrillar form, then be rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred. However, as disclosed in U.S. Pat. No. 5,614,587, fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The Crosslinkable Components:

The compositions of the invention also comprise a plurality of crosslinkable components. Each of the crosslinkable components participates in a reaction that results in a crosslinked matrix. Prior to completion of the crosslinking reaction, the crosslinkable components provide the necessary adhesive qualities that enable the methods of the invention.

The crosslinkable components are selected so that crosslinking gives rise to a biocompatible, nonimmunogenic matrix useful in a variety of contexts including adhesion prevention, biologically active agent delivery, tissue augmentation, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A, or nucleophilic and capable of reaction with the electrophilic groups of component B may also be present. Thus, the total number of functional groups present on components A, B and C, when present, in combination is ≧5; that is, the total functional groups given by m+n+p must be ≧5, where p is the number of functional groups on component C and, as indicated, is ≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

The m nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae
(I) R¹(-[Q¹]_{q}-X)ₘ (component A),
(II) R²(-[Q²]ᵣ-Y)ₙ (component B), and
(III) R³(-[Q³]ₛ-Fn)ₚ (optional component C),
   wherein:
R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;
X represents one of the m nucleophilic groups of component A, and the various X moieties on A may be the same or different;
Y represents one of the n electrophilic groups of component B, and the various Y moieties on A may be the same or different;
Fn represents a functional group on optional component C;
Q¹, Q² and Q³ are linking groups;
m≧2, n≧2, m+n is ≧4, q, and r are independently zero or 1, and when optional component C is present, p≧1, and s is independently zero or 1.

Reactive Groups:

X may be virtually any nucleophilic group, so long as reaction can occur with the electrophilic group Y. Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities —R⁶MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones. This class of sulfhydryl reactive groups is particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE

| REPRESENTATIVE | | |
|---|---|---|
| NUCLEOPHILIC | | |
| COMPONENT | REPRESENTATIVE | |
| (A, optional | ELECTROPHILIC | |
| component C | COMPONENT | |
| element FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyl carbonate | |
| | terminus) | |
| R¹—SH | R²—O—(CO)—O—N(COCH₂) | R¹—S—(CO)—O—R² |
| R¹—OH | R²—O—(CO)—O—N(COCH₂) | R¹—O—(CO)—R² |
| R¹—NH₂ | R²—O(CO)—CH═CH₂ | R¹—NH—CH₂CH₂—(CO)—O—R² |
| | (acrylate terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂—N(COCH₂) | R¹—NH—(CO)—(CH₂)₃—(CO)—OR² |
| | (succinimidyl glutarate | |
| | terminus) | |
| R¹—SH | R²—O(CO)—(CH₂)₃—CO₂—N(COCH₂) | R¹—S—(CO)—(CH₂)₃—(CO)—OR² |
| R¹—OH | R²—O(CO)—(CH₂)₃—CO₂—N(COCH₂) | R¹—O—(CO)—(CH₂)₃—(CO)—OR² |
| R¹—NH₂ | R²—O—CH₂—CO₂—N(COCH₂) | R¹—NH—(CO)—CH₂—OR² |
| | (succinimidyl acetate | |
| | terminus) | |
| R¹—SH | R²—O—CH₂—CO₂—N(COCH₂) | R¹—S—(CO)—CH₂—OR² |
| R¹—OH | R²—O—CH₂—CO₂—N(COCH₂) | R¹—O—(CO)—CH₂—OR² |
| R¹—NH₂ | R²—O—NH(CO)—(CH₂)₂—CO₂—N(COCHP₂) | R¹—NH—(CO)—(CH₂)₂—(CO)—NH—OR² |
| | (succinimidyl succinamide | |
| | terminus) | |
| R¹—SH | R²—O—NH(CO)—(CH₂)₂—CO₂—N(COCH₂) | R¹—S—(CO)—(CH₂)₂—(CO)—NH—OR² |
| R¹—OH | R²—O—NH(CO)—(CH₂)₂—CO₂—N(COCH₂) | R¹—O—(CO)—(CH₂)₂—(CO)—NH—OR² |
| R¹—NH₂ | R²—O—(CH₂)₂—CHO | R¹—NH—(CO)—(CH₂)₂—OR² |
| | (propionaldehyde | |
| | terminus) | |
| R¹—NH₂ | 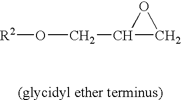 | R¹—NH—CH₂—CH(OH)—CH₂—OR²and R¹—N[CH₂—CH(OH)—CH₂—OR²]₂ |
| R¹—NH₂ | R²—O—(CH₂)₂—N═C═O | R¹—NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹—SH | R²—SO₂—CH═CH₂ | R¹S—CH₂CH₂—SO₂—R² |

Linking Groups:

The functional groups X and Y and FN on optional component C may be directly attached to the compound core (R¹, R² or R³ on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, International Patent Publication No. WO 97/22371. Linking groups are useful to avoid steric hindrance problems that are sometimes associated with the formation of direct linkages between molecules. Linking groups may additionally be used to link several multifunctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia: ester linkages; anhydride linkages, such as obtained by incorporation of glutarate and succinate; ortho ester linkages; ortho carbonate linkages such as trimethylene carbonate; amide linkages; phosphoester linkages; α-hydroxy acid linkages, such as may be obtained by incorporation of lactic acid and glycolic acid; lactone-based linkages, such as may be obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group may be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) may increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in the following Table:

TABLE

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ—Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ—Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ—Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ—Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ—Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ—Z |
| —(CO)—O—(CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ—Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ—Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷—Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH—Z |

In the above Table, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows: If higher molecular weight components are to be used, they preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

The Component Core:

The “core” of each crosslinkable component is comprised of the molecular structure to which the nucleophilic or electrophilic groups are bound. Using the formulae (I) R¹-[Q¹]_{q}—X)ₘ, for component A, (II) R²(-[Q²]ᵣ-Y)ₙ for component B, and (III) R³(-[Q³]-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to 2 heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

Hydrophilic Crosslinkable Components

In one aspect, the crosslinkable component(s) is (are) hydrophilic polymers. The term “hydrophilic polymer” as used herein refers to a synthetic polymer having an average molecular weight and composition effective to render the polymer “hydrophilic” as defined above. As discussed above, synthetic crosslinkable hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic crosslinkable hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

The synthetic crosslinkable hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic crosslinkable hydrophilic polymers can be used in the present compositions, as indicated above, preferred synthetic crosslinkable hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and do not typically interfere with the enzymatic activities and/or conformations of peptides. A particularly preferred synthetic crosslinkable hydrophilic polymer for certain applications is a polyethylene glycol (PEG) having a molecular weight within the range of about 100 to about 100,000 mol. wt., although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 mol. wt., more preferably within the range of about 7,500 to about 20,000 mol. wt. Most preferably, the polyethylene glycol has a molecular weight of approximately 10,000 mol. wt.

Naturally occurring crosslinkable hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see Abuchowski et al., Enzymes as Drugs, John Wiley & Sons: New York, N.Y. (1981) pp. 367-383; and Dreborg et al., Crit. Rev. Therap. Drug Carrier Syst. (1990) 6: 315), peptide chemistry (see Mutter et al., The Peptides, Academic: New York, N.Y. 2: 285-332; and Zalipsky et al., Int. J. Peptide Protein Res. (1987) 30:740), and the synthesis of polymeric drugs (see Zalipsky et al., Eur. Polym. J. (1983) 19: 1177; and Ouchi et al., J. Macromol. Sci. Chem. (1987) A24: 1011).

Activated forms of PEG, including multifunctionally activated PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992); and Shearwater Polymers, Inc. Catalog, Polyethylene Glycol Derivatives, Huntsville, Ala. (1997-1998).

Structures for some specific, tetrafunctionally activated forms of PEG are shown in FIGS. 1 to 10 of U.S. Pat. No. 5,874,500, as are generalized reaction products obtained by reacting the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

Hydrophobic Polymers:

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

Low Molecular Weight Components:

As indicated above, the molecular core of one or more of the crosslinkable components can also be a low molecular weight compound, i.e., a C₂-C₁₄ hydrocarbyl group containing zero to 2 heteroatoms selected from N, O, S and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di- and poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di- and poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives. The aforementioned compounds are commercially available from Pierce (Rockford, Ill.). Such di- and poly-electrophiles can also be synthesized from di- and polyacids, for example by reaction with an appropriate molar amount of N-hydroxysuccinimide in the presence of DCC. Polyols such as trimethylolpropane and di(trimethylol propane) can be converted to carboxylic acid form using various known techniques, then further derivatized by reaction with NHS in the presence of DCC to produce trifunctionally and tetrafunctionally activated polymers.

Delivery Systems:

Suitable delivery systems for the homogeneous dry powder composition (containing at least two crosslinkable polymers) and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration.

The homogeneous dry powder composition and the two buffer solutions may be conveniently formed under aseptic conditions by placing each of the three ingredients (dry powder, acidic buffer solution and basic buffer solution) into separate syringe barrels. For example, the composition, first buffer solution and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution. In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. No. 6,454,786 to Holm et al.; U.S. Pat. No. 6,461,325 to Delmotte et al.; U.S. Pat. No. 5,585,007 to Antanavich et al.; U.S. Pat. No. 5,116,315 to Capozzi et al.; and U.S. Pat. No. 4,631,055 to Redi et al.

Storage and Handling:

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1-dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

Collagen+Fibrinogen and/or Thrombin (e.g., Costasis)

In yet another aspect, the polymer composition may include collagen in combination with fibrinogen and/or thrombin. (See, e.g., U.S. Pat. Nos. 5,290,552; 6,096,309; and 5,997,811). For example, an aqueous composition may include a fibrinogen and FXIII, particularly plasma, collagen in an amount sufficient to thicken the composition, thrombin in an amount sufficient to catalyze polymerization of fibrinogen present in the composition, and Ca²⁺ and, optionally, an antifibrinolytic agent in amount sufficient to retard degradation of the resulting adhesive clot. The composition may be formulated as a two-part composition that may be mixed together just prior to use, in which fibrinogen/FXIII and collagen constitute the first component, and thrombin together with an antifibrinolytic agent, and Ca²⁺ constitute the second component.

Plasma, which provides a source of fibrinogen, may be obtained from the patient for which the composition is to be delivered. The plasma can be used “as is” after standard preparation which includes centrifuging out cellular components of blood. Alternatively, the plasma can be further processed to concentrate the fibrinogen to prepare a plasma cryoprecipitate. The plasma cryoprecipitate can be prepared by freezing the plasma for at least about an hour at about −20° C., and then storing the frozen plasma overnight at about 4° C. to slowly thaw. The thawed plasma is centrifuged and the plasma cryoprecipitate is harvested by removing approximately four-fifths of the plasma to provide a cryoprecipitate comprising the remaining one-fifth of the plasma. Other fibrinogen/FXIII preparations may be used, such as cryoprecipitate, patient autologous fibrin sealant, fibrinogen analogs or other single donor or commercial fibrin sealant materials. Approximately 0.5 ml to about 1.0 ml of either the plasma or the plasma-cryoprecipitate provides about 1 to 2 ml of adhesive composition which is sufficient for use in middle ear surgery. Other plasma proteins (e.g., albumin, plasminogen, von Willebrands factor, Factor VIII, etc.) may or may not be present in the fibrinogen/FXII separation due to wide variations in the formulations and methods to derive them.

Collagen, preferably hypoallergenic collagen, is present in the composition in an amount sufficient to thicken the composition and augment the cohesive properties of the preparation. The collagen may be atelopeptide collagen or telopeptide collagen, e.g., native collagen. In addition to thickening the composition, the collagen augments the fibrin by acting as a macromolecular lattice work or scaffold to which the fibrin network adsorbs. This gives more strength and durability to the resulting glue clot with a relatively low concentration of fibrinogen in comparison to the various concentrated autogenous fibrinogen glue formulations (i.e., AFGs).

The form of collagen which is employed may be described as at least “near native” in its structural characteristics. It may be further characterized as resulting in insoluble fibers at a pH above 5; unless crosslinked or as part of a complex composition, e.g., bone, it will generally consist of a minor amount by weight of fibers with diameters greater than 50 nm, usually from about 1 to 25 volume % and there will be substantially little, if any, change in the helical structure of the fibrils. In addition, the collagen composition must be able to enhance gelation in the surgical adhesion composition.

A number of commercially available collagen preparations may be used. ZYDERM Collagen Implant (ZCI) has a fibrillar diameter distribution consisting of 5 to 10 nm diameter fibers at 90% volume content and the remaining 10% with greater than about 50 nm diameter fibers. ZCI is available as a fibrillar slurry and solution in phosphate buffered isotonic saline, pH 7.2, and is injectable with fine gauge needles. As distinct from ZCI, cross-linked collagen available as ZYPLAST may be employed. ZYPLAST is essentially an exogenously crosslinked (glutaraldehyde) version of ZCI. The material has a somewhat higher content of greater than about 50 nm diameter fibrils and remains insoluble over a wide pH range. Crosslinking has the effect of mimicking in vivo endogenous crosslinking found in many tissues.

Thrombin acts as a catalyst for fibrinogen to provide fibrin, an insoluble polymer and is present in the composition in an amount sufficient to catalyze polymerization of fibrinogen present in the patient plasma. Thrombin also activates FXIII, a plasma protein that catalyzes covalent crosslinks in fibrin, rendering the resultant clot insoluble. Usually the thrombin is present in the adhesive composition in concentration of from about 0.01 to about 1000 or greater NIH units (NIHu) of activity, usually about i to about 500 NIHu, most usually about 200 to about 500 NIHu. The thrombin can be from a variety of host animal sources, conveniently bovine. Thrombin is commercially available from a variety of sources including Parke-Davis, usually lyophilized with buffer salts and stabilizers in vials which provide thrombin activity ranging from about 1000 NIHu to 10,000 NIHu. The thrombin is usually prepared by reconstituting the powder by the addition of either sterile distilled water or isotonic saline. Alternately, thrombin analogs or reptile-sourced coagulants may be used.

The composition may additionally comprise an effective amount of an antifibrinolytic agent to enhance the integrity of the glue clot as the healing processes occur. A number of antifibrinolytic agents are well known and include aprotinin, C1-esterase inhibitor and ε-amino-n-caproic acid (EACA). ε-amino-n-caproic acid, the only antifibrinolytic agent approved by the FDA, is effective at a concentration of from about 5 mg/ml to about 40 mg/ml of the final adhesive composition, more usually from about 20 to about 30 mg/ml. EACA is commercially available as a solution having a concentration of about 250 mg/ml. Conveniently, the commercial solution is diluted with distilled water to provide a solution of the desired concentration. That solution is desirably used to reconstitute lyophilized thrombin to the desired thrombin concentration.

Other examples of in situ forming materials based on the crosslinking of proteins are described, e.g., in U.S. Pat. Nos. RE38158; 4,839,345; 5,514,379, 5,583,114; 6,458,147; 6,371,975; 5,290,552; 6,096,309; U.S. Patent Application Publication Nos 2002/0161399; 2001/0018598 and PCT Publication Nos. WO 03/090683; WO 01/45761; WO 99/66964 and WO 96/03159).

Self-Reactive Compounds

In one aspect, the therapeutic agent is released from a crosslinked matrix formed, at least in part, from a self-reactive compound. As used herein, a self-reactive compound comprises a core substituted with a minimum of three reactive groups. The reactive groups may be directed attached to the core of the compound, or the reactive groups may be indirectly attached to the compound's core, e.g., the reactive groups are joined to the core through one or more linking groups.

Each of the three reactive groups that are necessarily present in a self-reactive compound can undergo a bond-forming reaction with at least one of the remaining two reactive groups. For clarity it is mentioned that when these compounds react to form a crosslinked matrix, it will most often happen that reactive groups on one compound will reactive with reactive groups on another compound. That is, the term “self-reactive” is not intended to mean that each self-reactive compound necessarily reacts with itself, but rather that when a plurality of identical self-reactive compounds are in combination and undergo a crosslinking reaction, then these compounds will react with one another to form the matrix. The compounds are “self-reactive” in the sense that they can react with other compounds having the identical chemical structure as themselves.

The self-reactive compound comprises at least four components: a core and three reactive groups. In one embodiment, the self-reactive compound can be characterized by the formula (I), where R is the core, the reactive groups are represented by X¹, X² and X³, and a linker (L) is optionally present between the core and a functional group.

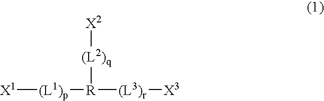

The core R is a polyvalent moiety having attachment to at least three groups (i.e., it is at least trivalent) and may be, or may contain, for example, a hydrophilic polymer, a hydrophobic polymer, an amphiphilic polymer, a C₂₋₁₄ hydrocarbyl, or a C₂₋₁₄ hydrocarbyl which is heteroatom-containing. The linking groups L¹, L², and L³ may be the same or different. The designators p, q and r are either 0 (when no linker is present) or 1 (when a linker is present). The reactive groups X¹, X² and X³ may be the same or different. Each of these reactive groups reacts with at least one other reactive group to form a three-dimensional matrix. Therefore X¹ can react with X² and/or X³, X² can react with X¹ and/or X³, X³ can react with X¹ and/or X² and so forth. A trivalent core will be directly or indirectly bonded to three functional groups, a tetravalent core will be directly or indirectly bonded to four functional groups, etc.

Each side chain typically has one reactive group. However, the invention also encompasses self-reactive compounds where the side chains contain more than one reactive group. Thus, in another embodiment of the invention, the self-reactive compound has the formula (II):
[X′-(L⁴)ₐ-Y′-(L⁵)_{b}]_{c}-R′
where: a and b are integers from 0-1; c is an integer from 3-12; R′ is selected from hydrophilic polymers, hydrophobic polymers, amphiphilic polymers, C₂₋₁₄ hydrocarbyls, and heteroatom-containing C₂₋₁₄ hydrocarbyls; X′ and Y′ are reactive groups and can be the same or different; and L⁴ and L⁵ are linking groups. Each reactive group inter-reacts with the other reactive group to form a three-dimensional matrix. The compound is essentially non-reactive in an initial environment but is rendered reactive upon exposure to a modification in the initial environment that provides a modified environment such that a plurality of the self-reactive compounds inter-react in the modified environment to form a three-dimensional matrix. In one preferred embodiment, R is a hydrophilic polymer. In another preferred embodiment, X′ is a nucleophilic group and Y′ is an electrophilic group.

The following self-reactive compound is one example of a compound of formula (II): 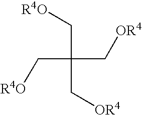
where R⁴ has the formula:

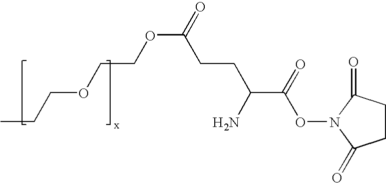

Thus, in formula (II), a and b are 1; c is 4; the core R′ is the hydrophilic polymer, tetrafunctionally activated polyethylene glycol, (C(CH₂—O—)₄; X′ is the electrophilic reactive group, succinimidyl; Y′ is the nucleophilic reactive group —CH—NH₂; L⁴ is —C(O)—O—; and L⁵ is —(CH₂—CH₂—O—CH₂)ₓ—CH₂—O—C(O)—(CH₂)₂—.

The self-reactive compounds of the invention are readily synthesized by techniques that are well known in the art. An exemplary synthesis is set forth below:

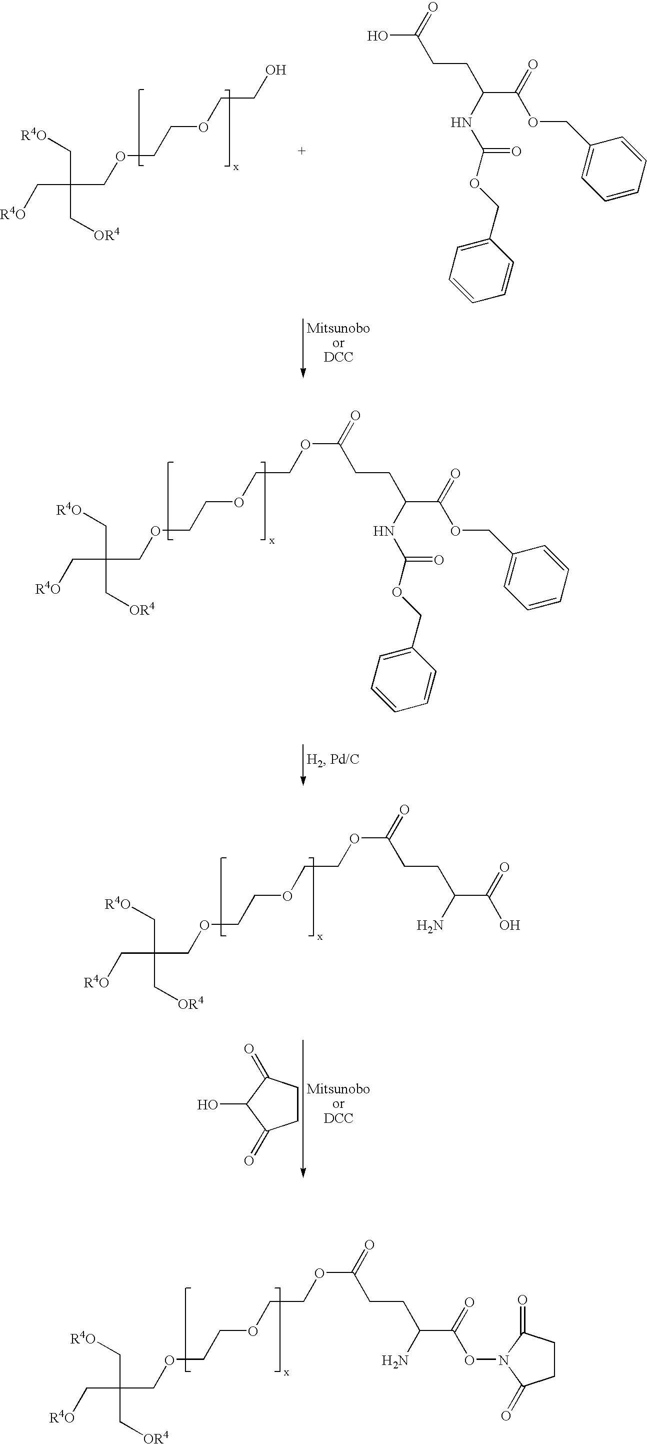

The reactive groups are selected so that the compound is essentially non-reactive in an initial environment. Upon exposure to a specific modification in the initial environment, providing a modified environment, the compound is rendered reactive and a plurality of self-reactive compounds are then able to inter-react in the modified environment to form a three-dimensional matrix. Examples of modification in the initial environment are detailed below, but include the addition of an aqueous medium, a change in pH, exposure to ultraviolet radiation, a change in temperature, or contact with a redox initiator.

The core and reactive groups can also be selected so as to provide a compound that has one of more of the following features: are biocompatible, are non-immunogenic, and do not leave any toxic, inflammatory or immunogenic reaction products at the site of administration. Similarly, the core and reactive groups can also be selected so as to provide a resulting matrix that has one or more of these features.

In one embodiment of the invention, substantially immediately or immediately upon exposure to the modified environment, the self-reactive compounds inter-react form a three-dimensional matrix. The term “substantially immediately” is intended to mean within less than five minutes, preferably within less than two minutes, and the term “immediately” is intended to mean within less than one minute, preferably within less than 30 seconds.

In one embodiment, the self-reactive compound and resulting matrix are not subject to enzymatic cleavage by matrix metalloproteinases such as collagenase, and are therefore not readily degradable in vivo. Further, the self-reactive compound may be readily tailored, in terms of the selection and quantity of each component, to enhance certain properties, e.g., compression strength, swellability, tack, hydrophilicity, optical clarity, and the like.

In one preferred embodiment, R is a hydrophilic polymer. In another preferred embodiment, X is a nucleophilic group, Y is an electrophilic group and Z is either an electrophilic or a nucleophilic group. Additional embodiments are detailed below.

A higher degree of inter-reaction, e.g., crosslinking, may be useful when a less swellable matrix is desired or increased compressive strength is desired. In those embodiments, it may be desirable to have n be an integer from 2-12. In addition, when a plurality of self-reactive compounds are utilized, the compounds may be the same or different.

A. Reactive Groups

Prior to use, the self-reactive compound is stored in an initial environment that insures that the compound remain essentially non-reactive until use. Upon modification of this environment, the compound is rendered reactive and a plurality of compounds will then inter-react to form the desired matrix. The initial environment, as well as the modified environment, is thus determined by the nature of the reactive groups involved.

The number of reactive groups can be the same or different. However, in one embodiment of the invention, the number of reactive groups is approximately equal. As used in this context, the term “approximately” refers to a 2:1 to 1:2 ratio of moles of one reactive group to moles of a different reactive groups. A 1:1:1 molar ratio of reactive groups is generally preferred.

In general, the concentration of the self-reactive compounds in the modified environment, when liquid in nature, will be in the range of about 1 to 50 wt %, generally about 2 to 40 wt %. The preferred concentration of the compound in the liquid will depend on a number of factors, including the type of compound (i.e., type of molecular core and reactive groups), its molecular weight, and the end use of the resulting three-dimensional matrix. For example, use of higher concentrations of the compounds, or using highly functionalized compounds, will result in the formation of a more tightly crosslinked network, producing a stiffer, more robust gel. As such, compositions intended for use in tissue augmentation will generally employ concentrations of self-reactive compounds that fall toward the higher end of the preferred concentration range. Compositions intended for use as bioadhesives or in adhesion prevention do not need to be as firm and may therefore contain lower concentrations of the self-reactive compounds.

1. Electrophilic and Nucleophilic Reactive Groups

In one embodiment of the invention, the reactive groups are electrophilic and nucleophilic groups, which undergo a nucleophilic substitution reaction, a nucleophilic addition reaction, or both. The term “electrophilic” refers to a reactive group that is susceptible to nucleophilic attack, i.e., susceptible to reaction with an incoming nucleophilic group. Electrophilic groups herein are positively charged or electron-deficient, typically electron-deficient. The term “nucleophilic” refers to a reactive group that is electron rich, has an unshared pair of electrons acting as a reactive site, and reacts with a positively charged or electron-deficient site. For such reactive groups, the modification in the initial environment comprises the addition of an aqueous medium and/or a change in pH.

In one embodiment of the invention, X1 (also referred to herein as X) can be a nucleophilic group and X2 (also referred to herein as Y) can be an electrophilic group or vice versa, and X3 (also referred to herein as Z) can be either an electrophilic or a nucleophilic group.

X may be virtually any nucleophilic group, so long as reaction can occur with the electrophilic group Y and also with Z, when Z is electrophilic (Z_{EL}). Analogously, Y may be virtually any electrophilic group, so long as reaction can take place with X and also with Z when Z is nucleophilic (Z_{NU}). The only limitation is a practical one, in that reaction between X and Y, and X and Z_{EL}, or Y and Z_{NU} should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. In one embodiment, the reactions between X and Y, and between either X and Z_{EL} or Y and Z_{NU}, are complete in under 60 minutes, preferably under 30 minutes. Most preferably, the reaction occurs in about 5 to 15 minutes or less.

Examples of nucleophilic groups suitable as X or Fn_{NU} include, but are not limited to: —NH₂, —NHR¹, —N(R¹)₂, —SH, —OH, —COOH, —C₆H₄—OH, —H, —PH₂, —PHR¹, —P(R¹)₂, —NH—NH₂, —CO—NH—NH₂, —C₅H₄N, etc. wherein R¹ is a hydrocarbyl group and each R¹ may be the same or different. R¹ is typically alkyl or monocyclic aryl, preferably alkyl, and most preferably lower alkyl. Organometallic moieties are also useful nucleophilic groups for the purposes of the invention, particularly those that act as carbanion donors. Examples of organometallic moieties include: Grignard functionalities —R²MgHal wherein R² is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophilic group. For example, when there are nucleophilic sulfhydryl and hydroxyl groups in the self-reactive compound, the compound must be admixed with an aqueous base in order to remove a proton and provide an —S⁻ or —O⁻ species to enable reaction with the electrophilic group. Unless it is desirable for the base to participate in the reaction, a non-nucleophilic base is preferred. In some embodiments, the base may be present as a component of a buffer solution. Suitable bases and corresponding crosslinking reactions are described herein.

The selection of electrophilic groups provided on the self-reactive compound, must be made so that reaction is possible with the specific nucleophilic groups. Thus, when the X reactive groups are amino groups, the Y and any Z_{EL} groups are selected so as to react with amino groups. Analogously, when the X reactive groups are sulfhydryl moieties, the corresponding electrophilic groups are sulfhydryl-reactive groups, and the like. In general, examples of electrophilic groups suitable as Y or Z_{EL} include, but are not limited to, —CO—Cl, —(CO)—O—(CO)—R (where R is an alkyl group), —CH═CH—CH═O and —CH═CH—C(CH₃)═O, halo, —N═C═O, —N═C═S, —SO₂CH═CH₂, —O(CO)—C═CH₂, —O(CO)—C(CH₃)═CH₂, —S—S—(C₅H₄N), —O(CO)—C(CH₂CH₃)═CH₂, —CH═CH—C═N H, —COOH, —(CO)O—N(COCH₂)₂, —CHO, —(CO)O—N(COCH₂)₂—S(O)₂OH, and —N(COCH)₂.

When X is amino (generally although not necessarily primary amino), the electrophilic groups present on Y and Z_{EL} are amine-reactive groups. Exemplary amine-reactive groups include, by way of example and not limitation, the following groups, or radicals thereof: (1) carboxylic acid esters, including cyclic esters and “activated” esters; (2) acid chloride groups (—CO—Cl); (3) anhydrides (—(CO)—O—(CO)—R, where R is an alkyl group); (4) ketones and aldehydes, including α,β-unsaturated aldehydes and ketones such as —CH═CH—CH═O and —CH═CH—C(CH₃)═O; (5) halo groups; (6) isocyanate group (—N═C=O); (7) thioisocyanato group (—N═C=S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—O(CO)—C═CH₂), methacrylate (—O(CO)—C(CH₃)═CH₂), ethyl acrylate (—O(CO)—C(CH₂CH₃)═CH₂), and ethyleneimino (—CH═CH—C═NH).

In one embodiment the amine-reactive groups contain an electrophilically reactive carbonyl group susceptible to nucleophilic attack by a primary or secondary amine, for example the carboxylic acid esters and aldehydes noted above, as well as carboxyl groups (—COOH).

Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Accordingly, in one embodiment, the amine-reactive groups are selected from succinimidyl ester (—O(CO)—N(COCH₂)₂), sulfosuccinimidyl ester (—O(CO)—N(COCH₂)₂—S(O)₂OH), maleimido (—N(COCH)₂), epoxy, isocyanato, thioisocyanato, and ethenesulfonyl.

Analogously, when X is sulfhydryl, the electrophilic groups present on Y and Z_{EL} are groups that react with a sulfhydryl moiety. Such reactive groups include those that form thioester linkages upon reaction with a sulfhydryl group, such as those described in WO 00/62827 to Wallace et al. As explained in detail therein, sulfhydryl reactive groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and α,β-unsaturated aldehydes and ketones.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophilic group such as an epoxide group, an aziridine group, an acyl halide, an anhydride, and so forth.

When X is an organometallic nucleophilic group such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophilic or as electrophilic groups, depending on the selected reaction partner and/or the reaction conditions. For example, a carboxylic acid group can act as a nucleophilic group in the presence of a fairly strong base, but generally acts as an electrophilic group allowing nucleophilic attack at the carbonyl carbon and concomitant replacement of the hydroxyl group with the incoming nucleophilic group.

These, as well as other embodiments are illustrated below, where the covalent linkages in the matrix that result upon covalent binding of specific nucleophilic reactive groups to specific electrophilic reactive groups on the self-reactive compound include, solely by way of example, the following Table:

TABLE

| Representative | | |
|---|---|---|
| Nucleophilic | Representative Electrophilic | |
| Group (X, Z_{NU}) | Group (Y, Z_{EL}) | Resulting Linkage |
| —NH₂ | —O—(CO)—O—N(COCH₂)₂ | —NH—(CO)—O— |
| | succinimidyl carbonate terminus | |
| —SH | —O—(CO)—O—N(COCH₂)₂ | —S—(CO)—O— |
| —OH | —O—(CO)—O—N(COCH₂)₂ | —O—(CO)— |
| —NH₂ | —O(CO)—CH═CH₂ | —NH—CH₂CH2—(CO)—O— |
| | acrylate terminus | |
| —SH | —O—(CO)—CH═CH₂ | —S—CH₂CH₂—(CO)—O— |
| —OH | —O—(CO)—CH═CH₂ | —O—CH₂CH₂—(CO)—O— |
| —NH₂ | —O(CO)—(CH₂)₃—CO₂—N(COCH₂)₂ | —NH—(CO)—(CH₂)₃—(CO)—O— |
| | succinimidyl glutarate terminus | |
| —SH | —O(CO)—(CH₂)₃—CO₂—N(COCH₂)₂ | —S—(CO)—(CH₂)₃—(CO)—O— |
| —OH | —O(CO)—(CH₂)₃—CO₂—N(COCH₂)₂ | —O—(CO)—(CH₂)₃—(CO)—O— |
| —NH₂ | —O—CH₂—CO₂—N(COCH₂)₂ | —NH—(CO)—CH₂—O— |
| | succinimidyl acetate terminus | |
| —SH | —O—CH₂—CO₂—N(COCH₂)₂ | —S—(CO)—CH₂—O— |
| —OH | —O—CH₂—CO₂—N(COCH₂)₂ | —O—(CO)—CH₂—O— |
| —NH₂ | —O—NH(CO)—(CH₂)₂—CO₂—N(COCH₂)₂ | —NH—(CO)—(CH₂)₂—(CO)—NH—O— |
| | succinimidyl succinamide | |
| | terminus | |
| —SH | —O—NH(CO)—(CH₂)₂—CO₂—N(COCH₂)₂ | —S—(CO)—(CH₂)₂—(CO)—NH—O— |
| —OH | —O—NH(CO)—(CH₂)₂—CO₂—N(COCH₂)₂ | —O—(CO)—(CH₂)₂—(CO)—NH—O— |
| —NH₂ | —O—(CH₂)₂—CHO | —NH—(CO)—(CH₂)₂—O— |
| | propionaldehyde terminus | |
| —NH₂ | 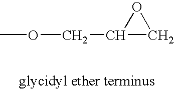 | —NH—CH₂—CH(OH)—CH₂—O—and —N[CH₂—CH(OH)—CH₂—O—]₂ |
| —NH₂ | —O—(CH₂)₂—N═C═O | —NH—(CO)—NH—CH₂—O— |
| | (isocyanate terminus) | |
| —NH₂ | —SO₂—CH═CH₂ | —NH—CH₂CH₂—SO₂— |
| | vinyl sulfone terminus | |
| —SH | —SO₂—CH═CH₂ | —S—CH₂CH₂—SO₂— |

For self-reactive compounds containing electrophilic and nucleophilic reactive groups, the initial environment typically can be dry and sterile. Since electrophilic groups react with water, storage in sterile, dry form will prevent hydrolysis. The dry synthetic polymer may be compression molded into a thin sheet or membrane, which can then be sterilized using gamma or e-beam irradiation. The resulting dry membrane or sheet can be cut to the desired size or chopped into smaller size particulates. The modification of a dry initial environment will typically comprise the addition of an aqueous medium.

In one embodiment, the initial environment can be an aqueous medium such as in a low pH buffer, i.e., having a pH less than about 6.0, in which both electrophilic and nucleophilic groups are non-reactive. Suitable liquid media for storage of such compounds include aqueous buffer solutions such as monobasic sodium phosphate/dibasic sodium phosphate, sodium carbonate/sodium bicarbonate, glutamate or acetate, at a concentration of 0.5 to 300 mM. Modification of an initial low pH aqueous environment will typically comprise increasing the pH to at least pH 7.0, more preferably increasing the pH to at least pH 9.5.

In another embodiment the modification of a dry initial environment comprises dissolving the self-reactive compound in a first buffer solution having a pH within the range of about 1.0 to 5.5 to form a homogeneous solution, and (II) adding a second buffer solution having a pH within the range of about 6.0 to 11.0 to the homogeneous solution. The buffer solutions are aqueous and can be any pharmaceutically acceptable basic or acid composition. The term “buffer” is used in a general sense to refer to an acidic or basic aqueous solution, where the solution may or may not be functioning to provide a buffering effect (i.e., resistance to change in pH upon addition of acid or base) in the compositions of the present invention. For example, the self-reactive compound can be in the form of a homogeneous dry powder. This powder is then combined with a buffer solution having a pH within the range of about 1.0 to 5.5 to form a homogeneous acidic aqueous solution, and this solution is then combined with a buffer solution having a pH within the range of about 6.0 to 11.0 to form a reactive solution. For example, 0.375 grams of the dry powder can be combined with 0.75 grams of the acid buffer to provide, after mixing, a homogeneous solution, where this solution is combined with 1.1 grams of the basic buffer to provide a reactive mixture that substantially immediately forms a three-dimensional matrix.

Acidic buffer solutions having a pH within the range of about 1.0 to 5.5, include by way of illustration and not limitation, solutions of: citric acid, hydrochloric acid, phosphoric acid, sulfuric acid, AMPSO (3-[(1,1-dimethyl-2-hydroxyethyl)amino]2-hydroxy-propane-sulfonic acid), acetic acid, lactic acid, and combinations thereof. In a preferred embodiment, the acidic buffer solution, is a solution of citric acid, hydrochloric acid, phosphoric acid, sulfuric acid, and combinations thereof. Regardless of the precise acidifying agent, the acidic buffer preferably has a pH such that it retards the reactivity of the nucleophilic groups on the core. For example, a pH of 2.1 is generally sufficient to retard the nucleophilicity of thiol groups. A lower pH is typically preferred when the core contains amine groups as the nucleophilic groups. In general, the acidic buffer is an acidic solution that, when contacted with nucleophilic groups, renders those nucleophilic groups relatively non-nucleophilic.

An exemplary acidic buffer is a solution of hydrochloric acid, having a concentration of about 6.3 mM and a pH in the range of 2.1 to 2.3. This buffer may be prepared by combining concentrated hydrochloric acid with water, i.e., by diluting concentrated hydrochloric acid with water. Similarly, this buffer A may also be conveniently prepared by diluting 1.23 grams of concentrated hydrochloric acid to a volume of 2 liters, or diluting 1.84 grams of concentrated hydrochloric acid to a volume to 3 liters, or diluting 2.45 grams of concentrated hydrochloric acid to a volume of 4 liters, or diluting 3.07 grams concentrated hydrochloric acid to a volume of 5 liters, or diluting 3.68 grams of concentrated hydrochloric acid to a volume to 6 liters. For safety reasons, the concentrated acid is preferably added to water.

Basic buffer solutions having a pH within the range of about 6.0 to 11.0, include by way of illustration and not limitation, solutions of: glutamate, acetate, carbonate and carbonate salts (e.g., sodium carbonate, sodium carbonate monohydrate and sodium bicarbonate), borate, phosphate and phosphate salts (e.g., monobasic sodium phosphate monohydrate and dibasic sodium phosphate), and combinations thereof. In a preferred embodiment, the basic buffer solution is a solution of carbonate salts, phosphate salts, and combinations thereof.

In general, the basic buffer is an aqueous solution that neutralizes the effect of the acidic buffer, when it is added to the homogeneous solution of the compound and first buffer, so that the nucleophilic groups on the core regain their nucleophilic character (that has been masked by the action of the acidic buffer), thus allowing the nucleophilic groups to inter-react with the electrophilic groups on the core.

An exemplary basic buffer is an aqueous solution of carbonate and phosphate salts. This buffer may be prepared by combining a base solution with a salt solution. The salt solution may be prepared by combining 34.7 g of monobasic sodium phosphate monohydrate, 49.3 g of sodium carbonate monohydrate, and sufficient water to provide a solution volume of 2 liter. Similarly, a 6 liter solution may be prepared by combining 104.0 g of monobasic sodium phosphate monohydrate, 147.94 g of sodium carbonate monohydrate, and sufficient water to provide 6 liter of the salt solution. The basic buffer may be prepared by combining 7.2 g of sodium hydroxide with 180.0 g of water. The basic buffer is typically prepared by adding the base solution as needed to the salt solution, ultimately to provide a mixture having the desired pH, e.g., a pH of 9.65 to 9.75.

In general, the basic species present in the basic buffer should be sufficiently basic to neutralize the acidity provided by the acidic buffer, but should not be so nucleophilic itself that it will react substantially with the electrophilic groups on the core. For this reason, relatively “soft” bases such as carbonate and phosphate are preferred in this embodiment of the invention.

To illustrate the preparation of a three-dimensional matrix of the present invention, one may combine an admixture of the self-reactive compound with a first, acidic, buffer (e.g., an acid solution, e.g., a dilute hydrochloric acid solution) to form a homogeneous solution. This homogeneous solution is mixed with a second, basic, buffer (e.g., a basic solution, e.g., an aqueous solution containing phosphate and carbonate salts) whereupon the reactive groups on the core of the self-reactive compound substantially immediately inter-react with one another to form a three-dimensional matrix.

2. Redox Reactive Groups

In one embodiment of the invention, the reactive groups are vinyl groups such as styrene derivatives, which undergo a radical polymerization upon initiation with a redox initiator. The term “redox” refers to a reactive group that is susceptible to oxidation-reduction activation. The term “vinyl” refers to a reactive group that is activated by a redox initiator, and forms a radical upon reaction. X, Y and Z can be the same or different vinyl groups, for example, methacrylic groups.

For self-reactive compounds containing vinyl reactive groups, the initial environment typically will be an aqueous environment. The modification of the initial environment involves the addition of a redox initiator.

3. Oxidative Coupling Reactive Groups

In one embodiment of the invention, the reactive groups undergo an oxidative coupling reaction. For example, X, Y and Z can be a halo group such as chloro, with an adjacent electron-withdrawing group on the halogen-bearing carbon (e.g., on the “L” linking group). Exemplary electron-withdrawing groups include nitro, aryl, and so forth.

For such reactive groups, the modification in the initial environment comprises a change in pH. For example, in the presence of a base such as KOH, the self-reactive compounds then undergo a de-hydro, chloro coupling reaction, forming a double bond between the carbon atoms, as illustrated below:

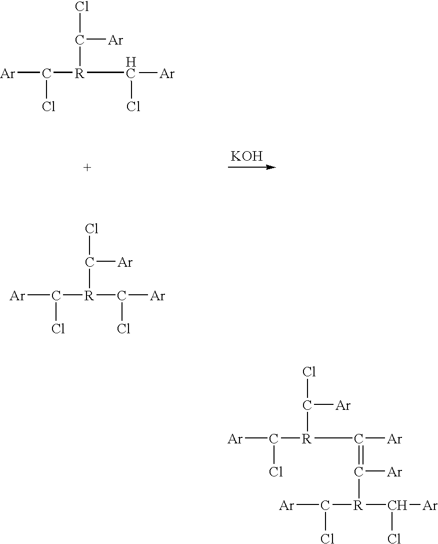

For self-reactive compounds containing oxidative coupling reactive groups, the initial environment typically can be can be dry and sterile, or a non-basic medium. The modification of the initial environment will typically comprise the addition of a base.

4. Photoinitiated Reactive Groups

In one embodiment of the invention, the reactive groups are photoinitiated groups. For such reactive groups, the modification in the initial environment comprises exposure to ultraviolet radiation.

In one embodiment of the invention, X can be an azide (—N₃) group and Y can be an alkyl group such as —CH(CH₃)₂ or vice versa. Exposure to ultraviolet radiation will then form a bond between the groups to provide for the following linkage: —NH—C(CH₃)₂—CH₂—. In another embodiment of the invention, X can be a benzophenone (—(C₆H₄)—C(O)—(C₆H₅)) group and Y can be an alkyl group such as —CH(CH₃)₂ or vice versa. Exposure to ultraviolet radiation will then form a bond between the groups to provide for the following linkage:

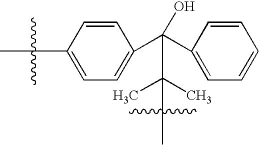

For self-reactive compounds containing photoinitiated reactive groups, the initial environment typically will be in an ultraviolet radiation-shielded environment. This can be for example, storage within a container that is impermeable to ultraviolet radiation.

The modification of the initial environment will typically comprise exposure to ultraviolet radiation.

5. Temperature-Sensitive Reactive Groups

In one embodiment of the invention, the reactive groups are temperature-sensitive groups, which undergo a thermochemical reaction. For such reactive groups, the modification in the initial environment thus comprises a change in temperature. The term “temperature-sensitive” refers to a reactive group that is chemically inert at one temperature or temperature range and reactive at a different temperature or temperature range.

In one embodiment of the invention, X, Y, and Z are the same or different vinyl groups.

For self-reactive compounds containing reactive groups that are temperature-sensitive, the initial environment typically will be within the range of about 10 to 30° C.

The modification of the initial environment will typically comprise changing the temperature to within the range of about 20 to 40° C.

B. Linking Groups

The reactive groups may be directly attached to the core, or they may be indirectly attached through a linking group, with longer linking groups also termed “chain extenders.” In the formula (I) shown above, the optional linker groups are represented by L¹, L², and L³, wherein the linking groups are present when p, q and r are equal to 1.

Suitable linking groups are well known in the art. See, for example, WO 97/22371 to Rhee et al. Linking groups are useful to avoid steric hindrance problems that can sometimes associated with the formation of direct linkages between molecules. Linking groups may additionally be used to link several self-reactive compounds together to make larger molecules. In one embodiment, a linking group can be used to alter the degradative properties of the compositions after administration and resultant gel formation. For example, linking groups can be used to promote hydrolysis, to discourage hydrolysis, or to provide a site for enzymatic degradation.

Examples of linking groups that provide hydrolyzable sites, include, inter alia: ester linkages; anhydride linkages, such as those obtained by incorporation of glutarate and succinate; ortho ester linkages; ortho carbonate linkages such as trimethylene carbonate; amide linkages; phosphoester linkages; a-hydroxy acid linkages, such as those obtained by incorporation of lactic acid and glycolic acid; lactone-based linkages, such as those obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, WO 99/07417 to Coury et al. Examples of enzymatically degradable linkages include Leu-Gly-Pro-Ala, which is degraded by collagenase; and Gly-Pro-Lys, which is degraded by plasmin.

Linking groups can also be included to enhance or suppress the reactivity of the various reactive groups. For example, electron-withdrawing groups within one or two carbons of a sulfhydryl group may be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) may increase the reactivity of the carbonyl carbon with respect to an incoming nucleophilic group. By contrast, sterically bulky groups in the vicinity of a reactive group can be used to diminish reactivity and thus reduce the coupling rate as a result of steric hindrance. By way of example, particular linking groups and corresponding formulas are indicated in the following Table:

TABLE

| Linking group | Component structure |
|---|---|
| —O—(CH₂)ₓ— | —O—(CH₂)ₓ—X |
| | —O—(CH₂)ₓ—Y |
| | —O—(CH₂)ₓ—Z |
| —S—(CH₂)ₓ— | —S—(CH₂)ₓ—X |
| | —S—(CH₂)ₓ—Y |
| | —S—(CH₂)ₓ—Z |
| —NH—(CH₂)ₓ— | —NH—(CH₂)ₓ—X |
| | —NH—(CH₂)ₓ—Y |
| | —NH—(CH₂)ₓ—Z |
| —O—(CO)—NH—(CH₂)ₓ— | —O—(CO)—NH—(CH₂)ₓ—X |
| | —O—(CO)—NH—(CH₂)ₓ—Y |
| | —O—(CO)—NH—(CH₂)ₓ—Z |
| —NH—(CO)—O—(CH₂)ₓ— | —NH—(CO)—O—(CH₂)ₓ—X |
| | —NH—(CO)—O—(CH₂)ₓ—Y |
| | —NH—(CO)—O—(CH₂)ₓ—Z |
| —O—(CO)—(CH₂)ₓ— | —O—(CO)—(CH₂)ₓ—X |
| | —O—(CO)—(CH₂)ₓ—Y |
| | —O—(CO)—(CH₂)ₓ—Z |
| —(CO)—O—(CH₂)ₓ— | —(CO)—O—(CH₂)ₙ—X |
| | —(CO)—O—(CH₂)ₙ—Y |
| | —(CO)—O—(CH₂)ₙ—Z |
| —O—(CO)—O—(CH₂)ₓ— | —O—(CO)—O—(CH₂)ₓ—X |
| | —O—(CO)—O—(CH₂)ₓ—Y |
| | —O—(CO)—O—(CH₂)ₓ—Z |
| —O—(CO)—CHR²— | —O—(CO)—CHR²—X |
| | —O—(CO)—CHR²—Y |
| | —O—(CO)—CHR²—Z |
| —O—R³—(CO)—NH— | —O—R³—(CO)—NH—X |
| | —O—R³—(CO)—NH—Y |
| | —O—R³—(CO)—NH—Z |

In the above Table, x is generally in the range of 1 to about 10; R² is generally hydrocarbyl, typically alkyl or aryl, preferably alkyl, and most preferably lower alkyl; and R³ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows. If a higher molecular weight self-reactive compound is to be used, it will preferably have biodegradable linkages as described above, so that fragments larger than 20,000 mol. wt. are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

C. The Core

The “core” of each self-reactive compound is comprised of the molecular structure to which the reactive groups are bound. The molecular core can be a polymer, which includes synthetic polymers and naturally occurring polymers. In one embodiment, the core is a polymer containing repeating monomer units. The polymers can be hydrophilic, hydrophobic, or amphiphilic. The molecular core can also be a low molecular weight component such as a C₂₋₁₄ hydrocarbyl or a heteroatom-containing C₂₋₁₄ hydrocarbyl. The heteroatom-containing C₂₋₁₄ hydrocarbyl can have 1 or 2 heteroatoms selected from N, O and S. In a preferred embodiment, the self-reactive compound comprises a molecular core of a synthetic hydrophilic polymer.

1. Hydrophilic Polymers

As mentioned above, the term “hydrophilic polymer” as used herein refers to a polymer having an average molecular weight and composition that naturally renders, or is selected to render the polymer as a whole “hydrophilic.” Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions.

Synthetic hydrophilic polymers may be homopolymers, block copolymers including di-block and tri-block copolymers, random copolymers, or graft copolymers. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments preferably degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like. Other biodegradable segments that may form part of the hydrophilic polymer core include polyesters such as polylactide, polyethers such as polyalkylene oxide, polyamides such as a protein, and polyurethanes. For example, the core of the self-reactive compound can be a diblock copolymer of tetrafunctionally activated polyethylene glycol and polylactide.

Synthetic hydrophilic polymers that are useful herein include, but are not limited to: polyalkylene oxides, particularly polyethylene glycol (PEG) and poly(ethylene oxide)-poly(propylene oxide) copolymers, including block and random copolymers; polyols such as glycerol, polyglycerol (PG) and particularly highly branched polyglycerol, propylene glycol; poly(oxyalkylene)-substituted diols, and poly(oxyalkylene)-substituted polyols such as mono-, di- and tri-polyoxyethylated glycerol, mono- and di-polyoxyethylated propylene glycol, and mono- and di-polyoxyethylated trimethylene glycol; polyoxyethylated sorbitol, polyoxyethylated glucose; poly(acrylic acids) and analogs and copolymers thereof, such as polyacrylic acid per se, polymethacrylic acid, poly(hydroxyethylmethacrylate), poly(hydroxyethylacrylate), poly(methylalkylsulfoxide methacrylates), poly(methylalkylsulfoxide acrylates) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), poly(N-isopropyl-acrylamide), and copolymers thereof; poly(olefinic alcohols) such as poly(vinyl alcohols) and copolymers thereof; poly(N-vinyl lactams) such as poly(vinyl pyrrolidones), poly(N-vinyl caprolactams), and copolymers thereof; polyoxazolines, including poly(methyloxazoline) and poly(ethyloxazoline); and polyvinylamines; as well as copolymers of any of the foregoing. It must be emphasized that the aforementioned list of polymers is not exhaustive, and a variety of other synthetic hydrophilic polymers may be used, as will be appreciated by those skilled in the art.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories, Inc. (Belmont, Calif.).

Although a variety of different synthetic hydrophilic polymers can be used in the present compounds, preferred synthetic hydrophilic polymers are PEG and PG, particularly highly branched PG. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a PEG having a molecular weight within the range of about 100 to about 100,000, although for highly branched PEG, far higher molecular weight polymers can be employed, up to 1,000,000 or more, providing that biodegradable sites are incorporated ensuring that all degradation products will have a molecular weight of less than about 30,000. For most PEGs, however, the preferred molecular weight is about 1,000 to about 20,000, more preferably within the range of about 7,500 to about 20,000. Most preferably, the polyethylene glycol has a molecular weight of approximately 10,000.

Naturally occurring hydrophilic polymers include, but are not limited to: proteins such as collagen, fibronectin, albumins, globulins, fibrinogen, fibrin and thrombin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are preferred naturally occurring hydrophilic polymers for use herein.

Unless otherwise specified, the term “collagen” as used herein refers to all forms of collagen, including those, which have been processed or otherwise modified. Thus, collagen from any source may be used in the compounds of the invention; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. For example, U.S. Pat. No. 5,428,022 to Palefsky et al. discloses methods of extracting and purifying collagen from the human placenta, and U.S. Pat. No. 5,667,839 to Berg discloses methods of producing recombinant human collagen in the milk of transgenic animals, including transgenic cows. Non-transgenic, recombinant collagen expression in yeast and other cell lines) is described in U.S. Pat. No. 6,413,742 to Olsen et al., 6,428,978 to Olsen et al., and 6,653,450 to Berg et al.

Collagen of any type, including, but not limited to, types I, II, III, IV, or any combination thereof, may be used in the compounds of the invention, although type I is generally preferred. Either atelopeptide or telopeptide-containing collagen may be used; however, when collagen from a natural source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the invention, although previously crosslinked collagen may be used.

Collagens for use in the present invention are generally, although not necessarily, in aqueous suspension at a concentration between about 20 mg/ml to about 120 mg/ml, preferably between about 30 mg/ml to about 90 mg/ml. Although intact collagen is preferred, denatured collagen, commonly known as gelatin, can also be used. Gelatin may have the added benefit of being degradable faster than collagen.

Nonfibrillar collagen is generally preferred for use in compounds of the invention, although fibrillar collagens may also be used. The term “nonfibrillar collagen” refers to any modified or unmodified collagen material that is in substantially nonfibrillar form, i.e., molecular collagen that is not tightly associated with other collagen molecules so as to form fibers. Typically, a solution of nonfibrillar collagen is more transparent than is a solution of fibrillar collagen. Collagen types that are nonfibrillar (or microfibrillar) in native form include types IV, VI, and VII.

Chemically modified collagens that are in nonfibrillar form at neutral pH include succinylated collagen and methylated collagen, both of which can be prepared according to the methods described in U.S. Pat. No. 4,164,559 to Miyata et al. Methylated collagen, which contains reactive amine groups, is a preferred nucleophile-containing component in the compositions of the present invention. In another aspect, methylated collagen is a component that is present in addition to first and second components in the matrix-forming reaction of the present invention. Methylated collagen is described in, for example, in U.S. Pat. No. 5,614,587 to Rhee et al.

Collagens for use in the compositions of the present invention may start out in fibrillar form, then can be rendered nonfibrillar by the addition of one or more fiber disassembly agent. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

Fibrillar collagen is less preferred for use in the compounds of the invention. However, as disclosed in U.S. Pat. No. 5,614,587 to Rhee et al., fibrillar collagen, or mixtures of nonfibrillar and fibrillar collagen, may be preferred for use in compounds intended for long-term persistence in vivo.

2. Hydrophobic Polymers

The core of the self-reactive compound may also comprise a hydrophobic polymer, including low molecular weight polyfunctional species, although for most uses hydrophilic polymers are preferred. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing, for example, multiple nucleophilic groups. Thus, use of short-chain oligomers can avoid solubility-related problems during reaction. Polylactic acid and polyglycolic acid are examples of two particularly suitable hydrophobic polymers.

3. Amphiphilic Polymers

Generally, amphiphilic polymers have a hydrophilic portion and a hydrophobic (or lipophilic) portion. The hydrophilic portion can be at one end of the core and the hydrophobic portion at the opposite end, or the hydrophilic and hydrophobic portions may be distributed randomly (random copolymer) or in the form of sequences or grafts (block copolymer) to form the amphiphilic polymer core of the self-reactive compound. The hydrophilic and hydrophobic portions may include any of the aforementioned hydrophilic and hydrophobic polymers.

Alternately, the amphiphilic polymer core can be a hydrophilic polymer that has been modified with hydrophobic moieties (e.g., alkylated PEG or a hydrophilic polymer modified with one or more fatty chains), or a hydrophobic polymer that has been modified with hydrophilic moieties (e.g., “PEGylated” phospholipids such as polyethylene glycolated phospholipids).

4. Low Molecular Weight Components

As indicated above, the molecular core of the self-reactive compound can also be a low molecular weight compound, defined herein as being a C₂₋₁₄ hydrocarbyl or a heteroatom-containing C₂₋₁₄ hydrocarbyl, which contains 1 to 2 heteroatoms selected from N, O, S and combinations thereof. Such a molecular core can be substituted with any of the reactive groups described herein.

Alkanes are suitable C₂₋₁₄ hydrocarbyl molecular cores. Exemplary alkanes, for substituted with a nucleophilic primary amino group and a Y electrophilic group, include, ethyleneamine (H₂N—CH₂CH₂—Y), tetramethyleneamine (H₂N—(CH₄)—Y), pentamethyleneamine (H₂N—(CH₅)—Y), and hexamethyleneamine (H₂N—(CH₆)—Y).

Low molecular weight diols and polyols are also suitable C₂₋₁₄ hydrocarbyls and include trimethylolpropane, di(trimethylol propane), pentaerythritol, and diglycerol. Polyacids are also suitable C₂₋₁₄ hydrocarbyls, and include trimethylolpropane-based tricarboxylic acid, di(trimethylol propane)-based tetracarboxylic acid, heptanedioic acid, octanedioic acid (suberic acid), and hexadecanedioic acid (thapsic acid).

Low molecular weight di- and poly-electrophiles are suitable heteroatom-containing C₂₋₁₄ hydrocarbyl molecular cores. These include, for example, disuccinimidyl suberate (DSS), bis(sulfosuccinimidyl) suberate (BS₃), dithiobis(succinimidylpropionate) (DSP), bis(2-succinimidooxycarbonyloxy) ethyl sulfone (BSOCOES), and 3,3′-dithiobis(sulfosuccinimidylpropionate (DTSPP), and their analogs and derivatives.

In one embodiment of the invention, the self-reactive compound of the invention comprises a low-molecular weight material core, with a plurality of acrylate moieties and a plurality of thiol groups.

D. Preparation

The self-reactive compounds are readily synthesized to contain a hydrophilic, hydrophobic or amphiphilic polymer core or a low molecular weight core, functionalized with the desired functional groups, i.e., nucleophilic and electrophilic groups, which enable crosslinking. For example, preparation of a self-reactive compound having a polyethylene glycol (PEG) core is discussed below. However, it is to be understood that the following discussion is for purposes of illustration and analogous techniques may be employed with other polymers.

With respect to PEG, first of all, various functionalized PEGs have been used effectively in fields such as protein modification (see Abuchowski et al., Enzymes as Drugs, John Wiley & Sons: New York, N.Y. (1981) pp. 367-383; and Dreborg et al. (1990) Crit. Rev. Therap. Drug Carrier Syst. 6: 315), peptide chemistry (see Mutter et al., The Peptides, Academic: New York, N.Y. 2: 285-332; and Zalipsky et al. (1987) Int. J. Peptide Protein Res. 30: 740), and the synthesis of polymeric drugs (see Zalipsky et al. (1983) Eur. Polym. J. 19: 1177; and Ouchi et al. (1987) J. Macromol. Sci. Chem. A24: 1011).

Functionalized forms of PEG, including multi-functionalized PEG, are commercially available, and are also easily prepared using known methods. For example, see Chapter 22 of Poly(ethylene Glycol) Chemistry: Biotechnical and Biomedical Applications, J. Milton Harris, ed., Plenum Press, NY (1992).

Multi-functionalized forms of PEG are of particular interest and include, PEG succinimidyl glutarate, PEG succinimidyl propionate, succinimidyl butylate, PEG succinimidyl acetate, PEG succinimidyl succinamide, PEG succinimidyl carbonate, PEG propionaldehyde, PEG glycidyl ether, PEG-isocyanate, and PEG-vinylsulfone. Many such forms of PEG are described in U.S. Pat. Nos. 5,328,955 and 6,534,591, both to Rhee et al. Similarly, various forms of multi-amino PEG are commercially available from sources such as PEG Shop, a division of SunBio of South Korea (www.sunbio.com), Nippon Oil and Fats (Yebisu Garden Place Tower, 20-3 Ebisu 4-chome, Shibuya-ku, Tokyo), Nektar Therapeutics (San Carlos, Calif., formerly Shearwater Polymers, Huntsville, Ala.) and from Huntsman's Performance Chemicals Group (Houston, Tex.) under the name Jeffamine® polyoxyalkyleneamines. Multi-amino PEGs useful in the present invention include the Jeffamine diamines (“D” series) and triamines (“T” series), which contain two and three primary amino groups per molecule. Analogous poly(sulfhydryl) PEGs are also available from Nektar Therapeutics, e.g., in the form of pentaerythritol poly(ethylene glycol) ether tetra-sulfhydryl (molecular weight 10,000). These multi-functionalized forms of PEG can then be modified to include the other desired reactive groups.

Reaction with succinimidyl groups to convert terminal hydroxyl groups to reactive esters is one technique for preparing a core with electrophilic groups. This core can then be modified include nucleophilic groups such as primary amines, thiols, and hydroxyl groups. Other agents to convert hydroxyl groups include carbonyldiimidazole and sulfonyl chloride. However, as discussed herein, a wide variety of electrophilic groups may be advantageously employed for reaction with corresponding nucleophilic groups. Examples of such electrophilic groups include acid chloride groups; anhydrides, ketones, aldehydes, isocyanate, isothiocyanate, epoxides, and olefins, including conjugated olefins such as ethenesulfonyl (—SO₂CH═CH₂) and analogous functional groups.

Other In Situ Crosslinking Materials

Numerous other types of in situ forming materials have been described which may be used in combination with an anti-scarring agent in accordance with the invention. The in situ forming material may be a biocompatible crosslinked polymer that is formed from water soluble precursors having electrophilic and nucleophilic groups capable of reacting and crosslinking in situ (see, e.g., U.S. Pat. No. 6,566,406). The in situ forming material may be hydrogel that may be formed through a combination of physical and chemical crosslinking processes, where physical crosslinking is mediated by one or more natural or synthetic components that stabilize the hydrogel-forming precursor solution at a deposition site for a period of time sufficient for more resilient chemical crosslinks to form (see, e.g., U.S. Pat. No. 6,818,018). The in situ forming material may be formed upon exposure to an aqueous fluid from a physiological environment from dry hydrogel precursors (see, e.g., U.S. Pat. No. 6,703,047). The in situ forming material may be a hydrogel matrix that provides controlled release of relatively low molecular weight therapeutic species by first dispersing or dissolving the therapeutic species within relatively hydrophobic rate modifying agents to form a mixture; the mixture is formed into microparticles that are dispersed within bioabsorbable hydrogels, so as to release the water soluble therapeutic agents in a controlled fashion (see, e.g., 6,632,457). The in situ forming material may be a multi-component hydrogel system (see, e.g., U.S. Pat. No. 6,379,373). The in situ forming material may be a multi-arm block copolymer that includes a central core molecule, such as a residue of a polyol, and at least three copolymer arms covalently attached to the central core molecule, each copolymer arm comprising an inner hydrophobic polymer segment covalently attached to the central core molecule and an outer hydrophilic polymer segment covalently attached to the hydrophobic polymer segment, wherein the central core molecule and the hydrophobic polymer segment define a hydrophobic core region (see, e.g., U.S. Pat. No. 6,730,334). The in situ forming material may include a gel-forming macromer that includes at least four polymeric blocks, at least two of which are hydrophobic and at least one of which is hydrophilic, and including a crosslinkable group (see, e.g., U.S. Pat. No. 6,639,014). The in situ forming material may be a water-soluble macromer that includes at least one hydrolysable linkage formed from carbonate or dioxanone groups, at least one water-soluble polymeric block, and at least one polymerizable group (see, e.g., U.S. Pat. No. 6,177,095). The in situ forming material may comprise polyoxyalkylene block copolymers that form weak physical crosslinks to provide gels having a paste-like consistency at physiological temperatures. (see, e.g., U.S. Pat. No. 4,911,926). The in situ forming material may be a thermo-irreversible gel made from polyoxyalkylene polymers and ionic polysaccharides (see, e.g., U.S. Pat. No. 5,126,141). The in situ forming material may be a gel forming composition that includes chitin derivatives (see, e.g., U.S. Pat. No. 5,093,319), chitosan-coagulum (see, e.g., U.S. Pat. No. 4,532,134), or hyaluronic acid (see, e.g., U.S. Pat. No. 4,141,973). The in situ forming material may be an in situ modification of alginate (see, e.g., U.S. Pat. No. 5,266,326). The in situ forming material may be formed from ethylenically unsaturated water soluble macromers that can be crosslinked in contact with tissues, cells, and bioactive molecules to form gels (see, e.g., U.S. Pat. No. 5,573,934). The in situ forming material may include urethane prepolymers used in combination with an unsaturated cyano compound containing a cyano group attached to a carbon atom, such as cyano(meth)acrylic acids and esters thereof (see, e.g., U.S. Pat. No. 4,740,534). The in situ forming material may be a biodegradable hydrogel that polymerizes by a photoinitiated free radical polymerization from water soluble macromers (see, e.g., U.S. Pat. No. 5,410,016). The in situ forming material may be formed from a two component mixture including a first part comprising a serum albumin protein in an aqueous buffer having a pH in a range of about 8.0-11.0, and a second part comprising a water-compatible or water-soluble bifunctional crosslinking agent. (see, e.g., U.S. Pat. No. 5,583,114).

In another aspect, in situ forming materials that can be used include those based on the crosslinking of proteins. For example, the in situ forming material may be a biodegradable hydrogel composed of a recombinant or natural human serum albumin and poly(ethylene) glycol polymer solution whereby upon mixing the solution cross-links to form a mechanical non-liquid covering structure which acts as a sealant. See, e.g., U.S. Pat. Nos. 6,458,147 and 6,371,975. The in situ forming material may be composed of two separate mixtures based on fibrinogen and thrombin which are dispensed together to form a biological adhesive when intermixed either prior to or on the application site to form a fibrin sealant. See, e.g., U.S. Pat. No. 6,764,467. The in situ forming material may be composed of ultrasonically treated collagen and albumin which form a viscous material that develops adhesive properties when crosslinked chemically with glutaraldehyde and amino acids or peptides. See, e.g., U.S. Pat. No. 6,310,036. The in situ forming material may be a hydrated adhesive gel composed of an aqueous solution consisting essentially of a protein having amino groups at the side chains (e.g., gelatin, albumin) which is crosslinked with an N-hydroxyimidoester compound. See, e.g., U.S. Pat. No. 4,839,345. The in situ forming material may be a hydrogel prepared from a protein or polysaccharide backbone (e.g., albumin or polymannuronic acid) bonded to a cross-linking agent (e.g., polyvalent derivatives of polyethylene or polyalkylene glycol). See, e.g., U.S. Pat. No. 5,514,379. The in situ forming material may be composed of a polymerizable collagen composition that is applied to the tissue and then exposed to an initiator to polymerize the collagen to form a seal over a wound opening in the tissue. See, e.g., U.S. Pat. No. 5,874,537. The in situ forming material may be a two component mixture composed of a protein (e.g., serum albumin) in an aqueous buffer having a pH in the range of about 8.0-11.0 and a water-soluble bifunctional polyethylene oxide type crosslinking agent, which transforms from a liquid to a strong, flexible bonding composition to seal tissue in situ. See, e.g., U.S. Pat. Nos. 5,583,114 and RE38158 and PCT Publication No. WO 96/03159. The in situ forming material may be composed of a protein, a surfactant, and a lipid in a liquid carrier, which is crosslinked by adding a crosslinker and used as a sealant or bonding agent in situ. See, e.g., U.S. Patent Application No. 2004/0063613A1 and PCT Publication Nos. WO 01/45761 and WO 03/090683. The in situ forming material may be composed of two enzyme-free liquid components that are mixed by dispensing the components into a catheter tube deployed at the vascular puncture site, wherein, upon mixing, the two liquid components chemically cross-link to form a mechanical non-liquid matrix that seals a vascular puncture site. See, e.g., U.S. Patent Application Nos. 2002/0161399A1 and 2001/0018598A1. The in situ forming material may be a cross-linked albumin composition composed of an albumin preparation and a carbodiimide preparation which are mixed under conditions that permit crosslinking of the albumin for use as a bioadhesive or sealant. See, e.g., PCT Publication No. WO 99/66964. The in situ forming material may be composed of collagen and a peroxidase and hydrogen peroxide, such that the collagen is crosslinked to from a semi-solid gel that seals a wound. See, e.g., PCT Publication No. WO 01/35882.

In another aspect, in situ forming materials that can be used include those based on isocyanate or isothiocyanate capped polymers. For example, the in situ forming material may be composed of isocyanate-capped polymers that are liquid compositions which form into a solid adhesive coating by in situ polymerization and crosslinking upon contact with body fluid or tissue. See, e.g., PCT Publication No. WO 04/021983. The in situ forming material may be a moisture-curing sealant composition composed of an active isocyanato-terminated isocyanate prepolymer containing a polyol component with a molecular weight of 2,000 to 20,000 and an isocyanurating catalyst agent. See, e.g., U.S. Pat. No. 5,206,331.

In another embodiment, the anti-fibrosing agent can be coated onto the entire device or a portion of the device. In certain embodiments, the agent is present as part of a coating on a surface of the implantable sensor or implantable pump. The coating may partially cover or may completely cover the surface of the implantable sensor or implantable pump. Further, the coating may directly or indirectly contact the implantable sensor or implantable pump. For example, the Implantable sensor or implantable pump may be coated with a first coating and then coated with a second coating that includes the anti-scarring agent.

Implantable sensors and implantable pumps may be coated using a variety of coating methods, including by dipping, spraying, painting, by vacuum deposition, or by any other method known to those of ordinary skill in the art.

As described above, the anti-fibrosing agent can be coated onto the appropriate implantable sensors and implantable pumps using the polymeric coatings described above. In addition to the coating compositions and methods described above, there are various other coating compositions and methods that are known in the art. Representative examples of these coating compositions and methods are described in U.S. Pat. Nos. 6,610,016; 6,358,557; 6,306,176; 6,110,483; 6,106,473; 5,997,517; 5,800,412; 5,525,348; 5,331,027; 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267; U.S. Patent Application Publication Nos. 2002/0146581, 2003/0129130, 2001/0026834; 2003/0190420; 2001/0000785; 2003/0059631; 2003/0190405; 2002/0146581; 2003/020399; 2001/0026834; 2003/0190420; 2001/0000785; 2003/0059631; 2003/0190405; and 2003/020399; and PCT Publication Nos. WO 02/055121; WO 01/57048; WO 01/52915; and WO 01/01957.

Within another aspect of the invention, the biologically active fibrosis-inhibiting agent can be delivered with non-polymeric agents. These non-polymeric agents can include sucrose derivatives (e.g., sucrose acetate isobutyrate, sucrose oleate), sterols such as cholesterol, stigmasterol, beta-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites, and combinations and mixtures thereof.

Representative examples of patents relating to non-polymeric delivery systems and their preparation include U.S. Pat. Nos. 5,736,152; 5,888,533; 6,120,789; 5,968,542; and 5,747,058.

The fibrosis-inhibiting agent may be delivered as a solution. The fibrosis-inhibiting agent can be incorporated directly into the solution to provide a homogeneous solution or dispersion. In certain embodiments, the solution is an aqueous solution. The aqueous solution may futher include buffer salts, as well as viscosity modifying agents (e.g., hyaluronic acid, alginates, CMC, and the like). In another aspect of the invention, the solution can include a biocompatible solvent, such as ethanol, DMSO, glycerol, PEG-200, PEG-300 or NMP.

Within another aspect of the invention, the fibrosis-inhibiting agent can further comprise a secondary carrier. The secondary carrier can be in the form of microspheres (e.g., PLGA, PLLA, PDLLA, PCL, gelatin, polydioxanone, poly(alkylcyanoacrylate), nanospheres (e.g., PLGA, PLLA, PDLLA, PCL, gelatin, polydioxanone, poly(alkylcyanoacrylate)), liposomes, emulsions, microemulsions, micelles (e.g., SDS, block copolymers of the form X-Y, X-Y-X or Y-X-Y where X is a poly(alkylene oxide) or alkyl ether thereof and Y is a polyester (e.g., PLGA, PLLA, PDLLA, PCL polydioxanone)), zeolites or cyclodextrins.

Within another aspect of the invention, these fibrosis-inhibiting agent/secondary carrier compositions can be a) incorporated directly into, or onto, the implantable sensor or implantable pump, b) incorporated into a solution, c) incorporated into a gel or viscous solution, d) incorporated into the composition used for coating the implantable sensor or implantable pump, or e) incorporated into, or onto, the implantable sensor or implantable pump following coating of the implantable sensor or implantable pump with a coating composition.

For example, fibrosis-inhibiting agent loaded PLGA microspheres may be incorporated into a polyurethane coating solution which is then coated onto the implantable sensor or implantable pump.

In yet another example, the implantable sensor or implantable pump can be coated with a polyurethane and then allowed to partially dry such that the surface is still tacky. A particulate form of the fibrosis-inhibiting agent or fibrosis-inhibiting agent/secondary carrier can then be applied to all or a portion of the tacky coating after which the device is dried.

In yet another example, the implantable sensor or implantable pump can be coated with one of the coatings described above. A thermal treatment process can then be used to soften the coating, afterwhich the fibrosis-inhibiting agent or the fibrosis-inhibiting agent/secondary carrier is applied to the entire implantable sensor or implantable pump or to a portion of the implantable sensor or implantable pump (e.g., outer surface).

Within another aspect of the invention, the coated Implantable sensor or implantable pump which inhibits or reduces an in vivo fibrotic reaction is further coated with a compound or compositions which delay the release of and/or activity of the fibrosis-inhibiting agent. Representative examples of such agents include biologically inert materials such as gelatin, PLGA/MePEG film, PLA, polyurethanes, silicone rubbers, surfactants, lipids, or polyethylene glycol, as well as biologically active materials such as heparin (e.g., to induce coagulation).

For example, in one embodiment of the invention the fibrosis-inhibiting active agent on the implantable sensor or implantable pump is top-coated with a physical barrier. Such barriers can include non-degradable materials or biodegradable materials such as gelatin, PLGA/MePEG film, PLA, or polyethylene glycol among others. In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the blood or body fluids, the MePEG will dissolve out of the PLGA, leaving channels through the PLGA to an underlying layer containing the fibrosis-inhibiting agent, which then can then diffuse into the tissue and initiate its biological activity.

In another embodiment of the invention, for example, a particulate form of the active fibrosis-inhibiting agent may be coated onto the implantable sensor or implantable pump using a polymer (e.g., PLG, PLA, polyurethane). A second polymer that dissolves slowly or degrades (e.g., MePEG-PLGA or PLG) and that does not contain the active agent may be coated over the first layer. Once the top layer dissolves or degrades, it exposes the under coating which allows the active agent to be exposed to the treatment site or to be released from the coating.

Within another aspect of the invention, the outer layer of the coating of a coated Implantable sensor or implantable pump which inhibits an in vivo fibrotic response is further treated to crosslink the outer layer of the coating. This can be accomplished by subjecting the coated implantable sensor or implantable pump to a plasma treatment process. The degree of crosslinking and nature of the surface modification can be altered by changing the RF power setting, the location with respect to the plasma, the duration of treatment as well as the gas composition introduced into the plasma chamber.

Protection of a biologically active surface can also be utilized by coating the implantable sensor or implantable pump surface with an inert molecule that prevents access to the active site through steric hindrance, or by coating the surface with an inactive form of the fibrosis-inhibiting agent, which is later activated. For example, the implantable sensor or implantable pump can be coated with an enzyme, which causes either release of the fibrosis-inhibiting agent or activates the fibrosis-inhibiting agent.

Another example of a suitable implantable sensor or implantable pump surface coating includes an anticoagulant such as heparin or heparin quaternary amine complexes (e.g., heparin-benzalkonium chloride complex), which can be coated on top of the fibrosis-inhibiting agent. The presence of the anticoagulant delays coagulation. As the anticoagulant dissolves away, the anticoagulant activity may stop, and the newly exposed fibrosis-inhibiting agent may inhibit or reduce fibrosis from occurring in the adjacent tissue or coating the implantable sensor or implantable pump.

Another example of a suitable implantable sensor or implantable pump surface coating (particularly coatings for drug delivery catheters used in implantable pumps) includes an anti-infective agent such as an antibiotic, 5-FU, mitoxantrone, methotrexate, and/or doxyrubicin which can be incorporated into a coating that may, or may not, also contain a fibrosis-inhibiting agent. The presence of the anti-infective agent prevents infection in the tissues around the implant and can help prevent serious device-related infections (e.g., meningitis with intrathecal drug delivery pumps, peritonitis with intraperitoneal drug delivery pumps, endocarditis with cardiac drug delivery pumps).

In another aspect, the implantable sensor or implantable pump can be coated with an inactive form of the fibrosis-inhibiting agent, which is then activated once the device is deployed. Such activation may be achieved by injecting another material into the treatment area after the implantable sensor or implantable pump (as desribed below) is implanted or after the fibrosis-inhibiting agent has been administered to the treatment area (via injections, spray, wash, drug delivery catheters or balloons). In this aspect, the implantable sensor or implantable pump may be coated with an inactive form of the fibrosis-inhibiting agent. Once the implantable sensor or implantable pump is implanted, the activating substance is injected or applied into, or onto, the treatment site where the inactive form of the fibrosis-inhibiting agent has been applied.

One example of this method includes coating an implantable sensor or implantable pump with a biologically active fibrosis-inhibiting agent, in the usual manner. The coating containing the active fibrosis-inhibiting agent may then be covered with polyethylene glycol and these two substances may then be bonded through an ester bond using a condensation reaction. Prior to the deployment of the implantable sensor or implantable pump, an esterase is injected into the tissue around the outside of the device, which will cleave the bond between the ester and the fibrosis-inhibiting therapeutic, allowing the agent to initiate fibrosis inhibition.

In yet another aspect, anti-scarring agent may be located within pores or voids of the implantable sensor or implantable pump. For example, a implantable sensors and implantable pumps may be constructed to have cavities (e.g., divets or holes), grooves, lumen(s), pores, channels, and the like, which form voids or pores in the body of the implantable sensor or implantable pump. These voids may be filled (partially or completely) with a fibrosis-inhibiting agent or a composition that comprises a fibrosis-inhibiting agent.

In another aspect, an implantable sensor or implantable pump may include a plurality of reservoirs within its structure, each reservoir configured to house and protect a therapeutic drug. The reservoirs may be formed from divets in the device surface or micropores or channels in the device body. In one aspect, the reservoirs are formed from voids in the structure of the device. The reservoirs may house a single type of drug or more than one type of drug. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

Within certain embodiments of the invention, the therapeutic compositions may also comprise additional ingredients such as surfactants (e.g., PLURONICS, such as F-127, L-122, L-101, L-92, L-81, and L-61), anti-inflammatory agents (e.g., dexamethasone or asprin), anti-thrombotic agents (e.g., heparin, high activity heparin, heparin quaternary amine complexes (e.g., heparin benzalkonium chloride complex)), anti-infective agents (e.g., 5-fluorouracil, triclosan, rifamycim, and silver compounds), preservatives, anti-oxidants and/or anti-platelet agents.

Within certain embodiments of the invention, the device or therapeutic composition can also comprise radio-opaque, echogenic materials and magnetic resonance imaging (MRI) responsive materials (i.e., MRI contrast agents) to aid in visualization of the device under ultrasound, fluoroscopy and/or MRI. For example, a device may be made with or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a device by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium(III) chelates or iron oxide compounds) may be incorporated into or onto the device, such as, for example, as a component in a coating or within the void volume of the device (e.g., within a lumen, reservoir, or within the structural material used to form the device). In some embodiments, a medical device may include radio-opaque or MRI visible markers (e.g., bands) that may be used to orient and guide the device during the implantation procedure.

In another embodiment, these agents can be contained within the same coating layer as the therapeutic agent or they may be contained in a coating layer (as described above) that is either applied before or after the therapeutic agent containing layer.

Implantable pumps and sensor may, alternatively, or in addition, be visualized under visible light, using fluorescence, or by other spectroscopic means. Visualization agents that can be included for this purpose include dyes, pigments, and other colored agents. In one aspect, the medical implant may further include a colorant to improve visualization of the implant in vivo and/or ex vivo. Frequently, implants can be difficult to visualize upon insertion, especially at the margins of implant. A coloring agent can be incorporated into a medical implant to reduce or eliminate the incidence or severity of this problem. The coloring agent provides a unique color, increased contrast, or unique fluorescence characteristics to the device. In one aspect, a solid implant is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the devices and composition of the present invention may include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the devices and compositions of the present invention include one or more preservatives or bacteriostatic agents, present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, 5-fluorouracil, methotrexate, doxorubicin, mitoxantrone, rifamycin, chlorocresol, benzalkonium chlorides, and the like. Examples of the preservative include paraoxybenzoic acid esters, chlorobutanol, benzylalcohol, phenethyl alcohol, dehydroacetic acid, sorbic acid, etc. In one aspect, the compositions of the present invention include one or more bactericidal (also known as bacteriacidal) agents.

In one aspect, the devices and compositions of the present invention include one or more antioxidants, present in an effective amount. Examples of the antioxidant include sulfites, alpha-tocopherol and ascorbic acid.

Within certain aspects of the present invention, the devices and therapeutic compositions of the present invention should be biocompatible, and release one or more fibrosis-inhibiting agents over a period of several hours, days, or, months. As described above, “release of an agent” refers to any statistically significant presence of the agent, or a subcomponent thereof, which has disassociated from the compositions and/or remains active on the surface of (or within) the composition. The compositions of the present invention may release the anti-scarring agent at one or more phases, the one or more phases having similar or different performance (e.g., release) profiles. The therapeutic agent may be made available to the tissue at amounts which may be sustainable, intermittent, or continuous; in one or more phases; and/or rates of delivery; effective to reduce or inhibit any one or more components of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue).

Thus, release rate may be programmed to impact fibrosis (or scarring) by releasing anti-scarring agent at a time such that at least one of the components of fibrosis is inhibited or reduced. Moreover, the predetermined release rate may reduce agent loading and/or concentration as well as potentially providing minimal drug washout and thus, increases efficiency of drug effect. Any one of the at least one anti-scarring agents may perform one or more functions, including inhibiting the formation of new blood vessels (angiogenesis), inhibiting the migration and proliferation of connective tissue cells (such as fibroblasts or smooth muscle cells), inhibiting the deposition of extracellular matrix (ECM), and inhibiting remodeling (maturation and organization of the fibrous tissue). In one embodiment, the rate of release may provide a sustainable level of the anti-scarring agent to the susceptible tissue site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, and substantially non-releasing.

The total amount of anti-scarring agent made available on, in or near the device may be in an amount ranging from about 0.01 μg (micrograms) to about 2500 mg (milligrams). Generally, the anti-scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The surface area amount of anti-scarring agent on, in or near the device may be in an amount ranging from less than 0.01 μg to about 2500 μg per mm of device surface area. Generally, the anti-scarring agent may be in the amount ranging from less than 0.01 μg; or from 0.01 μg to about 10 μg; or from 10 μg to about 250 μg; or from 250 μg to about 2500 μg per mm².

The anti-scarring agent that is on, in or near the device may be released from the composition in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The amount of anti-scarring agent released from the composition as a function of time may be determined based on the in vitro release characteristics of the agent from the composition. The in vitro release rate may be determined by placing the anti-scarring agent within the composition or device in an appropriate buffer such as 0.1 M phosphate buffer (pH 7.4)) at 37° C. Samples of the buffer solution are then periodically removed for analysis by HPLC, and the buffer is replaced to avoid any saturation effects.

Based on the in vitro release rates, the release of anti-scarring agent per day may range from an amount ranging from about 0.01 μg (micrograms) to about 2500 mg (milligrams). Generally, the anti-scarring agent that may be released in a day may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

In one embodiment, the anti-scarring agent is made available to the susceptible tissue site in a programmed, sustained, and/or controlled manner which results in increased efficiency and/or efficacy. Further, the release rates may vary during either or both of the initial and subsequent release phases. There may also be additional phase(s) for release of the same substance(s) and/or different substance(s).

Further, therapeutic compositions and devices of the present invention should preferably have a stable shelf-life of at least several months and be capable of being produced and maintained under sterile conditions. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed asceptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Filtration may be accomplished using a filter with suitable pore size, for example 0.22 μm and of a suitable material, for instance polytetrafluoroethylene (e.g., TEFLON from E.I. DuPont De Nemours and Company, Wilmington, Del.).

In another aspect, the compositions and devices of the present invention are contained in a container that allows them to be used for their intended purpose, i.e., as a pharmaceutical composition. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

In one embodiment, the product containers can be thermoformed plastics. In another embodiment, a seconday package can be used for the product. In another embodiment, product can be in a sterile container that is placed in a box that is labeled to describe the contents of the box.

1. Coating Implantable Sensors and Pumps with Fibrosis-Inhibiting Agents

As described above, a range of polymeric and non-polymeric materials can be used to incorporate the fibrosis-inhibiting agent onto or into an implantable sensor or implantable pump. Coating the implantable sensor or implantable pump with these fibrosis-inhibiting agent-containing compositions, or with the fibrosis-inhibiting agent only, is one process that can be used to incorporate the fibrosis-inhibiting agent into or onto the implantable sensor or implantable pump.

a. Dip Coating

Dip coating is an example of coating process that can be used to associate the anti-scarring agent with the implantable sensor or implantable pump. In one embodiment, the fibrosis-inhibiting agent is dissolved in a solvent for the fibrosis-inhibiting agent and is then coated onto the implantable sensor or implantable pump (or part of the sensor or pump such as the body, the detector, the semipermeable membrane, the drug delivery catheter, or the drug delivery port).

Fibrosis-Inhibiting Agent with an Inert Solvent

In one embodiment, the solvent is an inert solvent for the implantable sensor or implantable pump such that the solvent does not dissolve the implantable device to any great extent and is not absorbed by the implantable device to any great extent. The implantable sensor or implantable pump (or part of the sensor or pump such as the body, the detector, the semipermeable membrane, the drug delivery catheter, or the drug delivery port) can be immersed, either partially or completely, in the fibrosis-inhibiting agent/solvent solution for a specific period of time. The rate of immersion into the fibrosis-inhibiting agent/solvent solution can be altered (e.g., 0.001 cm per sec to 50 cm per sec). The implantable sensor or implantable pump can then be removed from the solution. The rate at which the implantable sensor or implantable pump is withdrawn from the solution can be altered (e.g., 0.001 cm per sec to 50 cm per sec). The coated implantable sensor or implantable pump can be air-dried. The dipping process can be repeated one or more times depending on the specific application, where higher repetitions generally increase the amount of agent that is coated onto the implantable sensor or implantable pump (or part of the sensor or pump such as the body, the detector, the semipermeable membrane, the drug delivery catheter, or the drug delivery port). The implantable sensor or implantable pump can be dried under vacuum to reduce residual solvent levels. This process will result in the fibrosis-inhibiting agent being coated on the surface of the device.

Fibrosis-inhibiting Agent with a Swelling Solvent

In one embodiment, the solvent is one that will not dissolve the implantable sensor or implantable pump but will be absorbed by the device (or part of the sensor or pump such as the body, the detector, the semipermeable membrane, the drug delivery catheter, or the drug delivery port). In certain cases, these solvents can swell the implantable sensor or implantable pump to some extent. The implantable sensor or implantable pump can be immersed, either partially or completely, in the fibrosis-inhibiting agent/solvent solution for a specific period of time (seconds to days). The rate of immersion into the fibrosis-inhibiting agent/solvent solution can be altered (e.g., 0.001 cm per sec to 50 cm per sec). The implantable sensor or implantable pump can then be removed from the solution. The rate at which the implantable sensor or implantable pump is withdrawn from the solution can be altered (e.g., 0.001 cm per sec to 50 cm per sec). The coated implantable sensor or implantable pump can be air-dried. The dipping process can be repeated one or more times depending on the specific application. The implantable sensor or implantable pump can be dried under vacuum to reduce residual solvent levels. This process will result in the fibrosis-inhibiting agent being adsorbed into the implantable sensor or implantable pump (or part of the sensor or pump such as the body, the detector, the semipermeable membrane, the drug delivery catheter, or the drug delivery port). The fibrosis-inhibiting agent may also be present on the surface of the implantable sensor or implantable pump (or part of the sensor or pump such as the body, the detector, the semipermeable membrane, the drug delivery catheter, or the drug delivery port). The amount of surface associated fibrosis-inhibiting agent may be reduced by dipping the coated implantable sensor or implantable pump into a solvent for the fibrosis-inhibiting agent, or by spraying the implantable sensor or implantable pump with a solvent for the fibrosis-inhibiting agent.

Fibrosis-Inhibiting Agent with a Solvent

In one embodiment, the solvent is one that will be absorbed by the implantable sensor or implantable pump and that will dissolve the implantable sensor or implantable pump (or part of the sensor or pump such as the body, the detector, the semipermeable membrane, the drug delivery catheter, or the drug delivery port). The implantable sensor or implantable pump can be immersed, either partially or completely, in the fibrosis-inhibiting agent/solvent solution for a specific period of time (seconds to hours). The rate of immersion into the fibrosis-inhibiting agent/solvent solution can be altered (e.g., 0.001 cm per sec to 50 cm per sec). The implantable sensor or implantable pump can then be removed from the solution. The rate at which the implantable sensor or implantable pump is withdrawn from the solution can be altered (e.g., 0.001 cm per sec to 50 cm per sec). The coated implantable sensor or implantable pump can be air-dried. The dipping process can be repeated one or more times depending on the specific application. The implantable sensor or implantable pump can be dried under vacuum to reduce residual solvent levels. This process will result in the fibrosis-inhibiting agent being adsorbed into the implantable sensor or implantable pump (or part of the sensor or pump such as the body, the detector, the semipermeable membrane, the drug delivery catheter, or the drug delivery port) as well as being surface associated. Preferably, the exposure time of implantable sensor or implantable pump to the solvent does not incur significant permanent dimensional changes to the device (or part of the sensor or pump such as the body, the detector, the semipermeable membrane, the drug delivery catheter, or the drug delivery port). The fibrosis-inhibiting agent may also be present on the surface of the implantable sensor and implantable pump. The amount of surface associated fibrosis-inhibiting agent may be reduced by dipping the implantable sensor or implantable pump into a solvent for the fibrosis-inhibiting agent or by spraying the coated implantable sensor or implantable pump with a solvent for the fibrosis-inhibiting agent.

In one embodiment, the fibrosis-inhibiting agent and a polymer are dissolved in a solvent, for both the polymer and the fibrosis-inhibiting agent, and are then coated onto the implantable sensor and implantable pump (or part of the sensor or pump such as the body, the detector, the semipermeable membrane, the drug delivery catheter, or the drug delivery port).

In the above description the implantable sensor or implantable pump (or part of the sensor or pump such as the body, the detector, the semipermeable membrane, the drug delivery catheter, or the drug delivery port) can be one that has not been modified or one that has been further modified by coating with a polymer, surface treated by plasma treatment, flame treatment, corona treatment, surface oxidation or reduction, surface etching, mechanical smoothing or roughening, or grafting prior to the coating process.

In any one the above dip coating methods, the surface of the implantable sensor or implantable pump (or part of the sensor or pump such as the body, the detector, the semipermeable membrane, the drug delivery catheter, or the drug delivery port) can be treated with a plasma polymerization method prior to coating of the fibrosis-inhibiting agent or fibrosis-inhibiting agent-containing composition, such that a thin polymeric layer is deposited onto the implantable sensor or implantable pump surface. Examples of such methods include parylene coating of devices and the use of various monomers such hydrocyclosiloxane monomers. Parylene coating may be especially advantageous if the device, or portions of the device (such as the body, the detector, the semipermeable membrane, the drug delivery catheter, or the drug delivery port), are composed of materials (e.g., stainless steel, nitinol) that do not allow incorporation of the therapeutic agent(s) into the surface layer using one of the above methods. A parylene primer layer may be deposited onto the implantable sensor or implantable pump using a parylene coater (e.g., PDS 2010 LABCOTER2 from Cookson Electronics) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of Parylene N, and PARYLENE D, a dichlorinated derivative of PARYLENE N).

b. Spray Coating Implantable Sensors and Implantable Pumps

Spray coating is another coating process that can be used. In the spray coating process, a solution or suspension of the fibrosis-inhibiting agent, with or without a polymeric or non-polymeric carrier, is nebulized and directed to the implantable sensor or implantable pump (or part of the sensor or pump such as the body, the detector, the semipermeable membrane, the drug delivery catheter, or the drug delivery port) to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part # 14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosis-inhibiting agent is dissolved in a solvent for the fibrosis agent and is then sprayed onto the implantable sensor or implantable pump (or part of the sensor or pump such as the body, the detector, the semipermeable membrane, the drug delivery catheter, or the drug delivery port).

Fibrosis-Inhibiting Agent with an Inert Solvent

In one embodiment, the solvent is an inert solvent for the implantable sensor or implantable pump such that the solvent does not dissolve the medical implantable sensor or implantable pump to any great extent and is not absorbed to any great extent. The implantable sensor or implantable pump can be held in place or mounted onto a mandrel or rod that has the ability to move in an X, Y or Z plane or a combination of these planes. Using one of the above described spray devices, the implantable sensor or implantable pump (or part of the sensor or pump such as the body, the detector, the semipermeable membrane, the drug delivery catheter, or the drug delivery port) can be spray coated such that it is either partially or completely coated with the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated implantable sensor or implantable pump can be air-dried. The spray coating process can be repeated one or more times depending on the specific application. The implantable sensor or implantable pump can be dried under vacuum to reduce residual solvent levels. This process will result in the fibrosis-inhibiting agent being coated on the surface of the implantable sensor or implantable pump (or part of the sensor or pump such as the body, the detector, the semipermeable membrane, the drug delivery catheter, or the drug delivery port).

Fibrosis-Inhibiting Agent with a Swelling solvent

In one embodiment, the solvent is one that will not dissolve the implantable sensor or implantable pump but will be absorbed by it. These solvents can thus swell the implantable sensor or implantable pump to some extent. The implantable sensor or implantable pump (or part of the sensor or pump such as the body, the detector, the semipermeable membrane, the drug delivery catheter, or the drug delivery port) can be spray coated, either partially or completely, in the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated implantable sensor or implantable pump can be air-dried. The spray coating process can be repeated one or more times depending on the specific application. The implantable sensor or implantable pump can be dried under vacuum to reduce residual solvent levels. This process will result in the fibrosis-inhibiting agent being adsorbed into the implantable sensor or implantable pump (or part of the sensor or pump such as the body, the detector, the semipermeable membrane, the drug delivery catheter, or the drug delivery port). The fibrosis-inhibiting agent may also be present on the surface of the implantable sensor or implantable pump. The amount of surface associated fibrosis-inhibiting agent may be reduced by dipping the coated implantable sensor or implantable pump into a solvent for the fibrosis-inhibiting agent, or by spraying the coated implantable sensor or implantable pump with a solvent for the fibrosis-inhibiting agent.

Fibrosis-Inhibiting Agent with a Solvent

In one embodiment, the solvent is one that will be absorbed by the implantable sensor or implantable pump and that will dissolve it. The implantable sensor or implantable pump (or part of the sensor or pump such as the body, the detector, the semipermeable membrane, the drug delivery catheter, or the drug delivery port) can be spray coated, either partially or completely, in the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated implantable sensor or implantable pump can be air-dried. The spray coating process can be repeated one or more times depending on the specific application. The implantable sensor or implantable pump can be dried under vacuum to reduce residual solvent levels. This process will result in the fibrosis-inhibiting agent being adsorbed into the implantable sensor or implantable pump (or part of the sensor or pump such as the body, the detector, the semipermeable membrane, the drug delivery catheter, or the drug delivery port) as well as being surface associated. In the preferred embodiment, the exposure time of the implantable sensor or implantable pump to the solvent may not incur significant permanent dimensional changes to it. The fibrosis-inhibiting agent may also be present on the surface of the implantable sensor or implantable pump. The amount of surface associated fibrosis-inhibiting agent may be reduced by dipping the coated implantable sensor or implantable pump into a solvent for the fibrosis-inhibiting agent, or by spraying the coated implantable sensor or implantable pump with a solvent for the fibrosis-inhibiting agent.

In the above description the implantable sensor or implantable pump (or part of the sensor or pump such as the body, the detector, the semipermeable membrane, the drug delivery catheter, or the drug delivery port) can be one that has not been modified as well as one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosis-inhibiting agent and a polymer are dissolved in a solvent, for both the polymer and the anti-fibrosing agent, and are then spray coated onto the implantable sensor or implantable pump (or part of the sensor or pump such as the body, the detector, the semipermeable membrane, the drug delivery catheter, or the drug delivery port).

Fibrosis-Inhibiting Agent/Polymer with an Inert Solvent

In one embodiment, the solvent is an inert solvent for the implantable sensor or implantable pump such that the solvent does not dissolve it to any great extent and is not absorbed by it to any great extent. The implantable sensor or implantable pump (or part of the sensor or pump such as the body, the detector, the semipermeable membrane, the drug delivery catheter, or the drug delivery port) can be spray coated, either partially or completely, in the fibrosis-inhibiting agent/polymer/solvent solution for a specific period of time. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated implantable sensor or implantable pump can be air-dried. The spray coating process can be repeated one or more times depending on the specific application. The implantable sensor or implantable pump can be dried under vacuum to reduce residual solvent levels. This process will result in the fibrosis-inhibiting agent/polymer being coated on the surface of the device (or part of the sensor or pump such as the body, the detector, the semipermeable membrane, the drug delivery catheter, or the drug delivery port).

Fibrosis-Inhibiting Agent/Polymer with a Swelling Solvent

In one embodiment, the solvent is one that will not dissolve the implantable sensor or implantable pump but will be absorbed by it. These solvents can thus swell the implantable sensor or implantable pump to some extent. The implantable sensor or implantable pump (or part of the sensor or pump such as the body, the detector, the semipermeable membrane, the drug delivery catheter, or the drug delivery port) can be spray coated, either partially or completely, in the fibrosis-inhibiting agent/polymer/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated implantable sensor or implantable pump can be air-dried. The spray coating process can be repeated one or more times depending on the specific application. The implantable sensor or implantable pump can be dried under vacuum to reduce residual solvent levels. This process will result in the fibrosis-inhibiting agent/polymer being coated onto the surface of the implantable sensor or implantable pump as well as the potential for the fibrosis-inhibiting agent being adsorbed into the medical device (or part of the sensor or pump such as the body, the detector, the semipermeable membrane, the drug delivery catheter, or the drug delivery port). The fibrosis-inhibiting agent may also be present on the surface of the device. The amount of surface associated fibrosis-inhibiting agent may be reduced by dipping the coated implantable sensor or implantable pump into a solvent for the fibrosis-inhibiting agent or by spraying the coated implantable sensor or implantable pump with a solvent for the fibrosis-inhibiting agent.

Fibrosis-Inhibiting Agent/Polymer with a Solvent

In one embodiment, the solvent is one that will be absorbed by the implantable sensor or implantable pump and that will dissolve it. The implantable sensor or implantable pump (or part of the sensor or pump such as the body, the detector, the semipermeable membrane, the drug delivery catheter, or the drug delivery port) can be spray coated, either partially or completely, in the fibrosis-inhibiting agent/solvent solution. The rate of spraying of the fibrosis-inhibiting agent/solvent solution can be altered (e.g., 0.001 mL per sec to 10 mL per sec) to ensure that a good coating of the fibrosis-inhibiting agent is obtained. The coated implantable sensor or implantable pump can be air-dried. The spray coating process can be repeated one or more times depending on the specific application. The implantable sensor or implantable pump can be dried under vacuum to reduce residual solvent levels. In the preferred embodiment, the exposure time of the implantable sensor or implantable pump to the solvent may not incur significant permanent dimensional changes to it (other than those associated with the coating itself). The fibrosis-inhibiting agent may also be present on the surface of the device (or part of the sensor or pump such as the body, the detector, the semipermeable membrane, the drug delivery catheter, or the drug delivery port). The amount of surface associated fibrosis-inhibiting agent may be reduced by dipping the coated implantable sensor or implantable pump into a solvent for the fibrosis-inhibiting agent or by spraying the coated implantable sensor or implantable pump with a solvent for the fibrosis-inhibiting agent.

In the above description the implantable sensor or implantable pump (or part of the sensor or pump such as the body, the detector, the semipermeable membrane, the drug delivery catheter, or the drug delivery port) can be one that has not been modified as well as one that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In another embodiment, a suspension of the fibrosis-inhibiting agent in a polymer solution can be prepared. The suspension can be prepared by choosing a solvent that can dissolve the polymer but not the fibrosis-inhibiting agent, or a solvent that can dissolve the polymer and in which the fibrosis-inhibiting agent is above its solubility limit. In similar processes described above, the suspension of the fibrosis-inhibiting and polymer solution can be sprayed onto the implantable sensor or implantable pump (or part of the sensor or pump such as the body, the detector, the semipermeable membrane, the drug delivery catheter, or the drug delivery port) such that it is coated with a polymer that has a fibrosis-inhibiting agent suspended within it.

The present invention, in various aspects and embodiments, provides the following devices:

1. Sensor

In one aspect, the present invention provides a device, comprising a sensor and an anti-scarring agent or a composition comprising an anti-scarring agent, wherein the agent inhibits scarring between the device and a host into which the device is implanted.

Such a sensor may be defined by one, two, or more of the following features: the sensor is a blood or tissue glucose monitor; the sensor is an electrolyte sensor; the sensor is a blood constituent sensor; the sensor is a temperature sensor; the sensor is a pH sensor; the sensor is an optical sensor; the sensor is an amperometric sensor; the sensor is a pressure sensor; the sensor is a biosensor; the sensor is a sensing transponder; the sensor is a strain sensor; the sensor is a magnetoresistive sensor; the sensor is a cardiac sensor; the sensor is a respiratory sensor; the sensor is an auditory sensor; the sensor is a metabolite sensor; the sensor detects mechanical changes; the sensor detects physical changes; the sensor detects electrochemical changes; the sensor detects magnetic changes; the sensor detects acceleration changes; the sensor detects ionizing radiation changes; the sensor detects acoustic wave changes; the sensor detects chemical changes; the sensor detects drug concentration changes; and the sensor detects hormone changes; the sensor detects barometric changes.

2. Blood or Tissue Glucose Monitor (i.e., a Sensor)

In one aspect, the present invention provides a device, comprising a blood or tissue glucose monitor (i.e., a sensor) and an anti-scarring agent or a composition comprising an anti-scarring agent, wherein the agent inhibits scarring between the device and a host into which the device is implanted.

Such a device may be further defined by one, two, or more of the following features: the device is deliverable to the vascular system transluminally using a catheter on a stent platform; the device is composed of glucose sensitive living cells that monitor blood glucose levels and produce a detectable electrical or optical signal in response to changes in glucose concentrations; the device is an electrode composed of an analyte responsive enzyme; the device is a closed loop insulin delivery system that comprises a sensing means that detects the host's blood glucose level and stimulates an insulin pump to supply insulin; and the device is a closed loop insulin delivery system that comprises a sensing means that detects the host's blood glucose level and stimulates the pancreas to supply insulin.

3. Pressure or Stress Sensor

In one aspect, the present invention provides a device, comprising a pressure or stress sensor and an anti-scarring agent or a composition comprising an anti-scarring agent, wherein the agent inhibits scarring between the device and a host into which the device is implanted.

Such a device may be further defined by one, two, or more of the following features: the device monitors blood pressure; the device monitors fluid flow; the device monitors pressure within an aneurysm sac; the device monitors intracranial pressure; the device monitors mechanical pressure associated with a bone fracture; the device monitors barometric pressure; the device monitors eye tremors; the device monitors the depth of a corneal implant; the device monitors intraocular pressure; the device is a passive sensor with an inductor-capacitor circuit; the device is a self-powered strain sensing system; and the sensor comprises a lead, a sensor module, and a sensor circuit and means for providing voltage.

4. Cardiac Sensor

In one aspect, the present invention provides a device, comprising a cardiac sensor and an anti-scarring agent or a composition comprising an anti-scarring agent, wherein the agent inhibits scarring between the device and a host into which the device is implanted.

Such a device may be further defined by one, two, or more of the following features: the device monitors cardiac output; the device monitors ejection fraction; the device monitors blood pressure in a heart chamber; the device monitors ventricular wall motions; the device monitors blood flow to a transplanted organ; and the device monitors heart rate.

5. Respiratory Sensor

In one aspect, the present invention provides a device, comprising a respiratory sensor and an anti-scarring agent or a composition comprising an anti-scarring agent, wherein the agent inhibits scarring between the device and a host into which the device is implanted.

In one embodiment, the device monitors pulmonary functions.

6. Auditory Sensor

In one aspect, the present invention provides a device, comprising an auditory sensor and an anti-scarring agent or a composition comprising an anti-scarring agent, wherein the agent inhibits scarring between the device and a host into which the device is implanted.

Such a device may be further defined by one, two, or more of the following features: the device is adapted for delivering an electrical signal to an implantable electromechanical transducer that acts on the middle or inner ear; the device generates an electrical audio signal; the device is a capacitive sensor that is coupled to a vibrating auditory element; and the device is an electromagnetic sensor.

7. Electrolyte or Metabolite Sensor

In one aspect, the present invention provides a device, comprising an electrolyte or metabolite sensor and an anti-scarring agent or a composition comprising an anti-scarring agent, wherein the agent inhibits scarring between the device and a host into which the device is implanted.

Such a device may be further defined by one, two, or more of the following features: the device emits a source of radiation directed towards blood to interact with a plurality of detectors that provide an output signal; the device is a biosensing transponder composed of a dye that has optical properties that change in response to changes in the environment, a photosensor to sense the optical changes, and a transponder for transmitting data to a remote reader; and the device is a monolithic bioelectronic device for detecting at least one analyte within the host.

8. Pump

The present invention provides a device, comprising a pump and an anti-scarring agent or a composition comprising an anti-scarring agent, wherein the agent inhibits scarring between the device and a host into which the device is implanted.

Such a device may be further defined by one, two or more the following features: the device is adapted for delivering insulin; the device is adapted for delivering a narcotic; the device is adapted for delivering a chemotherapeutic agent; the device is adapted for delivering an anti-arrhythmic drug; the device is adapted for delivering an anti-spasmotic drug; the device is adapted for delivering an anti-spastic agent; the device is adapted for delivering an antibiotic; the device is adapted for delivering a drug only when changes in the host are detected; the device is adapted for delivering a drug as a continuous slow release; the device is adapted for delivering a drug at prescribed dosages in a pulsatile manner; the device is a programmable drug delivery pump; the device is adapted for intraocularly delivering a drug; the device is adapted for intrathecally delivering a drug; the device is adapted for intraperitoneally delivering a drug; the device is adapted for intra-arterially delivering a drug; the device is adapted for intracardiac delivery of a drug; the device is an implantable osmotic pump; the device is an ocular drug delivery pump; the device is metering system; the device is a peristaltic (roller) pump; the device is an electronically driven pump; the device is an elastomeric pump; the device is a spring contraction pump; the device is a gas-driven pump; the device is a hydraulic pump; the device is a piston-dependent pump; the device is a non-piston-dependent pump; the device is a dispensing chamber; the device is an infusion pump; and the device is a passive pump.

9. Implantable Insulin Pump

In one aspect, the present invention provides a device, comprising an implantable insulin pump and an anti-scarring agent or a composition comprising an anti-scarring agent, wherein the agent inhibits scarring between the device and a host into which the device is implanted.

In one embodiment, the implantable insulin pump comprises a single channel catheter with a sensor implanted in a vessel that transmits blood chemistry to the implantable insulin pump to dispense mediation through the catheter.

10. Intrathecal Durg Delivery Pump

In one aspect, the present invention provides a device, comprising an intrathecal drug delivery pump and an anti-scarring agent or a composition comprising an anti-scarring agent, wherein the agent inhibits scarring between the device and a host into which the device is implanted.

Such a device may be further defined by one, two or more the following features: the device is adapted for delivering pain medication directly into the cerebrospinal fluid of the intrathecal space surrounding the spinal cord; the device is adapted for delivering a drug to the brain; the device is adapted for intrathecal delivering baclofen; the device further comprises an intraspinal catheter; the device further comprises a second intrathecal drug delivery pump; and the device further comprises a catheter and an electrode.

11. Implantable Drug Delivery Pump for Chemotherapy

In one aspect, the present invention provides a device, comprising an implantable drug delivery pump for chemotherapy and an anti-scarring agent or a composition comprising an anti-scarring agent, wherein the agent inhibits scarring between the device and a host into which the device is implanted.

Such a medical device may be further defined by one, two, or more of the following features: the device is adapted for delivering 2′-deoxy 5-fluorouridine; the host has a solid tumor, and the device is adapted for infusing a chemotherapeutic agent to the solid tumor; the host has a tumor, and the device is adapted for infusing a chemotherapeutic agent to the blood vessels that supply the tumor; and the host has a hepatic tumor, and the device is adapted for delivering a chemotherapeutic agent to the artery that provides blood supply to the liver of the host.

12. Drug Delivery Pump for Treating Heart Disease

In one aspect, the present invention provides a device, comprising a drug delivery pump for treating heart disease and an anti-scarring agent or a composition comprising an anti-scarring agent, wherein the agent inhibits scarring between the device and a host into which the device is implanted.

In one embodiment, the device is an implantable cardiac electrode that delivers stimulation energy and dispenses drug adjacent to the stimulation site.

13. Drug Delivery Implant (i.e., a Pump)

In one aspect, the present invention provides a device, comprising a drug delivery implant (i.e., a pump) and an anti-scarring agent or a composition comprising an anti-scarring agent, wherein the agent inhibits scarring between the device and a host into which the device is implanted.

Additional Features Related to Sensors

The sensors described above may also be defined by one, two or more of the following features: the agent inhibits cell regeneration; the agent inhibits angiogenesis; the agent inhibits fibroblast migration; the agent inhibits fibroblast proliferation; the agent inhibits deposition of extracellular matrix; the agent inhibits tissue remodeling; the agent is an angiogenesis inhibitor; the agent is a 5-lipoxygenase inhibitor or antagonist; the agent is a chemokine receptor antagonist; the agent is a cell cycle inhibitor; the agent is a taxane; the agent is an anti-microtubule agent; the agent is paclitaxel; the agent is not paclitaxel; the agent is an analogue or derivative of paclitaxel; the agent is a vinca alkaloid; the agent is camptothecin or an analogue or derivative thereof; the agent is a podophyllotoxin; the agent is a podophyllotoxin, wherein the podophyllotoxin is etoposide or an analogue or derivative thereof; the agent is an anthracycline; the agent is an anthracycline, wherein the anthracycline is doxorubicin or an analogue or derivative thereof; the agent is an anthracycline, wherein the anthracycline is mitoxantrone or an analogue or derivative thereof; the agent is a platinum compound; the agent is a nitrosourea; the agent is a nitroimidazole; the agent is a folic acid antagonist; the agent is a cytidine analogue; the agent is a pyrimidine analogue; the agent is a fluoropyrimidine analogue; the agent is a purine analogue; the agent is a nitrogen mustard or an analogue or derivative thereof; the agent is a hydroxyurea; the agent is a mytomicin or an analogue or derivative thereof; the agent is an alkyl sulfonate; the agent is a benzamide or an analogue or derivative thereof; the agent is a nicotinamide or an analogue or derivative thereof; the agent is a halogenated sugar or an analogue or derivative thereof; the agent is a DNA alkylating agent; the agent is an anti-microtubule agent; the agent is a topoisomerase inhibitor; the agent is a DNA cleaving agent; the agent is an antimetabolite; the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; the agent is a cyclin dependent protein kinase inhibitor; the agent is an epidermal growth factor kinase inhibitor; the agent is an elastase inhibitor; the agent is a factor Xa inhibitor; the agent is a farnesyltransferase inhibitor; the agent is a fibrinogen antagonist; the agent is a guanylate cyclase stimulant; the agent is a heat shock protein 90 antagonist; the agent is a heat shock protein 90 antagonist, wherein the heat shock protein 90 antagonist is geldanamycin or an analogue or derivative thereof; the agent is a guanylate cyclase stimulant; the agent is a HMGCoA reductase inhibitor; the agent is a HMGCoA reductase inhibitor, wherein the HMGCoA reductase inhibitor is simvastatin or an analogue or derivative thereof; the agent is a hydroorotate dehydrogenase inhibitor; the agent is an IKK2 inhibitor; the agent is an IL-1 antagonist; the agent is an ICE antagonist; the agent is an IRAK antagonist; the agent is an IL-4 agonist; the agent is an immunomodulatory agent; the agent is sirolimus or an analogue or derivative thereof; the agent is not sirolimus; the agent is everolimus or an analogue or derivative thereof; the agent is tacrolimus or an analogue or derivative thereof; the agent is not tacrolimus; the agent is biolmus or an analogue or derivative thereof; the agent is tresperimus or an analogue or derivative thereof; the agent is auranofin or an analogue or derivative thereof; the agent is 27-O-demethylrapamycin or an analogue or derivative thereof; the agent is gusperimus or an analogue or derivative thereof; the agent is pimecrolimus or an analogue or derivative thereof; the agent is ABT-578 or an analogue or derivative thereof; the agent is an inosine monophosphate dehydrogenase (IMPDH) inhibitor; the agent is an IMPDH inhibitor, wherein the IMPDH inhibitor is mycophenolic acid or an analogue or derivative thereof; the agent is an IMPDH inhibitor, wherein the IMPDH inhibitor is 1-alpha-25 dihydroxy vitamin D3 or an analogue or derivative thereof; the agent is a leukotriene inhibitor; the agent is a MCP-1 antagonist; the agent is a MMP inhibitor; the agent is an NF kappa B inhibitor; the agent is an NF kappa B inhibitor, wherein the NF kappa B inhibitor is Bay 11-7082; the agent is an NO antagonist; the agent is a p38 MAP kinase inhibitor; the agent is a p38 MAP kinase inhibitor, wherein the p38 MAP kinase inhibitor is SB 202190; the agent is a phosphodiesterase inhibitor; the agent is a TGF beta inhibitor; the agent is a thromboxane A2 antagonist; the agent is a TNFa antagonist; the agent is a TACE inhibitor; the agent is a tyrosine kinase inhibitor; the agent is a vitronectin inhibitor; the agent is a fibroblast growth factor inhibitor; the agent is a protein kinase inhibitor; the agent is a PDGF receptor kinase inhibitor; the agent is an endothelial growth factor receptor kinase inhibitor; the agent is a retinoic acid receptor antagonist; the agent is a platelet derived growth factor receptor kinase inhibitor; the agent is a fibronogin antagonist; the agent is an antimycotic agent; the agent is an antimycotic agent, wherein the antimycotic agent is sulconizole; the agent is a bisphosphonate; the agent is a phospholipase A1 inhibitor; the agent is a histamine H1/H2/H3 receptor antagonist; the agent is a macrolide antibiotic; the agent is a GPIIb/IIIa receptor antagonist; the agent is an endothelin receptor antagonist; the agent is a peroxisome proliferator-activated receptor agonist; the agent is an estrogen receptor agent; the agent is a somastostatin analogue; the agent is a neurokinin 1 antagonist; the agent is a neurokinin 3 antagonist; the agent is a VLA-4 antagonist; the agent is an osteoclast inhibitor; the agent is a DNA topoisomerase ATP hydrolyzing inhibitor; the agent is an angiotensin I converting enzyme inhibitor; the agent is an angiotensin II antagonist; the agent is an enkephalinase inhibitor; the agent is a peroxisome proliferator-activated receptor gamma agonist insulin sensitizer; the agent is a protein kinase C inhibitor; the agent is a ROCK (rho-associated kinase) inhibitor; the agent is a CXCR3 inhibitor; the agent is an Itk inhibitor; the agent is a cytosolic phospholipase A2-alpha inhibitor; the agent is a PPAR agonist; the agent is an immunosuppressant; the agent is an Erb inhibitor; the agent is an apoptosis agonist; the agent is a lipocortin agonist; the agent is a VCAM-1 antagonist; the agent is a collagen antagonist; the agent is an alpha 2 integrin antagonist; the agent is a TNF alpha inhibitor; the agent is a nitric oxide inhibitor the agent is a cathepsin inhibitor; the agent is not an anti-inflammatory agent; the agent is not a steroid; the agent is not a glucocorticosteroid; the agent is not dexamethasone, beclomethasone, or dipropionate; the agent is not an anti-infective agent; the agent is not an antibiotic; the agent is not an anti-fugal agent; the agent is not beclomethasone; the agent is not dipropionate, the device further comprises a coating, wherein the coating comprises the anti-scarring agent and a polymer; the device further comprises a coating, wherein the coating comprises the anti-scarring agent; the device further comprises a coating, wherein the coating is disposed on a surface of the device; the device further comprises a coating, wherein the coating directly contacts the device; the device further comprises a coating, wherein the coating indirectly contacts the device; the device further comprises a coating, wherein the coating partially covers the device; the device further comprises a coating, wherein the coating completely covers the device; the device further comprises a coating, wherein the coating is a uniform coating; the device further comprises a coating, wherein the coating is a non-uniform coating; the device further comprises a coating, wherein the coating is a discontinuous coating; the device further comprises a coating, wherein the coating is a patterned coating; the device further comprises a coating, wherein the coating has a thickness of 100 μm or less; the device further comprises a coating, wherein the coating has a thickness of 10 μm or less; the device further comprises a coating, wherein the coating adheres to the surface of the device upon deployment of the device; the device further comprises a coating, wherein the coating is stable at room temperature for a period of 1 year; the device further comprises a coating, wherein the anti-scarring agent is present in the coating in an amount ranging between about 0.0001% to about 1% by weight; the device further comprises a coating, wherein the anti-scarring agent is present in the coating in an amount ranging between about 1% to about 10% by weight; the device further comprises a coating, wherein the anti-scarring agent is present in the coating in an amount ranging between about 10% to about 25% by weight; the device further comprises a coating, wherein the anti-scarring agent is present in the coating in an amount ranging between about 25% to about 70% by weight; the device further comprises a coating, wherein the coating further comprises a polymer; the device further comprises a first coating having a first composition and the second coating having a second composition; the device further comprises a first coating having a first composition and the second coating having a second composition, wherein the first composition and the second composition are different; the device further comprises a polymer; the device further comprises a polymeric carrier; the device further comprises a polymeric carrier, wherein the polymeric carrier comprises a copolymer; the device further comprises a polymeric carrier, wherein the polymeric carrier comprises a block copolymer; the device further comprises a polymeric carrier, wherein the polymeric carrier comprises a random copolymer, the device further comprises a polymeric carrier, wherein the polymeric carrier comprises a biodegradable polymer; the device further comprises a polymeric carrier, wherein the polymeric carrier comprises a non-biodegradable polymer; the device further comprises a polymeric carrier, wherein the polymeric carrier comprises a hydrophilic polymer; the device further comprises a polymeric carrier, wherein the polymeric carrier comprises a hydrophobic polymer; the device further comprises a polymeric carrier, wherein the polymeric carrier comprises a polymer having hydrophilic domains; the device further comprises a polymeric carrier, wherein the polymeric carrier comprises a polymer having hydrophobic domains; the device further comprises a polymeric carrier, wherein the polymeric carrier comprises a non-conductive polymer; the device further comprises a polymeric carrier, wherein the polymeric carrier comprises an elastomer; the device further comprises a polymeric carrier, wherein the polymeric carrier comprises a hydrogel; the device further comprises a polymeric carrier, wherein the polymeric carrier comprises a silicone polymer; the device further comprises a polymeric carrier, wherein the polymeric carrier comprises a hydrocarbon polymer; the device further comprises a polymeric carrier, wherein the polymeric carrier comprises a styrene-derived polymer; the device further comprises a polymeric carrier, wherein the polymeric carrier comprises a butadiene polymer; the device further comprises a polymeric carrier, wherein the polymeric carrier comprises a macromer; the device further comprises a polymeric carrier, wherein the polymeric carrier comprises a poly(ethylene glycol) polymer; the device further comprises a polymeric carrier, wherein the polymeric carrier comprises an amorphous polymer; the device further comprises a lubricious coating; the anti-scarring agent is located within pores or holes of the device; the anti-scarring agent is located within a channel, lumen, or divet of the device; the device further comprises a second pharmaceutically active agent; the device further comprises an anti-inflammatory agent; the device further comprises an agent that inhibits infection; the device further comprises an agent that inhibits infection, wherein the agent is an anthracycline; the device further comprises an agent that inhibits infection, wherein the agent is doxorubicin; the device further comprises an agent that inhibits infection, wherein the agent is mitoxantrone; the device further comprises an agent that inhibits infection, wherein the agent is a fluoropyrimidine; the device further comprises an agent that inhibits infection, wherein the agent is 5-fluorouracil (5-FU); the device further comprises an agent that inhibits infection, wherein the agent is a folic acid antagonist; the device further comprises an agent that inhibits infection, wherein the agent is methotrexate; the device further comprises an agent that inhibits infection, wherein the agent is a podophylotoxin; the device further comprises an agent that inhibits infection, wherein the agent is etoposide; the device further comprises an agent that inhibits infection, wherein the agent is a camptothecin; the device further comprises an agent that inhibits infection, wherein the agent is a hydroxyurea; the device further comprises an agent that inhibits infection, wherein the agent is a platinum complex; the device further comprises an agent that inhibits infection, wherein the agent is cisplatin; the device further comprises an anti-thrombotic agent; the device further comprises a visualization agent; the device further comprises a visualization agent, wherein the visualization agent is a radiopaque material, wherein the radiopaque material comprises a metal, a halogenated compound, or a barium containing compound; the device further comprises a visualization agent, wherein the visualization agent is a radiopaque material, wherein the radiopaque material comprises barium, tantalum, or technetium; the device further comprises a visualization agent, wherein the visualization agent is a MRI responsive material; the device further comprises a visualization agent, wherein the visualization agent comprises a gadolinium chelate; the device further comprises a visualization agent, wherein the visualization agent comprises iron, magnesium, manganese, copper, or chromium; the device further comprises a visualization agent, wherein the visualization agent comprises an iron oxide compound; the device further comprises a visualization agent, wherein the visualization agent comprises a dye, pigment, or colorant; the device further comprises an echogenic material; the device further comprises an echogenic material, wherein the echogenic material is in the form of a coating; the device is sterile; the anti-scarring agent inhibits adhesion between the device and a host into which the device is implanted; the device delivers the anti-scarring agent locally to tissue proximate to the device; the anti-scarring agent is released into tissue in the vicinity of the device after deployment of the device; the anti-scarring agent is released into tissue in the vicinity of the device after deployment of the device, wherein the tissue is connective tissue; the anti-scarring agent is released into tissue in the vicinity of the device after deployment of the device, wherein the tissue is muscle tissue; the anti-scarring agent is released into tissue in the vicinity of the device after deployment of the device, wherein the tissue is nerve tissue; the anti-scarring agent is released into tissue in the vicinity of the device after deployment of the device, wherein the tissue is epithelium tissue; the anti-scarring agent is released in effective concentrations from the device over a period ranging from the time of deployment of the device to about 1 year; the anti-scarring agent is released in effective concentrations from the device over a period ranging from about 1 month to 6 months; the anti-scarring agent is released in effective concentrations from the device over a period ranging from about 1-90 days; the anti-scarring agent is released in effective concentrations from the device at a constant rate; the anti-scarring agent is released in effective concentrations from the device at an increasing rate; the anti-scarring agent is released in effective concentrations from the device at a decreasing rate; the anti-scarring agent is released in effective concentrations from the composition comprising the anti-scarring agent by diffusion over a period ranging from the time of deployment of the device to about 90 days; the anti-scarring agent is released in effective concentrations from the composition comprising the anti-scarring agent by erosion of the composition over a period ranging from the time of deployment of the device to about 90 days; the device comprises about 0.01 μg to about 10 μg of the anti-scarring agent; the device comprises about 10 μg to about 10 mg of the anti-scarring agent; the device comprises about 10 mg to about 250 mg of the anti-scarring agent; the device comprises about 250 mg to about 1000 mg of the anti-scarring agent; the device comprises about 1000 mg to about 2500 mg of the anti-scarring agent; a surface of the device comprises less than 0.01 μg of the anti-scarring agent per mm² of device surface to which the anti-scarring agent is applied; a surface of the device comprises about 0.01 μg to about 1 μg of the anti-scarring agent per mm² of device surface to which the anti-scarring agent is applied; a surface of the device comprises about 1 μg to about 10 μg of the anti-scarring agent per mm² of device surface to which the anti-scarring agent is applied; a surface of the device comprises about 10 μg to about 250 μg of the anti-scarring agent per mm² of device surface to which the anti-scarring agent is applied; a surface of the device comprises about 250 μg to about 1000 μg of the anti-scarring agent of anti-scarring agent per mm² of device surface to which the anti-scarring agent is applied; a surface of the device comprises about 1000 μg to about 2500 μg of the anti-scarring agent per mm² of device surface to which the anti-scarring agent is applied; the agent or the composition is affixed to the sensor; the agent or the composition is covalently attached to the sensor; the agent or the composition is non-covalently attached to the sensor; the device further comprises a coating that absorbs the agent or the composition; the sensor is interweaved with a thread composed of, or coated with, the agent or the composition; a portion of the sensor is covered with a sleeve that contains the agent or the composition; the sensor is completely covered with a sleeve that contains the agent or the composition; a portion of the sensor is covered with a mesh that contains the agent or the composition; the sensor is completely covered with a mesh that contains the agent or the composition; and the device further comprises a pump that is linked to the sensor.

Additional Features Related to Pumps

The pumps described above may also be defined by one, two or more of the following features: the agent inhibits cell regeneration; the agent inhibits angiogenesis; the agent inhibits fibroblast migration; the agent inhibits fibroblast proliferation; the agent inhibits deposition of extracellular matrix; the agent inhibits tissue remodeling; the agent is an angiogenesis inhibitor; the agent is a 5-lipoxygenase inhibitor or antagonist; the agent is a chemokine receptor antagonist; the agent is a cell cycle inhibitor; the agent is a taxane; the agent is an anti-microtubule agent; the agent is paclitaxel; the agent is not paclitaxel; the agent is an analogue or derivative of paclitaxel; the agent is a vinca alkaloid; the agent is camptothecin or an analogue or derivative thereof; the agent is a podophyllotoxin; the agent is a podophyllotoxin, wherein the podophyllotoxin is etoposide or an analogue or derivative thereof; the agent is an anthracycline; the agent is an anthracycline, wherein the anthracycline is doxorubicin or an analogue or derivative thereof; the agent is an anthracycline, wherein the anthracycline is mitoxantrone or an analogue or derivative thereof; the agent is a platinum compound; the agent is a nitrosourea; the agent is a nitroimidazole; the agent is a folic acid antagonist; the agent is a cytidine analogue; the agent is a pyrimidine analogue; the agent is a fluoropyrimidine analogue; the agent is a purine analogue; the agent is a nitrogen mustard or an analogue or derivative thereof; the agent is a hydroxyurea; the agent is a mytomicin or an analogue or derivative thereof; the agent is an alkyl sulfonate; the agent is a benzamide or an analogue or derivative thereof; the agent is a nicotinamide or an analogue or derivative thereof; the agent is a halogenated sugar or an analogue or derivative thereof; the agent is a DNA alkylating agent; the agent is an anti-microtubule agent; the agent is a topoisomerase inhibitor; the agent is a DNA cleaving agent; the agent is an antimetabolite; the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; the agent is a cyclin dependent protein kinase inhibitor; the agent is an epidermal growth factor kinase inhibitor; the agent is an elastase inhibitor; the agent is a factor Xa inhibitor; the agent is a farnesyltransferase inhibitor; the agent is a fibrinogen antagonist; the agent is a guanylate cyclase stimulant; the agent is a heat shock protein 90 antagonist; the agent is a heat shock protein 90 antagonist, wherein the heat shock protein 90 antagonist is geldanamycin or an analogue or derivative thereof; the agent is a guanylate cyclase stimulant; the agent is a HMGCoA reductase inhibitor; the agent is a HMGCoA reductase inhibitor, wherein the HMGCoA reductase inhibitor is simvastatin or an analogue or derivative thereof; the agent is a hydroorotate dehydrogenase inhibitor; the agent is an IKK2 inhibitor; the agent is an IL-1 antagonist; the agent is an ICE antagonist; the agent is an IRAK antagonist; the agent is an IL-4 agonist; the agent is an immunomodulatory agent; the agent is sirolimus or an analogue or derivative thereof; the agent is not sirolimus; the agent is everolimus or an analogue or derivative thereof; the agent is tacrolimus or an analogue or derivative thereof; the agent is not tacrolimus; the agent is biolmus or an analogue or derivative thereof; the agent is tresperimus or an analogue or derivative thereof; the agent is auranofin or an analogue or derivative thereof; the agent is 27-O-demethylrapamycin or an analogue or derivative thereof; the agent is gusperimus or an analogue or derivative thereof; the agent is pimecrolimus or an analogue or derivative thereof; the agent is ABT-578 or an analogue or derivative thereof; the agent is an inosine monophosphate dehydrogenase (IMPDH) inhibitor; the agent is an IMPDH inhibitor, wherein the IMPDH inhibitor is mycophenolic acid or an analogue or derivative thereof; the agent is an IMPDH inhibitor, wherein the IMPDH inhibitor is 1-alpha-25 dihydroxy vitamin D3 or an analogue or derivative thereof; the agent is a leukotriene inhibitor; the agent is a MCP-1 antagonist; the agent is a MMP inhibitor; the agent is an NF kappa B inhibitor; the agent is an NF kappa B inhibitor, wherein the NF kappa B inhibitor is Bay 11-7082; the agent is an NO antagonist; the agent is a p38 MAP kinase inhibitor; the agent is a p38 MAP kinase inhibitor, wherein the p38 MAP kinase inhibitor is SB 202190; the agent is a phosphodiesterase inhibitor; the agent is a TGF beta inhibitor; the agent is a thromboxane A2 antagonist; the agent is a TNFa antagonist; the agent is a TACE inhibitor; the agent is a tyrosine kinase inhibitor; the agent is a vitronectin inhibitor; the agent is a fibroblast growth factor inhibitor; the agent is a protein kinase inhibitor; the agent is a PDGF receptor kinase inhibitor; the agent is an endothelial growth factor receptor kinase inhibitor; the agent is a retinoic acid receptor antagonist; the agent is a platelet derived growth factor receptor kinase inhibitor; the agent is a fibronogin antagonist; the agent is an antimycotic agent; the agent is an antimycotic agent, wherein the antimycotic agent is sulconizole; the agent is a bisphosphonate; the agent is a phospholipase A1 inhibitor; the agent is a histamine H1/H2/H3 receptor antagonist; the agent is a macrolide antibiotic; the agent is a GPIIb/IIIa receptor antagonist; the agent is an endothelin receptor antagonist; the agent is a peroxisome proliferator-activated receptor agonist; the agent is an estrogen receptor agent; the agent is a somastostatin analogue; the agent is a neurokinin 1 antagonist; the agent is a neurokinin 3 antagonist; the agent is a VLA-4 antagonist; the agent is an osteoclast inhibitor; the agent is a DNA topoisomerase ATP hydrolyzing inhibitor; the agent is an angiotensin I converting enzyme inhibitor; the agent is an angiotensin II antagonist; the agent is an enkephalinase inhibitor; the agent is a peroxisome proliferator-activated receptor gamma agonist insulin sensitizer; the agent is a protein kinase C inhibitor; the agent is a ROCK (rho-associated kinase) inhibitor; the agent is a CXCR3 inhibitor; the agent is an Itk inhibitor; the agent is a cytosolic phospholipase A2-alpha inhibitor; the agent is a PPAR agonist; the agent is an immunosuppressant; the agent is an Erb inhibitor; the agent is an apoptosis agonist; the agent is a lipocortin agonist; the agent is a VCAM-1 antagonist; the agent is a collagen antagonist; the agent is an alpha 2 integrin antagonist; the agent is a TNF alpha inhibitor; the agent is a nitric oxide inhibitor the agent is a cathepsin inhibitor; the agent is not an anti-inflammatory agent; the agent is not a steroid; the agent is not a glucocorticosteroid; the agent is not dexamethasone, beclomethasone, or dipropionate; the agent is not an anti-infective agent; the agent is not an antibiotic; the agent is not an anti-fugal agent; the agent is not beclomethasone; the agent is not dipropionate; the device further comprises a coating, wherein the coating comprises the anti-scarring agent and a polymer; the device further comprises a coating, wherein the coating comprises the anti-scarring agent; the device further comprises a coating, wherein the coating is disposed on a surface of the device; the device further comprises a coating, wherein the coating directly contacts the device; the device further comprises a coating, wherein the coating indirectly contacts the device; the device further comprises a coating, wherein the coating partially covers the device; the device further comprises a coating, wherein the coating completely covers the device; the device further comprises a coating, wherein the coating is a uniform coating; the device further comprises a coating, wherein the coating is a non-uniform coating; the device further comprises a coating, wherein the coating is a discontinuous coating; the device further comprises a coating, wherein the coating is a patterned coating; the device further comprises a coating, wherein the coating has a thickness of 100 μm or less; the device further comprises a coating, wherein the coating has a thickness of 10 μm or less; the device further comprises a coating, wherein the coating adheres to the surface of the device upon deployment of the device; the device further comprises a coating, wherein the coating is stable at room temperature for a period of 1 year; the device further comprises a coating, wherein the anti-scarring agent is present in the coating in an amount ranging between about 0.0001% to about 1% by weight; the device further comprises a coating, wherein the anti-scarring agent is present in the coating in an amount ranging between about 1% to about 10% by weight; the device further comprises a coating, wherein the anti-scarring agent is present in the coating in an amount ranging between about 10% to about 25% by weight; the device further comprises a coating, wherein the anti-scarring agent is present in the coating in an amount ranging between about 25% to about 70% by weight; the device further comprises a coating, wherein the coating further comprises a polymer; the device further comprises a first coating having a first composition and the second coating having a second composition; the device further comprises a first coating having a first composition and the second coating having a second composition, wherein the first composition and the second composition are different; the device further comprises a polymer; the device further comprises a polymeric carrier; the device further comprises a polymeric carrier, wherein the polymeric carrier comprises a copolymer; the device further comprises a polymeric carrier, wherein the polymeric carrier comprises a block copolymer; the device further comprises a polymeric carrier, wherein the polymeric carrier comprises a random copolymer; the device further comprises a polymeric carrier, wherein the polymeric carrier comprises a biodegradable polymer; the device further comprises a polymeric carrier, wherein the polymeric carrier comprises a non-biodegradable polymer; the device further comprises a polymeric carrier, wherein the polymeric carrier comprises a hydrophilic polymer; the device further comprises a polymeric carrier, wherein the polymeric carrier comprises a hydrophobic polymer; the device further comprises a polymeric carrier, wherein the polymeric carrier comprises a polymer having hydrophilic domains; the device further comprises a polymeric carrier, wherein the polymeric carrier comprises a polymer having hydrophobic domains; the device further comprises a polymeric carrier, wherein the polymeric carrier comprises a non-conductive polymer; the device further comprises a polymeric carrier, wherein the polymeric carrier comprises an elastomer; the device further comprises a polymeric carrier, wherein the polymeric carrier comprises a hydrogel; the device further comprises a polymeric carrier, wherein the polymeric carrier comprises a silicone polymer; the device further comprises a polymeric carrier, wherein the polymeric carrier comprises a hydrocarbon polymer; the device further comprises a polymeric carrier, wherein the polymeric carrier comprises a styrene-derived polymer; the device further comprises a polymeric carrier, wherein the polymeric carrier comprises a butadiene polymer; the device further comprises a polymeric carrier, wherein the polymeric carrier comprises a macromer; the device further comprises a polymeric carrier, wherein the polymeric carrier comprises a poly(ethylene glycol) polymer; the device further comprises a polymeric carrier, wherein the polymeric carrier comprises an amorphous polymer; the device further comprises a lubricious coating; the anti-scarring agent is located within pores or holes of the device; the anti-scarring agent is located within a channel, lumen, or divet of the device; the device further comprises a second pharmaceutically active agent; the device further comprises an anti-inflammatory agent; the device further comprises an agent that inhibits infection; the device further comprises an agent that inhibits infection, wherein the agent is an anthracycline; the device further comprises an agent that inhibits infection, wherein the agent is doxorubicin; the device further comprises an agent that inhibits infection, wherein the agent is mitoxantrone; the device further comprises an agent that inhibits infection, wherein the agent is a fluoropyrimidine; the device further comprises an agent that inhibits infection, wherein the agent is 5-fluorouracil (5-FU); the device further comprises an agent that inhibits infection, wherein the agent is a folic acid antagonist; the device further comprises an agent that inhibits infection, wherein the agent is methotrexate; the device further comprises an agent that inhibits infection, wherein the agent is a podophylotoxin; the device further comprises an agent that inhibits infection, wherein the agent is etoposide; the device further comprises an agent that inhibits infection, wherein the agent is a camptothecin; the device further comprises an agent that inhibits infection, wherein the agent is a hydroxyurea; the device further comprises an agent that inhibits infection, wherein the agent is a platinum complex; the device further comprises an agent that inhibits infection, wherein the agent is cisplatin; the device further comprises an anti-thrombotic agent; the device further comprises a visualization agent; the device further comprises a visualization agent, wherein the visualization agent is a radiopaque material, wherein the radiopaque material comprises a metal, a halogenated compound, or a barium containing compound; the device further comprises a visualization agent, wherein the visualization agent is a radiopaque material, wherein the radiopaque material comprises barium, tantalum, or technetium; the device further comprises a visualization agent, wherein the visualization agent is a MRI responsive material; the device further comprises a visualization agent, wherein the visualization agent comprises a gadolinium chelate; the device further comprises a visualization agent, wherein the visualization agent comprises iron, magnesium, manganese, copper, or chromium; the device further comprises a visualization agent, wherein the visualization agent comprises an iron oxide compound; the device further comprises a visualization agent, wherein the visualization agent comprises a dye, pigment, or colorant; the device further comprises an echogenic material; the device further comprises an echogenic material, wherein the echogenic material is in the form of a coating; the device is sterile; the anti-scarring agent inhibits adhesion between the device and a host into which the device is implanted; the device delivers the anti-scarring agent locally to tissue proximate to the device; the anti-scarring agent is released into tissue in the vicinity of the device after deployment of the device; the anti-scarring agent is released into tissue in the vicinity of the device after deployment of the device, wherein the tissue is connective tissue; the anti-scarring agent is released into tissue in the vicinity of the device after deployment of the device, wherein the tissue is muscle tissue; the anti-scarring agent is released into tissue in the vicinity of the device after deployment of the device, wherein the tissue is nerve tissue; the anti-scarring agent is released into tissue in the vicinity of the device after deployment of the device, wherein the tissue is epithelium tissue; the anti-scarring agent is released in effective concentrations from the device over a period ranging from the time of deployment of the device to about 1 year; the anti-scarring agent is released in effective concentrations from the device over a period ranging from about 1 month to 6 months; the anti-scarring agent is released in effective concentrations from the device over a period ranging from about 1-90 days; the anti-scarring agent is released in effective concentrations from the device at a constant rate; the anti-scarring agent is released in effective concentrations from the device at an increasing rate; the anti-scarring agent is released in effective concentrations from the device at a decreasing rate; the anti-scarring agent is released in effective concentrations from the composition comprising the anti-scarring agent by diffusion over a period ranging from the time of deployment of the device to about 90 days; the anti-scarring agent is released in effective concentrations from the composition comprising the anti-scarring agent by erosion of the composition over a period ranging from the time of deployment of the device to about 90 days; the device comprises about 0.01 μg to about 10 μg of the anti-scarring agent; the device comprises about 10 μg to about 10 mg of the anti-scarring agent; the device comprises about 10 mg to about 250 mg of the anti-scarring agent; the device comprises about 250 mg to about 1000 mg of the anti-scarring agent; the device comprises about 1000 mg to about 2500 mg of the anti-scarring agent; a surface of the device comprises less than 0.01 μg of the anti-scarring agent per mm² of device surface to which the anti-scarring agent is applied; a surface of the device comprises about 0.01 μg to about 1 μg of the anti-scarring agent per mm² of device surface to which the anti-scarring agent is applied; a surface of the device comprises about 1 μg to about 10 μg of the anti-scarring agent per mm² of device surface to which the anti-scarring agent is applied; a surface of the device comprises about 10 μg to about 250 μg of the anti-scarring agent per mm² of device surface to which the anti-scarring agent is applied; a surface of the device comprises about 250 μg to about 1000 μg of the anti-scarring agent of anti-scarring agent per mm² of device surface to which the anti-scarring agent is applied; a surface of the device comprises about 1000 μg to about 2500 μg of the anti-scarring agent per mm² of device surface to which the anti-scarring agent is applied; the agent or the composition is affixed to the pump; the agent or the composition is covalently attached to the pump; the agent or the composition is non-covalently attached to the pump; the device further comprises a coating that absorbs the agent or the composition; the pump is interweaved with a thread composed of, or coated with, the agent or the composition; a portion of the pump is covered with a sleeve that contains the agent or the composition; the pump is completely covered with a sleeve that contains the agent or the composition; a portion of the pump is covered with a mesh that contains the agent or the composition; the pump is completely covered with a mesh that contains the agent or the composition; and the device further comprises a sensor that is linked to the pump.

The present invention, in various aspects and embodiments, provides the following methods for inhibiting scarring:

1. Sensor

In one aspect, the present invention provides a method for inhibiting scarring comprising placing a sensor and an anti-scarring agent or a composition comprising an anti-scarring agent into an animal host, wherein the agent inhibits scarring.

Such a method may be defined by one, two, or more of the following features: the sensor is a blood or tissue glucose monitor; the sensor is an electrolyte sensor; the sensor is a blood constituent sensor; the sensor is a temperature sensor; the sensor is a pH sensor; the sensor is an optical sensor; the sensor is an amperometric sensor; the sensor is a pressure sensor; the sensor is a biosensor; the sensor is a sensing transponder; the sensor is a strain sensor; the sensor is a magnetoresistive sensor; the sensor is a cardiac sensor; the sensor is a respiratory sensor; the sensor is an auditory sensor; the sensor is a metabolite sensor; the sensor detects mechanical changes; the sensor detects physical changes; the sensor detects electrochemical changes; the sensor detects magnetic changes; the sensor detects acceleration changes; the sensor detects ionizing radiation changes; the sensor detects acoustic wave changes; the sensor detects chemical changes; the sensor detects drug concentration changes; the sensor detects hormone changes; and the sensor detects barometric changes.

2. Blood or Tissue Glucose Monitoror

In one aspect, the present invention provides a method for inhibiting scarring comprising placing a blood or tissue glucose monitor (i.e., a sensor) and an anti-scarring agent or a composition comprising an anti-scarring agent into an animal host, wherein the agent inhibits scarring.

Such a method may be further defined by one, two, or more of the following features: the device is deliverable to the vascular system transluminally using a catheter on a stent platform; the device is composed of glucose sensitive living cells that monitor blood glucose levels and produce a detectable electrical or optical signal in response to changes in glucose concentrations; the device is an electrode composed of an analyte responsive enzyme; the device is a closed loop insulin delivery system that comprises a sensing means that detects the host's blood glucose level and stimulates an insulin pump to supply insulin; and the device is a closed loop insulin delivery system that comprises a sensing means that detects the host's blood glucose level and stimulates the pancreas to supply insulin.

3. Pressure or Stress Sensor

In one aspect, the present invention provides a method for inhibiting scarring comprising placing a pressure or stress sensor and an anti-scarring agent or a composition comprising an anti-scarring agent into an animal host, wherein the agent inhibits scarring.

Such a method may be further defined by one, two, or more of the following features: the device monitors blood pressure; the device monitors fluid flow; the device monitors pressure within an aneurysm sac; the device monitors intracranial pressure; the device monitors mechanical pressure associated with a bone fracture; the device monitors barometric pressure; the device monitors eye tremors; the device monitors the depth of a corneal implant; the device monitors intraocular pressure; the device is a passive sensor with an inductor-capacitor circuit; the device is a self-powered strain sensing system; the sensor comprises a lead, a sensor module, and a sensor circuit and means for providing voltage.

4. Cardiac Sensor

In one aspect, the present invention provides a method for for inhibiting scarring comprising placing a cardiac sensor and an anti-scarring agent or a composition comprising an anti-scarring agent into an animal host, wherein the agent inhibits scarring.

Such a method may be further defined by one, two, or more of the following features: the device monitors cardiac output; the device monitors ejection fraction; the device monitors blood pressure in a heart chamber; the device monitors ventricular wall motions; the device monitors blood flow to a transplanted organ; and the device monitors heart rate.

5. Respiratory Sensor

In one aspect, the present invention provides a method for inhibiting scarring comprising placing a respiratory sensor and an anti-scarring agent or a composition comprising an anti-scarring agent into an animal host, wherein the agent inhibits scarring.

In one embodiment, the device monitors pulmonary functions.

6. Auditory Sensor

In one aspect, the present invention provides a method for inhibiting scarring comprising placing a respiratory sensor and an anti-scarring agent or a composition comprising an anti-scarring agent into an animal host, wherein the agent inhibits scarring.

Such a method may be further defined by one, two, or more of the following features: the device is adapted for delivering an electrical signal to an implantable electromechanical transducer that acts on the middle or inner ear; the device generates an electrical audio signal; the device is a capacitive sensor that is coupled to a vibrating auditory element; and the device is an electromagnetic sensor.

7. Electrolyte or Metabolite Sensor

In one aspect, the present invention provides a method for inhibiting scarring comprising placing an electrolyte or metabolite sensor and an anti-scarring agent or a composition comprising an anti-scarring agent into an animal host, wherein the agent inhibits scarring.

Such a method may be further defined by one, two, or more of the following features: the device emits a source of radiation directed towards blood to interact with a plurality of detectors that provide an output signal; the device is a biosensing transponder composed of a dye that has optical properties that change in response to changes in the environment, a photosensor to sense the optical changes, and a transponder for transmitting data to a remote reader; and the device is a monolithic bioelectronic device for detecting at least one analyte within the host.

8. Pump

The present invention provides a method for inhibiting scarring comprising placing a pump and an anti-scarring agent or a composition comprising an anti-scarring agent into an animal host, wherein the agent inhibits scarring.

Such a method may be further defined by one, two or more the following features: the device is adapted for delivering insulin; the device is adapted for delivering a narcotic; the device is adapted for delivering a chemotherapeutic agent; the device is adapted for delivering an anti-arrhythmic drug; the device is adapted for delivering an anti-spasmotic drug; the device is adapted for delivering an anti-spastic agent; the device is adapted for delivering an antibiotic; the device is adapted for delivering a drug only when changes in the host are detected; the device is adapted for delivering a drug as a continuous slow release; the device is adapted for delivering a drug at prescribed dosages in a pulsatile manner; the device is a programmable drug delivery pump; the device is adapted for intraocularly delivering a drug; the device is adapted for intrathecally delivering a drug; the device is adapted for intraperitoneally delivering a drug; the device is adapted for intra-arterially delivering a drug; the device is adapted for intracardiac delivery of a drug; the device is an implantable osmotic pump; the device is an ocular drug delivery pump; the device is metering system; the device is a peristaltic (roller) pump; the device is an electronically driven pump; the device is an elastomeric pump; the device is a spring contraction pump; the device is a gas-driven pump; the device is a hydraulic pump; the device is a piston-dependent pump; the device is a non-piston-dependent pump; the device is a dispensing chamber; the device is an infusion pump; and the device is a passive pump.

9. Implantable Insulin Pump

In one aspect, the present invention provides a method for for inhibiting scarring comprising placing an implantable insulin pump and an anti-scarring agent or a composition comprising an anti-scarring agent into an animal host, wherein the agent inhibits scarring.

In one embodiment, the implantable insulin pump comprises a single channel catheter with a sensor implanted in a vessel that transmits blood chemistry to the implantable insulin pump to dispense mediation through the catheter.

10. Intrathecal Durg Delivery Pump

In one aspect, the present invention provides a method for for inhibiting scarring comprising placing an intrathecal pump and an anti-scarring agent or a composition comprising an anti-scarring agent into an animal host, wherein the agent inhibits scarring.

Such a method may be further defined by one, two, or more of the following features: the device is adapted for delivering pain medication directly into the cerebrospinal fluid of the intrathecal space surrounding the spinal cord; the device is adapted for delivering a drug to the brain; the device is adapted for intrathecal delivering baclofen; the device further comprises an intraspinal catheter; the device further comprises a second intrathecal drug delivery pump; and the device further comprises a catheter and an electrode.

11. Implantable Drug Delivery Pump for Chemotherapy

In one aspect, the present invention provides a method for inhibiting scarring comprising placing an implantable drug delivery pump for chemotherapy and an anti-scarring agent or a composition comprising an anti-scarring agent into an animal host, wherein the agent inhibits scarring.

Such a method may be further defined by one, two, or more of the following features: the device is adapted for delivering 2′-deoxy 5-fluorouridine; the host has a solid tumor, and the device is adapted for infusing a chemotherapeutic agent to the solid tumor; the host has a tumor, and the device is adapted for infusing a chemotherapeutic agent to the blood vessels that supply the tumor; and the host has a hepatic tumor, and the device is adapted for delivering a chemotherapeutic agent to the artery that provides blood supply to the liver of the host.

12. Drug Delivery Pump for Treating Heart Disease

In one aspect, the present invention provides a method for for inhibiting scarring comprising placing a drug delivery pump for treating heart disease and an anti-scarring agent or a composition comprising an anti-scarring agent into an animal host, wherein the agent inhibits scarring.

In one embodiment, the device is an implantable cardiac electrode that delivers stimulation energy and dispenses drug adjacent to the stimulation site.

13. Drug Delivery Implant (i.e., a Pump)

In one aspect, the present invention provides a method for inhibiting scarring comprising placing a drug delivery implant (i.e., a pump) and an anti-scarring agent or a composition comprising an anti-scarring agent into an animal host, wherein the agent inhibits scarring.

Additional Features Related to Methods for Inhibiting Scarring Using a Sensor

The methods for inhibiting scarring may also be further defined by one, two, or more of the following features: the agent inhibits cell regeneration; the agent inhibits angiogenesis; the agent inhibits fibroblast migration; the agent inhibits fibroblast proliferation; the agent inhibits deposition of extracellular matrix; the agent inhibits tissue remodeling; the agent is an angiogenesis inhibitor; the agent is a 5-lipoxygenase inhibitor or antagonist; the agent is a chemokine receptor antagonist; the agent is a cell cycle inhibitor; the agent is a taxane; the agent is an anti-microtubule agent; the agent is paclitaxel; the agent is not paclitaxel; the agent is an analogue or derivative of paclitaxel; the agent is a vinca alkaloid; the agent is camptothecin or an analogue or derivative thereof; the agent is a podophyllotoxin; the agent is a podophyllotoxin, wherein the podophyllotoxin is etoposide or an analogue or derivative thereof; the agent is an anthracycline; the agent is an anthracycline, wherein the anthracycline is doxorubicin or an analogue or derivative thereof; the agent is an anthracycline, wherein the anthracycline is mitoxantrone or an analogue or derivative thereof; the agent is a platinum compound; the agent is a nitrosourea; the agent is a nitroimidazole; the agent is a folic acid antagonist; the agent is a cytidine analogue; the agent is a pyrimidine analogue; the agent is a fluoropyrimidine analogue; the agent is a purine analogue; the agent is a nitrogen mustard or an analogue or derivative thereof; the agent is a hydroxyurea; the agent is a mytomicin or an analogue or derivative thereof; the agent is an alkyl sulfonate; the agent is a benzamide or an analogue or derivative thereof; the agent is a nicotinamide or an analogue or derivative thereof; the agent is a halogenated sugar or an analogue or derivative thereof; the agent is a DNA alkylating agent; the agent is an anti-microtubule agent; the agent is a topoisomerase inhibitor; the agent is a DNA cleaving agent; the agent is an antimetabolite; the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; the agent is a cyclin dependent protein kinase inhibitor; the agent is an epidermal growth factor kinase inhibitor; the agent is an elastase inhibitor; the agent is a factor Xa inhibitor; the agent is a farnesyltransferase inhibitor; the agent is a fibrinogen antagonist; the agent is a guanylate cyclase stimulant; the agent is a heat shock protein 90 antagonist; the agent is a heat shock protein 90 antagonist, wherein the heat shock protein 90 antagonist is geldanamycin or an analogue or derivative thereof; the agent is a guanylate cyclase stimulant; the agent is a HMGCoA reductase inhibitor; the agent is a HMGCoA reductase inhibitor, wherein the HMGCoA reductase inhibitor is simvastatin or an analogue or derivative thereof; the agent is a hydroorotate dehydrogenase inhibitor; the agent is an IKK2 inhibitor; the agent is an IL-1 antagonist; the agent is an ICE antagonist; the agent is an IRAK antagonist; the agent is an IL-4 agonist; the agent is an immunomodulatory agent; the agent is sirolimus or an analogue or derivative thereof; the agent is not sirolimus; the agent is everolimus or an analogue or derivative thereof; the agent is tacrolimus or an analogue or derivative thereof; the agent is not tacrolimus; the agent is biolmus or an analogue or derivative thereof; the agent is tresperimus or an analogue or derivative thereof; the agent is auranofin or an analogue or derivative thereof; the agent is 27-O-demethylrapamycin or an analogue or derivative thereof; the agent is gusperimus or an analogue or derivative thereof; the agent is pimecrolimus or an analogue or derivative thereof; the agent is ABT-578 or an analogue or derivative thereof; the agent is an inosine monophosphate dehydrogenase (IMPDH) inhibitor; the agent is an IMPDH inhibitor, wherein the IMPDH inhibitor is mycophenolic acid or an analogue or derivative thereof; the agent is an IMPDH inhibitor, wherein the IMPDH inhibitor is 1-alpha-25 dihydroxy vitamin D3 or an analogue or derivative thereof; the agent is a leukotriene inhibitor; the agent is a MCP-1 antagonist; the agent is a MMP inhibitor; the agent is an NF kappa B inhibitor; the agent is an NF kappa B inhibitor, wherein the NF kappa B inhibitor is Bay 11-7082; the agent is an NO antagonist; the agent is a p38 MAP kinase inhibitor; the agent is a p38 MAP kinase inhibitor, wherein the p38 MAP kinase inhibitor is SB 202190; the agent is a phosphodiesterase inhibitor; the agent is a TGF beta inhibitor; the agent is a thromboxane A2 antagonist; the agent is a TNFa antagonist; the agent is a TACE inhibitor; the agent is a tyrosine kinase inhibitor; the agent is a vitronectin inhibitor; the agent is a fibroblast growth factor inhibitor; the agent is a protein kinase inhibitor; the agent is a PDGF receptor kinase inhibitor; the agent is an endothelial growth factor receptor kinase inhibitor; the agent is a retinoic acid receptor antagonist; the agent is a platelet derived growth factor receptor kinase inhibitor; the agent is a fibronogin antagonist; the agent is an antimycotic agent; the agent is an antimycotic agent, wherein the antimycotic agent is sulconizole; the agent is a bisphosphonate; the agent is a phospholipase A1 inhibitor; the agent is a histamine H1/H2/H3 receptor antagonist; the agent is a macrolide antibiotic; the agent is a GPIIb/IIIa receptor antagonist; the agent is an endothelin receptor antagonist; the agent is a peroxisome proliferator-activated receptor agonist; the agent is an estrogen receptor agent; the agent is a somastostatin analogue; the agent is a neurokinin 1 antagonist; the agent is a neurokinin 3 antagonist; the agent is a VLA-4 antagonist; the agent is an osteoclast inhibitor; the agent is a DNA topoisomerase ATP hydrolyzing inhibitor; the agent is an angiotensin I converting enzyme inhibitor; the agent is an angiotensin II antagonist; the agent is an enkephalinase inhibitor; the agent is a peroxisome proliferator-activated receptor gamma agonist insulin sensitizer; the agent is a protein kinase C inhibitor; the agent is a ROCK (rho-associated kinase) inhibitor; the agent is a CXCR3 inhibitor; the agent is an Itk inhibitor; the agent is a cytosolic phospholipase A2-alpha inhibitor; the agent is a PPAR agonist; the agent is an immunosuppressant; the agent is an Erb inhibitor; the agent is an apoptosis agonist; the agent is a lipocortin agonist; the agent is a VCAM-1 antagonist; the agent is a collagen antagonist; the agent is an alpha 2 integrin antagonist; the agent is a TNF alpha inhibitor; the agent is a nitric oxide inhibitor the agent is a cathepsin inhibitor; the agent is not an anti-inflammatory agent; the agent is not a steroid; the agent is not a glucocorticosteroid; the agent is not dexamethasone; the agent is not beclomethasone; the agent is not dipropionate; the agent is not an anti-infective agent; the agent is not an antibiotic; the agent is not an anti-fungal agent; the method, wherein the composition comprises a polymer; the method, wherein the composition comprises a polymer, and the polymer is, or comprises, a copolymer; the method, wherein the composition comprises a polymer, and the polymer is, or comprises, a block copolymer; the method, wherein the composition comprises a polymer, and the polymer is, or comprises, a random copolymer; the method, wherein the composition comprises a polymer, and the polymer is, or comprises, a biodegradable polymer; the method, wherein the composition comprises a polymer, and the polymer is, or comprises, a non-biodegradable polymer; the method, wherein the composition comprises a polymer, and the polymer is, or comprises, a hydrophilic polymer; the method, wherein the composition comprises a polymer, and the polymer is, or comprises, a hydrophobic polymer; the method, wherein the composition comprises a polymer, and the polymer is, or comprises, a polymer having hydrophilic domains; the method, wherein the composition comprises a polymer, and the polymer is, or comprises, a polymer having hydrophobic domains; the method, wherein the composition comprises a polymer, and the polymer is, or comprises, a non-conductive polymer; the method, wherein the composition comprises a polymer, and the polymer is, or comprises, an elastomer; the method, wherein the composition comprises a polymer, and the polymer is, or comprises, a hydrogel; the method, wherein the composition comprises a polymer, and the polymer is, or comprises, a silicone polymer; the method, wherein the composition comprises a polymer, and the polymer is, or comprises, a hydrocarbon polymer; the method, wherein the composition comprises a polymer, and the polymer is, or comprises, a styrene-derived polymer; the method, wherein the composition comprises a polymer, and the polymer is, or comprises, a butadiene-derived polymer; the method, wherein the composition comprises a polymer, and the polymer is, or comprises, a macromer; the method, wherein the composition comprises a polymer, and the polymer is, or comprises, a poly(ethylene glycol) polymer; the method, wherein the composition comprises a polymer, and the polymer is, or comprises, an amorphous polymer; the method, wherein the composition further comprises a second pharmaceutically active agent; the method, wherein the composition further comprises an anti-inflammatory agent; the method, wherein the composition further comprises an agent that inhibits infection; the method, wherein the composition further comprises an anthracycline; the method, wherein the composition further comprises doxorubicin; the composition further comprises mitoxantrone; the composition further comprises a fluoropyrimidine; the method, wherein the composition further comprises 5-fluorouracil (5-FU); the method, wherein the composition further comprises a folic acid antagonist; the method, wherein the composition further comprises methotrexate; the method, wherein the composition further comprises a podophylotoxin; the method, wherein the composition further comprises etoposide; the method, wherein the composition further comprises camptothecin; the method, wherein the composition further comprises a hydroxyurea; the method, wherein the composition further comprises a platinum complex; the method, wherein the composition further comprises cisplatin; the composition further comprises an anti-thrombotic agent; the method, wherein the composition further comprises a visualization agent; the method, wherein the composition further comprises a visualization agent, and the visualization agent is a radiopaque material, wherein the radiopaque material comprises a metal, a halogenated compound, or a barium containing compound; the method, wherein the composition further comprises a visualization agent, and the visualization agent is, or comprises, barium, tantalum, or technetium; the method, wherein the composition further comprises a visualization agent, and the visualization agent is, or comprises, an MRI responsive material; the method, wherein the composition further comprises a visualization agent, and the visualization agent is, or comprises, a gadolinium chelate; the method, wherein the composition further comprises a visualization agent, and the visualization agent is, or comprises, iron, magnesium, manganese, copper, or chromium; the method, wherein the composition further comprises a visualization agent, and the visualization agent is, or comprises, iron oxide compound; the method, wherein the composition further comprises a visualization agent, and the visualization agent is, or comprises, a dye, pigment, or colorant; the agent is released in effective concentrations from the composition comprising the agent by diffusion over a period ranging from the time of administration to about 90 days; the agent is released in effective concentrations from the composition comprising the agent by erosion of the composition over a period ranging from the time of administration to about 90 days; the composition further comprises an inflammatory cytokine; the composition further comprises an agent that stimulates cell proliferation; the composition further comprises a polymeric carrier; the composition is in the form of a gel, paste, or spray; the sensor is partially constructed with the agent or the composition; the sensor is impregnated with the agent or the composition; the method, wherein the agent or the composition forms a coating, and the coating directly contacts the sensor; the method, wherein the agent or the composition forms a coating, and the coating indirectly contacts the sensor; the agent or the composition forms a coating, and the coating partially covers the sensor; the method, wherein the agent or the composition forms a coating, and the coating completely covers the sensor; the agent or the composition is located within pores or holes of the sensor; the agent or the composition is located within a channel, lumen, or divet of the sensor; the sensor further comprises an echogenic material; the sensor further comprises an echogenic material, wherein the echogenic material is in the form of a coating; the sensor is sterile; the agent is delivered from the sensor, wherein the agent is released into tissue in the vicinity of the sensor after deployment of the sensor; the agent is delivered from the sensor, wherein the agent is released into tissue in the vicinity of the sensor after deployment of the sensor, wherein the tissue is connective tissue; the agent is delivered from the sensor, wherein the agent is released into tissue in the vicinity of the sensor after deployment of the sensor, wherein the tissue is muscle tissue; the agent is delivered from the sensor, wherein the agent is released into tissue in the vicinity of the sensor after deployment of the sensor, wherein the tissue is nerve tissue; the agent is delivered from the sensor, wherein the agent is released into tissue in the vicinity of the sensor after deployment of the sensor, wherein the tissue is epithelium tissue; the agent is delivered from the sensor, wherein the agent is released in effective concentrations from the sensor over a period ranging from the time of deployment of the sensor to about 1 year; the agent is delivered from the sensor, wherein the agent is released in effective concentrations from the sensor over a period ranging from about 1 month to 6 months; the agent is delivered from the sensor, wherein the agent is released in effective concentrations from the sensor over a period ranging from about 1-90 days; the agent is delivered from the sensor, wherein the agent is released in effective concentrations from the sensor at a constant rate; the agent is delivered from the sensor, wherein the agent is released in effective concentrations from the sensor at an increasing rate; the agent is delivered from the sensor, wherein the agent is released in effective concentrations from the sensor at a decreasing rate; the agent is delivered from the sensor, wherein the sensor comprises about 0.01 μg to about 10 μg of the agent; the agent is delivered from the sensor, wherein the sensor comprises about 10 μg to about 10 mg of the agent; the agent is delivered from the sensor, wherein the sensor comprises about 10 mg to about 250 mg of the agent; the agent is delivered from the sensor, wherein the sensor comprises about 250 mg to about 1000 mg of the agent; the agent is delivered from the sensor, wherein the sensor comprises about 1000 mg to about 2500 mg of the agent; the agent is delivered from the sensor, wherein a surface of the sensor comprises less than 0.01 μg of the agent per mm² of sensor surface to which the agent is applied; the agent is delivered from the sensor, wherein a surface of the sensor comprises about 0.01 μg to about 1 μg of the agent per mm² of sensor surface to which the agent is applied; the agent is delivered from the sensor, wherein a surface of the sensor comprises about 1 μg to about 10 μg of the agent per mm² of sensor surface to which the agent is applied; the agent is delivered from the sensor, wherein a surface of the sensor comprises about 10 μg to about 250 μg of the agent per mm² of sensor surface to which the agent is applied; the agent is delivered from the sensor, wherein a surface of the sensor comprises about 250 μg to about 1000 μg of the agent per mm of sensor surface to which the agent is applied; the agent is delivered from the sensor, wherein a surface of the sensor comprises about 1000 μg to about 2500 μg of the agent per mm² of sensor surface to which the agent is applied; the method, wherein the sensor further comprises a coating, and the coating is a uniform coating; the method, wherein the sensor further comprises a coating, and the coating is a non-uniform coating; the method, wherein the sensor further comprises a coating, and the coating is a discontinuous coating; the method, wherein the sensor further comprises a coating, and the coating is a patterned coating; the method, wherein the sensor further comprises a coating, and the coating has a thickness of 100 μm or less; the method, wherein the sensor further comprises a coating, and the coating has a thickness of 10 μm or less; the method, wherein the sensor further comprises a coating, and the coating adheres to the surface of the sensor upon deployment of the sensor; the method, wherein the sensor further comprises a coating, and the coating is stable at room temperature for a period of at least 1 year; the method, wherein the sensor further comprises a coating, and the agent is present in the coating in an amount ranging between about 0.0001% to about 1% by weight; the method, wherein the sensor further comprises a coating, and the agent is present in the coating in an amount ranging between about 1% to about 10% by weight; the method, wherein the sensor further comprises a coating, and the agent is present in the coating in an amount ranging between about 10% to about 25% by weight; the method, wherein the sensor further comprises a coating, and the agent is present in the coating in an amount ranging between about 25% to about 70% by weight; the method, wherein the sensor further comprises a coating, and the coating comprises a polymer; the method, wherein the sensor comprises a first coating having a first composition and a second coating having a second composition; the method, wherein the sensor comprises a first coating having a first composition and a second coating having a second composition, wherein the first composition and the second composition are different; the agent or the composition is affixed to the sensor; the agent or the composition is covalently attached to the sensor; the agent or the composition is non-covalently attached to the sensor; the sensor comprises a coating that absorbs the agent or the composition; the sensor is interweaved with a thread composed of, or coated with, the agent or the composition; a portion of the sensor is covered with a sleeve that contains the agent or the composition; the sensor is completely covered with a sleeve that contains the agent or the composition; a portion of the sensor is covered with a mesh that contains the agent or the composition; the sensor is completely covered with a mesh that contains the agent or the composition; the sensor is linked to a pump; the agent or the composition is applied to the sensor surface prior to to the placing of the sensor into the host; the agent or the composition is applied to the sensor surface during the placing of the sensor into the host; the agent or the composition is applied to the sensor surface immediately after the placing of the sensor into the host; the agent or the composition is applied to the surface of the tissue in the host surrounding the sensor prior to to the placing of the sensor into the host; the agent or the composition is applied to the surface of the tissue in the host surrounding the sensor during the placing of the sensor into the host; the agent or the composition is applied to the surface of the tissue in the host surrounding the sensor immediately after the placing of the sensor into the host; the agent or the composition is topically applied into the anatomical space where the sensor is placed; and the agent or the composition is percutaneously injected into the tissue in the host surrounding the sensor.

Additional Features Related to Methods for Inhibiting Scarring Using a Pump

The methods for inhibiting scarring may also be further defined by one, two, or more of the following features: the agent inhibits cell regeneration; the agent inhibits angiogenesis; the agent inhibits fibroblast migration; the agent inhibits fibroblast proliferation; the agent inhibits deposition of extracellular matrix; the agent inhibits tissue remodeling; the agent is an angiogenesis inhibitor; the agent is a 5-lipoxygenase inhibitor or antagonist; the agent is a chemokine receptor antagonist; the agent is a cell cycle inhibitor; the agent is a taxane; the agent is an anti-microtubule agent; the agent is paclitaxel; the agent is not paclitaxel; the agent is an analogue or derivative of paclitaxel; the agent is a vinca alkaloid; the agent is camptothecin or an analogue or derivative thereof; the agent is a podophyllotoxin; the agent is a podophyllotoxin, wherein the podophyllotoxin is etoposide or an analogue or derivative thereof; the agent is an anthracycline; the agent is an anthracycline, wherein the anthracycline is doxorubicin or an analogue or derivative thereof; the agent is an anthracycline, wherein the anthracycline is mitoxantrone or an analogue or derivative thereof; the agent is a platinum compound; the agent is a nitrosourea; the agent is a nitroimidazole; the agent is a folic acid antagonist; the agent is a cytidine analogue; the agent is a pyrimidine analogue; the agent is a fluoropyrimidine analogue; the agent is a purine analogue; the agent is a nitrogen mustard or an analogue or derivative thereof; the agent is a hydroxyurea; the agent is a mytomicin or an analogue or derivative thereof; the agent is an alkyl sulfonate; the agent is a benzamide or an analogue or derivative thereof; the agent is a nicotinamide or an analogue or derivative thereof; the agent is a halogenated sugar or an analogue or derivative thereof; the agent is a DNA alkylating agent; the agent is an anti-microtubule agent; the agent is a topoisomerase inhibitor; the agent is a DNA cleaving agent; the agent is an antimetabolite; the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; the agent is a cyclin dependent protein kinase inhibitor; the agent is an epidermal growth factor kinase inhibitor; the agent is an elastase inhibitor; the agent is a factor Xa inhibitor; the agent is a farnesyltransferase inhibitor; the agent is a fibrinogen antagonist; the agent is a guanylate cyclase stimulant; the agent is a heat shock protein 90 antagonist; the agent is a heat shock protein 90 antagonist, wherein the heat shock protein 90 antagonist is geldanamycin or an analogue or derivative thereof; the agent is a guanylate cyclase stimulant; the agent is a HMGCoA reductase inhibitor; the agent is a HMGCoA reductase inhibitor, wherein the HMGCoA reductase inhibitor is simvastatin or an analogue or derivative thereof; the agent is a hydroorotate dehydrogenase inhibitor; the agent is an IKK2 inhibitor; the agent is an IL-1 antagonist; the agent is an ICE antagonist; the agent is an IRAK antagonist; the agent is an IL-4 agonist; the agent is an immunomodulatory agent; the agent is sirolimus or an analogue or derivative thereof; the agent is not sirolimus; the agent is everolimus or an analogue or derivative thereof; the agent is tacrolimus or an analogue or derivative thereof; the agent is not tacrolimus; the agent is biolmus or an analogue or derivative thereof; the agent is tresperimus or an analogue or derivative thereof; the agent is auranofin or an analogue or derivative thereof; the agent is 27-O-demethylrapamycin or an analogue or derivative thereof; the agent is gusperimus or an analogue or derivative thereof; the agent is pimecrolimus or an analogue or derivative thereof; the agent is ABT-578 or an analogue or derivative thereof; the agent is an inosine monophosphate dehydrogenase (IMPDH) inhibitor; the agent is an IMPDH inhibitor, wherein the IMPDH inhibitor is mycophenolic acid or an analogue or derivative thereof; the agent is an IMPDH inhibitor, wherein the IMPDH inhibitor is 1-alpha-25 dihydroxy vitamin D3 or an analogue or derivative thereof; the agent is a leukotriene inhibitor; the agent is a MCP-1 antagonist; the agent is a MMP inhibitor; the agent is an NF kappa B inhibitor; the agent is an NF kappa B inhibitor, wherein the NF kappa B inhibitor is Bay 11-7082; the agent is an NO antagonist; the agent is a p38 MAP kinase inhibitor; the agent is a p38 MAP kinase inhibitor, wherein the p38 MAP kinase inhibitor is SB 202190; the agent is a phosphodiesterase inhibitor; the agent is a TGF beta inhibitor; the agent is a thromboxane A2 antagonist; the agent is a TNFa antagonist; the agent is a TACE inhibitor; the agent is a tyrosine kinase inhibitor; the agent is a vitronectin inhibitor; the agent is a fibroblast growth factor inhibitor; the agent is a protein kinase inhibitor; the agent is a PDGF receptor kinase inhibitor; the agent is an endothelial growth factor receptor kinase inhibitor; the agent is a retinoic acid receptor antagonist; the agent is a platelet derived growth factor receptor kinase inhibitor; the agent is a fibronogin antagonist; the agent is an antimycotic agent; the agent is an antimycotic agent, wherein the antimycotic agent is sulconizole; the agent is a bisphosphonate; the agent is a phospholipase A1 inhibitor; the agent is a histamine H1/H2/H3 receptor antagonist; the agent is a macrolide antibiotic; the agent is a GPIIb/IIIa receptor antagonist; the agent is an endothelin receptor antagonist; the agent is a peroxisome proliferator-activated receptor agonist; the agent is an estrogen receptor agent; the agent is a somastostatin analogue; the agent is a neurokinin 1 antagonist; the agent is a neurokinin 3 antagonist; the agent is a VLA-4 antagonist; the agent is an osteoclast inhibitor; the agent is a DNA topoisomerase ATP hydrolyzing inhibitor; the agent is an angiotensin I converting enzyme inhibitor; the agent is an angiotensin II antagonist; the agent is an enkephalinase inhibitor; the agent is a peroxisome proliferator-activated receptor gamma agonist insulin sensitizer; the agent is a protein kinase C inhibitor; the agent is a ROCK (rho-associated kinase) inhibitor; the agent is a CXCR3 inhibitor; the agent is an ltk inhibitor; the agent is a cytosolic phospholipase A2-alpha inhibitor; the agent is a PPAR agonist; the agent is an immunosuppressant; the agent is an Erb inhibitor; the agent is an apoptosis agonist; the agent is a lipocortin agonist; the agent is a VCAM-1 antagonist; the agent is a collagen antagonist; the agent is an alpha 2 integrin antagonist; the agent is a TNF alpha inhibitor; the agent is a nitric oxide inhibitor the agent is a cathepsin inhibitor; the agent is not an anti-inflammatory agent; the agent is not a steroid; the agent is not a glucocorticosteroid; the agent is not dexamethasone; the agent is not beclomethasone; the agent is not dipropionate; the agent is not an anti-infective agent; the agent is not an antibiotic; the agent is not an anti-fungal agent; the method wherein the composition comprises a polymer; the method wherein the composition comprises a polymer, and the polymer is, or comprises, a copolymer; the method wherein the composition comprises a polymer, and the polymer is, or comprises, a block copolymer; the method wherein the composition comprises a polymer, and the polymer is, or comprises, a random copolymer; the method wherein the composition comprises a polymer, and the polymer is, or comprises, a biodegradable polymer; the method wherein the composition comprises a polymer, and the polymer is, or comprises, a non-biodegradable polymer; the method wherein the composition comprises a polymer, and the polymer is, or comprises, a hydrophilic polymer; the method wherein the composition comprises a polymer, and the polymer is, or comprises, a hydrophobic polymer; the method wherein the composition comprises a polymer, and the polymer is, or comprises, a polymer having hydrophilic domains; the method wherein the composition comprises a polymer, and the polymer is, or comprises, a polymer having hydrophobic domains; the method wherein the composition comprises a polymer, and the polymer is, or comprises, a non-conductive polymer; the method wherein the composition comprises a polymer, and the polymer is, or comprises, an elastomer; the method wherein the composition comprises a polymer, and the polymer is, or comprises, a hydrogel; the method wherein the composition comprises a polymer, and the polymer is, or comprises, a silicone polymer; the method wherein the composition comprises a polymer, and the polymer is, or comprises, a hydrocarbon polymer; the method wherein the composition comprises a polymer, and the polymer is, or comprises, a styrene-derived polymer; the method wherein the composition comprises a polymer, and the polymer is, or comprises, a butadiene-derived polymer; the method wherein the composition comprises a polymer, and the polymer is, or comprises, a macromer; the method wherein the composition comprises a polymer, and the polymer is, or comprises, a poly(ethylene glycol) polymer; the method wherein the composition comprises a polymer, and the polymer is, or comprises, an amorphous polymer; the method wherein the composition further comprises a second pharmaceutically active agent; the method wherein the composition further comprises an anti-inflammatory agent; the method wherein the composition further comprises an agent that inhibits infection; the method wherein the composition further comprises an anthracycline; the method wherein the composition further comprises doxorubicin; the composition further comprises mitoxantrone; the composition further comprises a fluoropyrimidine; the method wherein the composition further comprises 5-fluorouracil (5-FU); the method wherein the composition further comprises a folic acid antagonist; the method wherein the composition further comprises methotrexate; the method wherein the composition further comprises a podophylotoxin; the method wherein the composition further comprises etoposide; the method wherein the composition further comprises camptothecin; the method wherein the composition further comprises a hydroxyurea; the method wherein the composition further comprises a platinum complex; the method wherein the composition further comprises cisplatin; the composition further comprises an anti-thrombotic agent; the method wherein the composition further comprises a visualization agent; the method wherein the composition further comprises a visualization agent, and the visualization agent is a radiopaque material, wherein the radiopaque material comprises a metal, a halogenated compound, or a barium containing compound; the method wherein the composition further comprises a visualization agent, and the visualization agent is, or comprises, barium, tantalum, or technetium; the method wherein the composition further comprises a visualization agent, and the visualization agent is, or comprises, an MRI responsive material; the method wherein the composition further comprises a visualization agent, and the visualization agent is, or comprises, a gadolinium chelate; the method wherein the composition further comprises a visualization agent, and the visualization agent is, or comprises, iron, magnesium, manganese, copper, or chromium; the method wherein the composition further comprises a visualization agent, and the visualization agent is, or comprises, iron oxide compound; the method wherein the composition further comprises a visualization agent, and the visualization agent is, or comprises, a dye, pigment, or colorant; the agent is released in effective concentrations from the composition comprising the agent by diffusion over a period ranging from the time of administration to about 90 days; the agent is released in effective concentrations from the composition comprising the agent by erosion of the composition over a period ranging from the time of administration to about 90 days; the composition further comprises an inflammatory cytokine; the composition further comprises an agent that stimulates cell proliferation; the composition further comprises a polymeric carrier; the composition is in the form of a gel, paste, or spray; the pump is partially constructed with the agent or the composition; the pump is impregnated with the agent or the composition; the method wherein the agent or the composition forms a coating, and the coating directly contacts the pump; the method wherein the agent or the composition forms a coating, and the coating indirectly contacts the pump; the agent or the composition forms a coating, and the coating partially covers the pump; the method wherein the agent or the composition forms a coating, and the coating completely covers the pump; the agent or the composition is located within pores or holes of the pump; the agent or the composition is located within a channel, lumen, or divet of the pump; the pump further comprises an echogenic material; the pump further comprises an echogenic material, wherein the echogenic material is in the form of a coating; the pump is sterile; the agent is delivered from the pump, wherein the agent is released into tissue in the vicinity of the pump after deployment of the pump; the agent is delivered from the pump, wherein the agent is released into tissue in the vicinity of the pump after deployment of the pump, wherein the tissue is connective tissue; the agent is delivered from the pump, wherein the agent is released into tissue in the vicinity of the pump after deployment of the pump, wherein the tissue is muscle tissue; the agent is delivered from the pump, wherein the agent is released into tissue in the vicinity of the pump after deployment of the pump, wherein the tissue is nerve tissue; the agent is delivered from the pump, wherein the agent is released into tissue in the vicinity of the pump after deployment of the pump, wherein the tissue is epithelium tissue; the agent is delivered from the pump, wherein the agent is released in effective concentrations from the pump over a period ranging from the time of deployment of the pump to about 1 year; the agent is delivered from the pump, wherein the agent is released in effective concentrations from the pump over a period ranging from about 1 month to 6 months; the agent is delivered from the pump, wherein the agent is released in effective concentrations from the pump over a period ranging from about 1-90 days; the agent is delivered from the pump, wherein the agent is released in effective concentrations from the pump at a constant rate; the agent is delivered from the pump, wherein the agent is released in effective concentrations from the pump at an increasing rate; the agent is delivered from the pump, wherein the agent is released in effective concentrations from the pump at a decreasing rate; the agent is delivered from the pump, wherein the pump comprises about 0.01 μg to about 10 μg of the agent; the agent is delivered from the pump, wherein the pump comprises about 10 μg to about 10 mg of the agent; the agent is delivered from the pump, wherein the pump comprises about 10 mg to about 250 mg of the agent; the agent is delivered from the pump, wherein the pump comprises about 250 mg to about 1000 mg of the agent; the agent is delivered from the pump, wherein the pump comprises about 1000 mg to about 2500 mg of the agent; the agent is delivered from the pump, wherein a surface of the pump comprises less than 0.01 μg of the agent per mm² of pump surface to which the agent is applied; the agent is delivered from the pump, wherein a surface of the pump comprises about 0.01 μg to about 1 μg of the agent per mm² of pump surface to which the agent is applied; the agent is delivered from the pump, wherein a surface of the pump comprises about 1 μg to about 10 μg of the agent per mm² of pump surface to which the agent is applied; the agent is delivered from the pump, wherein a surface of the pump comprises about 10 μg to about 250 μg of the agent per mm² of pump surface to which the agent is applied; the agent is delivered from the pump, wherein a surface of the pump comprises about 250 μg to about 1000 μg of the agent per mm² of pump surface to which the agent is applied; the agent is delivered from the pump, wherein a surface of the pump comprises about 1000 μg to about 2500 μg of the agent per mm² of pump surface to which the agent is applied; the method wherein the pump further comprises a coating, and the coating is a uniform coating; the method wherein the pump further comprises a coating, and the coating is a non-uniform coating; the method wherein the pump further comprises a coating, and the coating is a discontinuous coating; the method wherein the pump further comprises a coating, and the coating is a patterned coating; the method wherein the pump further comprises a coating, and the coating has a thickness of 100 μm or less; the method wherein the pump further comprises a coating, and the coating has a thickness of 10 μm or less; the method wherein the pump further comprises a coating, and the coating adheres to the surface of the pump upon deployment of the pump; the method wherein the pump further comprises a coating, and the coating is stable at room temperature for a period of at least 1 year; the method wherein the pump further comprises a coating, and the agent is present in the coating in an amount ranging between about 0.0001% to about 1% by weight; the method wherein the pump further comprises a coating, and the agent is present in the coating in an amount ranging between about 1% to about 10% by weight; the method wherein the pump further comprises a coating, and the agent is present in the coating in an amount ranging between about 10% to about 25% by weight; the method wherein the pump further comprises a coating, and the agent is present in the coating in an amount ranging between about 25% to about 70% by weight; the method wherein the pump further comprises a coating, and the coating comprises a polymer; the method wherein the pump comprises a first coating having a first composition and a second coating having a second composition; the method wherein the pump comprises a first coating having a first composition and a second coating having a second composition, wherein the first composition and the second composition are different; the agent or the composition is affixed to the pump; the agent or the composition is covalently attached to the pump; the agent or the composition is non-covalently attached to the pump; the pump comprises a coating that absorbs the agent or the composition; the pump is interweaved with a thread composed of, or coated with, the agent or the composition; a portion of the pump is covered with a sleeve that contains the agent or the composition; the pump is completely covered with a sleeve that contains the agent or the composition; a portion of the pump is covered with a mesh that contains the agent or the composition; the pump is completely covered with a mesh that contains the agent or the composition; the pump is linked to a sensor; the agent or the composition is applied to the pump surface prior to to the placing of the pump into the host; the agent or the composition is applied to the pump surface during the placing of the pump into the host; the agent or the composition is applied to the pump surface immediately after the placing of the pump into the host; the agent or the composition is applied to the surface of the tissue in the host surrounding the pump prior to to the placing of the pump into the host; the agent or the composition is applied to the surface of the tissue in the host surrounding the pump during the placing of the pump into the host; the agent or the composition is applied to the surface of the tissue in the host surrounding the pump immediately after the placing of the pump into the host; the agent or the composition is topically applied into the anatomical space where the pump is placed; and the agent or the composition is percutaneously injected into the tissue in the host surrounding the pump.

The present invention, in various aspects and embodiments, provides the following methods for making devices:

1. Sensor

In one aspect, the present invention provides a method for making a device comprising: combining a sensor and an anti-scarring agent or a composition comprising an anti-scarring agent, wherein the agent inhibits scarring between the device and a host into which the device is implanted.

Such a method may be defined by one, two, or more of the following features: the sensor is a blood or tissue glucose monitor; the sensor is an electrolyte sensor; the sensor is a blood constituent sensor; the sensor is a temperature sensor; the sensor is a pH sensor; the sensor is an optical sensor; the sensor is an amperometric sensor; the sensor is a pressure sensor; the sensor is a biosensor; the sensor is a sensing transponder; the sensor is a strain sensor; the sensor is a magnetoresistive sensor; the sensor is a cardiac sensor; the sensor is a respiratory sensor; the sensor is an auditory sensor; the sensor is a metabolite sensor; the sensor detects mechanical changes; the sensor detects physical changes; the sensor detects electrochemical changes; the sensor detects magnetic changes; the sensor detects acceleration changes; the sensor detects ionizing radiation changes; the sensor detects acoustic wave changes; the sensor detects chemical changes; the sensor detects drug concentration changes; the sensor detects hormone changes; and the sensor detects barometric changes.

2. Blood or Tissue Glucose Monitor (i.e., a Sensor)

In one aspect, the present invention provides a method for making a device comprising: combining a blood or tissue glucose monitor (i.e., a sensor) and an anti-scarring agent or a composition comprising an anti-scarring agent, wherein the agent inhibits scarring between the device and a host into which the device is implanted.

Such a method may be further defined by one, two, or more of the following features: the device is deliverable to the vascular system transluminally using a catheter on a stent platform; the device is composed of glucose sensitive living cells that monitor blood glucose levels and produce a detectable electrical or optical signal in response to changes in glucose concentrations; the device is an electrode composed of an analyte responsive enzyme; the device is a closed loop insulin delivery system that comprises a sensing means that detects the host's blood glucose level and stimulates an insulin pump to supply insulin; and the device is a closed loop insulin delivery system that comprises a sensing means that detects the host's blood glucose level and stimulates the pancreas to supply insulin.

3. Pressure or Stress Sensor

In one aspect, the present invention provides a method for making a device comprising: combining a pressure or stress sensor and an anti-scarring agent or a composition comprising an anti-scarring agent, wherein the agent inhibits scarring between the device and a host into which the device is implanted.

Such a method may be further defined by one, two, or more of the following features: the device monitors blood pressure; the device monitors fluid flow; the device monitors pressure within an aneurysm sac; the device monitors intracranial pressure; the device monitors mechanical pressure associated with a bone fracture; the device monitors barometric pressure; the device monitors eye tremors; the device monitors the depth of a corneal implant; the device monitors intraocular pressure; the device is a passive sensor with an inductor-capacitor circuit; the device is a self-powered strain sensing system; and the sensor comprises a lead, a sensor module, and a sensor circuit and means for providing voltage.

4. Cardiac Sensor

In one aspect, the present invention provides a method making a device comprising: combining a cardiac sensor and an anti-scarring agent or a composition comprising an anti-scarring agent, wherein the agent inhibits scarring between the device and a host into which the device is implanted.

Such a method may be further defined by one, two, or more of the following features: the device monitors cardiac output; the device monitors ejection fraction; the device monitors blood pressure in a heart chamber; the device monitors ventricular wall motions; the device monitors blood flow to a transplanted organ; and the device monitors heart rate.

5. Respiratory Sensor

In one aspect, the present invention provides a method for making a device comprising: combining a respiratory sensor and an anti-scarring agent or a composition comprising an anti-scarring agent, wherein the agent inhibits scarring between the device and a host into which the device is implanted.

In one embodiment, the device monitors pulmonary functions.

6. Auditory Sensor

In one aspect, the present invention provides a method for making a device comprising: combining an auditory sensor and an anti-scarring agent or a composition comprising an anti-scarring agent, wherein the agent inhibits scarring between the device and a host into which the device is implanted.

Such a method may be further defined by one, two, or more of the following features: the device is adapted for delivering an electrical signal to an implantable electromechanical transducer that acts on the middle or inner ear; the device generates an electrical audio signal; the device is a capacitive sensor that is coupled to a vibrating auditory element; and the device is an electromagnetic sensor.

7. Electrolyte or Metabolite Sensor

In one aspect, the present invention provides a method for making a device comprising: combining an electrolyte or metabolite sensor and an anti-scarring agent or a composition comprising an anti-scarring agent, wherein the agent inhibits scarring between the device and a host into which the device is implanted.

Such a method may be further defined by one, two, or more of the following features: the device emits a source of radiation directed towards blood to interact with a plurality of detectors that provide an output signal; the device is a biosensing transponder composed of a dye that has optical properties that change in response to changes in the environment, a photosensor to sense the optical changes, and a transponder for transmitting data to a remote reader; and the device is a monolithic bioelectronic device for detecting at least one analyte within the host.

8. Pump

The present invention provides a method for making a device comprising: combining a pump and an anti-scarring agent or a composition comprising an anti-scarring agent, wherein the agent inhibits scarring between the device and a host into which the device is implanted.

Such a method may be further defined by one, two or more the following features: the device is adapted for delivering insulin; the device is adapted for delivering a narcotic; the device is adapted for delivering a chemotherapeutic agent; the device is adapted for delivering an anti-arrhythmic drug; the device is adapted for delivering an anti-spasmotic drug; the device is adapted for delivering an anti-spastic agent; the device is adapted for delivering an antibiotic; the device is adapted for delivering a drug only when changes in the host are detected; the device is adapted for delivering a drug as a continuous slow release; the device is adapted for delivering a drug at prescribed dosages in a pulsatile manner; the device is a programmable drug delivery pump; the device is adapted for intraocularly delivering a drug; the device is adapted for intrathecally delivering a drug; the device is adapted for intraperitoneally delivering a drug; the device is adapted for intra-arterially delivering a drug; the device is adapted for intracardiac delivery of a drug; the device is an implantable osmotic pump; the device is an ocular drug delivery pump; the device is metering system; the device is a peristaltic (roller) pump; the device is an electronically driven pump; the device is an elastomeric pump; the device is a spring contraction pump; the device is a gas-driven pump; the device is a hydraulic pump; the device is a piston-dependent pump; the device is a non-piston-dependent pump; the device is a dispensing chamber; the device is an infusion pump; and the device is a passive pump.

9. Implantable Insulin Pump

In one aspect, the present invention provides a method for making a device comprising: combining an implantable insulin pump and an anti-scarring agent or a composition comprising an anti-scarring agent, wherein the agent inhibits scarring between the device and a host into which the device is implanted.

In one embodiment, the implantable insulin pump comprises a single channel catheter with a sensor implanted in a vessel that transmits blood chemistry to the implantable insulin pump to dispense mediation through the catheter.

10. Intrathecal Durg Delivery Pump

In one aspect, the present invention provides a method for making a device comprising: combining an intrathecal drug delivery pump and an anti-scarring agent or a composition comprising an anti-scarring agent, wherein the agent inhibits scarring between the device and a host into which the device is implanted.

Such a method may be further defined by one, two or more the following features: the device is adapted for delivering pain medication directly into the cerebrospinal fluid of the intrathecal space surrounding the spinal cord; the device is adapted for delivering a drug to the brain; the device is adapted for intrathecal delivering baclofen; the device further comprises an intraspinal catheter; the device further comprises a second intrathecal drug delivery pump; and the device further comprises a catheter and an electrode.

11. Implantable Drug Delivery Pump for Chemotherapy

In one aspect, the present invention provides a method for making a medical device comprising: combining an implantable drug delivery pump for chemotherapy and an anti-scarring agent or a composition comprising an anti-scarring agent, wherein the agent inhibits scarring between the device and a host into which the device is implanted.

Such a method may be further defined by one, two, or more of the following features: the device is adapted for delivering 2′-deoxy 5-fluorouridine; the host has a solid tumor, and the device is adapted for infusing a chemotherapeutic agent to the solid tumor; the host has a tumor, and the device is adapted for infusing a chemotherapeutic agent to the blood vessels that supply the tumor; and the host has a hepatic tumor, and the device is adapted for delivering a chemotherapeutic agent to the artery that provides blood supply to the liver of the host.

12. Drug Delivery Pump for Treating Heart Disease

In one aspect, the present invention provides a method for making a device comprising: combining a drug delivery pump for treating heart disease and an anti-scarring agent or a composition comprising an anti-scarring agent, wherein the agent inhibits scarring between the device and a host into which the device is implanted.

In one embodiment, the device is an implantable cardiac electrode that delivers stimulation energy and dispenses drug adjacent to the stimulation site.

13. Drug Delivery Implant (i.e., a Pump)

In one aspect, the present invention provides a method for making a device comprising: combining a drug delivery pump and an anti-scarring agent or a composition comprising an anti-scarring agent, wherein the agent inhibits scarring between the device and a host into which the device is implanted.

Additional Features Related to Methods for Making Sensors

The methods for making the sensors as described above may also be further defined by one, two, or more of the following features: the agent inhibits cell regeneration; the agent inhibits angiogenesis; the agent inhibits fibroblast migration; the agent inhibits fibroblast proliferation; the agent inhibits deposition of extracellular matrix; the agent inhibits tissue remodeling; the agent is an angiogenesis inhibitor; the agent is a 5-lipoxygenase inhibitor or antagonist; the agent is a chemokine receptor antagonist; the agent is a cell cycle inhibitor; the agent is a taxane; the agent is an anti-microtubule agent; the agent is paclitaxel; the agent is not paclitaxel; the agent is an analogue or derivative of paclitaxel; the agent is a vinca alkaloid; the agent is camptothecin or an analogue or derivative thereof; the agent is a podophyllotoxin; the agent is a podophyllotoxin, wherein the podophyllotoxin is etoposide or an analogue or derivative thereof; the agent is an anthracycline; the agent is an anthracycline, wherein the anthracycline is doxorubicin or an analogue or derivative thereof; the agent is an anthracycline, wherein the anthracycline is mitoxantrone or an analogue or derivative thereof; the agent is a platinum compound; the agent is a nitrosourea; the agent is a nitroimidazole; the agent is a folic acid antagonist; the agent is a cytidine analogue; the agent is a pyrimidine analogue; the agent is a fluoropyrimidine analogue; the agent is a purine analogue; the agent is a nitrogen mustard or an analogue or derivative thereof; the agent is a hydroxyurea; the agent is a mytomicin or an analogue or derivative thereof; the agent is an alkyl sulfonate; the agent is a benzamide or an analogue or derivative thereof; the agent is a nicotinamide or an analogue or derivative thereof; the agent is a halogenated sugar or an analogue or derivative thereof; the agent is a DNA alkylating agent; the agent is an anti-microtubule agent; the agent is a topoisomerase inhibitor; the agent is a DNA cleaving agent; the agent is an antimetabolite; the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; the agent is a cyclin dependent protein kinase inhibitor; the agent is an epidermal growth factor kinase inhibitor; the agent is an elastase inhibitor; the agent is a factor Xa inhibitor; the agent is a farnesyltransferase inhibitor; the agent is a fibrinogen antagonist; the agent is a guanylate cyclase stimulant; the agent is a heat shock protein 90 antagonist; the agent is a heat shock protein 90 antagonist, wherein the heat shock protein 90 antagonist is geldanamycin or an analogue or derivative thereof; the agent is a guanylate cyclase stimulant; the agent is a HMGCoA reductase inhibitor; the agent is a HMGCoA reductase inhibitor, wherein the HMGCoA reductase inhibitor is simvastatin or an analogue or derivative thereof; the agent is a hydroorotate dehydrogenase inhibitor; the agent is an IKK2 inhibitor; the agent is an IL-1 antagonist; the agent is an ICE antagonist; the agent is an IRAK antagonist; the agent is an IL-4 agonist; the agent is an immunomodulatory agent; the agent is sirolimus or an analogue or derivative thereof; the agent is not sirolimus; the agent is everolimus or an analogue or derivative thereof; the agent is tacrolimus or an analogue or derivative thereof; the agent is not tacrolimus; the agent is biolmus or an analogue or derivative thereof; the agent is tresperimus or an analogue or derivative thereof; the agent is auranofin or an analogue or derivative thereof; the agent is 27-O-demethylrapamycin or an analogue or derivative thereof; the agent is gusperimus or an analogue or derivative thereof; the agent is pimecrolimus or an analogue or derivative thereof; the agent is ABT-578 or an analogue or derivative thereof; the agent is an inosine monophosphate dehydrogenase (IMPDH) inhibitor; the agent is an IMPDH inhibitor, wherein the IMPDH inhibitor is mycophenolic acid or an analogue or derivative thereof; the agent is an IMPDH inhibitor, wherein the IMPDH inhibitor is 1-alpha-25 dihydroxy vitamin D3 or an analogue or derivative thereof; the agent is a leukotriene inhibitor; the agent is a MCP-1 antagonist; the agent is a MMP inhibitor; the agent is an NF kappa B inhibitor; the agent is an NF kappa B inhibitor, wherein the NF kappa B inhibitor is Bay 11-7082; the agent is an NO antagonist; the agent is a p38 MAP kinase inhibitor; the agent is a p38 MAP kinase inhibitor, wherein the p38 MAP kinase inhibitor is SB 202190; the agent is a phosphodiesterase inhibitor; the agent is a TGF beta inhibitor; the agent is a thromboxane A2 antagonist; the agent is a TNFa antagonist; the agent is a TACE inhibitor; the agent is a tyrosine kinase inhibitor; the agent is a vitronectin inhibitor; the agent is a fibroblast growth factor inhibitor; the agent is a protein kinase inhibitor; the agent is a PDGF receptor kinase inhibitor; the agent is an endothelial growth factor receptor kinase inhibitor; the agent is a retinoic acid receptor antagonist; the agent is a platelet derived growth factor receptor kinase inhibitor; the agent is a fibronogin antagonist; the agent is an antimycotic agent; the agent is an antimycotic agent, wherein the antimycotic agent is sulconizole; the agent is a bisphosphonate; the agent is a phospholipase A1 inhibitor; the agent is a histamine H1/H2/H3 receptor antagonist; the agent is a macrolide antibiotic; the agent is a GPIIb/IIIa receptor antagonist; the agent is an endothelin receptor antagonist; the agent is a peroxisome proliferator-activated receptor agonist; the agent is an estrogen receptor agent; the agent is a somastostatin analogue; the agent is a neurokinin 1 antagonist; the agent is a neurokinin 3 antagonist; the agent is a VLA-4 antagonist; the agent is an osteoclast inhibitor; the agent is a DNA topoisomerase ATP hydrolyzing inhibitor; the agent is an angiotensin I converting enzyme inhibitor; the agent is an angiotensin II antagonist; the agent is an enkephalinase inhibitor; the agent is a peroxisome proliferator-activated receptor gamma agonist insulin sensitizer; the agent is a protein kinase C inhibitor; the agent is a ROCK (rho-associated kinase) inhibitor; the agent is a CXCR3 inhibitor; the agent is an Itk inhibitor; the agent is a cytosolic phospholipase A2-alpha inhibitor; the agent is a PPAR agonist; the agent is an immunosuppressant; the agent is an Erb inhibitor; the agent is an apoptosis agonist; the agent is a lipocortin agonist; the agent is a VCAM-1 antagonist; the agent is a collagen antagonist; the agent is an alpha 2 integrin antagonist; the agent is a TNF alpha inhibitor; the agent is a nitric oxide inhibitor the agent is a cathepsin inhibitor; the agent is not an anti-inflammatory agent; the agent is not a steroid; the agent is not a glucocorticosteroid; the agent is not dexamethasone; the agent is not beclomethasone; the agent is not dipropionate; the agent is not an anti-infective agent; the agent is not an antibiotic; the agent is not an anti-fungal agent; the composition comprises a polymer; the composition comprises a polymeric carrier; the anti-scarring agent inhibits adhesion between the device and a host into which the device is implanted; the device delivers the anti-scarring agent locally to tissue proximate to the device; the device has a coating that comprises the anti-scarring agent; the device has a coating that comprises the agent and is disposed on a surface of the sensor; the device has a coating that comprises the agent and directly contacts the sensor; the device has a coating that comprises the agent and indirectly contacts the sensor; the device has a coating that comprises the agent and partially covers the sensor; the device has a coating that comprises the agent and completely covers the sensor; the device has a uniform coating; the device has a non-uniform coating; the device has a discontinuous coating; the device has a patterned coating; the device has a coating with a thickness of 100 μm or less; the device has a coating with a thickness of 10 μm or less; the device has a coating, and the coating adheres to the surface of the sensor upon deployment of the sensor; the device has a coating, and wherein the coating is stable at room temperature for a period of 1 year; the device has a coating, and wherein the anti-scarring agent is present in the coating in an amount ranging between about 0.0001% to about 1% by weight; the device has a coating, and wherein the anti-scarring agent is present in the coating in an amount ranging between about 1% to about 10% by weight; the device has a coating, and wherein the anti-scarring agent is present in the coating in an amount ranging between about 10% to about 25% by weight; the device has a coating, and wherein the anti-scarring agent is present in the coating in an amount ranging between about 25% to about 70% by weight; the device has a coating, and wherein the coating further comprises a polymer; the device has a first coating having a first composition and a second coating having a second composition; the device has a first coating having a first composition and a second coating having a second composition, wherein the first composition and the second composition are different; the composition comprises a polymer; the composition comprises a polymeric carrier; the composition comprises a polymeric carrier, and wherein the polymeric carrier comprises a copolymer; the composition comprises a polymeric carrier, and wherein the polymeric carrier comprises a block copolymer; the composition comprises a polymeric carrier, and wherein the polymeric carrier comprises a random copolymer; the composition comprises a polymeric carrier, and wherein the polymeric carrier comprises a biodegradable polymer; the composition comprises a polymeric carrier, and wherein the polymeric carrier comprises a non-biodegradable polymer; the composition comprises a polymeric carrier, and wherein the polymeric carrier comprises a hydrophilic polymer; the composition comprises a polymeric carrier, and wherein the polymeric carrier comprises a hydrophobic polymer; the composition comprises a polymeric carrier, and wherein the polymeric carrier comprises a polymer having hydrophilic domains; the composition comprises a polymeric carrier, and wherein the polymeric carrier comprises a polymer having hydrophobic domains; the composition comprises a polymeric carrier, and wherein the polymeric carrier comprises a non-conductive polymer; the composition comprises a polymeric carrier, and wherein the polymeric carrier comprises an elastomer; the composition comprises a polymeric carrier, and wherein the polymeric carrier comprises a hydrogel; the composition comprises a polymeric carrier, and wherein the polymeric carrier comprises a silicone polymer; the composition comprises a polymeric carrier, and wherein the polymeric carrier comprises a hydrocarbon polymer; the composition comprises a polymeric carrier, and wherein the polymeric carrier comprises a styrene-derived polymer; the composition comprises a polymeric carrier, and wherein the polymeric carrier comprises a butadiene polymer; the composition comprises a polymeric carrier, and wherein the polymeric carrier comprises a macromer; the composition comprises a polymeric carrier, and wherein the polymeric carrier comprises a poly(ethylene glycol) polymer; the composition comprises a polymeric carrier, and wherein the polymeric carrier comprises an amorphous polymer; the device comprises a lubricious coating; the anti-scarring agent is located within pores or holes of the device; the anti-scarring agent is located within a channel, lumen, or divet of the device; the device comprises a second pharmaceutically active agent; the device comprises an anti-inflammatory agent; the device comprises an agent that inhibits infection; the device comprises an agent that inhibits infection, and wherein the agent is an anthracycline; the device comprises an agent that inhibits infection, and wherein the agent is doxorubicin; the device comprises an agent that inhibits infection, and wherein the agent is mitoxantrone; the device comprises an agent that inhibits infection, and wherein the agent is a fluoropyrimidine; the device comprises an agent that inhibits infection, and wherein the agent is 5-fluorouracil (5-FU); the device comprises an agent that inhibits infection, and wherein the agent is a folic acid antagonist; the device comprises an agent that inhibits infection, and wherein the agent is methotrexate; the device comprises an agent that inhibits infection, and wherein the agent is a podophylotoxin; the device comprises an agent that inhibits infection, and wherein the agent is etoposide; the device comprises an agent that inhibits infection, and wherein the agent is a camptothecin; the device comprises an agent that inhibits infection, and wherein the agent is a hydroxyurea; the device comprises an agent that inhibits infection, and wherein the agent is a platinum complex; the device comprises an agent that inhibits infection, and wherein the agent is cisplatin; the method further comprises an anti-thrombotic agent; the device comprises a visualization agent; the device comprises a visualization agent, wherein the visualization agent is a radiopaque material, and wherein the radiopaque material comprises a metal, a halogenated compound, or a barium containing compound; the device comprises a visualization agent, wherein the visualization agent is a radiopaque material, and wherein the radiopaque material comprises barium, tantalum, or technetium; the device comprises a visualization agent, and wherein the visualization agent is a MRI responsive material; the device comprises a visualization agent, and wherein the visualization agent comprises a gadolinium chelate; the device comprises a visualization agent, and wherein the visualization agent comprises iron, magnesium, manganese, copper, or chromium; the device comprises a visualization agent, and wherein the visualization agent comprises an iron oxide compound; the device comprises a visualization agent, and wherein the visualization agent comprises a dye, pigment, or colorant; the device comprises an echogenic material; the device comprises an echogenic material, and wherein the echogenic material is in the form of a coating; the device is sterile; the anti-scarring agent is released into tissue in the vicinity of the device after deployment of the device; the anti-scarring agent is released into tissue in the vicinity of the device after deployment of the device, and wherein the tissue is connective tissue; the anti-scarring agent is released into tissue in the vicinity of the device after deployment of the device, and wherein the tissue is muscle tissue; the anti-scarring agent is released into tissue in the vicinity of the device after deployment of the device, and wherein the tissue is nerve tissue; the anti-scarring agent is released into tissue in the vicinity of the device after deployment of the device, and wherein the tissue is epithelium tissue; the anti-scarring agent is released in effective concentrations from the device over a period ranging from the time of deployment of the device to about 1 year; the anti-scarring agent is released in effective concentrations from the device over a period ranging from about 1 month to 6 months; the anti-scarring agent is released in effective concentrations from the device over a period ranging from about 1-90 days; the anti-scarring agent is released in effective concentrations from the device at a constant rate; the anti-scarring agent is released in effective concentrations from the device at an increasing rate; the anti-scarring agent is released in effective concentrations from the device at a decreasing rate; the anti-scarring agent is released in effective concentrations from the composition comprising the anti-scarring agent by diffusion over a period ranging from the time of deployment of the device to about 90 days; the anti-scarring agent is released in effective concentrations from the composition comprising the anti-scarring agent by erosion of the composition over a period ranging from the time of deployment of the device to about 90 days; the device comprises about 0.01 μg to about 10 μg of the anti-scarring agent; the device comprises about 10 μg to about 10 mg of the anti-scarring agent; the device comprises about 10 mg to about 250 mg of the anti-scarring agent; the device comprises about 250 mg to about 1000 mg of the anti-scarring agent; the device comprises about 1000 mg to about 2500 mg of the anti-scarring agent; a surface of the device comprises less than 0.01 μg of the anti-scarring agent per mm² of device surface to which the anti-scarring agent is applied; a surface of the device comprises about 0.01 μg to about 1 μg of the anti-scarring agent per mm² of device surface to which the anti-scarring agent is applied; a surface of the device comprises about 1 μg to about 10 μg of the anti-scarring agent per mm² of device surface to which the anti-scarring agent is applied; a surface of the device comprises about 10 μg to about 250 μg of the anti-scarring agent per mm² of device surface to which the anti-scarring agent is applied; a surface of the device comprises about 250 μg to about 1000 μg of the anti-scarring agent of anti-scarring agent per mm² of device surface to which the anti-scarring agent is applied; a surface of the device comprises about 1000 μg to about 2500 μg of the anti-scarring agent per mm² of device surface to which the anti-scarring agent is applied; the combining is performed by direct affixing the agent or the composition to the sensor; the combining is performed by spraying the agent or the component onto the sensor; the combining is performed by electrospraying the agent or the composition onto the sensor; the combining is performed by dipping the sensor into a solution comprising the agent or the composition; the combining is performed by covalently attaching the agent or the composition to the sensor; the combining is performed by non-covalently attaching the agent or the composition to the sensor; the combining is performed by coating the sensor with a substance that contains the agent or the composition; the combining is performed by coating the sensor with a substance that absorbs the agent; the combining is performed by interweaving a thread composed of, or coated with, the agent or the composition; the combining is performed by completely covering the sensor with a sleeve that contains the agent or the composition; the combining is performed by covering a portion of the sensor with a sleeve that contains the agent or the composition; the combining is performed by completely covering the sensor with a cover that contains the agent or the composition; the combining is performed by covering a portion of the sensor with a cover that contains the agent or the composition; the combining is performed by completely covering the sensor with an electrospun fabric that contains the agent or the composition; the combining is performed by covering a portion of the sensor with an electrospun fabric that contains the agent or the composition; the combining is performed by completely covering the sensor with a mesh that contains the agent or the composition; the combining is performed by covering a portion of the sensor with a mesh that contains the agent or the composition; the combining is performed by constructing a portion of the sensor with the agent or the composition; the combining is performed by impregnating the sensor with the agent or the composition; the combining is performed by constructing a portion of the sensor from a degradable polymer that releases the agent; the combining is performed by dipping the sensor into a solution that comprise the agent and an inert solvent for the sensor; the combining is performed by dipping the sensor into a solution that comprises the agent and a solvent that will swill the sensor; the combining is performed by dipping the sensor into a solution that comprises the agent and a solvent that will dissolve the sensor; the combining is performed by dipping the sensor into a solution that comprises the agent, a polymer and an inert solvent for the sensor; the combining is performed by dipping the sensor into a solution that comprises the agent, a polymer and a solvent that will swill the sensor; the combining is performed by dipping the sensor into a solution that comprises the agent, a polymer and a solvent that will dissolve the sensor; the combining is performed by spraying the sensor into a solution that comprises the agent and an inert solvent for the sensor; the combining is performed by spraying the sensor into a solution that comprises the agent and a solvent that will swill the sensor; the combining is performed by spraying the sensor into a solution that comprises the agent and a solvent that will dissolve the sensor; the combining is performed by spraying the sensor into a solution that comprises the agent, a polymer and an inert solvent for the sensor; the combining is performed by spraying the sensor into a solution that comprises the agent, a polymer and a solvent that will swill the sensor; the combining is performed by spraying the sensor into a solution that comprises the agent, a polymer and a solvent that will dissolve the sensor.

Additional Features Related to Methods for Making Pumps

The methods for making the pumps as described above may also be further defined by one, two, or more of the following features: the agent inhibits cell regeneration; the agent inhibits angiogenesis; the agent inhibits fibroblast migration; the agent inhibits fibroblast proliferation; the agent inhibits deposition of extracellular matrix; the agent inhibits tissue remodeling; the agent is an angiogenesis inhibitor; the agent is a 5-lipoxygenase inhibitor or antagonist; the agent is a chemokine receptor antagonist; the agent is a cell cycle inhibitor; the agent is a taxane; the agent is an anti-microtubule agent; the agent is paclitaxel; the agent is not paclitaxel; the agent is an analogue or derivative of paclitaxel; the agent is a vinca alkaloid; the agent is camptothecin or an analogue or derivative thereof; the agent is a podophyllotoxin; the agent is a podophyllotoxin, wherein the podophyllotoxin is etoposide or an analogue or derivative thereof; the agent is an anthracycline; the agent is an anthracycline, wherein the anthracycline is doxorubicin or an analogue or derivative thereof; the agent is an anthracycline, wherein the anthracycline is mitoxantrone or an analogue or derivative thereof; the agent is a platinum compound; the agent is a nitrosourea; the agent is a nitroimidazole; the agent is a folic acid antagonist; the agent is a cytidine analogue; the agent is a pyrimidine analogue; the agent is a fluoropyrimidine analogue; the agent is a purine analogue; the agent is a nitrogen mustard or an analogue or derivative thereof; the agent is a hydroxyurea; the agent is a mytomicin or an analogue or derivative thereof; the agent is an alkyl sulfonate; the agent is a benzamide or an analogue or derivative thereof; the agent is a nicotinamide or an analogue or derivative thereof; the agent is a halogenated sugar or an analogue or derivative thereof; the agent is a DNA alkylating agent; the agent is an anti-microtubule agent; the agent is a topoisomerase inhibitor; the agent is a DNA cleaving agent; the agent is an antimetabolite; the agent inhibits adenosine deaminase; the agent inhibits purine ring synthesis; the agent is a nucleotide interconversion inhibitor; the agent inhibits dihydrofolate reduction; the agent blocks thymidine monophosphate; the agent causes DNA damage; the agent is a DNA intercalation agent; the agent is a RNA synthesis inhibitor; the agent is a pyrimidine synthesis inhibitor; the agent inhibits ribonucleotide synthesis or function; the agent inhibits thymidine monophosphate synthesis or function; the agent inhibits DNA synthesis; the agent causes DNA adduct formation; the agent inhibits protein synthesis; the agent inhibits microtubule function; the agent is a cyclin dependent protein kinase inhibitor; the agent is an epidermal growth factor kinase inhibitor; the agent is an elastase inhibitor; the agent is a factor Xa inhibitor; the agent is a farnesyltransferase inhibitor; the agent is a fibrinogen antagonist; the agent is a guanylate cyclase stimulant; the agent is a heat shock protein 90 antagonist; the agent is a heat shock protein 90 antagonist, wherein the heat shock protein 90 antagonist is geldanamycin or an analogue or derivative thereof; the agent is a guanylate cyclase stimulant; the agent is a HMGCoA reductase inhibitor; the agent is a HMGCoA reductase inhibitor, wherein the HMGCoA reductase inhibitor is simvastatin or an analogue or derivative thereof; the agent is a hydroorotate dehydrogenase inhibitor; the agent is an IKK2 inhibitor; the agent is an IL-1 antagonist; the agent is an ICE antagonist; the agent is an IRAK antagonist; the agent is an IL-4 agonist; the agent is an immunomodulatory agent; the agent is sirolimus or an analogue or derivative thereof; the agent is not sirolimus; the agent is everolimus or an analogue or derivative thereof; the agent is tacrolimus or an analogue or derivative thereof; the agent is not tacrolimus; the agent is biolmus or an analogue or derivative thereof; the agent is tresperimus or an analogue or derivative thereof; the agent is auranofin or an analogue or derivative thereof; the agent is 27-O-demethylrapamycin or an analogue or derivative thereof; the agent is gusperimus or an analogue or derivative thereof; the agent is pimecrolimus or an analogue or derivative thereof; the agent is ABT-578 or an analogue or derivative thereof; the agent is an inosine monophosphate dehydrogenase (IMPDH) inhibitor; the agent is an IMPDH inhibitor, wherein the IMPDH inhibitor is mycophenolic acid or an analogue or derivative thereof; the agent is an IMPDH inhibitor, wherein the IMPDH inhibitor is 1-alpha-25 dihydroxy vitamin D3 or an analogue or derivative thereof; the agent is a leukotriene inhibitor; the agent is a MCP-1 antagonist; the agent is a MMP inhibitor; the agent is an NF kappa B inhibitor; the agent is an NF kappa B inhibitor, wherein the NF kappa B inhibitor is Bay 11-7082; the agent is an NO antagonist; the agent is a p38 MAP kinase inhibitor; the agent is a p38 MAP kinase inhibitor, wherein the p38 MAP kinase inhibitor is SB 202190; the agent is a phosphodiesterase inhibitor; the agent is a TGF beta inhibitor; the agent is a thromboxane A2 antagonist; the agent is a TNFa antagonist; the agent is a TACE inhibitor; the agent is a tyrosine kinase inhibitor; the agent is a vitronectin inhibitor; the agent is a fibroblast growth factor inhibitor; the agent is a protein kinase inhibitor; the agent is a PDGF receptor kinase inhibitor; the agent is an endothelial growth factor receptor kinase inhibitor; the agent is a retinoic acid receptor antagonist; the agent is a platelet derived growth factor receptor kinase inhibitor; the agent is a fibronogin antagonist; the agent is an antimycotic agent; the agent is an antimycotic agent, wherein the antimycotic agent is sulconizole; the agent is a bisphosphonate; the agent is a phospholipase A1 inhibitor; the agent is a histamine H1/H2/H3 receptor antagonist; the agent is a macrolide antibiotic; the agent is a GPIIb/IIIa receptor antagonist; the agent is an endothelin receptor antagonist; the agent is a peroxisome proliferator-activated receptor agonist; the agent is an estrogen receptor agent; the agent is a somastostatin analogue; the agent is a neurokinin 1 antagonist; the agent is a neurokinin 3 antagonist; the agent is a VLA-4 antagonist; the agent is an osteoclast inhibitor; the agent is a DNA topoisomerase ATP hydrolyzing inhibitor; the agent is an angiotensin I converting enzyme inhibitor; the agent is an angiotensin II antagonist; the agent is an enkephalinase inhibitor; the agent is a peroxisome proliferator-activated receptor gamma agonist insulin sensitizer; the agent is a protein kinase C inhibitor; the agent is a ROCK (rho-associated kinase) inhibitor; the agent is a CXCR3 inhibitor; the agent is an Itk inhibitor; the agent is a cytosolic phospholipase A2-alpha inhibitor; the agent is a PPAR agonist; the agent is an immunosuppressant; the agent is an Erb inhibitor; the agent is an apoptosis agonist; the agent is a lipocortin agonist; the agent is a VCAM-1 antagonist; the agent is a collagen antagonist; the agent is an alpha 2 integrin antagonist; the agent is a TNF alpha inhibitor; the agent is a nitric oxide inhibitor the agent is a cathepsin inhibitor; the agent is not an anti-inflammatory agent; the agent is not a steroid; the agent is not a glucocorticosteroid; the agent is not dexamethasone; the agent is not beclomethasone; the agent is not dipropionate; the agent is not an anti-infective agent; the agent is not an antibiotic; the agent is not an anti-fungal agent; the composition comprises a polymer; the composition comprises a polymeric carrier; the anti-scarring agent inhibits adhesion between the device and a host into which the device is implanted; the device delivers the anti-scarring agent locally to tissue proximate to the device; the device has a coating that comprises the anti-scarring agent; the device has a coating that comprises the agent and is disposed on a surface of the pump; the device has a coating that comprises the agent and directly contacts the pump; the device has a coating that comprises the agent and indirectly contacts the pump; the device has a coating that comprises the agent and partially covers the pump; the device has a coating that comprises the agent and completely covers the pump; the device has a uniform coating; the device has a non-uniform coating; the device has a discontinuous coating; the device has a patterned coating; the device has a coating with a thickness of 100 μm or less; the device has a coating with a thickness of 10 μm or less; the device has a coating, and the coating adheres to the surface of the pump upon deployment of the pump; the device has a coating, and wherein the coating is stable at room temperature for a period of 1 year; the device has a coating, and wherein the anti-scarring agent is present in the coating in an amount ranging between about 0.0001% to about 1% by weight; the device has a coating, and wherein the anti-scarring agent is present in the coating in an amount ranging between about 1% to about 10% by weight; the device has a coating, and wherein the anti-scarring agent is present in the coating in an amount ranging between about 10% to about 25% by weight; the device has a coating, and wherein the anti-scarring agent is present in the coating in an amount ranging between about 25% to about 70% by weight; the device has a coating, and wherein the coating further comprises a polymer; the device has a first coating having a first composition and a second coating having a second composition; the device has a first coating having a first composition and a second coating having a second composition, wherein the first composition and the second composition are different; the composition comprises a polymer; the composition comprises a polymeric carrier; the composition comprises a polymeric carrier, and wherein the polymeric carrier comprises a copolymer; the composition comprises a polymeric carrier, and wherein the polymeric carrier comprises a block copolymer; the composition comprises a polymeric carrier, and wherein the polymeric carrier comprises a random copolymer; the composition comprises a polymeric carrier, and wherein the polymeric carrier comprises a biodegradable polymer; the composition comprises a polymeric carrier, and wherein the polymeric carrier comprises a non-biodegradable polymer; the composition comprises a polymeric carrier, and wherein the polymeric carrier comprises a hydrophilic polymer; the composition comprises a polymeric carrier, and wherein the polymeric carrier comprises a hydrophobic polymer; the composition comprises a polymeric carrier, and wherein the polymeric carrier comprises a polymer having hydrophilic domains; the composition comprises a polymeric carrier, and wherein the polymeric carrier comprises a polymer having hydrophobic domains; the composition comprises a polymeric carrier, and wherein the polymeric carrier comprises a non-conductive polymer; the composition comprises a polymeric carrier, and wherein the polymeric carrier comprises an elastomer; the composition comprises a polymeric carrier, and wherein the polymeric carrier comprises a hydrogel; the composition comprises a polymeric carrier, and wherein the polymeric carrier comprises a silicone polymer; the composition comprises a polymeric carrier, and wherein the polymeric carrier comprises a hydrocarbon polymer; the composition comprises a polymeric carrier, and wherein the polymeric carrier comprises a styrene-derived polymer; the composition comprises a polymeric carrier, and wherein the polymeric carrier comprises a butadiene polymer; the composition comprises a polymeric carrier, and wherein the polymeric carrier comprises a macromer; the composition comprises a polymeric carrier, and wherein the polymeric carrier comprises a poly(ethylene glycol) polymer; the composition comprises a polymeric carrier, and wherein the polymeric carrier comprises an amorphous polymer; the device comprises a lubricious coating; the anti-scarring agent is located within pores or holes of the device; the anti-scarring agent is located within a channel, lumen, or divet of the device; the device comprises a second pharmaceutically active agent; the device comprises an anti-inflammatory agent; the device comprises an agent that inhibits infection; the device comprises an agent that inhibits infection, and wherein the agent is an anthracycline; the device comprises an agent that inhibits infection, and wherein the agent is doxorubicin; the device comprises an agent that inhibits infection, and wherein the agent is mitoxantrone; the device comprises an agent that inhibits infection, and wherein the agent is a fluoropyrimidine; the device comprises an agent that inhibits infection, and wherein the agent is 5-fluorouracil (5-FU); the device comprises an agent that inhibits infection, and wherein the agent is a folic acid antagonist; the device comprises an agent that inhibits infection, and wherein the agent is methotrexate; the device comprises an agent that inhibits infection, and wherein the agent is a podophylotoxin; the device comprises an agent that inhibits infection, and wherein the agent is etoposide; the device comprises an agent that inhibits infection, and wherein the agent is a camptothecin; the device comprises an agent that inhibits infection, and wherein the agent is a hydroxyurea; the device comprises an agent that inhibits infection, and wherein the agent is a platinum complex; the device comprises an agent that inhibits infection, and wherein the agent is cisplatin; the method further comprises an anti-thrombotic agent; the device comprises a visualization agent; the device comprises a visualization agent, wherein the visualization agent is a radiopaque material, and wherein the radiopaque material comprises a metal, a halogenated compound, or a barium containing compound; the device comprises a visualization agent, wherein the visualization agent is a radiopaque material, and wherein the radiopaque material comprises barium, tantalum, or technetium; the device comprises a visualization agent, and wherein the visualization agent is a MRI responsive material; the device comprises a visualization agent, and wherein the visualization agent comprises a gadolinium chelate; the device comprises a visualization agent, and wherein the visualization agent comprises iron, magnesium, manganese, copper, or chromium; the device comprises a visualization agent, and wherein the visualization agent comprises an iron oxide compound; the device comprises a visualization agent, and wherein the visualization agent comprises a dye, pigment, or colorant; the device comprises an echogenic material; the device comprises an echogenic material, and wherein the echogenic material is in the form of a coating; the device is sterile; the anti-scarring agent is released into tissue in the vicinity of the device after deployment of the device; the anti-scarring agent is released into tissue in the vicinity of the device after deployment of the device, and wherein the tissue is connective tissue; the anti-scarring agent is released into tissue in the vicinity of the device after deployment of the device, and wherein the tissue is muscle tissue; the anti-scarring agent is released into tissue in the vicinity of the device after deployment of the device, and wherein the tissue is nerve tissue; the anti-scarring agent is released into tissue in the vicinity of the device after deployment of the device, and wherein the tissue is epithelium tissue; the anti-scarring agent is released in effective concentrations from the device over a period ranging from the time of deployment of the device to about 1 year; the anti-scarring agent is released in effective concentrations from the device over a period ranging from about 1 month to 6 months; the anti-scarring agent is released in effective concentrations from the device over a period ranging from about 1-90 days; the anti-scarring agent is released in effective concentrations from the device at a constant rate; the anti-scarring agent is released in effective concentrations from the device at an increasing rate; the anti-scarring agent is released in effective concentrations from the device at a decreasing rate; the anti-scarring agent is released in effective concentrations from the composition comprising the anti-scarring agent by diffusion over a period ranging from the time of deployment of the device to about 90 days; the anti-scarring agent is released in effective concentrations from the composition comprising the anti-scarring agent by erosion of the composition over a period ranging from the time of deployment of the device to about 90 days; the device comprises about 0.01 μg to about 10 μg of the anti-scarring agent; the device comprises about 10 μg to about 10 mg of the anti-scarring agent; the device comprises about 10 mg to about 250 mg of the anti-scarring agent; the device comprises about 250 mg to about 1000 mg of the anti-scarring agent; the device comprises about 1000 mg to about 2500 mg of the anti-scarring agent; a surface of the device comprises less than 0.01 μg of the anti-scarring agent per mm² of device surface to which the anti-scarring agent is applied; a surface of the device comprises about 0.01 μg to about 1 μg of the anti-scarring agent per mm² of device surface to which the anti-scarring agent is applied; a surface of the device comprises about 1 μg to about 10 μg of the anti-scarring agent per mm² of device surface to which the anti-scarring agent is applied; a surface of the device comprises about 10 μg to about 250 μg of the anti-scarring agent per mm² of device surface to which the anti-scarring agent is applied; a surface of the device comprises about 250 μg to about 1000 μg of the anti-scarring agent of anti-scarring agent per mm² of device surface to which the anti-scarring agent is applied; a surface of the device comprises about 1000 μg to about 2500 μg of the anti-scarring agent per mm² of device surface to which the anti-scarring agent is applied; the combining is performed by direct affixing the agent or the composition to the pump; the combining is performed by spraying the agent or the component onto the pump; the combining is performed by electrospraying the agent or the composition onto the pump; the combining is performed by dipping the pump into a solution comprising the agent or the composition; the combining is performed by covalently attaching the agent or the composition to the pump; the combining is performed by non-covalently attaching the agent or the composition to the pump; the combining is performed by coating the pump with a substance that contains the agent or the composition; the combining is performed by coating the pump with a substance that absorbs the agent; the combining is performed by interweaving the pump with a thread composed of, or coated with, the agent or the composition; the combining is performed by completely covering the pump with a sleeve that contains the agent or the composition; the combining is performed by covering a portion of the pump with a sleeve that contains the agent or the composition; the combining is performed by completely covering the pump with a cover that contains the agent or the composition; the combining is performed by covering a portion of the pump with a cover that contains the agent or the composition; the combining is performed by completely covering the pump with an electrospun fabric that contains the agent or the composition; the combining is performed by covering a portion of the pump with an electrospun fabric that contains the agent or the composition; the combining is performed by completely covering the pump with a mesh that contains the agent or the composition; the combining is performed by covering a portion of the pump with a mesh that contains the agent or the composition; the combining is performed by constructing a portion of the pump with the agent or the composition; the combining is performed by impregnating the pump with the agent or the composition; the combining is performed by constructing a portion of the pump from a degradable polymer that releases the agent; the combining is performed by dipping the pump into a solution that comprise the agent and an inert solvent for the pump; the combining is performed by dipping the pump into a solution that comprises the agent and a solvent that will swill the pump; the combining is performed by dipping the pump into a solution that comprises the agent and a solvent that will dissolve the pump; the combining is performed by dipping the pump into a solution that comprises the agent, a polymer and an inert solvent for the pump; the combining is performed by dipping the pump into a solution that comprises the agent, a polymer and a solvent that will swill the pump; the combining is performed by dipping the pump into a solution that comprises the agent, a polymer and a solvent that will dissolve the pump; the combining-is performed by spraying the pump into a solution that comprises the agent and an inert solvent for the pump; the combining is performed by spraying the pump into a solution that comprises the agent and a solvent that will swill the pump; the combining is performed by spraying the pump into a solution that comprises the agent and a solvent that will dissolve the pump; the combining is performed by spraying the pump into a solution that comprises the agent, a polymer and an inert solvent for the pump; the combining is performed by spraying the pump into a solution that comprises the agent, a polymer and a solvent that will swill the pump; and the combining is performed by spraying the pump into a solution that comprises the agent, a polymer and a solvent that will dissolve the pump.

The following examples are offered by way of illustration, and not by way of limitation.

### EXAMPLES

### Example 1

### Parylene Coating

A metallic portion of a housing of the device (e.g., MiniMed 2007 implantable insulin pump, Medtronic, Inc.) is washed by dipping it into HPLC grade isopropanol. A parylene primer layer (about 1 to 10 um) is deposited onto the cleaned device using a parylene coater (e.g., PDS 2010 LABCOATER 2 from Cookson Electronics) and di-p-xylylene (PARYLENE N) or dichloro-di-p-xylylene (PARYLENE D) (both available from Specialty Coating Systems, Indianapolis, Ind.) as the coating feed material.

### Example 2

### Paclitaxel Coating—Partial Coating

Paclitaxel solutions are prepared by dissolving paclitaxel (5 mg, 10 mg, 50 mg, 100 mg, 200 mg and 500 mg) in 5 ml HPLC grade THF. A coated portion of a parylene-coated device (as prepared in, e.g., Example 1) is dipped into a paclitaxel/THF solution. After a selected incubation time, the device is removed from the solution and dried in a forced air oven (50° C.). The device then is further dried in a vacuum oven overnight. The amount of paclitaxel used in each solution and the incubation time is varied such that the amount of paclitaxel coated onto the device is in the range of 0.06 μg/mm² to 10 μg/mm² (μg paclitaxel/mm² of the device which is coated with paclitaxel after being placed in the THF/paclitaxel solution). The time during which the device is maintained in the paclitaxel/THF solution may be varied, where longer soak times generally provide for more paclitaxel to be adsorbed onto the device. In additional examples, one of the following exemplary compounds may be used in lieu of paclitaxel: mitoxantrone, doxorubicin, epithilone B, etoposide, TAXOTERE, tubercidin, vinblastine, geldanamycin, simvastatin, sirolimus, everolimus, halifuginone, mycophenolic acid, mithramycin, pimecrolimus, 1-alpha-25 dihydroxy vitamin D₃, Bay 11-7082, SB202190, and sulconizole.

### Example 3

### Paclitaxel Coating—Complete Coating

Paclitaxel solutions are prepared by dissolving paclitaxel (5 mg, 10 mg, 50 mg, 100 mg, 200 mg and 500 mg) in 5 ml HPLC grade THF. An entire parylene coated device (coated as in, e.g., Example 1) is then dipped into the paclitaxel/THF solution. After a selected incubation time, the device is removed and dried in a forced air oven (50° C.). The device is then further dried in a vacuum oven overnight. The amount of paclitaxel used in each solution and the incubation time is varied such that the amount of paclitaxel coated onto the device is in the range of 0.06 μg/mm² to 10 μg/mm². In additional examples, one of the following exemplary compounds may be used in lieu of paclitaxel: mitoxantrone, doxorubicin, epithilone B, etoposide, TAXOTERE, tubercidin, halifuginone, vinblastine, geldanamycin, simvastatin, sirolimus, everolimus, mithramycin, pimecrolimus, mycophenolic acid, 1-alpha-25 dihydroxy vitamin D₃, Bay 11-7082, SB202190, and sulconizole.

### Example 4

### Application of a Parylene Overcoat

A paclitaxel coated device (prepared as in, e.g., Example 2 or 3) is placed in a parylene coater and an additional thin layer of parylene is deposited on the paclitaxel coated device using the procedure described in Example 1. The coating duration is selected to provide a parylene top-coat thickness that will cause the device to have a desired elution profile for the paclitaxel.

### Example 5

### Application of an Echogenic Coating Layer

DESMODUR (an isocyanate pre-polymer Bayer AG) (25% w/v) is dissolved in a 50:50 mixture of dimethylsulfoxide and tetrahydrofuran. A paclitaxel/parylene overcoated device (prepared as in, e.g., Example 4) is then dipped into the pre-polymer solution. The device is removed from the solution after a selected incubation time, and the coating is then partially dried at room temperature for 3 to 5 minutes. The device is then immersed in a beaker of water (room temperature) for 3-5 minutes to cause the polymerization reaction to occur rapidly. An echogenic coating is formed.

### Example 6

### Paclitaxel/Polymer Coating—Partial Coating

Several 5% solutions of poly(ethylene-co-vinyl acetate) {EVA} (60% vinyl acetate) are prepared using THF as the solvent. Selected amounts of paclitaxel (0.01%, 0.05%, 0.1%, 0.5%, 1%, 5%, 10%, 20%, 30% (w/w drug to polymer) are added to the EVA solutions. The catheter portion of an implantable pump device or a portion thereof is dipped into a paclitaxel/EVA solution. After removing the device from the solution, the coating is dried by placing the device in a forced air oven (40° C.) for 3 hours. The coated device is then further dried under vacuum for 24 hours. This dip coating process may be repeated to increase the amount of polymer/paclitaxel coated onto the device. In addition, higher paclitaxel concentrations in the polymer/THF/paclitaxel solution and/or a longer soak time may be used to increase the amount of polymer/paclitaxel that is coated onto the device. In additional examples, one of the following exemplary compounds may be used in lieu of paclitaxel: mitoxantrone, doxorubicin, epithilone B, etoposide, TAXOTERE, tubercidin, vinblastine, geldanamycin, simvastatin, mithramycin, pimecrolimus, halifuginone, sirolimus, everolimus, mycophenolic acid, 1-alpha-25 dihydroxy vitamin D₃, Bay 11-7082, SB202190, and sulconizole.

### Example 7

### Paclitaxel-Heparin Coating

Several 5% solutions of poly(ethylene-co-vinyl acetate) {EVA} (60% vinyl acetate) are prepared using THF as the solvent. Selected amounts (0.01%, 0.05%, 0.1%, 0.5%, 1%, 5%, 10%, 20%, 30% (w/w drug to polymer) of paclitaxel and a solution of tridodecyl methyl ammonium chloride-heparin complex (PolySciences) are added to each of the EVA solutions. All or a portion of a catheter portion of the device is dipped into the paclitaxel/EVA solution. After removing the device from the solution, the coating is dried by placing the device in a forced air oven (40° C.) for 3 hours. The coated device is then further dried under vacuum for 24 hours. The dip coating process may be repeated to increase the amount of polymer/heparin complex coated onto the device. In additional examples, one of the following exemplary compounds may be used in lieu of paclitaxel: mitoxantrone, doxorubicin, epithilone B, etoposide, TAXOTERE, tubercidin, halifuginone, vinblastine, geldanamycin, simvastatin, mithramycin, pimecrolimus, sirolimus, everolimus, mycophenolic acid, 1-alpha-25 dihydroxy vitamin D₃, Bay 11-7082, SB202190, and sulconizole.

### Example 8

### Paclitaxel—Heparin/Heparin Coating

An uncoated portion of a paclitaxel-heparin coated device (prepared as in, e.g., Example 7) is dipped into a 5% EVA/THF solution containing a selected amount of a tridodecyl methyl ammonium chloride-heparin complex solution (PolySciences) (0.1%, 0.5%, 1%, 2.5%, 5%, 10% (v/v)). After removing the device from the solution, the coating is dried by placing the device in a forced air oven (40° C.) for 3 hours. The coated device is then further dried under vacuum for 24 hours. This provides a device with a paclitaxel/heparin coating on one or more portions of the device and a heparin coating on one or more other parts of the device. In additional examples, one of the following exemplary compounds may be used in lieu of paclitaxel: mitoxantrone, doxorubicin, epithilone B, etoposide, mithramycin, pimecrolimus, TAXOTERE, tubercidin, vinblastine, geldanamycin, halifuginone, simvastatin, sirolimus, everolimus, mycophenolic acid, 1-alpha-25 dihydroxy vitamin D₃, Bay 11-7082, SB202190, and sulconizole.

### Example 9

### Paclitaxel/Polymer Coating—Partial Coating

Several 5% solutions of poly(styrene-co-isobutylene-styrene) (SIBS) are prepared using THF as the solvent. A selected amount of paclitaxel is added to each SIBS solution. One or more portions of the catheter portion of an implantable pump device are dipped into the paclitaxel/SIBS solution. After removing the device from the solution, the coating is dried by placing the device in a forced air oven (40° C.) for 3 hours. The coated device is then further dried under vacuum for 24 hours. The dip coating process may be repeated to increase the amount of polymer/paclitaxel coated onto the device. In addition, higher paclitaxel concentrations in the polymer/THF/paclitaxel solution and/or a longer soak time may be used to increase the amount of polymer/paclitaxel that is coated onto the device. In additional examples, one of the following exemplary compounds may be used in lieu of paclitaxel: mitoxantrone, doxorubicin, mithramycin, pimecrolimus, epithilone B, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 10

### Paclitaxel/Polymer Coating—Echogenic Overcoat

A paclitaxel-coated device prepared as in Example 9 is dipped into a DESMODUR solution (50% w/v) (50:50 mixture of dimethylsulfoxide and tetrahydrofuran). The device is then removed and the coating is partially dried at room temperature for 3 to 5 minutes. The device is then immersed in a beaker of water (room temperature) for 3-5 minutes to cause the polymerization reaction to occur rapidly. An echogenic coating is thereby formed. In additional examples, one of the following exemplary compounds may be used in lieu of paclitaxel: mitoxantrone, doxorubicin, epithilone B, mithramycin, pimecrolimus, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 11

### Polymer/Echogenic Coating

A 5% solution of poly(styrene-co-isobutylene-styrene) (SIBS) is prepared using THF as the solvent. The catheter portion of an implantable pump device is dipped into the SIBS solution. After a selected incubation time, the device is removed from the solution, and the coating is dried by placing the device in a forced air oven (40° C.) for 3 hours. The coated device is then further dried under vacuum for 24 hours.

A coated device is dipped into a DESMODUR solution (50:50 mixture of dimethylsulfoxide and tetrahydrofuran). The device is then removed and the coating is then partially dried at room temperature for 3 to 5 minutes. The device is then immersed in a beaker of water (room temperature) for 3-5 minutes to cause the polymerization reaction to occur rapidly. The device is dried under vacuum for 24 hours at room temperature. All or a portion of the coated device is immersed into a solution of paclitaxel (5% w/v in methanol). The device is removed and dried at 40° C. for 1 hour and then under vacuum for 24 hours.

The amount of paclitaxel absorbed by the polymeric coating can be altered by changing the paclitaxel concentration, the immersion time as well as the solvent composition of the paclitaxel solution. In additional examples, one of the following exemplary compounds may be used in lieu of paclitaxel: mitoxantrone, doxorubicin, epithilone B, etoposide, TAXOTERE, mithramycin, pimecrolimus, tubercidin, vinblastine, geldanamycin, halifuginone, simvastatin, sirolimus, everolimus, mycophenolic acid, 1-alpha-25 dihydroxy vitamin D₃, Bay 11-7082, SB202190, and sulconizole.

### Example 12

### Paclitaxel/Siloxane Coating—Partial Coating

The housing of an implantable pump device is coated with a silioxane layer by exposing the device to gaseous tetramethylcyclotetrasiloxane that is then polymerized by low energy plasma polymerization onto the device surface. The thickness of the siloxane layer can be increased by increasing the polymerization time. After polymerization, a portion of the coated device is then immersed into a paclitaxel/THF solution (5% w/v) for a selected period of time to allow the paclitaxel to absorb into the siloxane coating. The device is then removed from the solution and is dried for 2 hours at 40° C. in a forced air oven. The device is then further dried under vacuum at room temperature for 24 hours. The amount of paclitaxel coated onto the device can be varied by altering the concentration of the paclitaxel/THF solution and by altering the immersion time of the device in the paclitaxel THF solution. In additional examples, one of the following exemplary compounds may be used in lieu of paclitaxel: mitoxantrone, doxorubicin, epithilone B, etoposide, TAXOTERE, tubercidin, mithramycin, pimecrolimus, vinblastine, geldanamycin, halifuginone, simvastatin, sirolimus, everolimus, mycophenolic acid, 1-alpha-25 dihydroxy vitamin D₃, Bay 11-7082, SB202190, and sulconizole.

### Example 13

### Spray-Coated Devices

Several 2% solutions of poly(styrene-co-isobutylene-styrene) (SIBS) (50 ml) are prepared using THF as the solvent. A selected amount of paclitaxel (0.01%, 0.05%, 0.1%, 0.5%, 1%, 2.5%, 5%, 10% and 20% (w/w with respect to the polymer)) is added to each solution. An implantable pump device is held with a pair of tweezers and is then spray coated with one of the paclitaxel/polymer solutions using an airbrush. The device is then air-dried. The device is then held in a new location using the tweezers and a second coat of a paclitaxel/polymer solution having the same concentration is applied to the device. The device is air-dried and is then dried under vacuum at room temperature overnight. The total amount of paclitaxel coated onto the device can be altered by changing the paclitaxel content in the solution as well as by increasing the number of coatings that are applied. In additional examples, one of the following exemplary compounds may be used in lieu of paclitaxel: mitoxantrone, doxorubicin, epithilone B, etoposide, TAXOTERE, tubercidin, mithramycin, pimecrolimus, vinblastine, geldanamycin, simvastatin, sirolimus, everolimus, mycophenolic acid, 1-alpha-25 dihydroxy vitamin D₃, Bay 11-7082, SB202190, and sulconizole.

### Example 14

### Drug Coated Device-Non-Degradable

The catheter portion of an implantable pump device is attached to a rotating mandrel. A solution of paclitaxel (5% w/w) in a polyurethane (CHRONOFLEX 85A; CardioTech Biomaterials)/THF solution (2.5% w/v) is then sprayed onto all or a portion of the outer surface of the device. The solution is sprayed on at a rate that ensures that the device is not damaged or saturated with the sprayed solution. The device is allowed to air dry after which it is dried under vacuum for 24 hours. In additional examples, one of the following exemplary compounds may be used in lieu of paclitaxel: mitoxantrone, doxorubicin, epithilone B, etoposide, TAXOTERE, tubercidin, mithramycin, pimecrolimus, vinblastine, geldanamycin, simvastatin, sirolimus, everolimus, mycophenolic acid, 1-alpha-25 dihydroxy vitamin D₃, Bay 11-7082, SB202190, and sulconizole.

### Example 15

### Drug Coated Device—Degradable

The catheter portion of an implantable pump device is attached to a rotating mandrel. A paclitaxel (5% w/w) in a PLGA/ethyl acetate solution (2.5% w/v) is then sprayed onto all or portion of the outer surface of the device. The solution is sprayed on at a rate that ensures that the device is not damaged or saturated with the sprayed solution. The device is allowed to air dry, after which it is dried under vacuum at room temperature for 24 hours. In additional examples, one of the following exemplary compounds may be used in lieu of paclitaxel: mitoxantrone, doxorubicin, epithilone B, etoposide, TAXOTERE, tubercidin, vinblastine, geldanamycin, simvastatin, sirolimus, mithramycin, pimecrolimus, everolimus, mycophenolic acid, 1-alpha-25 dihydroxy vitamin D₃, Bay 11-7082, SB202190, and sulconizole.

### Example 16

### Drug Coated Device—Degradable Overcoat

A drug-coated catheter portion of an implantable pump device prepared as in Example 14 or Example 15 is attached to a rotating mandrel. A PLGA/ethyl acetate solution (2.5% w/v) is then sprayed onto all or a portion of the outer surface of the device, such that a coating is formed over the first drug containing coating. The solution is sprayed on at a rate that ensures that the device is not damaged or saturated with the sprayed solution. The device is allowed to air dry after which it is dried under vacuum at room temperature for 24 hours.

### Example 17

### Drug-Loaded Microsphere Formulation

Paclitaxel (10% w/w) is added to a solution of PLGA (50/50, Mw≈54,000) in DCM (5% w/v). The solution is vortexed and then poured into a stirred (overhead stirrer with a 3 bladed TEFLON coated stirrer) aqueous PVA solution (approx. 89% hydrolyzed, Mw≈13,000, 2% w/v). The solution is stirred for 6 hours after which the solution is centrifuged to sediment the microspheres. The microspheres are resuspended in water. The centrifugation—ishing process is repeated 4 times. The final microsphere solution is flash frozen in an acetone/dry-ice bath. The frozen solution is then freeze-dried to produce a fine powder. The size of the microspheres formed can be altered by changing the stirring speed and/or the PVA solution concentration. The freeze dried powder can be resuspended in PBS or saline and can be used for direct injection, as an incubation fluid or as an irrigation fluid. In additional examples, one of the following exemplary compounds may be used in lieu of paclitaxel: mitoxantrone, doxorubicin, epithilone B, etoposide, TAXOTERE, mithramycin, pimecrolimus, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 18

### Drug Coated Device (Exterior Coating)

All or a portion of the catheter portion of an implantable pump device is dipped into a polyurethane (CHRONOFLEX 85A)/THF solution (2.5% w/v). The coated device is allowed to air dry for 10 seconds. The device is then rolled in powdered paclitaxel that has been spread thinly on a piece of release liner to provide a device coated with between 0.1 to 10 mg of paclitaxel. The rolling process is done in such a manner that the paclitaxel powder predominantly adheres to the exterior side of the coated device. The device is air-dried for 1 hour followed by vacuum drying at room temperature for 24 hours. In additional examples, one of the following exemplary compounds may be used in lieu of paclitaxel: mitoxantrone, doxorubicin, epithilone B, mithramycin, pimecrolimus, etoposide, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 19

### Drug Coated Device (Exterior Coating) with a Heparin Coating

A drug-coated device prepared as in Example 18 is further coated with a heparin coating. A device prepared as in Example 18 is dipped into a solution of heparin-benzalkonium chloride complex (1.5% (w/v) in isopropanol, STS Biopolymers). The device is removed from the solution and air-dried for 1 hour followed by vacuum drying for 24 hours. This process coats both the interior and exterior surfaces of the device with heparin.

### Example 20

### Partial Drug Coating of a Device

The catheter portion of an implantable pump device is attached to a rotating mandrel. A mask system is set up so that only a portion of the device surface is exposed. A solution of paclitaxel (5% w/w) in a polyurethane (CHRONOFLEX 85A)/THF solution (2.5% w/v) is then sprayed onto the exposed portion of the device. The solution is sprayed on at a rate that ensures that the device is not damaged or saturated with the sprayed solution. The device is allowed to air dry after which it is dried under vacuum at room temperature for 24 hours. In additional examples, one of the following exemplary compounds may be used in lieu of paclitaxel: mitoxantrone, doxorubicin, epithilone B, etoposide, TAXOTERE, tubercidin, vinblastine, geldanamycin, mithramycin, pimecrolimus, simvastatin, sirolimus, everolimus, mycophenolic acid, 1-alpha-25 dihydroxy vitamin D₃, Bay 11-7082, SB202190, and sulconizole.

### Example 21

### Drug—Dexamethasone Coated Device

The catheter portion of an implantable pump device is coated as in Example 20. The mask is then rearranged so that a previously masked portion of the device is exposed. The exposed portion of the device is then sprayed with a dexamethasone (10% w/w)/polyurethane (CHRONOFLEX 85A)/THF solution (2.5% w/v). The device is air dried, after which it is dried under vacuum at room temperature for 24 hours. In additional examples, one of the following exemplary compounds may be used in lieu of paclitaxelmitoxantrone, doxorubicin, epithilone B, etoposide, TAXOTERE, tubercidin, vinblastine, mithramycin, pimecrolimus, geldanamycin, simvastatin, sirolimus, everolimus, mycophenolic acid, 1-alpha-25 dihydroxy vitamin D₃, Bay 11-7082, SB202190, and sulconizole.

### Example 22

### Drug—Heparin Coated Device

The catheter portion of an implantable pump device is coated as in Example 20. The mask is then rearranged so that only a previously masked portion of the device is exposed. The exposed surface of the device is then sprayed with a heparin-benzalkonium chloride complex (1.5% (w/v) in isopropanol (STS Biopolymers). The sample is air dried after which it is dried under vacuum for 24 hours. In additional examples, one of the following exemplary compounds may be used in lieu of paclitaxel: mitoxantrone, doxorubicin, epithilone B, etoposide, TAXOTERE, tubercidin, vinblastine, geldanamycin, mithramycin, pimecrolimus, simvastatin, sirolimus, everolimus, mycophenolic acid, 1-alpha-25 dihydroxy vitamin D₃, Bay 11-7082, SB202190, and sulconizole.

### Example 23

### Drug-Dexamethaxone Coated Device

The catheter portion of an implantable pump device is attached to a rotating mandrel. A solution of paclitaxel (5% w/w) and dexamethazone (5% w/w) in a PLGA (50/50, Mw≈54,000)/ethyl acetate solution (2.5% w/v) is sprayed onto all or a portion of the device. The solution is sprayed on at a rate that ensures that the device is not damaged or saturated with the sprayed solution. The device is allowed to air-dry after which it is dried under vacuum at room temperature for 24 hours. In additional examples, one of the following exemplary compounds may be used in lieu of paclitaxel: mitoxantrone, doxorubicin, epithilone B, etoposide, TAXOTERE, tubercidin, vinblastine, geldanamycin, mithramycin, pimecrolimus, simvastatin, sirolimus, everolimus, mycophenolic acid, 1-alpha-25 dihydroxy vitamin D₃, Bay 11-7082, SB202190, and sulconizole.

### Example 24

### Drug-Dexamethasone Coated Device (Sequential Coating)

The catheter portion of an implantable pump device is attached to a rotating mandrel. A solution of paclitaxel (5% w/w) in a PLGA (50/50, Mw≈54,000)/ethyl acetate solution (2.5% w/v) is sprayed onto the outer surface of the device. The solution is sprayed on at a rate that ensures that the device is not damaged or saturated with the sprayed solution. The device is allowed to air dry. A methanol solution of dexamethasone (2% w/v) is then sprayed onto the outer surface of the device (at a rate that ensures that the device is not damaged or saturated with the sprayed solution). The device is allowed to air dry, after which it is dried under vacuum at room temperature for 24 hours. In additional examples, one of the following exemplary compounds may be used in lieu of paclitaxel: mitoxantrone, doxorubicin, epithilone B, etoposide, mithramycin, pimecrolimus, TAXOTERE, tubercidin, vinblastine, geldanamycin, simvastatin, sirolimus, everolimus, mycophenolic acid, 1-alpha-25 dihydroxy vitamin D₃, Bay 11-7082, SB202190, and sulconizole.

### Example 25

### Drug-Loading an Implantable Glucose Monitor—Paclitaxel Dipping

10 ml solutions of paclitaxel are prepared by weighing in 1 mg, 5 mg, 10 mg, 20 mg, 50 mg, 75 mg, 100 mg, 200 mg, and 500 mg paclitaxel into a 20 ml glass scintillation vial respectively and then adding HPLC grade methanol. The solutions are gently shaken on an orbital shaker for 1 hour at room temperature. The sensor tip of an implantable glucose sensor (DexCom, Inc.) is immersed to a depth of about 0.5 cm into the 0.1 mg/ml solution. After about 2 hours, the tip portion is removed from the solution and is allowed to air dry for 6 hour. The electrode is further dried under vacuum for 24 hours. The process is repeated for all the prepared paclitaxel solutions using a fresh sensor each time.

### Example 26

### Preparation of a Drug-Loaded Films for Implantable Glucose Sensors—Non-Woven Membranes

353 ml dimethylacetamide (DMAC) is added to a 2 liter glass beaker. 660 g of a polyurethane solution (CHRONOFLEX AR, 25% solids in DMAC, CardioTech Biomaterials, Inc) is added to the solution. The solution is stirred for 15 min using an overhead stirrer unit (Cole Palmer) with a TEFLON coated paddle type stirrer blade. 62.5 g poly(vinylpyrrolidone) (PLASDONE K-90D) is added to the solution. The solution is stirred for 6 hours until the polymers are all dissolved. Three sets of 5×15 g aliquots of the polymer solution is placed into 20 ml glass scintillation vials. To one set of the polymer solution, paclitaxel is added such that a paclitaxel to polymer ratio of 0.1%, 0.5%, 1%, 10% and 20% is obtained. For the second set of the polymer solutions, rapamycin is added such that a rapamycin to polymer ratio of 0.1%, 0.5%, 1%, 10% and 20% is obtained. For the third set of the polymer solutions, mythramycin is added such that a mythramycin to polymer ratio of 0.1%, 0.5%, 1%, 10% and 20% is obtained. The solutions are tumbled for 3 hours at 20 rpm. A non-woven DACRON fiber filtration membrane is placed on a silicone coated PET release liner. A film is cast over the filter membrane from each of the polymer solutions using a casting knife (0.006″). The cast solutions are allowed to air dry for 1 hour at room temperature. The films are further dried at 50° C. for 3 hours after which they are dried under vacuum for 24 hours. Each film is cut to size and is mechanically secured to an implantable glucose sensing device (DexCom, Inc) using an o-ring.

### Example 27

### Preparation of a Drug-Loaded Films for Implantable Glucose Sensors—Porous Membranes

353 ml dimethylacetamide (DMAC) is added to a 2L glass beaker. 660 g of a polyurethane solution (CHRONOFLEX AR, 25% solids in DMAC) is added to the solution. The solution is stirred for 15 min using an overhead stirrer unit (Cole Palmer) with a TEFLON coated paddle type stirrer blade. 62.5 g poly(vinylpyrrolidone) (PLASDONE K-90D) is added to the solution. The solution is stirred for 6 hours until the polymers are all dissolved. Three sets of 5×15 g aliquots of the polymer solution are placed into 20 ml glass scintillation vials. To one set of the polymer solution, paclitaxel is added such that a paclitaxel to polymer ratio of 0.1%, 0.5%, 1%, 10% and 20% is obtained. For the second set of the polymer solutions, rapamycin is added such that a rapamycin to polymer ratio of 0.1%, 0.5%, 1%, 10% and 20% is obtained For the third set of the polymer solutions, mythramycin is added such that a mythramycin to polymer ratio of 0.1%, 0.5%, 1%, 10% and 20% is obtained. The solutions are tumbled for 3 hours at 20 rpm. A film of each of the polymer solutions is cast on a silicone coated PET release liner using a casting knife (0.012″). The cast solutions are allowed to air dry for 1 hour at room temperature. The films are further dried at 50° C. for 3 hours after which they are dried under vacuum for 24 hours. Each film is then pressed onto a porous silicone membrane (Seare Biomatrix Systems, Inc). Each film laminate is cut to size and is mechanically secured to an implantable glucose sensing device (DexCom, Inc) using an o-ring.

### Example 28

### Drug-Loading a Membrane Used in an Implantable Glucose Monitor—Paclitaxel Dipping

10 ml solutions of paclitaxel are prepared by weighing in 1 mg, 5 mg, 10 mg, 20 mg, 50 mg, 75 mg, 100 mg, 200 mg, and 500 mg paclitaxel into a 20 ml glass scintillation vial respectively and then adding HPLC grade methanol. The solutions are gently shaken on an orbital shaker for 1 hour at room temperature. A CHRONOFLEX AR/PVP (Plasdone K-90D) (2.6:1 w/w) solution in DMAC is prepared as per Example 27. A non-woven DACRON fiber filtration membrane is placed on a silicone coated PET release liner. A film of the polymer solution is cast over the filter membrane using a casting knife. The cast solutions are allowed to air dry for 1 hour at room temperature. The films are further dried at 50° C. for 3 hours after which they are dried under vacuum for 24 hours. A film is immersed in the 0.1 mg paclitaxel solution for 2 hours. The film is removed from the solution and is air dried for 2 hours at 45° C. The film is then dried under vacuum for 24 hours. Each film is cut to size and is mechanically secured to an implantable glucose sensing device (DexCom, Inc) using an o-ring. This process is repeated using all the prepared paclitaxel solutions. In additional examples, one of the following exemplary compounds may be used in lieu of paclitaxel: rapamycin, mithramycin, everolimus, pimecrolimus, and halifuginone.

### Example 29

### Drug-Loading a Membrane Used in an Implantable Glucose Monitor—Paclitaxel Dipping

10 ml solutions of paclitaxel are prepared by weighing in 1 mg, 5 mg, 10 mg, 20 mg, 50 mg, 75 mg, 100 mg, 200 mg, and 500 mg paclitaxel into a 20 ml glass scintillation vial respectively and then adding HPLC grade methanol. The solutions are gently shaken on an orbital shaker for 1 hour at room temperature. A CHRONOFLEX AR/PVP (PLASDONE K-90D) [2.6:1 w/w] film used in a implantable glucose monitoring device (DexCom, Inc) in immersed in the 0.1 mg paclitaxel solution for 2 hour. The film is removed from the solution and is air dried for 2 hours at 45° C. The film is then dried under vacuum for 24 hours. Each film is then pressed onto a porous silicone membrane (Seare Biomatrix Systems, Inc). Each film laminate is cut to size and is mechanically secured to an implantable glucose sensing device (DexCom, Inc) using an o-ring. This process is repeated using all the prepared paclitaxel solutions. In additional examples, one of the following exemplary compounds may be used in lieu of paclitaxel: rapamycin, mithramycin, everolimus, pimecrolimus, and halifuginone.

### Example 30

### Coating of a Implantable Glucose Sensor

A polyurethane solution (CHRONOFLEX AL 85 A) is prepared by dissolving 20 g of the polyurethane in 400 ml tetrahydrofuran (THF). 15 ml aliquots of this solution are placed in 20 ml glass scintillation vials. 1 mg, 5 mg, 10 mg, 20 mg, 50 mg, 75 mg, 100 mg, and 200 mg paclitaxel are then added to each of the vials respectively. The solutions are tumbled for 3 hours at 20 rpm. An implantable glucose sensor device (DexCom, Inc) is held in a clamp. The clamp is then attached to an overhead stirrer (Cole Palmer) and the stirring speed is set to 40 rpm. One of the paclitaxel solutions is placed in a TLC spray device (Aldrich) that is attached to a nitrogen gas supply. The device is spray coated until a thin coating layer is obtained. The device is allowed to air dry for 5 hours. The device is removed from the clamp flipped 180 degrees and is again clamped. The coating process is then repeated. The entire coating process is repeated using each of the paclitaxel solutions and a new device each time. In additional examples, one of the following exemplary compounds may be used in lieu of paclitaxel: rapamycin, mithramycin, everolimus, pimecrolimus, and halifuginone.

### Example 31

### Drug-Loading the Catheter Portion of an Implantable Pump-Dipping

10 ml solutions of paclitaxel are prepared by weighing in 1 mg, 5 mg, 10 mg, 20 mg, 50 mg, 75 mg, 100 mg, 200 mg, and 500 mg paclitaxel into a 20 ml glass scintillation vial respectively and then adding HPLC grade methanol. The solutions are gently shaken on an orbital shaker for 1 hour at room temperature. The end segment of the catheter portion of an implantable pump (Medtronic) is immersed into the 0.1 mg/ml paclitaxel solution. After 2 hours the device is removed from the solution and is air dried for 24 hours at 37° C. The entire coating process is repeated using each of the paclitaxel solutions and a new device each time. In additional examples, one of the following exemplary compounds may be used in lieu of paclitaxel: rapamycin, mithramycin, everolimus, pimecrolimus, and halifuginone.

### Example 32

### Coating of an Implantable Pump

A polyurethane solution (CHRONOFLEX AL 85 A) is prepared by dissolving 20 g of the polyurethane in 400 ml tetrahydrofuran (THF). 15 ml aliquots of this solution are placed in 20 ml glass scintillation vials. 1 mg, 5 mg, 10 mg, 20 mg, 50 mg, 75 mg, 100 mg, and 200 mg paclitaxel are then added to each of the vials respectively. The solutions are tumbled for 3 hours at 20 rpm. An implantable pump device (Medtronic, Inc) is held in a clamp. The clamp is then attached to an overhead stirrer (Cole Palmer) and the stirring speed is set to 40 rpm. One of the paclitaxel solutions is placed in a TLC spray device (Aldrich) that is attached to a nitrogen gas supply. The device is spray coated until a thin coating layer is obtained. The device is allowed to air dry for 5 hours. The device is removed from the clamp flipped 180 degrees and is again clamped. The coating process is then repeated. The entire coating process is repeated using each of the paclitaxel solutions and a new device each time. In additional examples, one of the following exemplary compounds may be used in lieu of paclitaxel: rapamycin, mithramycin, everolimus, pimecrolimus, and halifuginone.

### Example 33

### Drug-Loading the Sensor Portion of a Cochlear Implant-Dipping

10 ml solutions of paclitaxel are prepared by weighing in 1 mg, 5 mg, 10 mg, 20 mg, 50 mg, 75 mg, 100 mg, 200 mg, and 500 mg paclitaxel into a 20 ml glass scintillation vial respectively and then adding HPLC grade methanol. The solutions are gently shaken on an orbital shaker for 1 hour at room temperature. The end segment of the sensor portion of a cochlear implant is immersed into the 0.1 mg/ml paclitaxel solution. After 2 hours the device is removed from the solution and is air dried for 24 hours at 37° C. The entire coating process is repeated using each of the paclitaxel solutions and a new device each time. In additional examples, one of the following exemplary compounds may be used in lieu of paclitaxel: rapamycin, mithramycin, everolimus, pimecrolimus, and halifuginone.

### Example 34

### Screening Assay for Assessing the Effect of Various Compounds on Nitric Oxide Production by Macrophages

The murine macrophage cell line RAW 264.7 was trypsinized to remove cells from flasks and plated in individual wells of a 6-well plate. Approximately 2×10⁶ cells were plated in 2 mL of media containing 5% heat-inactivated fetal bovine serum (FBS). RAW 264.7 cells were incubated at 37° C. for 1.5 hours to allow adherence to plastic. Mitoxantrone was prepared in DMSO at a concentration of 10⁻² M and serially diluted 10-fold to give a range of stock concentrations (10⁻⁸ M to 10⁻² M). Media was then removed and cells were incubated in 1 ng/mL of recombinant murine IFNγ and 5 ng/mL of LPS with or without mitoxantrone in fresh media containing 5% FBS. Mitoxantrone was added to cells by directly adding mitoxantrone DMSO stock solutions, prepared earlier, at a 1/1000 dilution, to each well. Plates containing IFNγ, LPS plus or minus mitoxantrone were incubated at 37° C. for 24 hours (Chem. Ber. (1879) 12: 426; J. AOAC (1977) 60-594; Ann. Rev. Biochem. (1994) 63: 175).

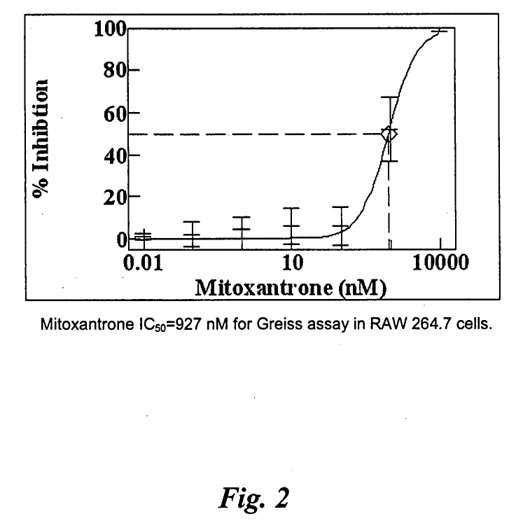

At the end of the 24 hour period, supernatants were collected from the cells and assayed for the production of nitrites. Each sample was tested in triplicate by aliquoting 50 μl of supernatant in a 96-well plate and adding 50 μl of Greiss Reagent A (0.5 g sulfanilamide, 1.5 mL H₃PO₄, 48.5 mL ddH₂O) and 50 μl of Greiss Reagent B (0.05 g N-(1-naphthyl)-ethylenediamine, 1.5 mL H₃PO₄, 48.5 mL ddH₂O). Optical density was read immediately on microplate spectrophotometer at 562 nm absorbance. Absorbance over triplicate wells was averaged after subtracting background and concentration values were obtained from the nitrite standard curve (1 μM to 2 mM). Inhibitory concentration of 50% (IC₅₀) was determined by comparing average nitrite concentration to the positive control (cell stimulated with IFNγ and LPS). An average of n=4 replicate experiments was used to determine IC₅₀ values for mitoxantrone (see, FIG. 2 (IC₅₀=927 nM)). The IC₅₀ values for the following additional compounds were determined using this assay: IC₅₀ (nM): paclitaxel, 7; CNI-1493, 249; halofuginone, 12; geldanamycin, 51; anisomycin, 68; 17-MG, 840; epirubicin hydrochloride, 769.

### Example 35

### Screening Assay for Assessing the Effect of Various Anti-Scarring Agents on TNF-Alpha Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g and resuspended in 4 mL of human serum for a final concentration of 5 mg/mL and incubated in a 37° C. water bath for 20 minutes to enable opsonization. Bay 11-7082 was prepared in DMSO at a concentration of 10⁻² M and serially diluted 10-fold to give a range of stock concentrations (10⁻⁸ M to 10⁻² M) (J. Immunol. (2000) 165: 411-418; J. Immunol. (2000) 164: 4804-4811; J. Immunol Meth. (2000) 235 (1-2): 33-40).

THP-1 cells were stimulated to produce TNFa by the addition of 1 mg/mL opsonized zymosan. Bay 11-7082 was added to THP-1 cells by directly adding DMSO stock solutions, prepared earlier, at a 1/1000 dilution, to each well. Each drug concentration was tested in triplicate wells. Plates were incubated at 37° C. for 24 hours.

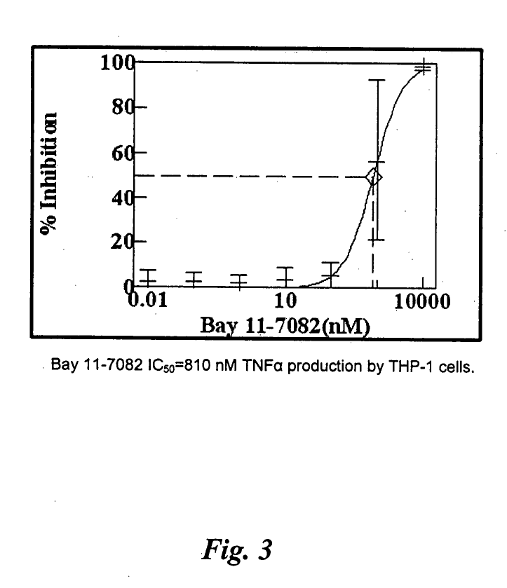

After a 24 hour stimulation, supernatants were collected to quantify TNFα production. TNFα concentrations in the supernatants were determined by ELISA using recombinant human TNFa to obtain a standard curve. A 96-well MaxiSorb plate was coated with 100 μl of anti-human TNFa Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted ⅛ and 1/16; (b) recombinant human TNFa was prepared at 500 μg/mL and serially diluted to yield as standard curve of 7.8 μg/mL to 500 μg/mL. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human TNFα detection antibody+avidin-HRP) for 1 hour at room temperature. Following this incubation, the plates were washed 7 times and 100 μl of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. TNFα concentration values were obtained from the standard curve. Inhibitory concentration of 50% (IC₅₀) was determined by comparing average TNFa concentration to the positive control (THP-1 cells stimulated with opsonized zymosan). An average of n=4 replicate experiments was used to determine IC₅₀ values for Bay 11-7082 (see FIG. 3; IC₅₀=810 nM)) and rapamycin (IC₅₀=51 nM; FIG. 4). The IC₅₀ values for the following additional compounds were determined using this assay: IC₅₀ (nM): geldanamycin, 14; mycophenolic acid, 756; mofetil, 792; chlorpromazine, 6; CNI-1493, 0.15; SKF 86002, 831; 15-deoxy prostaglandin J2, 742; fascaplysin, 701; podophyllotoxin, 75; mithramycin, 570; daunorubicin, 195; celastrol, 87; chromomycin A3, 394; vinorelbine, 605; vinblastine, 65.

### Example 36

### Surgical Adhesions Model to Assess Fibrosis Inhibiting Agents in Rats

The rat caecal sidewall model is used to as to assess the anti-fibrotic capacity of formulations in vivo. Sprague Dawley rats are anesthetized with halothane. Using aseptic precautions, the abdomen is opened via a midline incision. The caecum is exposed and lifted out of the abdominal cavity. Dorsal and ventral aspects of the caecum are successively scraped a total of 45 times over the terminal 1.5 cm using a #10 scalpel blade. Blade angle and pressure are controlled to produce punctate bleeding while avoiding severe tissue damage. The left side of the abdomen is retracted and everted to expose a section of the peritoneal wall that lies proximal to the caecum. The superficial layer of muscle (transverses abdominis) is excised over an area of 1×2 cm², leaving behind torn fibres from the second layer of muscle (internal oblique muscle). Abraded surfaces are tamponaded until bleeding stops. The abraded caecum is then positioned over the sidewall wound and attached by two sutures. The formulation is applied over both sides of the abraded caecum and over the abraded peritoneal sidewall. A further two sutures are placed to attach the caecum to the injured sidewall by a total of 4 sutures and the abdominal incision is closed in two layers. After 7 days, animals are evaluated post mortem with the extent and severity of adhesions being scored both quantitatively and qualitatively.

### Example 37

### Surgical Adhesions Model to Assess Fibrosis Inhibiting Agents in Rabbits

The rabbit uterine horn model is used to assess the anti-fibrotic capacity of formulations in vivo. Mature New Zealand White (NZW) female rabbits are placed under general anesthetic. Using aseptic precautions, the abdomen is opened in two layers at the midline to expose the uterus. Both uterine horns are lifted out of the abdominal cavity and assessed for size on the French Scale of catheters. Horns between #8 and #14 on the French Scale (2.5-4.5 mm diameter) are deemed suitable for this model. Both uterine horns and the opposing peritoneal wall are abraded with a #10 scalpel blade at a 45° angle over an area 2.5 cm in length and 0.4 cm in width until punctuate bleeding is observed. Abraded surfaces are tamponaded until bleeding stops. The individual horns are then opposed to the peritoneal wall and secured by two sutures placed 2 mm beyond the edges of the abraded area. The formulation is applied and the abdomen is closed in three layers. After 14 days, animals are evaluated post mortem with the extent and severity of adhesions being scored both quantitatively and qualitatively.

### Example 38

### Screening Assay for Assessing the Effect of Various Compounds on Cell Proliferation

Fibroblasts at 70-90% confluency were trypsinized, replated at 600 cells/well in media in 96-well plates and allowed to attach overnight. Mitoxantrone was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μl/well. Each drug concentration was tested in triplicate wells. Plates containing fibroblasts and mitoxantrone were incubated at 37° C. for 72 hours (In vitro toxicol. (1990) 3: 219; Biotech. Histochem. (1993) 68: 29; Anal. Biochem. (1993) 213: 426).

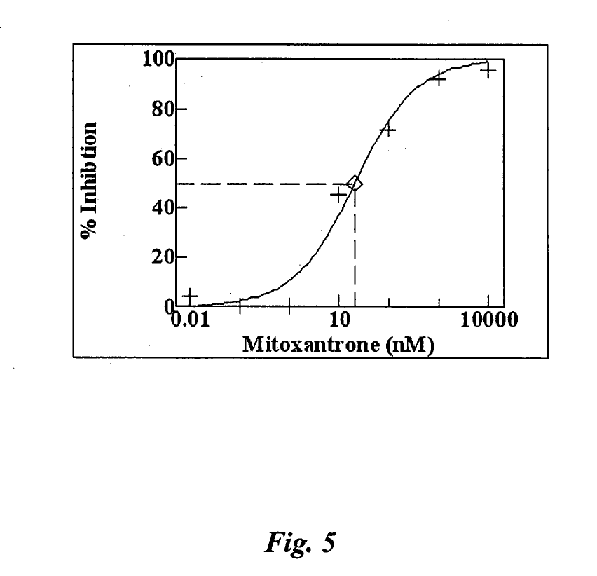

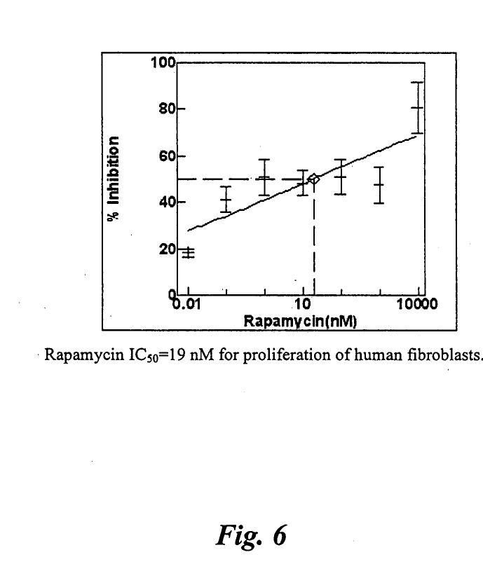

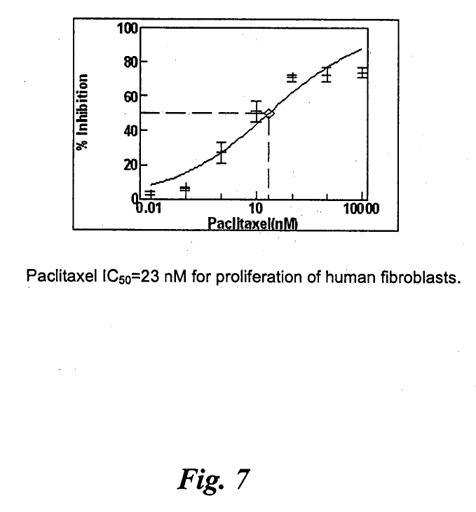

To terminate the assay, the media was removed by gentle aspiration. A 1/400 dilution of CYQUANT 400×GR dye indicator (Molecular Probes; Eugene, Oreg.) was added to 1× Cell Lysis buffer, and 200 μl of the mixture was added to the wells of the plate. Plates were incubated at room temperature, protected from light for 3-5 minutes. Fluorescence was read in a fluorescence microplate reader at 480 nm excitation wavelength and ˜520 nm emission maxima. Inhibitory concentration of 50% (IC₅₀) was determined by taking the average of triplicate wells and comparing average relative fluorescence units to the DMSO control. An average of n=4 replicate experiments was used to determine IC₅₀ values. The IC₅₀ values for the following compounds were determined using this assay: IC₅₀ (nM): mitoxantrone, 20 (FIG. 5); rapamycin, 19 (FIG. 6); paclitaxel, 23 (FIG. 7); mycophenolic acid, 550; mofetil, 601; GW8510, 98; simvastatin, 885; doxorubicin, 84; geldanamycin, 11; anisomycin, 435; 17-MG, 106; bleomycin, 86; halofuginone, 36; gemfibrozil, 164; ciprofibrate, 503; bezafibrate, 184; epirubicin hydrochloride, 57; topotecan, 81; fascaplysin, 854; tamoxifen, 13; etanidazole, 55; gemcitabine, 7; puromycin, 254; mithramycin, 156; daunorubicin, 51; L(−)-perillyl alcohol, 966; celastrol, 271; anacitabine, 225; oxalipatin, 380; chromomycin A3, 4; vinorelbine, 4; idarubicin, 34; nogalamycin, 5; 17-DMAG, 5; epothilone D, 2; vinblastine, 2; vincristine, 7; cytarabine, 137.

### Example 39

### Evaluation of Paclitaxel Containing Mesh on Intimal Hyperplasia Development in a Rat Balloon Injury Carotid Artery Model as an Example to Evaluate Fibrosis Inhibiting Agents

A rat balloon injury carotid artery model was used to demonstrate the efficacy of a paclitaxel containing mesh system on the development of intimal hyperplasia fourteen days following placement.

Control Group

Wistar rats weighing 400-500 g were anesthetized with 1.5% halothane in oxygen and the left external carotid artery was exposed. An A 2 French FOGARTY balloon embolectomy catheter (Baxter, Irvine, Calif.) was advanced through an arteriotomy in the external carotid artery down the left common carotid artery to the aorta. The balloon was inflated with enough saline to generate slight resistance (approximately 0.02 ml) and it was withdrawn with a twisting motion to the carotid bifurcation. The balloon was then deflated and the procedure repeated twice more. This technique produced distension of the arterial wall and denudation of the endothelium. The external carotid artery was ligated after removal of the catheter. The right common carotid artery was not injured and was used as a control.

Local Perivascular Paclitaxel Treatment

Immediately after injury of the left common carotid artery, a 1 cm long distal segment of the artery was exposed and treated with a 1×1 cm paclitaxel-containing mesh (345 μg paclitaxel in a 50:50 PLG coating on a 10:90 PLG meSH). The wound was then closed the animals were kept for 14 days.

Histology and Immunohistochemistry

At the time of sacrifice, the animals were euthanized with carbon dioxide and pressure perfused at 100 mmHg with 10% phosphate buffered formaldehyde for 15 minutes. Both carotid arteries were harvested and left overnight in fixative. The fixed arteries were processed and embedded in paraffin wax. Serial cross-sections were cut at 3 μm thickness every 2 mm within and outside the implant region of the injured left carotid artery and at corresponding levels in the control right carotid artery. Cross-sections were stained with Mayer's hematoxylin-and-eosin for cell count and with Movat's pentachrome stains for morphometry analysis and for extracellular matrix composition assessment.

Results

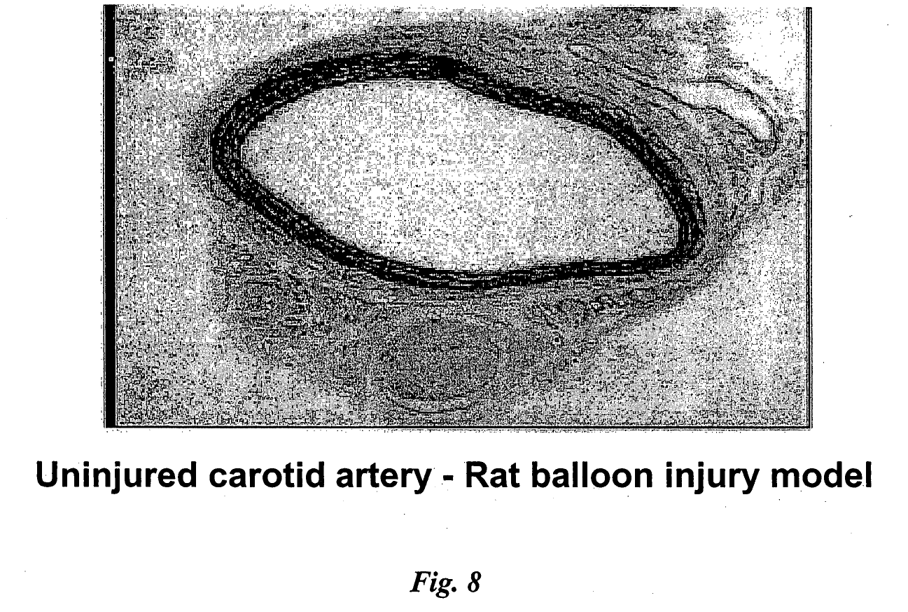

From FIGS. 8-10, it is evident that the perivascular delivery of paclitaxel using the paclitaxel mesh formulation resulted is a dramatic reduction in intimal hyperplasia.

### Example 40

### Effect of Paclitaxel and Other Anti-Microtubule Agents on Matrix Metalloproteinase Production

A. Materials and Methods

1. IL-1 Stimulated AP-1 Transcriptional Activity is Inhibited by Paclitaxel

Chondrocytes were transfected with constructs containing an AP-1 driven CAT reporter gene, and stimulated with IL-1, IL-1 (50 ng/ml) was added and incubated for 24 hours in the absence and presence of paclitaxel at various concentrations. Paclitaxel treatment decreased CAT activity in a concentration dependent manner (mean±SD). The data noted with an asterisk (*) have significance compared with IL-1-induced CAT activity according to a t-test, P<0.05. The results shown are representative of three independent experiments.

2. Effect of Paclitaxel on IL-1 Induced AP-1 DNA Binding Activity, AP-1 DNA

Binding activity was assayed with a radiolabeled human AP-1 sequence probe and gel mobility shift assay. Extracts from chondrocytes untreated or treated with various amounts of paclitaxel (10⁻⁷ to 10⁻⁵ M) followed by IL-1β (20 ng/ml) were incubated with excess probe on ice for 30 minutes, followed by non-denaturing gel electrophoresis. The “com” lane contains excess unlabeled AP-1 oligonucleotide. The results shown are representative of three independent experiments.

3. Effect of Paclitaxel on IL-1 Induced MMP-1 and MMP-3 mRNA Expression

Cells were treated with paclitaxel at various concentrations (10⁻⁷ to 10⁻⁵ M) for 24 hours, then treated with IL-1β (20 ng/ml) for additional 18 hours in the presence of paclitaxel. Total RNA was isolated, and the MMP-1 mRNA levels were determined by Northern blot analysis. The blots were subsequently stripped and reprobed with ³²P-radiolabeled rat GAPDH cDNA, which was used as a housekeeping gene. The results shown are representative of four independent experiments. Quantitation of collagenase-1 and stromelysin-expression mRNA levels was conducted. The MMP-1 and MMP-3 expression levels were normalized with GAPDH.

4. Effect of Other Anti-Microtubules on Collagenase Expression

Primary chondrocyte cultures were freshly isolated from calf cartilage. The cells were plated at 2.5×10⁶ per ml in 100×20 mm culture dishes and incubated in Ham's F12 medium containing 5% FBS overnight at 37° C. The cells were starved in serum-free medium overnight and then treated with anti-microtubule agents at various concentrations for 6 hours. IL-1 (20 ng/ml) was then added to each plate and the plates incubated for an additional 18 hours. Total RNA was isolated by the acidified guanidine isothiocyanate method and subjected to electrophoresis on a denatured gel. Denatured RNA samples (15 μg) were analyzed by gel electrophoresis in a 1% denatured gel, transferred to a nylon membrane and hydridized with the ³²P-labeled collagenase cDNA probe. ³²P-labeled glyceraldehyde phosphate dehydrase (GAPDH) cDNA as an internal standard to ensure roughly equal loading. The exposed films were scanned and quantitatively analyzed with IMAGEQUANT.

B. Results

1. Promoters on the Family of Matrix Metalloproteinases

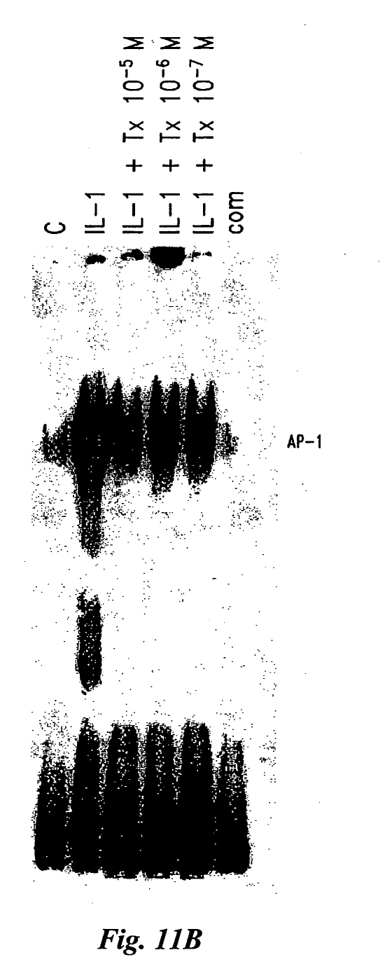

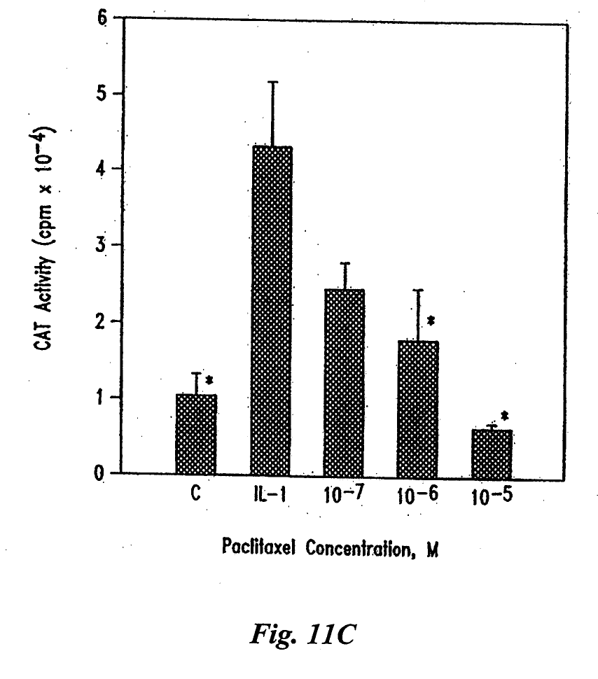

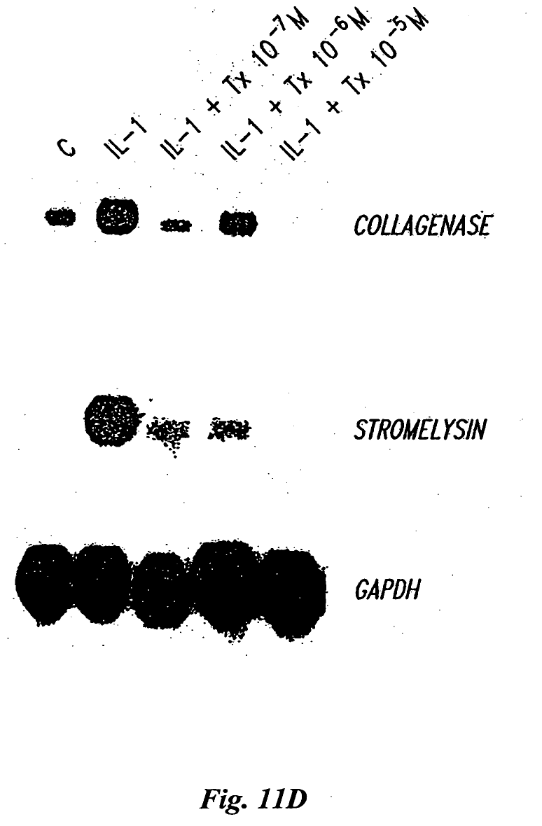

FIG. 11A shows that all matrix metalloproteinases contained the transcriptional elements AP-1 and PEA-3 with the exception of gelatinase B. It has been well established that expression of matrix metalloproteinases such as collagenases and stromelysins are dependent on the activation of the transcription factors AP-1. Thus inhibitors of AP-1 may inhibit the expression of matrix metalloproteinases.

2. Effect of Paclitaxel on AP-1 Transcriptional Activity

As demonstrated in FIG. 11B, IL-1 stimulated AP-1 transcriptional activity 5-fold. Pretreatment of transiently transfected chondrocytes with paclitaxel reduced IL-1 induced AP-1 reporter gene CAT activity. Thus, IL-1 induced AP-1 activity was reduced in chondrocytes by paclitaxel in a concentration dependent manner (10⁻⁷ to 10⁻⁵ M). These data demonstrated that paclitaxel was a potent inhibitor of AP-1 activity in chondrocytes.

3. Effect of Paclitaxel on AP-1 DNA Binding Activity

To confirm that paclitaxel inhibition of AP-1 activity was not due to nonspecific effects, the effect of paclitaxel on IL-1 induced AP-1 binding to oligonucleotides using chondrocyte nuclear lysates was examined. As shown in FIG. 11C, IL-1 induced binding activity decreased in lysates from chondrocyte which had been pretreated with paclitaxel at concentration 10⁻⁷ to 10⁻⁵ M for 24 hours. Paclitaxel inhibition of AP-1 transcriptional activity closely correlated with the decrease in AP-1 binding to DNA.

4. Effect of Paclitaxel on Collagenase and Stromelysin Expression

Since paclitaxel was a potent inhibitor of AP-1 activity, the effect of paclitaxel or IL-1 induced collagenase and stromelysin expression, two important matrix metalloproteinases involved in inflammatory diseases was examined. Briefly, as shown in FIG. 11D, IL-1 induction increases collagenase and stromelysin mRNA levels in chondrocytes. Pretreatment of chondrocytes with paclitaxel for 24 hours significantly reduced the levels of collagenase and stromelysin mRNA. At 10⁻⁵ M paclitaxel, there was complete inhibition. The results show that paclitaxel completely inhibited the expression of two matrix metalloproteinases at concentrations similar to which it inhibits AP-1 activity.

5. Effect of Other Anti-Microtubules on Collagenase Expression

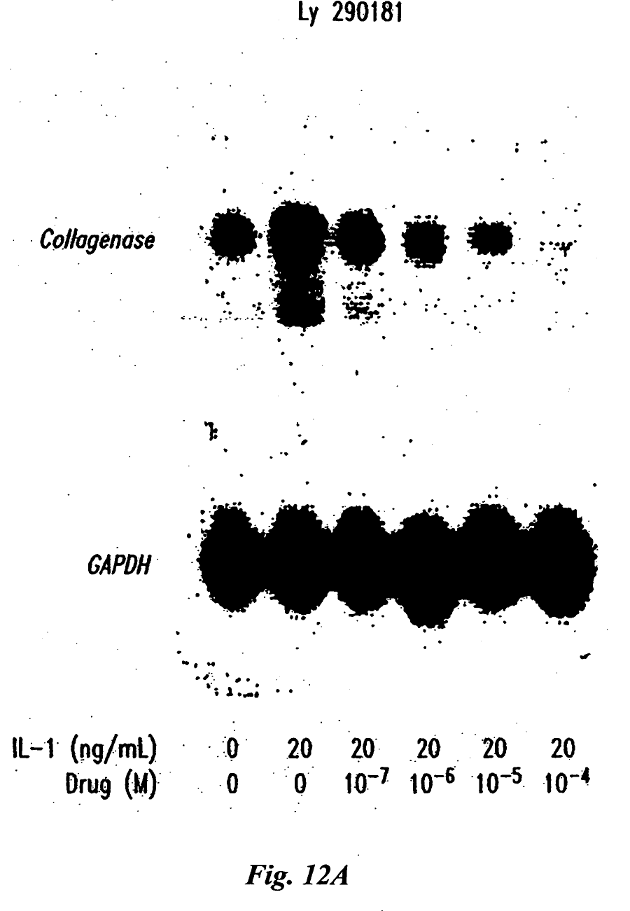

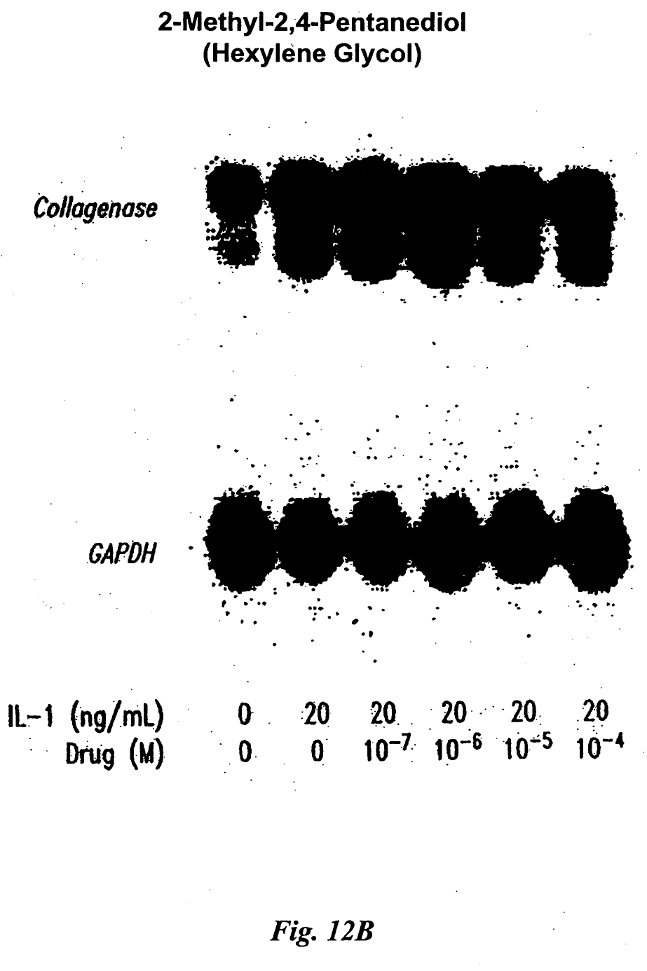

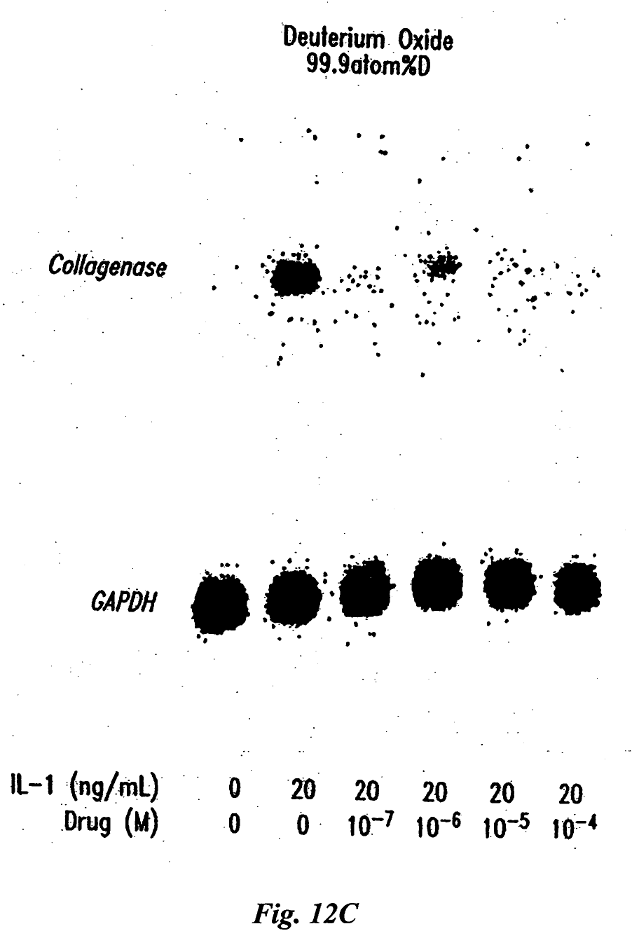

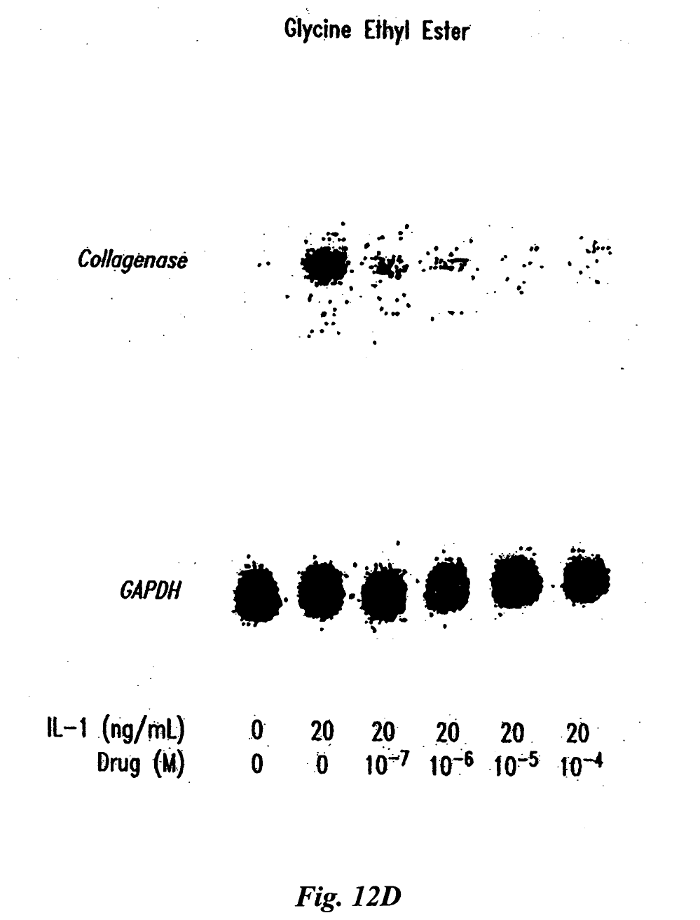

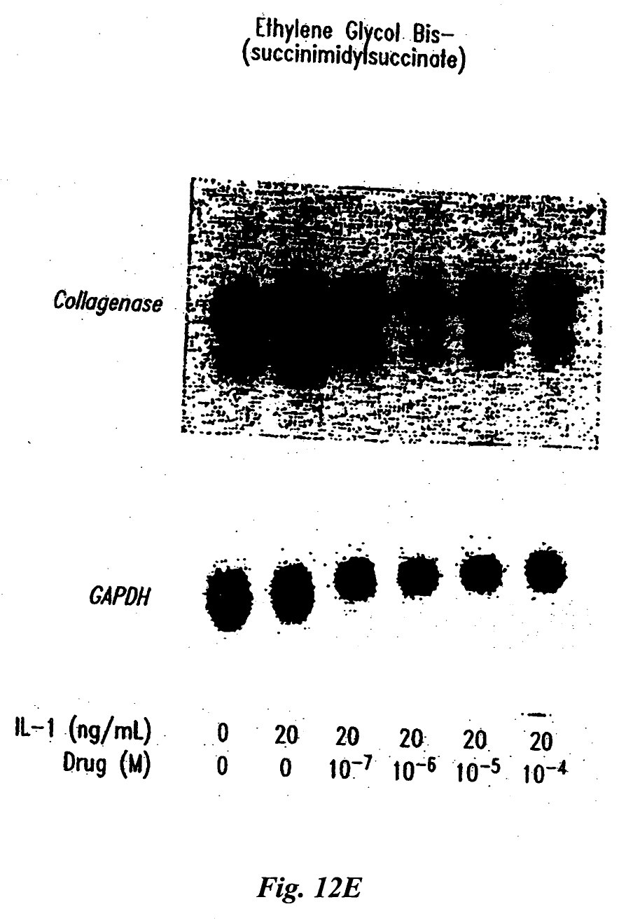

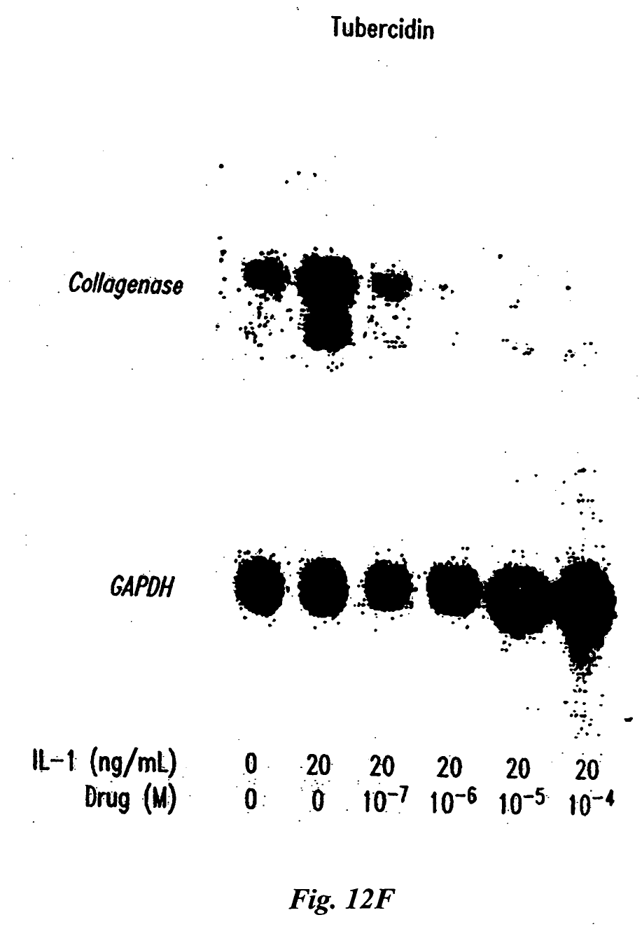

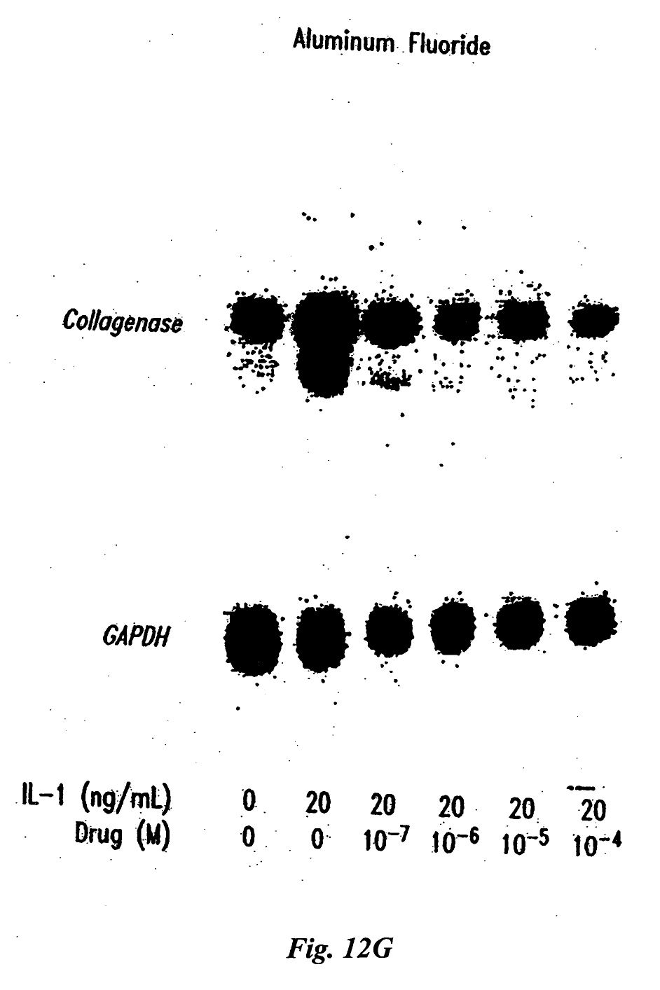

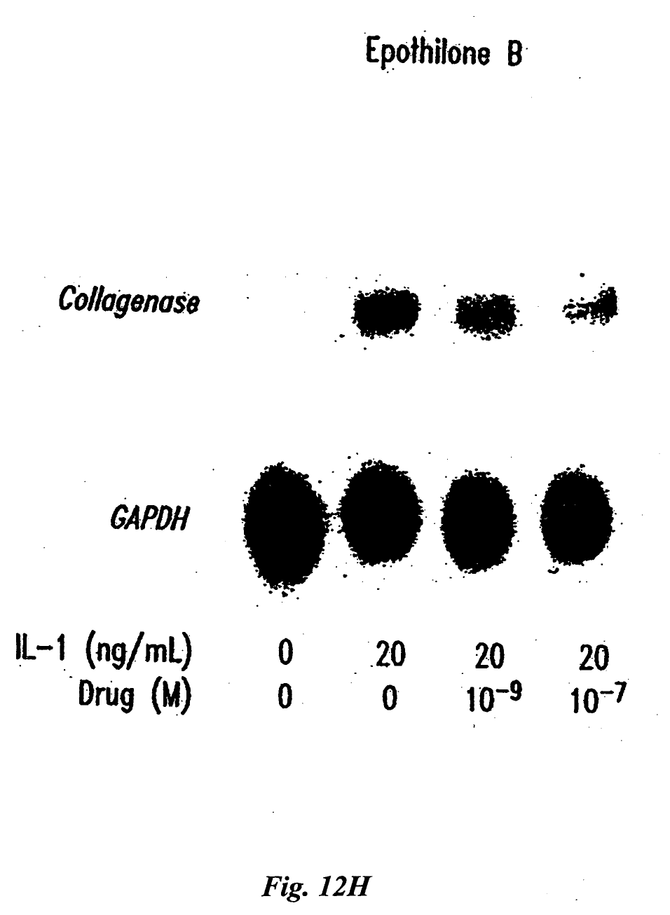

FIGS. 12A-H demonstrate that anti-microtubule agents inhibited collagenase expression. Expression of collagenase was stimulated by the addition of IL-1 which is a proinflammatory cytokine. Pre-incubation of chondrocytes with various anti-microtubule agents, specifically LY290181, hexylene glycol, deuterium oxide, glycine ethyl ester, ethylene glycol bis-(succinimidylsuccinate), tubercidin, AlF₃, and epothilone, all prevented IL-1-induced collagenase expression at concentrations as low as 1×10⁻⁷ M.

C. Discussion

Paclitaxel was capable of inhibiting collagenase and stromelysin expression in vitro at concentrations of 10⁻⁶ M. Since this inhibition may be explained by the inhibition of AP-1 activity, a required step in the induction of all matrix metalloproteinases with the exception of gelatinase B, it is expected that paclitaxel may inhibit other matrix metalloproteinases which are AP-1 dependent. The levels of these matrix metalloproteinases are elevated in all inflammatory diseases and play a principle role in matrix degradation, cellular migration and proliferation, and angiogenesis. Thus, paclitaxel inhibition of expression of matrix metalloproteinases such as collagenase and stromelysin can have a beneficial effect in inflammatory diseases.

In addition to paclitaxel's inhibitory effect on collagenase expression, LY290181, hexylene glycol, deuterium oxide, glycine ethyl ester, AlF₃, tubercidin epothilone, and ethylene glycol bis-(succinimidylsuccinate), all prevented IL-1-induced collagenase expression at concentrations as low as 1×10⁻⁷ M. Thus, anti-microtubule agents are capable of inhibiting the AP-1 pathway at varying concentrations.

### Example 41

### Inhibition of Angiogenesis by Paclitaxel

A. Chick Chorioallantoic Membrane (“CAM”) Assays

Fertilized, domestic chick embryos were incubated for 3 days prior to shell-less culturing. In this procedure, the egg contents were emptied by removing the shell located around the air space. The interior shell membrane was then severed and the opposite end of the shell was perforated to allow the contents of the egg to gently slide out from the blunted end. The egg contents were emptied into round-bottom sterilized glass bowls and covered with petri dish covers. These were then placed into an incubator at 90% relative humidity and 3% CO₂ and incubated for 3 days.

Paclitaxel (Sigma, St. Louis, Mich.) was mixed at concentrations of 0.25, 0.5, 1, 5, 10, 30 μg per 10 μl aliquot of 0.5% aqueous methylcellulose. Since paclitaxel is insoluble in water, glass beads were used to produce fine particles. Ten microliter aliquots of this solution were dried on parafilm for 1 hour forming disks 2 mm in diameter. The dried disks containing paclitaxel were then carefully placed at the growing edge of each CAM at day 6 of incubation. Controls were obtained by placing paclitaxel-free methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

The membranes of the 8 day-old shell-less embryo were flooded with 2% glutaraldehyde in 0.1 M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5 minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged noninvasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg, each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4 point avascular gradient (Table 1). This scale represents the degree of overall inhibition with maximal inhibition represented as a 3 on the avascular gradient scale. Paclitaxel was very consistent and induced a maximal avascular zone (6 mm in diameter or a 3 on the avascular gradient scale) within 48 hours depending on its concentration.

TABLE 1
Avascular Gradient

| | |
|---|---|
| 0 | normal vascularity |
| 1 | lacking some microvascular movement |
| 2* | small avascular zone approximately 2 mm in diameter |
| 3* | avascularity extending beyond the disk (6 mm in diameter) |

*indicates a positive antiangiogenesis response

The dose-dependent, experimental data of the effects of paclitaxel at different concentrations are shown in Table 2.

TABLE 2

| Agent | Delivery Vehicle | Concentration | Inhibition/n |
|---|---|---|---|
| paclitaxel | methylcellulose (10 μl) | 0.25 μg | 2/11 |
| | methylcellulose (10 μl) | 0.5 μg | 6/11 |
| | methylcellulose (10 μl) | 1 μg | 6/15 |
| | methylcellulose (10 μl) | 5 μg | 20/27 |
| | methylcellulose (10 μl) | 10 μg | 16/21 |
| | methylcellulose (10 μl) | 30 μg | 31/31 |

Typical paclitaxel-treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after paclitaxel application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

### Example 42

### Screening Assay for Assessing the Effect of Paclitaxel on Smooth Muscle Cell Migration

Primary human smooth muscle cells were starved of serum in smooth muscle cell basal media containing insulin and human basic fibroblast growth factor (bFGF) for 16 hours prior to the assay. For the migration assay, cells were trypsinized to remove cells from flasks, washed with migration media and diluted to a concentration of 2-2.5×10⁵ cells/mL in migration media. Migration media consists of phenol red free Dulbecco's Modified Eagle Medium (DMEM) containing 0.35% human serum albumin. A 100 μl volume of smooth muscle cells (approximately 20,000-25,000 cells) was added to the top of a Boyden chamber assembly (Chemicon QCM CHEMOTAXIS 96-well migration plate). To the bottom wells, the chemotactic agent, recombinant human platelet derived growth factor (rhPDGF-BB) was added at a concentration of 10 ng/mL in a total volume of 150 μl. Paclitaxel was prepared in DMSO at a concentration of 10⁻² M and serially diluted 10-fold to give a range of stock concentrations (10⁻⁸ M to 10⁻² M). Paclitaxel was added to cells by directly adding paclitaxel DMSO stock solutions, prepared earlier, at a 1/1000 dilution, to the cells in the top chamber. Plates were incubated for 4 hours to allow cell migration.

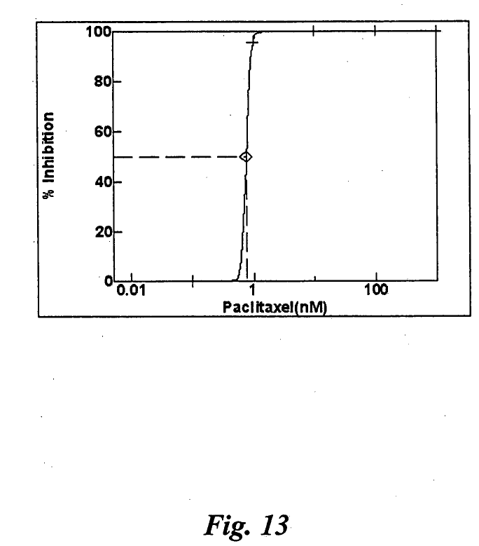

At the end of the 4 hour period, cells in the top chamber were discarded and the smooth muscle cells attached to the underside of the filter were detached for 30 minutes at 37° C. in Cell Detachment Solution (Chemicon). Dislodged cells were lysed in lysis buffer containing the DNA binding CYQUANT GR dye and incubated at room temperature for 15 minutes. Fluorescence was read in a fluorescence microplate reader at ˜480 nm excitation wavelength and 520 nm emission maxima. Relative fluorescence units from triplicate wells were averaged after subtracting background fluorescence (control chamber without chemoattractant) and average number of cells migrating was obtained from a standard curve of smooth muscle cells serially diluted from 25,000 cells/well down to 98 cells/well. Inhibitory concentration of 50% (IC₅₀) was determined by comparing the average number of cells migrating in the presence of paclitaxel to the positive control (smooth muscle cell chemotaxis in response to rhPDGF-BB). See FIG. 13 (IC₅₀=0.76 nM). References: Biotechniques (2000) 29: 81; J. Immunol Methods (2001) 254: 85.

### Example 43

### Screening Assay for Assessing the Effect of Various Compounds on IL-1β Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g and resuspended in 4 mL of human serum for a final concentration of 5 mg/mL and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-1 by the addition of 1 mg/mL opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

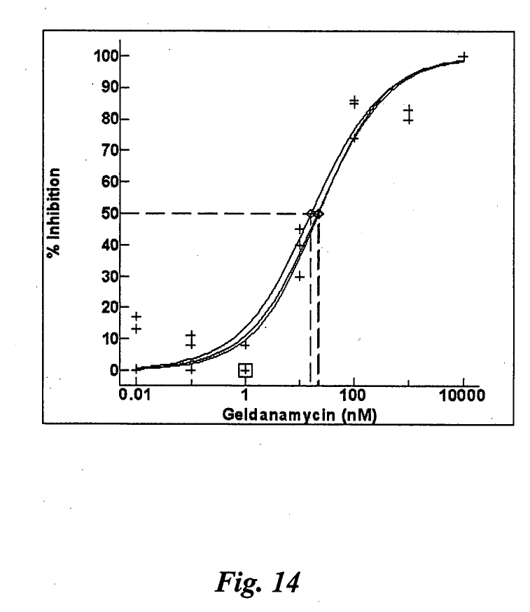

After a 24 hour stimulation, supernatants were collected to quantify IL-1β production. IL-1β concentrations in the supernatants were determined by ELISA using recombinant human IL-1β to obtain a standard curve. A 96-well MaxiSorb plate was coated with 100 μl of anti-human IL-1β Capture Antibody diluted in Coating Buffer (0.1 M Sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted ¼ and ⅛; (b) recombinant human IL-1β was prepared at 1000 μg/mL and serially diluted to yield as standard curve of 15.6 μg/mL to 1000 μg/mL. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human IL-1β detection antibody+avidin-HRP) for 1 hour at room temperature. Following this incubation, the plates were washed 7 times and 100 μl of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. IL-1β concentration values were obtained from the standard curve. Inhibitory concentration of 50% (IC₅₀) was determined by comparing average IL-1β concentration to the positive control (THP-1 cells stimulated with opsonized zymosan). An average of n=4 replicate experiments was used to determine IC₅₀ values for geldanamycin (IC₅₀=20 nM). See FIG. 14. The IC₅₀ values for the following additional compounds were determined using this assay: IC₅₀ (nM): mycophenolic acid 2888 nM); anisomycin, 127; rapamycin, 0.48; halofuginone, 919; IDN-6556, 642; epirubicin hydrochloride, 774; topotecan, 509; fascaplysin, 425; daunorubicin, 517; celastrol, 23; oxalipatin, 107; chromomycin A3,148.

References: J. Immunol. (2000) 165: 411-418; J. Immunol. (2000) 164: 4804-4811; J. Immunol Meth. (2000) 235 (1-2): 33-40.

### Example 44

### Screening Assay for Assessing the Effect of Various Compounds on IL-8 Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g, resuspended in 4 mL of human serum for a final concentration of 5 mg/mL, and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce IL-8 by the addition of 1 mg/mL opsonized zymosan. Geldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

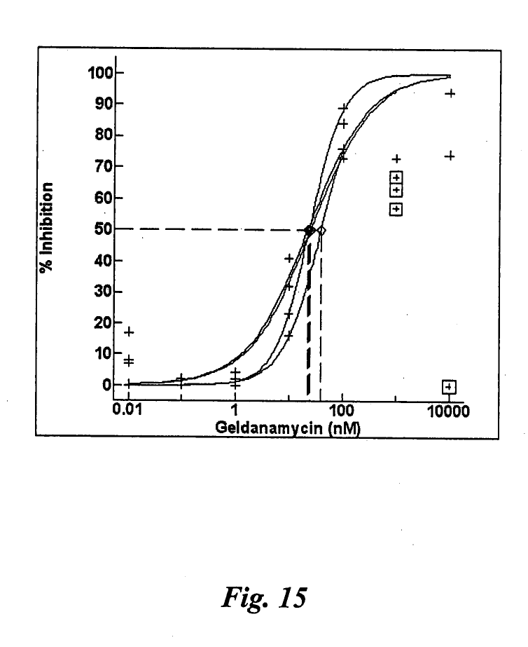

After a 24 hour stimulation, supernatants were collected to quantify IL-8 production. IL-8 concentrations in the supernatants were determined by ELISA using recombinant human IL-8 to obtain a standard curve. A 96-well MAXISORB plate was coated with 100 μl of anti-human IL-8 Capture Antibody diluted in Coating Buffer (0.1 M sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human IL-8 was prepared at 200 μg/mL and serially diluted to yield as standard curve of 3.1 μg/mL to 200 μg/mL. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human IL-8 detection antibody+avidin-HRP) for 1 hour at room temperature. Following this incubation, the plates were washed 7 times and 100 μl of Substrate Solution (Tetramethylbenzidine, H₂O₂) was added to plates and incubated for 30 minutes at room temperature. Stop Solution (2 N H₂SO₄) was then added to the wells and a yellow color reaction was read at 450 nm with A correction at 570 nm. Mean absorbance was determined from triplicate data readings and the mean background was subtracted. IL-8 concentration values were obtained from the standard curve. Inhibitory concentration of 50% (IC₅₀) was determined by comparing average IL-8 concentration to the positive control (THP-1 cells stimulated with opsonized zymosan). An average of n=4 replicate experiments was used to determine IC₅₀ values for geldanamycin (IC₅₀=27 nM). See FIG. 15. The IC₅₀ values for the following additional compounds were determined using this assay: IC₅₀ (nM): 17-AAG, 56; mycophenolic acid, 549; resveratrol, 507; rapamycin, 4; 41; SP600125, 344; halofuginone, 641; D-mannose-6-phosphate, 220; epirubicin hydrochloride, 654; topotecan, 257; mithramycin, 33; daunorubicin, 421; celastrol, 490; chromomycin A3, 36.

References: J. Immunol. (2000) 165: 411-418; J. Immunol. (2000) 164: 4804-4811; J. Immunol Meth. (2000) 235 (1-2): 33-40.

### Example 45

### Screening Assay for Assessing the Effect of Various Compounds on MCP-1 Production by Macrophages

The human macrophage cell line, THP-1 was plated in a 12 well plate such that each well contains 1×10⁶ cells in 2 mL of media containing 10% FCS. Opsonized zymosan was prepared by resuspending 20 mg of zymosan A in 2 mL of ddH₂O and homogenizing until a uniform suspension was obtained. Homogenized zymosan was pelleted at 250 g and resuspended in 4 mL of human serum for a final concentration of 5 mg/mL and incubated in a 37° C. water bath for 20 minutes to enable opsonization. Geldanamycin was prepared in DMSO at a concentration of 10⁻² M and serially diluted 10-fold to give a range of stock concentrations (10⁻⁸ M to 10⁻² M).

THP-1 cells were stimulated to produce MCP-1 by the addition of 1 mg/mL opsonized zymosan. Eldanamycin was added to THP-1 cells by directly adding DMSO stock solutions, prepared earlier, at a 1/1000 dilution, to each well. Each drug concentration was tested in triplicate wells. Plates were incubated at 37° C. for 24 hours.

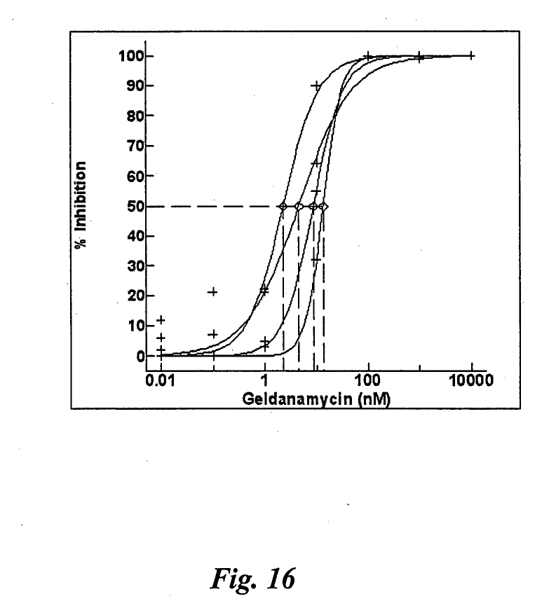

After a 24 hour stimulation, supernatants were collected to quantify MCP-1 production. MCP-1 concentrations in the supernatants were determined by ELISA using recombinant human MCP-1 to obtain a standard curve. A 96-well MaxiSorb plate was coated with 100 μl of anti-human MCP-1 Capture Antibody diluted in Coating Buffer (0.1 M Sodium carbonate pH 9.5) overnight at 4° C. The dilution of Capture Antibody used was lot-specific and was determined empirically. Capture antibody was then aspirated and the plate washed 3 times with Wash Buffer (PBS, 0.05% TWEEN-20). Plates were blocked for 1 hour at room temperature with 200 μl/well of Assay Diluent (PBS, 10% FCS pH 7.0). After blocking, plates were washed 3 times with Wash Buffer. Standards and sample dilutions were prepared as follows: (a) sample supernatants were diluted 1/100 and 1/1000; (b) recombinant human MCP-1 was prepared at 500 μg/mL and serially diluted to yield as standard curve of 7.8 μg/mL to 500 μg/mL. Sample supernatants and standards were assayed in triplicate and were incubated at room temperature for 2 hours after addition to the plate coated with Capture Antibody. The plates were washed 5 times and incubated with 100 μl of Working Detector (biotinylated anti-human MCP-1 detection antibody+avidin-HRP) for 1 hour at room temperature. Following this incubation, the plates were washed 7 times and 100 μl of Substrate Solution (tetramethylbenzidine, H₂O₂) was added to plates and incubated for 30 minutes at room temperature. Stop Solution (2 N H₂SO₄) was then added to the wells and a yellow color reaction was read at 450 nm with λ correction at 570 nm. Mean absorbance was determined from triplicate data readings and the mean background was subtracted. MCP-1 concentration values were obtained from the standard curve. Inhibitory concentration of 50% (IC₅₀) was determined by comparing average MCP-1 concentration to the positive control (THP-1 cells stimulated with opsonized zymosan). An average of n=4 replicate experiments was used to determine IC₅₀ values for geldanamycin (IC₅₀=7 nM). See FIG. 16. The IC₅₀ values for the following additional compounds were determined using this assay: IC₅₀ (nM): 17-AAG, 135; anisomycin, 71; mycophenolic acid, 764; mofetil, 217; mitoxantrone, 62; chlorpromazine, 0.011; 1-α-25 dihydroxy vitamin D₃, 1; Bay 58-2667, 216; 15-deoxy prostaglandin J2, 724; rapamycin, 0.05; CNI-1493, 0.02; BXT-51072, 683; halofuginone, 9; CYC 202, 306; topotecan, 514; fascaplysin, 215; podophyllotoxin, 28; gemcitabine, 50; puromycin, 161; mithramycin, 18; daunorubicin, 570; celastrol, 421; chromomycin A3, 37; vinorelbine, 69; tubercidin, 56; vinblastine, 19; vincristine, 16.

References: J. Immunol. (2000) 165: 411-418; J. Immunol. (2000) 164: 4804-4811; J. Immunol Meth. (2000) 235 (1-2): 33-40.

### Example 46

### Screening Assay for Assessing the Effect of Paclitaxel on Cell Proliferation

Smooth muscle cells at 70-90% confluency were trypsinized, replated at 600 cells/well in media in 96-well plates and allowed to attachment overnight. Paclitaxel was prepared in DMSO at a concentration of 10⁻² M and diluted 10-fold to give a range of stock concentrations (10⁻⁸ M to 10⁻² M). Drug dilutions were diluted 1/1000 in media and added to cells to give a total volume of 200 μl/well. Each drug concentration was tested in triplicate wells. Plates containing cells and paclitaxel were incubated at 37° C. for 72 hours.

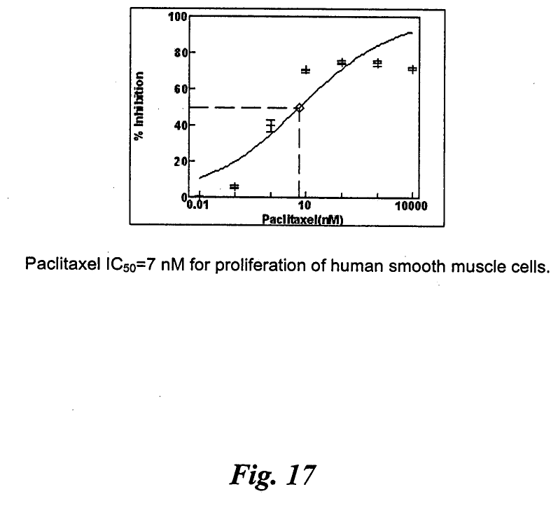

To terminate the assay, the media was removed by gentle aspiration. A 1/400 dilution of CYQUANT 400X GR dye indicator (Molecular Probes; Eugene, Oreg.) was added to 1× Cell Lysis buffer, and 200 μl of the mixture was added to the wells of the plate. Plates were incubated at room temperature, protected from light for 3-5 minutes. Fluorescence was read in a fluorescence microplate reader at ˜480 nm excitation wavelength and ˜520 nm emission maxima. Inhibitory concentration of 50% (IC₅₀) was determined by taking the average of triplicate wells and comparing average relative fluorescence units to the DMSO control. An average of n=3 replicate experiments was used to determine IC₅₀ values. See FIG. 17 (IC₅₀=7 nM). The IC₅₀ values for the following additional compounds were determined using this assay: IC₅₀ (nM): mycophenolic acid, 579; mofetil, 463; doxorubicin, 64; mitoxantrone, 1; geldanamycin, 5; anisomycin, 276; 17-AAG, 47; cytarabine, 85; halofuginone, 81; mitomycin C, 53; etoposide, 320; cladribine, 137; lovastatin, 978; epirubicin hydrochloride, 19; topotecan, 51; fascaplysin, 510; podophyllotoxin, 21; cytochalasin A, 221; gemcitabine, 9; puromycin, 384; mithramycin, 19; daunorubicin, 50; celastrol, 493; chromomycin A3, 12; vinorelbine, 15; idarubicin, 38; nogalamycin, 49; itraconazole, 795; 17-DMAG, 17; epothilone D, 5; tubercidin, 30; vinblastine, 3; vincristine, 9.

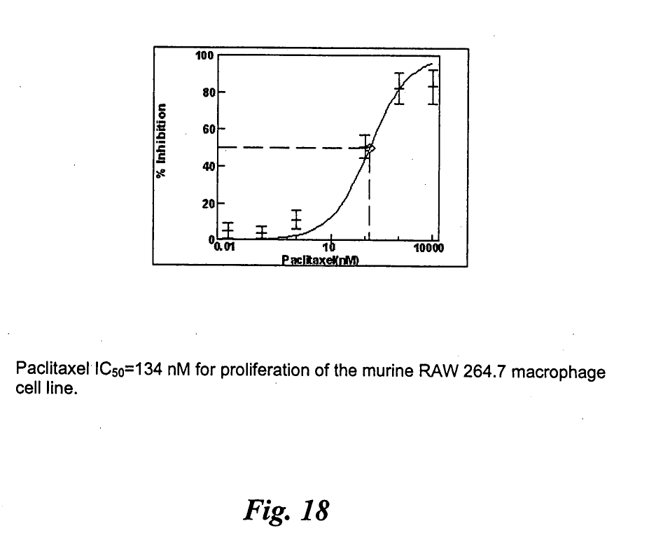

This assay also may be used assess the effect of compounds on proliferation of fibroblasts and murine macrophage cell line RAW 264.7. The results of the assay for assessing the effect of paclitaxel on proliferation of murine RAW 264.7 macrophage cell line were shown in FIG. 18 (IC₅₀=134 nM).

Reference: In vitro toxicol. (1990) 3: 219; Biotech. Histochem. (1993) 68: 29; Anal. Biochem. (1993) 213: 426.

### Example 47

### Perivascular Administration of Paclitaxel to Assess Inhibition of Fibrosis

WISTAR rats weighing 250-300 g are anesthetized by the intramuscular injection of Innovar (0.33 ml/kg). Once sedated, they are then placed under halothane anesthesia. After general anesthesia is established, fur over the neck region is shaved, the skin clamped and swabbed with betadine. A vertical incision is made over the left carotid artery and the external carotid artery exposed. Two ligatures are placed around the external carotid artery and a transverse arteriotomy is made. A number 2 French Fogarty balloon catheter is then introduced into the carotid artery and passed into the left common carotid artery and the balloon is inflated with saline. The catheter is passed up and down the carotid artery three times. The catheter is then removed and the ligature is tied off on the left external carotid artery.

Paclitaxel (33%) in ethelyne vinyl acetate (EVA) is then injected in a circumferential fashion around the common carotid artery in ten rats. EVA alone is injected around the common carotid artery in ten additional rats. (The paclitaxel may also be coated onto an EVA film which is then placed in a circumferential fashion around the common carotid artery.) Five rats from each group are sacrificed at 14 days and the final five at 28 days. The rats are observed for weight loss or other signs of systemic illness. After 14 or 28 days the animals are anesthetized and the left carotid artery is exposed in the manner of the initial experiment. The carotid artery is isolated, fixed at 10% buffered formaldehyde and examined for histology.

A statistically significant reduction in the degree of initimal hyperplasia, as measured by standard morphometric analysis, indicates a drug induced reduction in fibrotic response.

### Example 48

### In Vivo Evaluation of Silk Coated Perivascular PU Films to Assess the Ability of an Agent to Induce Scarring

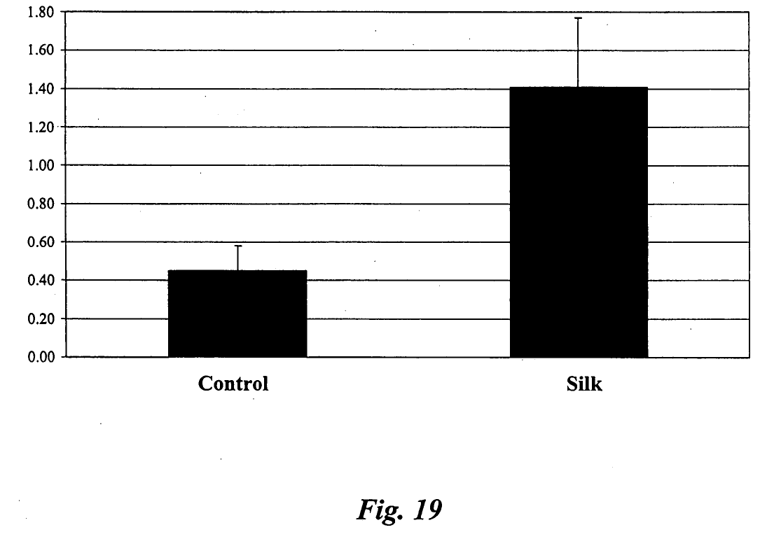

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk strands or a control uncoated PU film is wrapped around a distal segment of the common carotid artery. The wound is closed and the animal is recovered. After 28 days, the rats are sacrificed with carbon dioxide and pressure-perfused at 100 mmHg with 10% buffered formaldehyde. Both carotid arteries are harvested and processed for histology. Serial cross-sections can be cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Area of the granulation tissue is significantly higher in the silk coated group than in the control uncoated group. See FIG. 19.

### Example 49

### In Vivo Evaluation of Perivascular PU Films Coated with Different Silk Suture Material to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk sutures from one of three different manufacturers (3-0 Silk—Black Braided (Davis & Geck), 3-0 SOFSILK (U.S. Surgical/Davis & Geck), and 3-0 Silk—Black Braided (LIGAPAK) (Ethicon, Inc.) is wrapped around a distal segment of the common carotid artery. (The polyurethane film can also be coated with other agents to induce fibrosis.) The wound is closed and the animal is allowed to recover.

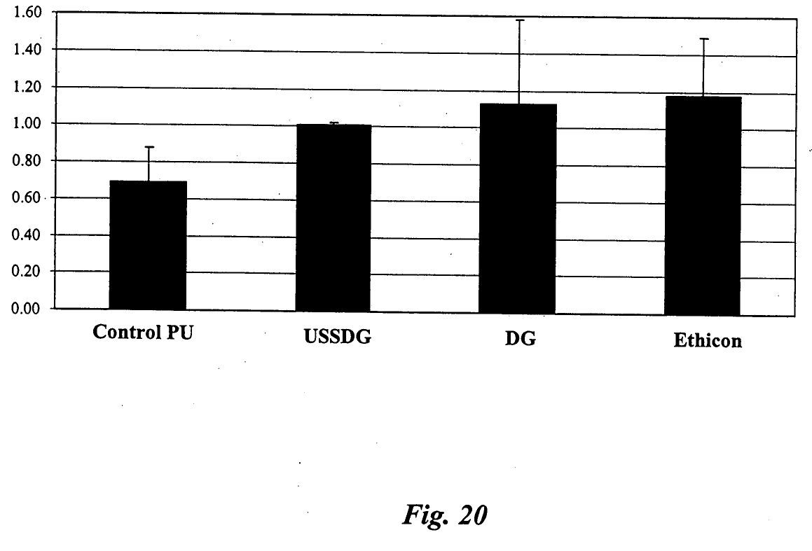

After 28 days, the rats are sacrificed with carbon dioxide and pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Thickness of the granulation tissue is the same in the three groups showing that tissue proliferation around silk suture is independent of manufacturing processes. See FIG. 20.

### Example 50

### In Vivo Evaluation of Perivascular Silk Powder to Assess the Capacity of an Agent to Induce Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Silk powder is sprinkled on the exposed artery that is then wrapped with a PU film. Natural silk powder or purified silk powder (without contaminant proteins) is used in different groups of animals. Carotids wrapped with PU films only are used as a control group. The wound is closed and the animal is allowed to recover. After 28 days, the rats are sacrificed with carbon dioxide and pressure-perfused at 100 mmHg with 10% buffered formaldehyde. Both carotid arteries are harvested and processed for histology. Serial cross-sections can be cut every 2 mm in the treated left carotid artery and at corresponding levels in the untreated right carotid artery. Sections are stained with H&E and Movat's stains to evaluate tissue growth around the carotid artery. Area of tunica intima, tunica media and perivascular granulation tissue is quantified by computer-assisted morphometric analysis.

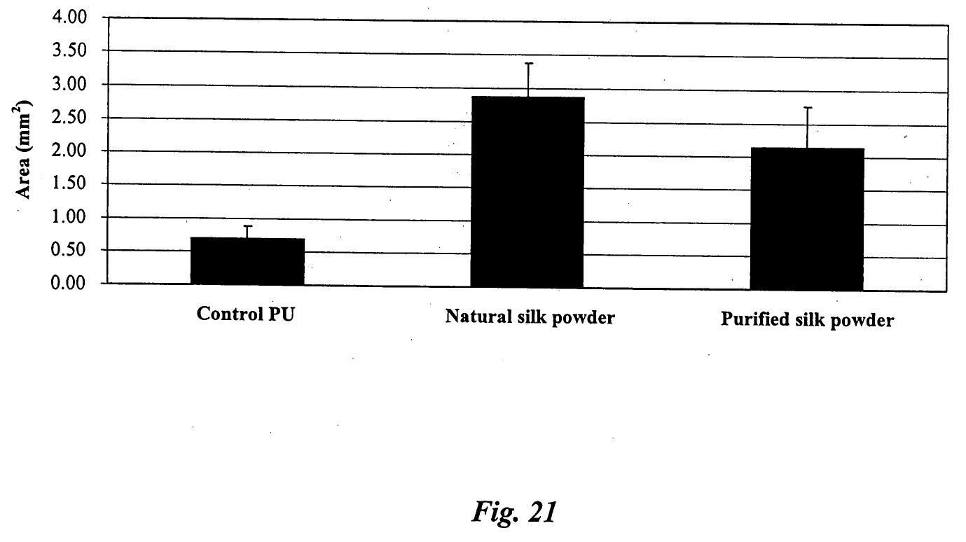

The natural silk caused a severe cellular inflammation consisting mainly of a neutrophil and lymphocyte infiltrate in a fibrin network without any extracellular matrix or blood vessels. In addition, the treated arteries were seriously damaged with hypocellular media, fragmented elastic laminae and thick intimal hyperplasia. Intimal hyperplasia contained many inflammatory cells and was occlusive in 2/6 cases. This severe immune response was likely triggered by antigenic proteins coating the silk protein in this formulation. On the other end, the regenerated silk powder triggered only a mild foreign body response surrounding the treated artery. This tissue response was characterized by inflammatory cells in extracellular matrix, giant cells and blood vessels. The treated artery was intact. These results show that removing the coating proteins from natural silk prevents the immune response and promotes benign tissue growth. Degradation of the regenerated silk powder was underway in some histology sections indicating that the tissue response can likely mature and heal over time. See FIG. 21.

### Example 51

### In Vivo Evaluation of Perivascular Talcum Powder to Assess the Capacity of an Agent to Induce Scarring

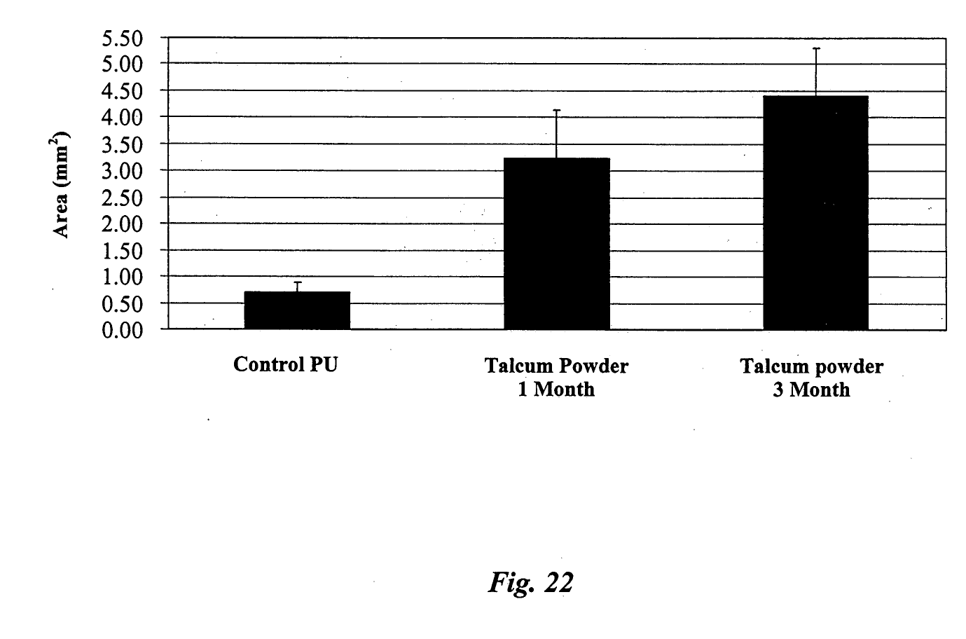

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Talcum powder is sprinkled on the exposed artery that is then wrapped with a PU film. Carotids wrapped with PU films only are used as a control group. The wound is closed and the animal is recovered. After 1 or 3 months, the rats are sacrificed with carbon dioxide and pressure-perfused at 100 mmHg with 10% buffered formaldehyde. Both carotid arteries are harvested and processed for histology. Serial cross-sections are cut every 2 mm in the treated left carotid artery and at corresponding levels in the untreated right carotid artery. Sections are stained with H&E and Movat's stains to evaluate tissue growth around the carotid artery. Thickness of tunica intima, tunica media and perivascular granulation tissue is quantified by computer-assisted morphometric analysis. Histopathology results and morphometric analysis showed the same local response to talcum powder at 1 month and 3 months. A large tissue reaction trapped the talcum powder at the site of application around the blood vessel. This tissue was characterized by a large number of macrophages within a dense extracellular matrix with few neutrophiles, lymphocytes and blood vessels. The treated blood vessel appeared intact and unaffected by the treatment. Overall, this result showed that talcum powder induced a mild long-lasting fibrotic reaction that was subclinical in nature and did not harm any adjacent tissue. See FIG. 22.

### Example 52

### MIC Determination by Microtitre Broth Dilution Method

A. MIC Assay of Various Gram Negative and Positive Bacteria

MIC assays were conducted essentially as described by Amsterdam, D. 1996, “Susceptibility testing of antimicrobials in liquid media”, p. 52-111, in Loman, V., ed. Antibiotics in laboratory medicine, 4th ed. Williams and Wilkins, Baltimore, Md. Briefly, a variety of compounds were tested for antibacterial activity against isolates of P. aeruginosa, K. pneumoniae, E. coli, S. epidermidis and S. aureus in the MIC (minimum inhibitory concentration assay under aerobic conditions using 96 well polystyrene microtitre plates (Falcon 1177), and Mueller Hinton broth at 37° C. incubated for 24 h. (MHB was used for most testing except C721 (S. pyogenes), which used Todd Hewitt broth, and Haemophilus influenzae, which used Haemophilus test medium (HTM)) Tests were conducted in triplicate. The results are provided below in Table 1.

TABLE 1
Minimum Inhibitory Concentrations of Therapeutic Agents
Against Various Gram Negative and Positive Bacteria

| | Bacterial Strain | | | | | |
|---|---|---|---|---|---|---|
| | P. aeruginosa | K. pneumoniae | E. coli | S. aureus | | |
| | PAE/K799 | ATCC13883 | UB1005 | ATCC25923 | S. epidermidis | S. pyogenes |
| | H187 | C238 | C498 | C622 | C621 | C721 |
| | Wt | wt | wt | wt | wt | wt |
| Drug | Gram − | Gram − | Gram − | Gram + | Gram + | Gram + |
| doxorubicin | 10⁻⁵ | 10⁻⁶ | 10⁻⁴ | 10⁻⁵ | 10⁻⁶ | 10⁻⁷ |
| mitoxantrone | 10⁻⁵ | 10⁻⁶ | 10⁻⁵ | 10⁻⁵ | 10⁻⁵ | 10⁻⁶ |
| 5-fluorouracil | 10⁻⁵ | 10⁻⁶ | 10⁻⁶ | 10⁻⁷ | 10⁻⁷ | 10⁻⁴ |
| methotrexate | N | 10⁻⁶ | N | 10⁻⁵ | N | 10⁻⁶ |
| etoposide | N | 10⁻⁵ | N | 10⁻⁵ | 10⁻⁶ | 10⁻⁵ |
| camptothecin | N | N | N | N | 10⁻⁴ | N |
| hydroxyurea | 10⁻⁴ | N | N | N | N | 10⁻⁴ |
| cisplatin | 10⁻⁴ | N | N | N | N | N |
| tubercidin | N | N | N | N | N | N |
| 2- | N | N | N | N | N | N |
| mercaptopurine | | | | | | |
| 6- | N | N | N | N | N | N |
| mercaptopurine | | | | | | |
| Cytarabine | N | N | N | N | N | N |

Activities are in Molar concentrations
Wt = wild type
N = No activity

B. MIC of Antibiotic-Resistant Bacteria

Various concentrations of the following compounds, mitoxantrone, cisplatin, tubercidin, methotrexate, 5-fluorouracil, etoposide, 2-mercaptopurine, doxorubicin, 6-mercaptopurine, camptothecin, hydroxyurea and cytarabine were tested for antibacterial activity against clinical isolates of a methicillin resistant S. aureus and a vancomycin resistant pediococcus clinical isolate in an MIC assay as described above. Compounds which showed inhibition of growth (MIC value of <1.0×10-3) included: mitoxantrone (both strains), methotrexate (vancomycin resistant pediococcus), 5-fluorouracil (both strains), etoposide (both strains), and 2-mercaptopurine (vancomycin resistant pediococcus).

### Example 53

### Preparation of Release Buffer

The release buffer is prepared by adding 8.22 g sodium chloride, 0.32 g sodium phosphate monobasic (monohydrate) and 2.60 g sodium phosphate dibasic (anhydrous) to a beaker. 1 L HPLC grade water is added and the solution is stirred until all the salts are dissolved. If required, the pH of the solution is adjusted to pH 7.4±0.2 using either 0.1 N NaOH or 0.1 N phosphoric acid.

### Example 54

### Release Study to Determine Release Profile of the Therapeutic Agent from a Coated Device

A sample of the therapeutic agent-loaded catheter is placed in a 15 ml culture tube. 15 ml release buffer (Example 53) is added to the culture tube. The tube is sealed with a TEFLON lined screw cap and is placed on a rotating wheel in a 37° C. oven. At various time points, the buffer is withdrawn from the culture tube and is replaced with fresh buffer. The withdrawn buffer is then analyzed for the amount of therapeutic agent contained in this buffer solution using HPLC.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-570. (canceled)

571. A device, comprising a pressure or stress sensor and an anti-scarring agent or a composition comprising an anti-scarring agent, wherein the agent inhibits scarring between the device and a host into which the device is implanted.

572. The device of claim 571 wherein the agent inhibits cell regeneration.

573. The device of claim 571 wherein the agent inhibits angiogenesis.

574. The device of claim 571 wherein the agent inhibits fibroblast migration.

575. The device of claim 571 wherein the agent inhibits fibroblast proliferation.

576. The device of claim 571 wherein the agent inhibits deposition of extracellular matrix.

577. The device of claim 571 wherein the agent inhibits tissue remodeling.

578. (canceled)

579. The device of claim 571 wherein the agent is a 5-lipoxygenase inhibitor or antagonist.

580. The device of claim 571 wherein the agent is a chemokine receptor antagonist.

581. The device of claim 571 wherein the agent is a cell cycle inhibitor.

582. The device of claim 571 wherein the agent is a taxane.

583. The device of claim 571 wherein the agent is an anti-microtubule agent.

584. The device of claim 571 wherein the agent is paclitaxel.

585. The device of claim 571 wherein the agent is not paclitaxel.

586. The device of claim 571 wherein the agent is an analogue or derivative of paclitaxel.

587. The device of claim 571 wherein the agent is a vinca alkaloid.

588. The device of claim 571 wherein the agent is camptothecin or an analogue or derivative thereof.

589. The device of claim 571 wherein the agent is a podophyllotoxin.

590. The device of claim 571 wherein the agent is a podophyllotoxin, wherein the podophyllotoxin is etoposide or an analogue or derivative thereof.

591. The device of claim 571 wherein the agent is an anthracycline.

592. The device of claim 571 wherein the agent is an anthracycline, wherein the anthracycline is doxorubicin or an analogue or derivative thereof.

593. The device of claim 571 wherein the agent is an anthracycline, wherein the anthracycline is mitoxantrone or an analogue or derivative thereof.

594. The device of claim 571 wherein the agent is a platinum compound.

595. The device of claim 571 wherein the agent is a nitrosourea.

596. The device of claim 571 wherein the agent is a nitroimidazole.

597. The device of claim 571 wherein the agent is a folic acid antagonist.

598. The device of claim 571 wherein the agent is a cytidine analogue.

599. The device of claim 571 wherein the agent is a pyrimidine analogue.

600. The device of claim 571 wherein the agent is a fluoropyrimidine analogue.

601. The device of claim 571 wherein the agent is a purine analogue.

602. The device of claim 571 wherein the agent is a nitrogen mustard or an analogue or derivative thereof.

603.-775. (canceled)

776. The device of claim 571, further comprising a second pharmaceutically active agent.

777. (canceled)

778. The device of claim 571, further comprising an agent that inhibits infection.

779.-4181. (canceled)

4182. A method for inhibiting scarring comprising placing a pressure or stress sensor and an anti-scarring agent or a composition comprising an anti-scarring agent into an animal host, wherein the agent inhibits scarring.

4183. The method of claim 4182 wherein the agent inhibits cell regeneration.

4184. The method of claim 4182 wherein the agent inhibits angiogenesis.

4185. The method of claim 4182 wherein the agent inhibits fibroblast migration.

4186. The method of claim 4182 wherein the agent inhibits fibroblast proliferation.

4187. The method of claim 4182 wherein the agent inhibits deposition of extracellular matrix.

4188. The method of claim 4182 wherein the agent inhibits tissue remodeling.

4189.-4190. (canceled)

4191. The method of claim 4182 wherein the agent is a chemokine receptor antagonist.

4192. The method of claim 4182 wherein the agent is a cell cycle inhibitor.

4193. The method of claim 4182 wherein the agent is a taxane.

4194. The method of claim 4182 wherein the agent is an anti-microtubule agent.

4195. The method of claim 4182 wherein the agent is paclitaxel.

4196. The method of claim 4182 wherein the agent is not paclitaxel.

4197. The method of claim 4182 wherein the agent is an analogue or derivative of paclitaxel.

4198. The method of claim 4182 wherein the agent is a vinca alkaloid.

4199. The method of claim 4182 wherein the agent is camptothecin or an analogue or derivative thereof.

4200. The method of claim 4182 wherein the agent is a podophyllotoxin.

4201. The method of claim 4182 wherein the agent is a podophyllotoxin, wherein the podophyllotoxin is etoposide or an analogue or derivative thereof.

4202. The method of claim 4182 wherein the agent is an anthracycline.

4203. The method of claim 4182 wherein the agent is an anthracycline, wherein the anthracycline is doxorubicin or an analogue or derivative thereof.

4204. The method of claim 4182 wherein the agent is an anthracycline, wherein the anthracycline is mitoxantrone or an analogue or derivative thereof.

4205. The method of claim 4182 wherein the agent is a platinum compound.

4206. The method of claim 4182 wherein the agent is a nitrosourea.

4207. The method of claim 4182 wherein the agent is a nitroimidazole.

4208. The method of claim 4182 wherein the agent is a folic acid antagonist.

4209. The method of claim 4182 wherein the agent is a cytidine analogue.

4210. The method of claim 4182 wherein the agent is a pyrimidine analogue.

4211. The method of claim 4182 wherein the agent is a fluoropyrimidine analogue.

4212. The method of claim 4182 wherein the agent is a purine analogue.

4213. The method of claim 4182 wherein the agent is a nitrogen mustard or an analogue or derivative thereof.

4214.-4360. (canceled)

4361. The method of claim 4182, wherein the composition further comprises a second pharmaceutically active agent.

4362. (canceled)

4363. The method of claim 4182, wherein the composition further comprises an agent that inhibits infection.

4364.-7901. (canceled)

7902. A method for making a device comprising: combining a pressure or stress sensor and an anti-scarring agent or a composition comprising an anti-scarring agent, wherein the agent inhibits scarring between the device and a host into which the device is implanted.

7903. The method of claim 7902 wherein the agent inhibits cell regeneration.

7904. The method of claim 7902 wherein the agent inhibits angiogenesis.

7905. The method of claim 7902 wherein the agent inhibits fibroblast migration.

7906. The method of claim 7902 wherein the agent inhibits fibroblast proliferation.

7907. The method of claim 7902 wherein the agent inhibits deposition of extracellular matrix.

7908. The method of claim 7902 wherein the agent inhibits tissue remodeling.

7909.-7910. (canceled)

7911. The method of claim 7902 wherein the agent is a chemokine receptor antagonist.

7912. The method of claim 7902 wherein the agent is a cell cycle inhibitor.

7913. The method of claim 7902 wherein the agent is a taxane.

7914. The method of claim 7902 wherein the agent is an anti-microtubule agent.

7915. The method of claim 7902 wherein the agent is paclitaxel.

7916. The method of claim 7902 wherein the agent is not paclitaxel.

7917. The method of claim 7902 wherein the agent is an analogue or derivative of paclitaxel.

7918. The method of claim 7902 wherein the agent is a vinca alkaloid.

7919. The method of claim 7902 wherein the agent is camptothecin or an analogue or derivative thereof.

7920. The method of claim 7902 wherein the agent is a podophyllotoxin.

7921. The method of claim 7902 wherein the agent is a podophyllotoxin, wherein the podophyllotoxin is etoposide or an analogue or derivative thereof.

7922. The method of claim 7902 wherein the agent is an anthracycline.

7923. The method of claim 7902 wherein the agent is an anthracycline, wherein the anthracycline is doxorubicin or an analogue or derivative thereof.

7924. The method of claim 7902 wherein the agent is an anthracycline, wherein the anthracycline is mitoxantrone or an analogue or derivative thereof.

7925. The method of claim 7902 wherein the agent is a platinum compound.

7926. The method of claim 7902 wherein the agent is a nitrosourea.

7927. The method of claim 7902 wherein the agent is a nitroimidazole.

7928. The method of claim 7902 wherein the agent is a folic acid antagonist.

7929. The method of claim 7902 wherein the agent is a cytidine analogue.

7930. The method of claim 7902 wherein the agent is a pyrimidine analogue.

7931. The method of claim 7902 wherein the agent is a fluoropyrimidine analogue.

7932. The method of claim 7902 wherein the agent is a purine analogue.

7933. The method of claim 7902 wherein the agent is a nitrogen mustard or an analogue or derivative thereof.

7934.-8109. (canceled)

8110. The method of claim 7902, wherein the device comprises a second pharmaceutically active agent.

8111. (canceled)

8112. The method of claim 7902 wherein the device comprises an agent that inhibits infection.

8113.-11180. (canceled)
